SEQUENCE LISTING (87 sequences)

SEQ ID NO: 1 - protein, 148 aa, Homo sapiens
MWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTS

SEQ ID NO: 2 - protein, 70 aa, Homo sapiens
MTGVLIRRGDLVTDMVACRIKTFRGHTEKAAICKTRKESSAETSPADSLILDFQPLQLMSSFSTLASLDK

SEQ ID NO: 3 - protein, 61 aa, Homo sapiens
MNTPNIVSLRAHQPEVGIIPSVLLMRPLRIKGVFHHIHSPLHGENQGFTLCLQGAPPSSSV

SEQ ID NO: 4 - protein, 64 aa, Homo sapiens
MAKTKGSLSVFRELHPAAAFDRAVHFLFLELWLPEPMLSSSPPSSTAPLLGSEPLRHWEASLSR

SEQ ID NO: 5 - protein, 44 aa, Homo sapiens
MLPRTPRPDLILLQLLPAGLRQLLQTSGPDNEQPIEQDLICNVC

SEQ ID NO: 6 - protein, 84 aa, Homo sapiens
MWNSLEARSPKSRCCSTGPWEAVQENLLWASLLAPGGATIFPDPWLLKASPSSLPHLHRTSPCACLCPNFPFYKDTVLLHQGPR

SEQ ID NO: 7 - protein, 77 aa, Homo sapiens [sequence omitted]

SEQ ID NO: 8 - protein, 38 aa, Homo sapiens
MCALQGRGASPAGAGLFHWTMSPFLLGSLYGHIHNEAV

SEQ ID NO: 9 - protein, 9 aa, Homo sapiens
VQQGWFFPR

SEQ ID NO: 10 - protein, 11 aa, Homo sapiens
VVRGGAGFAAR

SEQ ID NO: 11 - protein, 9 aa, Homo sapiens
HLADRKLSL

SEQ ID NO: 12 - protein, 9 aa, Homo sapiens
ARLQGSVTL

SEQ ID NO: 13 - protein, 10 aa, Homo sapiens
VPANTYNALK

SEQ ID NO: 14 - protein, 10 aa, Homo sapiens
RLGGCQAWWR

SEQ ID NO: 15 - protein, 10 aa, Homo sapiens
ANTYNALKSR

SEQ ID NO: 16 - protein, 9 aa, Homo sapiens
RLQGSVTLV

SEQ ID NO: 17 - protein, 9 aa, Homo sapiens
VPANTYNAL

SEQ ID NO: 18 - protein, 8 aa, Homo sapiens
ADSLILDF

SEQ ID NO: 19 - protein, 11 aa, Homo sapiens
SSFSTLASLDK

SEQ ID NO: 20 - protein, 10 aa, Homo sapiens
LVTDMVACRI

SEQ ID NO: 21 - protein, 10 aa, Homo sapiens
LILDFQPLQL

SEQ ID NO: 22 - protein, 10 aa, Homo sapiens
MSSFSTLASL

SEQ ID NO: 23 - protein, 11 aa, Homo sapiens
LMSSFSTLASL

SEQ ID NO: 24 - protein, 9 aa, Homo sapiens
LMSSFSTLA

SEQ ID NO: 25 - protein, 10 aa, Homo sapiens
QLMSSFSTLA

SEQ ID NO: 26 - protein, 10 aa, Homo sapiens
MVACRIKTFR

SEQ ID NO: 27 - protein, 10 aa, Homo sapiens
VTDMVACRIK

SEQ ID NO: 28 - protein, 8 aa, Homo sapiens
SPADSLIL

SEQ ID NO: 29 - protein, 9 aa, Homo sapiens
SLILDFQPL

SEQ ID NO: 30 - protein, 9 aa, Homo sapiens
QLMSSFSTL

SEQ ID NO: 31 - protein, 9 aa, Homo sapiens
NTPNIVSLR

SEQ ID NO: 32 - protein, 9 aa, Homo sapiens
RPLRIKGVF

SEQ ID NO: 33 - protein, 10 aa, Homo sapiens
NTPNIVSLRA

SEQ ID NO: 34 - protein, 10 aa, Homo sapiens
VLLMRPLRIK

SEQ ID NO: 35 - protein, 10 aa, Homo sapiens
MRPLRIKGVF

SEQ ID NO: 36 - protein, 10 aa, Homo sapiens
KTKGSLSVFR

SEQ ID NO: 37 - protein, 9 aa, Homo sapiens
AAFDRAVHF

SEQ ID NO: 38 - protein, 8 aa, Homo sapiens
AFDRAVHF

SEQ ID NO: 39 - protein, 9 aa, Homo sapiens
KTKGSLSVF

SEQ ID NO: 40 - protein, 8 aa, Homo sapiens
FLFLELWL

SEQ ID NO: 41 - protein, 9 aa, Homo sapiens
SVFRELHPA

SEQ ID NO: 42 - protein, 9 aa, Homo sapiens
SPPSSTAPL

SEQ ID NO: 43 - protein, 11 aa, Homo sapiens
FLELWLPEPML

SEQ ID NO: 44 - protein, 9 aa, Homo sapiens
APLLGSEPL

SEQ ID NO: 45 - protein, 11 aa, Homo sapiens
LPRTPRPDLIL

SEQ ID NO: 46 - protein, 9 aa, Homo sapiens
TPRPDLILL

SEQ ID NO: 47 - protein, 9 aa, Homo sapiens
RPDLILLQL

SEQ ID NO: 48 - protein, 11 aa, Homo sapiens
ATIFPDPWLLK

SEQ ID NO: 49 - protein, 9 aa, Homo sapiens
FPFYKDTVL

SEQ ID NO: 50 - protein, 10 aa, Homo sapiens
FPFYKDTVLL

SEQ ID NO: 51 - protein, 10 aa, Homo sapiens
TIFPDPWLLK

SEQ ID NO: 52 - protein, 9 aa, Homo sapiens
IVLDAPVTK

SEQ ID NO: 53 - protein, 8 aa, Homo sapiens
GHIHNEAV

SEQ ID NO: 54 - protein, 11 aa, Homo sapiens
SLYGHIHNEAV

SEQ ID NO: 55 - protein, 10 aa, Homo sapiens
SLYGHIHNEA

SEQ ID NO: 56 - DNA, 1063 bp, Homo sapiens
  source 1..1063: mol_type = other DNA
cggggccagtctttcccgtgctattctcgtgatagtgaataagtctcacaagatctgatgggtttatcaggggttttcattttgcttcttcctcattttcttttgctgctgtaatgtaagaaacgccttttgcctcctgccataattctgaggcctcacagccatgtggaacttcttcaggagagaattaacatccaatggattcccagaaaacttttccctcgatgtaccagcaaacacctacaatgccctgaaaagccgcctctgcgaccccaatgcagatcacacgtcctgtcccagcccctgcagcctccacgcggcgggtgcactgccaggcacgggaaggcagcgctggcgagtagaactggcccatctcgcagataggaagctgagcctcagggacgtttcacgccttcgtcaaggtggtgagaggaggagcgggattgccgtgaaggtggtgagaggaggagcggggtttgctgcccgacttcagggatctgtcaccctcgtccagcagggttggttcttcccgaggctgggaggatgccaagcctggtggaggatgggggcggtggtgtggtgtggggagcttctgacttgcacatcctgagggaaccttctgcagctgatgtgtgaactggaccccaggccgtgcctccgaggaatccccaaggctatggcccctcaggtcctgctggggtgttggcccccacctctgcctcagaatgcaggggttctgcagggaagccgcagaccagcctgctgccttgggccctagggacactgcagccccagaaagtactgtgggggacaaaagagttgtttctcgggggagaaaacacctgtgaggaaatgcaggtgccacagagggaaatcctcctggggaggagggtacctgttccatcctcggccgacacgggactgcctggtgcctggtacccacagccgctacctgccgcacgcatctctccatggtttgctaattacttccattagttttaaacaaacttgacaagagacagaagggtccagagagaaattaaatctaactgtttaaacatgt

SEQ ID NO: 57 - DNA, 2851 bp, Homo sapiens
  source 1..2851: mol_type = other DNA
cacctccatcactgcgaattataattcgacatgagatttgggagatgacacaaaaccaaaccatatcagtctttaaagagttaagttaaaataagctctttaaagtgggccctaatccagtatgactggtgttcttataagaagaggagatttggtcacagacatggttgcatgcagaataaagacttttcgaggacacactgagaaggcagccatctgcaaaacaaggaaagagtcctcagcagaaaccagtcctgcagactccttgatcttggacttccagccactgcaattgatgtcaagcttcagcacccttgcatctctggataaatgaaatgtcaccccagctgccgtccttgttcagatctgtgcaataaagagcaaagcataaaaccaagtcaaggctttgagggagtgacctacaaaatgcataatgtgaaacaatgcaaaagcgaaaggtgcaaaatccccatcaaagagctggaggctgacagatgcgccagtgataattcccatcttccaacacaggagcacagcttccattttccataacagaacaacagccagagcagctggaaggcagggccgcatcccagacttccaccaacaatgggatgagacttgacatctggaatcacaaccacaacagacctgagagacccaccagcttgatgacaagcttcttctttcaaagaaaggtatcagtctgggggacctagtgctgcaaaccatgacaaattaagtgtggcatccctcacttgcataatggaactcagtgatattttttaattaacaagagttatttttatgtaagcttctctcattcctccactgtgcgtgctcgggggctggtggtgaggaaaaagaaaacagctgtgcgggaagcatcaagaaaaggcaagtcatgaagtcttagagatcagtgacatgtaagaaaaagagtgaggagaaaaatattcctactaaagttttccatttgtttaccttccttgtcacatagacttccaagagttagaagtctaggatttgatctccaaatcttcctggcagattactcatcttcatttcattcatatagtccaggggtttgtacaaaggaagatgccagttcttccccaatcataactaagatatcaagagatattcttttgaaatgtaacaaaggagatctgaagttcatctgaaaaaaataaatggttttaggcggtcatgaccatgggatgctggaccaggatggtaagcttcaggaaacagaatctggagaatgcccagctgctcccacaggaagcatcagggaagaagaagaagaggtgtgaagtctgccttctgctctgctgggatccctttcacatctcctttgcctccaggcagttttggttcctggccatttccaggtgtgactcactcaggatggtaagcatcttctctcctacccagagtagaggatgaagacctcatctcagaggttgaagggagctccagagagaggtctcaaacttccagcattaactgctaaagaagcttcatgagctgctggagaacctgggaaatgaccaattatagggacagagctcaaatactctgggacactctagtagctgagaaagttccaactccagggtgatagaggactgcctggcaaaccatcatcaaagcagaagacctgatactaacatcacaggctatggtttattactgaagatcagtgcttacaccctgccagaggttcagaagcaaacttatcattgttctccctggagatgttggcccacattctgaaaagtgtggtcagtagtagcaacagaaagcaattgtgcttgccaagcacaatgtcactgtccccagcccttcccccaacacaacccagtaggtgcttcctggctgcaaacttgggaaagtcacttgacctgtctgaggttccacttcctaatctggcctggcgaagataagaaaaacagtttatttaaagtgtctagcaaagtgcttggaccaaaataggacctctgaaatggttatggtagtgctgttaaggtgatgttttaagtgctgatgagcacaaagatgggtaagatattccttctgttaaaatctacagtctaatgagagagaacaagatgaatgcacaataactgtcattcagaacaggattatgagaaggtgtgaatttctgtgaaaaatcagaacagggagtaatatgatcccaggtgattggcagggggtgggggtctggattcaactggagagggagctggcagggaaggcttcctggaggatgagagttcaacaaggggcaggtgtaggatgtgggtggccaagtgactgggcagaaggagctgcagaagtaagaccccaaatcaggaagacaagggcctgctgagaaacacgagctacaaagtgcaagtgcaggaagagttgggatgagattagaagggggtctggggccagactgtggaaggcccaaatgccgggctaaggagtttgtacttaattcagtggtcaacggggagtcattggaggctgttgagcaggagagttgctttctttacagctgtgccagactaaattaaacctaaacagtactttatagctggaaagggaaggcccaggaatagctcttgactcagaaacaggcattggggaaggtaatgagaaacagccgtgactgatcaaagcagagaggttaattaaatttgtaattattgtgaaaggccattaaaaaccctagttcactagagataactgctctagtggggcttcaaagacaaacgcttcttttaaccttgaatagggggatgtttgcttctctgtggaggagatatgattaagatacttaataaatggtagataaaca

SEQ ID NO: 58 - DNA, 2614 bp, Homo sapiens
  source 1..2614: mol_type = other DNA
aaacacactaagggctttgttatggactgaaatgtgtcctctccccagagtcacaggattatgaagccataatcctcaatgtgactatatttggagcaggggcctttacagacataattaaattaaatgaggtcataagagtggggccctagtctgataggactggtgtccttacaagaagagggagagtcctcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcaggtcggttgtattttgtgaaggccatcctagcaaatgaatactcctaacattgtctctttaagagctcaccagcctgaggtaggaatcattccatctgtgttactaatgagaccgctgaggatcaaaggggttttccaccacatccactcacctctacatggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttaggctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatatttgagagcttctttttcttttttgtggtttagttatttgatgatattgagattgtttctgagccatgtgcttcaacatcggattggggatttcagaaaaagttttagtcactgtgattccatttagcttccaaatgtgtctctgctaagagacttaaaagcactcataaatagcacgtgtgtcttctttgcagtgtttgctaattttgagtcacatctttttagaaaatcatgagatttggtgtcacagagactggaataaatatagtcaaacttattggtgaagatttcctttagctgttttcataatccatttccattgttatgattattgatgaataaaacattttctttaggtagatacttcttttttccccccaccttgatttaatgtttccactcttattgtcaagtttcttattactccctaataactctcaataaaataatgattcctgggagattattcctgctttcctactatcacctgttgatttgaaaagacagaacaataccgtagaagcttcactaatacattgaaagataaaatgataatactaaatactaaaatatgaaaagtgatactaaaagtggagtcctggcactagtatttttttttttgagtctttaaattttatttatttatttttgaattttttaaaattatatgttatgttctgggatacatgtgcagaacgtgcaggtttgttacataggtatacaggtctggcactagtattttgttgccacaaaatatcaagcatgtatccaaactgctcaagacacattaaagacacaggtaatctgtaggcatattcaggcttgtagtttgcattttttggttttcttgtggctttcagtgcaagttgaggtaattcatgggaaacagtcaccaaagaagtgccagtattagaaatccaagagccatttctctagcttcttccagaatcaagactttagaggtaatttctatcaacactggacatttcctgtctgcaattaacaatgaacacatagcattatgtttaattgcaacctgtttaaagcagattggatgctaaggtttaagaacactcttcagtcaaaaaggtcttttaatcaggtttttaatcttgagcacaatctaggacacagcatcatagactaactcattcgagaataggtgttgtcatctaatcctaaccacccccaccaccaacaagctgaatagctctgggctcagtatatacatttgtactgggctcagtacacacacctaagctgggttcagtatatgccactttatagtgagaggcattttgtaatgagagctctgggttcactatatacatttgtactgggctca

SEQ ID NO: 59 - DNA, 2172 bp, Homo sapiens
  source 1..2172: mol_type = other DNA
ctttcatctttaatttgaccaaaatggaaaccaggatcatgagaattcctcggggctggtgttgaaaggaatttcccctgctcttgccagagtctcgaggggtggcctccttccacgggtgagtaaccacaagtccatgtgaccgaacaaacagcatattcttttgttcaaaagagaaaaacaacattgaaggaaatcagctgaagaaaattgagatgaaagccagtccacgcctcagcatcctgaggaaatgttcttccttgatgctctgagctctctaagaagttaccacaaaaccaaacccatcagaagtttgcaggacgtccttgtttagagctgggaaataaaccacgaaacagcgcaaaggagagtccaggcctgccaatgcttccgcgaactcctcgccccgacctcatccttctccagctcctacctgcaggcctcagacaacttttgcagacctctggtccggacaatgaacaacccatagaacaagatctgatttgtaatgtttgctgatctccaaagtgtaaatactcccaccatgaccaattggaagccactgacaagtcctcactaaatgcagaattgagaagaaacacgaatagcacatcattgtatggtatttccactctaccaatggatgtgaataacctcaagaacacataatgttgagatgtggaaaaatcatcaggaagcctctccccttcaccctgcagtgtctctgcaatgctggtagagtgggctggcacagcccccgcctctggcctggtccgggtccaacctgcctgcctcctggggcagcagctccctgaacgaagagccgcggagaccgaagaactcagtgaatcagcagttctcccagatgaaacgctggcctgaaagcagcctcaagagctttggcccgtcaccttgccttgccttctcttccttcctcgtcctctgtttgttcatctttcctgaaaaaattaagtcagctgttccccttaaccaatttccctggcattctgaagggtaggccacatggcccacctgccagctactcccacctgccaagccttcctgactatatttaccctggtactcccatgtcccggggctgcttcagcagagccaaggacacacccaggtgtttgttttctaggtcagatttcctcagccatgggtgtatctgtgcttgtccctcaaaatcctcatagctcctttccccaccccaacttccaggccagacggggttcaggggtgtctcagctaaggttcccctgaagcagacacaaattagtacaaaagggacttattaggaggtgatccttaaaaatttgggcaggagaatggaaagggtggacacagacaaaaaggatgccaagtgagaatgtgatgattactgctgcagttttctacagctgcctgtcaaactatcccaacacagtggcattgaccagcagccattttcgctatgcccacagcttctgtgggtcaggcgtttggacagggcaatgggaacagcttgtttctgttccatagtgtgtgagggaggcctcaactggaaaactcaaagaccggggtggcttgatgactggaggctggaaccatccggaagcttcttgaagctgggttgacttacatgtggtcttgcatgtgagttgggcttttcacatcatggctgttgggttccaagaggcagtgtctggggaacaagagttccaagacaccaaggcagaagctgctagaccttttttgacctcgcttcggatgtcagcaatgaagttctgtggattcaactgcattctattggttaccagcagtcgcttgaatctgattagctccaaaactgagtgagcctctggcaagagtgcattgcagaagggcacatccaatgagaggcgctgctgtagtaacagggacctctccaatggcctgtttgagtgctctcagaatatggcacctgggttcctccagagcaagtgatctgagagagagcaaggagaaggccactctgcctttcatgactcatctcagaagtcacacactatcacaggttctgttctgttcattggaagtgagtcatgaagtccggcccacactcaagagaaaggaaattatgctccttcttttgaaatgagtgtaagaaagtaaaactttgtcaaatgtgtatgtgtgtgtgtgtgtgtg

SEQ ID NO: 60 - DNA, 3103 bp, Homo sapiens
  source 1..3103: mol_type = other DNA
gaggcctcacagccatgtggaactctctggaggccagaagtccaaaatcaagatgttgcagcactggtccctgggaggctgtgcaggagaatctgctctgggcgtctctcctggctcctggtggtgccacaatcttccctgatccttggcttttgaaggcatcccccagctctctgcctcatcttcacaggacttctccctgtgcctgtctgtgcccaaatttccccttctataaggacacagtcctactgcatcagggcccacgctaatcacttcttcttcactaattacatcggcaaaccccctatttccaagttaagtcatgttctgaggttaggatttcaatacctgaattttaggggacatagttcagcccaaaggacctggggggacagaaacacccaggaaagtccacaggtcagcgcctggacccgcctggccgtgggacattgctcggccgagttcatcctcataaacgctgacgtgtgctagaagatacaatattagtttcataaacaacagtttctttttttgcaaaccatacccacaggtcctattgccacttttcagtgctgcagcagctagtcagatgacaactgggtgagagcttggtctcgagtgtttcctgtgaggccagaatattgctctgattcctcaggggtgactcagccctccggtgggaaatatgtgacctcatttgcatttcctaattgaattgctatcagctttcctgcaagactgcaccaaaaaacagttcttccagattccagcactgcctgcattgcccacagaaaggctgcttttaaatagttgctaagagagggcgacaaaagaaggagaaaagttgccaaatttttatggttcatctaactgtaaagctgtcagttttacccagctccccagagtcgctaaaataatggacagaggaggcggcacttgaagaaacctcgacataagagaaatgaaatttacagaaccaggaaattaggggtccccatgcacctgccctggcagaatctgcttccaggctcctctcaataaacactgccctgggactcccagcgctctgtaaactgatgctttgaaggaggccatttgtttctccagaaccttctgccagcagaactcttgcttaccacgtgcccccatcagaactcccaccttctctaaacacagttaggtagccctctgcatgcatttgtttaaaaaaattaaattgtgagtaatagactataagtacaacagcgtacagaacaccagctaaaaaaatgatggcagaatgagcacctgtcttgccacaagactccctcccaccctctggtgtcccccagcatggcttcctgctgccccctgaaaggaaccctcaaccgactgtaactctgcacctcagtgccacccgaatttggacttcatgggagtgcggtcactcggctgacacgtgttcctgctgccaggcacctgcctgctctccttctttggtctgtttgtgagattcgctggctttgttgcttctagctgcagtttgttcacttttactgccgtataatattccactttaggagcccactgcaatttattgactcattgcaatggtgagggcatgtgtgttgtttccagcttttggtgattatgcgtaagatttctcttaacgtcccgtacagagttcttggtattacctgcctatactgctatgggcatacaccaaaaacgaggcatatgtatatttgactttaatagtaattctgaattgctgtctaaagtgtttgtaccattgtacactcccatcaagagtatacgagtgctgcaaatgtcctttcccagactctgccttattgtctcatttttaagatatatgttttgatgatcagaagttctttttttttttttttttttttgacaggcttttgctctgttgcccaggctggagtgcagtggcatgatctcagctcactgcaacctctacctctcgggctcaagtgattctcctgtttgagactccctagtagctgggattacaggtgtgcaccaccatgcctagctaatttttgtatttttagtggagacagggtttcaccatgttagccaggctggtctcgaactcctgaccttgggtgatctgcccaccttggcctcccaaagtgctgggattacaggcgtgagccaccgcacctggcagaagttcttatttttaataaagtccagtgatcattctttcccgtgtttcacgcttcacgtgccatgtgcgaggaagctttccacacccgggggtcagttcactcccagctgcattgctttgcctttcctgtttaggttttcagtccacctcaaatggattttggttgtggtgtgaggtaggggtcattttacttttttcttaaagatgccccattggcccagcatcatttattgaaaagacagtctttcctctgttgtttctgtagaaatgggcaaactttcccccaaatggccagatggtaaatattttaggcatctcaggttaaggggcaaaattgaggacatcgtataggtacttacataaccctttaaaatggaaccatttaaaaatgtaaaaaccagtcttagcttgctggccataaaaggcctgtgggctacagcatgctggcacctgctctacagcactgctcttataataaaaaatcaagcgtctgcaccctgggcatttgacaaggctcatcagattgtaaagatgaactgggtggattttgtagtatgagagttatacgtccatgaagctgatttttaaacacattacatgtctttttctgggctcattatttttgttccaagtgtctatctaatcatctttgcttcaaaatgacaccgttttaatggctgtagttcagtggttctcaaaagggggcagttttgccccccagggaacatttggcagtggctggaaagagctttggttgcatatggaggttatggggctttacctggcgtttagtgggtagaggccaggagtctggcttactatcctgtagcacacaggacagccccaacaacttgtctggaaggatgtgtcctcccatctttttcttgttctaggctaccttcgccctttccatttccattaaaaaat

SEQ ID NO: 61 - DNA, 1907 bp, Homo sapiens
  source 1..1907: mol_type = other DNA
gtgtgccctttgcagcaaagagcgtggttcctcctcttcctgggtgtaaactgcgattcaaaaagccaggtgggaagccctgtagtagggactctggctttgtccctgtttcccccttttcttcctcttcacccactaaaaccctgttttactcacggttcaaattgtttggcagcctgaattttcatggccatgggacaaagaaccccgtctttagctgaattaaggaaaagttctgcaacatttttgacgtgcaacttgggggctcaagcggagaagcgaagcagagcccctggaaaactcacatatgtgtcaacaatagtccttgatgcccctgtaacaaaacttgagcaaggcctggtgatgaagagatacaagattgtcacccaaggatttgattatacctctgttgaaagttaatgcacactcaaccgaatatggcaattggtactctcaattctatattctcaacaattacagtgaagatacaagtagaatacaatcactgccacattttttgaaattctgtgtaaactgtaatattctgtcaacattaatgacatttgaagtgtcctgtcaaaacaattgcagctactttatgtataaaacatattaaataggctcatccaacttgcattctttattaagcttattttcatattttttcctatggatgaacttaaaaataattttgtttcttaattaagattctatgcatgaagatgctgaataatttaagacaattgttcattcaaatagttgctaattacaccctcctggcatagttattgtattattactacatttaggaataatatgctgtactacttggacttgaaaatgtttctgacattttaatgaacacactacttagttatattttacaagggttttcagtgaaccacagaggattaaaaaatgtcattcaagggttgtagataattaaactgactgaatataagaagcctcatatggaagtgaaaattatgtatgaattttgttgagctggaaatgtgttttactaaatgacttcagatttgttacttttaaatacaagatgaagggaatcaaggggatactttcttctcacctcaatttgttccatttgcataagctattttatctttcaatatcccttttgatatttcattttggcccttgaatacactaaagattatttaaaataaataaatgttgactttgaaatactgctatatatattcattgcaatacatcaggtgggaaattggtgcaattcagtcacacacacaacctcatagatcagatttccagttaagttttaatagaaagagattttagatagacatgactttcaataaaatggaagaatttttattagtttcaaaataatgattttacttgcttatcaacacagttgacctttccccacagtactatgatcttcttagtacctctgctataattatattaatgtcttcctatatctagaaatttgattattattctatttttatggatcattaaatttttctattcaagaatgatccctatagtcaacttcctgagggatttctgcttgctattttctgtttcaattgtgtgcatttcatatttactattaaagattttaaataatgacacattgtttaagcggttgtaagtctgattgttataaagctctctggagaattctgtaagctttcacatctgatggagaacttcaatgagaacatctttaatgtggaattcttggacaagaaactacagattatccatggttcagaagatatgatgaattagtaactatttttccgtacataaaaagtcaatcttcctaaccagtttgttgttttagctaaatgaatcggtcaaatattttagttatattagcaatattttgtctctaaaatatcttgacataaaac

SEQ ID NO: 62 - DNA, 3091 bp, Homo sapiens
  source 1..3091: mol_type = other DNA
gggtccctggctcggctctacccccatggatctaggtgaggacaggcgctcctgctttcccgcccaaatgttgtattttccaagcctacccttgccggccacgcccccaacctgggcctataaaaaccgccccccgcaggccctagcgggcagacacactgaagtcgctagacatttgaggaacacatccggggaagaagacacaggtggctggtcatggagagcccgctgggggaagagcacacagacaggcaccggcaggccattgaccagcgggacaaggtggagtttggctggggcagtgggaggagagctggggccgccgagctgccctactccaggggaaaaccacctcccttctcgctcccccatcaccggagagctacttccactcaatacaactttgcactcattctccaagccacgtgtgaccaatttttccggtacaccaaggcgagaaatccggggatacagaaagccctctgtccttgcgataaggtagagggtccaattgagctaacacaagctgcctatagacggcaaactaagagagcacccagtaacacacgcctgctggggcttcaggagcacacacgtccactggggcttcgggagctgtaaacagtcagccctagacactgtcgtgggatcggagccccacagcctgcctgtctgtatgctcctctagaggtctgagcagcggggcgctgaagaaacgagccacactcccatcacacgccctgagaggaggacaagggaacccgtaccgtttcacttgtgatagagataaagttattattgttgtaattttaacttatagaactataatataatggtacaatgatatatattgacaatattcctctttcacccacattttgtatctgtgttcaagattaaatgaaaaaggatattctcaaaaagctagcaaaaccaaaagcaagacgttatgccaaatgtaaacatatttctgtttagcaaatagcaggagtgtataaaacatttctcttttcacataacagaatgttctatgcttactgtattagttaacaaattcaagtctgtttattttgtttgaaattccacttcatccataaattacagcattacacaataacaccagaaggacaatatcaccattgtttgcttttacagtcatctcagcccacaaaatgcttcccattgtcagcttgctatcatcaagctattgttgttttcctctttctggggtcttgtattaatatcattcaaattatttagacactcaacagtgtttttgctatcagtgcaaacctctaaagagctggagccccagcgtgatgaccaaataacccttgactatttatcttgccgtaagtcattatttcctgaggccctggagaagctgtgttgccatgtgtgcactgcagggacggggagcttctcctgccggagctggtttgttccactggacaatgagcccttttctgcttggctctctgtatgggcacatacacaatgaggcggtttagggaagaggggtgatgtgggtcattgataacaacaatccccgaacttcttaaagaattgttgagccccctaaaaatatgttgtctttatgaattatccttaaccctttttaattgcataaaaacttcaggcacttgaaaaaaattaaaaaacgaaaagtaagtctgtctctagtatccctcttctcctttgtagaattacatcctttattcactctgccactatttattatgtgcctcctgtgtgtaagacatactgttagtcattggaaatacagaaatgaatgggacagacacacttcttgccctcatggaacttagggcctaatgggagatacaatgttaaggttgtttctagtgtttttttttttttttaagaatatgttctacatgtatacatgtaatatgtattctctcccatttttaaaaaacataaatggtggtttactatgtgtacgctttttggttttgcattattcttttaacagtatgtagaggatgcattttctactgtatagtgttctgtcccccttaaatagcactctgattttattttgggggggaatcacgactttctaattttgcatactccttgtgggattgtaaatcaggtcccctgtcctcaagtagccaatggttaggtatgcaacccagcttatctctctttagactcaatctcaagcagatccagagttactcaggatcagaacaatatttgaaaggcattaccagaatccagacaagatgatggagcaatacctgatgcccagtggtctagggtagccgattcctgttctgctctccaggctcctgtccattctgtggatcaactcatattgctccaattcattttgttttgcttgcataagccagaattaacttctgttgcttgtaaacaaagagagttaaccaaagacatacagtatatcagagtatagagacctactttactctcggtcattgcctgcatgagtcttctttatatggatgtatcacagtttatttaaccactcccttgtaagtggacatttaagtctagtgttctgtaaataaaaggtcagaatatacgtctgtatacagtatatattcttgcatatccacctttggacagatgtgtgaatgtgttttgtagaaatacatttgtagaaatgcaactgctgggtcaaagaattagtagatttttaataacatcaaacagcgttgaaggcccccatataagaataacaactactgactgaagacataactaattaaaaaaattaattacagcttatttgtaataacttcttattgtcactgagtgaaaaggtaattctcgttgaatttacgaaaagtgactaataggaaatttagaaaactcagagaatatataaaaacacaaagaaaagccagccaccaagtcgcttataattctctcaccaacagtggcagaattacatttagtcatcatcattattcttacatccagttttatagttatttttgaagaggattattatcaactatcaactctgtcatagctggaagtagaggccactaaaacagatttcttaaactccaagtactgtatatggattttctac

SEQ ID NO: 63 - DNA, 444 bp, Homo sapiens
  source 1..444: mol_type = other DNA
atgtggaacttcttcaggagagaattaacatccaatggattcccagaaaacttttccctcgatgtaccagcaaacacctacaatgccctgaaaagccgcctctgcgaccccaatgcagatcacacgtcctgtcccagcccctgcagcctccacgcggcgggtgcactgccaggcacgggaaggcagcgctggcgagtagaactggcccatctcgcagataggaagctgagcctcagggacgtttcacgccttcgtcaaggtggtgagaggaggagcgggattgccgtgaaggtggtgagaggaggagcggggtttgctgcccgacttcagggatctgtcaccctcgtccagcagggttggttcttcccgaggctgggaggatgccaagcctggtggaggatgggggcggtggtgtggtgtggggagcttctgacttgcacatcc

SEQ ID NO: 64 - DNA, 210 bp, Homo sapiens
  source 1..210: mol_type = other DNA
atgactggtgttcttataagaagaggagatttggtcacagacatggttgcatgcagaataaagacttttcgaggacacactgagaaggcagccatctgcaaaacaaggaaagagtcctcagcagaaaccagtcctgcagactccttgatcttggacttccagccactgcaattgatgtcaagcttcagcacccttgcatctctggataaa

SEQ ID NO: 65 - DNA, 183 bp, Homo sapiens
  source 1..183: mol_type = other DNA
atgaatactcctaacattgtctctttaagagctcaccagcctgaggtaggaatcattccatctgtgttactaatgagaccgctgaggatcaaaggggttttccaccacatccactcacctctacatggcgaaaaccaagggttcactctctgtcttcagggagctccacccagcagcagcgtt

SEQ ID NO: 66 - DNA, 192 bp, Homo sapiens
  source 1..192: mol_type = other DNA
atggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttaggctcagagccactcagacattgggaagcaagtttgtcaaga

SEQ ID NO: 67 - DNA, 135 bp, Homo sapiens
  source 1..135: mol_type = other DNA
atgcttccgcgaactcctcgccccgacctcatccttctccagctcctacctgcaggcctcagacaacttttgcagacctctggtccggacaatgaacaacccatagaacaagatctgatttgtaatgtttgctga

SEQ ID NO: 68 - DNA, 255 bp, Homo sapiens
  source 1..255: mol_type = other DNA
atgtggaactctctggaggccagaagtccaaaatcaagatgttgcagcactggtccctgggaggctgtgcaggagaatctgctctgggcgtctctcctggctcctggtggtgccacaatcttccctgatccttggcttttgaaggcatcccccagctctctgcctcatcttcacaggacttctccctgtgcctgtctgtgcccaaatttccccttctataaggacacagtcctactgcatcagggcccacgctaa

SEQ ID NO: 69 - DNA, 234 bp, Homo sapiens
  source 1..234: mol_type = other DNA
atggccatgggacaaagaaccccgtctttagctgaattaaggaaaagttctgcaacatttttgacgtgcaacttgggggctcaagcggagaagcgaagcagagcccctggaaaactcacatatgtgtcaacaatagtccttgatgcccctgtaacaaaacttgagcaaggcctggtgatgaagagatacaagattgtcacccaaggatttgattatacctctgttgaaagttaa

SEQ ID NO: 70 - DNA, 117 bp, Homo sapiens
  source 1..117: mol_type = other DNA
atgtgtgcactgcagggacggggagcttctcctgccggagctggtttgttccactggacaatgagcccttttctgcttggctctctgtatgggcacatacacaatgaggcggtttag

SEQ ID NO: 71 - skipped

SEQ ID NO: 72 - protein, 4 aa, synthetic construct
  REGION 1..4: note = Linker sequence used in CLT Antigen Fusion Proteins 1, 2 and 4
GGGG

SEQ ID NO: 73 - skipped

SEQ ID NO: 74 - protein, 5 aa, synthetic construct
  REGION 1..5: note = Linker sequence used in CLT Antigen Fusion Proteins 1, 3 and 4
GGKGG

SEQ ID NO: 75 - protein, 6 aa, synthetic construct
  REGION 1..6: note = Linker sequence used in CLT Antigen Fusion Proteins 1, 2, 3 and 4
GGGKGG

SEQ ID NO: 76 - protein, 501 aa, synthetic construct
  REGION 1..501: note = Polypeptide sequence of CLT Antigen Fusion Protein 1
MWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTSGGGTGVLIRRGDLVTDMVACRIKTFRGHTEKAAICKTRKESSAETSPADSLILDFQPLQLMSSFSTLASLDKGGKGGMWNSLEARSPKSRCCSTGPWEAVQENLLWASLLAPGGATIFPDPWLLKASPSSLPHLHRTSPCACLCPNFPFYKDTVLLHQGPRGGGKGGMAMGQRTPSLAELRKSSATFLTCNLGAQAEKRSRAPGKLTYVSTIVLDAPVTKLEQGLVMKRYKIVTQGFDYTSVESKGGMAKTKGSLSVFRELHPAAAFDRAVHFLFLELWLPEPMLSSSPPSSTAPLLGSEPLRHWEASLSRGGGGMCALQGRGASPAGAGLFHWTMSPFLLGSLYGHIHNEAV

SEQ ID NO: 77 - protein, 503 aa, synthetic construct
  REGION 1..503: note = Polypeptide sequence of CLT Antigen Fusion Protein 2
MWNSLEARSPKSRCCSTGPWEAVQENLLWASLLAPGGATIFPDPWLLKASPSSLPHLHRTSPCACLCPNFPFYKDTVLLHQGPRGGGKGGMCALQGRGASPAGAGLFHWTMSPFLLGSLYGHIHNEAVGGGKGGMAMGQRTPSLAELRKSSATFLTCNLGAQAEKRSRAPGKLTYVSTIVLDAPVTKLEQGLVMKRYKIVTQGFDYTSVESGGGGTGVLIRRGDLVTDMVACRIKTFRGHTEKAAICKTRKESSAETSPADSLILDFQPLQLMSSFSTLASLDKGGGMAKTKGSLSVFRELHPAAAFDRAVHFLFLELWLPEPMLSSSPPSSTAPLLGSEPLRHWEASLSRGGGGMWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTS

SEQ ID NO: 78 - protein, 609 aa, synthetic construct
  REGION 1..609: note = Polypeptide sequence of CLT Antigen Fusion Protein 3
MWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTSGGKTGVLIRRGDLVTDMVACRIKTFRGHTEKAAICKTRKESSAETSPADSLILDFQPLQLMSSFSTLASLDKGGGMNTPNIVSLRAHQPEVGIIPSVLLMRPLRIKGVFHHIHSPLHGENQGFTLCLQGAPPSSSVGGKGGMAMGQRTPSLAELRKSSATFLTCNLGAQAEKRSRAPGKLTYVSTIVLDAPVTKLEQGLVMKRYKIVTQGFDYTSVESKGGMAKTKGSLSVFRELHPAAAFDRAVHFLFLELWLPEPMLSSSPPSSTAPLLGSEPLRHWEASLSRKGGLPRTPRPDLILLQLLPAGLRQLLQTSGPDNEQPIEQDLICNVCGGGWNSLEARSPKSRCCSTGPWEAVQENLLWASLLAPGGATIFPDPWLLKASPSSLPHLHRTSPCACLCPNFPFYKDTVLLHQGPRGGGKGGMCALQGRGASPAGAGLFHWTMSPFLLGSLYGHIHNEAV

SEQ ID NO: 79 - protein, 617 aa, synthetic construct
  REGION 1..617: note = Polypeptide sequence of CLT Antigen Fusion Protein 4
MWNSLEARSPKSRCCSTGPWEAVQENLLWASLLAPGGATIFPDPWLLKASPSSLPHLHRTSPCACLCPNFPFYKDTVLLHQGPRGGGGMNTPNIVSLRAHQPEVGIIPSVLLMRPLRIKGVFHHIHSPLHGENQGFTLCLQGAPPSSSVGGGKGGMWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTSGGGGMLPRTPRPDLILLQLLPAGLRQLLQTSGPDNEQPIEQDLICNVCGGGGMAKTKGSLSVFRELHPAAAFDRAVHFLFLELWLPEPMLSSSPPSSTAPLLGSEPLRHWEASLSRGGGGMCALQGRGASPAGAGLFHWTMSPFLLGSLYGHIHNEAVGGGKGGMAMGQRTPSLAELRKSSATFLTCNLGAQAEKRSRAPGKLTYVSTIVLDAPVTKLEQGLVMKRYKIVTQGFDYTSVESGGGGTGVLIRRGDLVTDMVACRIKTFRGHTEKAAICKTRKESSAETSPADSLILDFQPLQLMSSFSTLASLDK

SEQ ID NO: 80 - DNA, 1506 bp, synthetic construct
  misc_feature 1..1506: note = cDNA sequence encoding CLT Antigen Fusion Protein 1
  source 1..1506: mol_type = other DNA
atgtggaatttcttccggcgcgagctgaccagcaacggcttccctgagaacttcagcctggacgtgcccgccaacacctacaatgccctgaagtccagactgtgcgaccccaacgccgatcacaccagctgtccatctccatgttctctgcatgccgctggcgctctgcctggaacaggcagacaaagatggcgcgtggaactggcccatctggccgatagaaagctgagcctgagggatgtgtcccggctgagacaaggcggcgagagaagatctggaatcgccgtgaaggtcgtcagaggcggagctggatttgccgctagactgcagggaagcgtgaccctggttcagcaaggctggttcttccctagactcggcggttgtcaagcctggtggcgaatgggagctgttgtttggtgcggcgagctgctgacctgtacatctggcggaggaacaggcgtgctgatcagaagaggcgacctggtcacagacatggtggcctgcagaatcaagaccttcagaggccacacagagaaggccgccatctgcaagacccggaaagagtctagcgccgagacaagccctgccgactctctgatcctggacttccagcctctgcagctgatgagcagctttagcacactggctagcctggacaaaggcggaaaaggcggcatgtggaacagcctggaagccagatctcccaagagccggtgttgtagcacaggcccttgggaagctgtgcaagagaatctgctgtgggcctctctgcttgctcctggcggagccaccatctttccagatccttggctgctgaaggctagccccagctctctgcctcatctgcacagaacaagcccctgcgcctgcctgtgtcctaacttcccattctacaaggacaccgtgctgctgcatcagggccctagaggtggtggaaaaggtggaatggccatgggccagagaacaccttctctggctgagctgagaaagagcagcgccaccttcctgacctgcaatctgggagcccaggccgagaagagatctagagcccctggcaagctgacctacgtgtccaccattgtgctggacgcccctgtgaccaagctcgaacagggactcgtgatgaagcggtacaagatcgtgacccagggcttcgactacaccagcgtggaatctaaaggcggaatggctaagaccaagggcagcctgagcgtgttcagagaactgcatcctgccgccgctttcgacagagccgtgcacttcctgtttctggaactgtggctgcccgagcctatgctgtctagcagccctcctagctctacagcccctctgctgggatctgagcctctgagacactgggaagccagcctgtctagaggcggtggcggaatgtgtgctctgcaaggcagaggcgcttctcctgctggtgccggactgttccactggacaatgagcccatttctgctggggagcctgtacggccacatccacaatgaggccgtctga

SEQ ID NO: 81 - DNA, 1512 bp, synthetic construct
  misc_feature 1..1512: note = cDNA sequence encoding CLT Antigen Fusion Protein 2
  source 1..1512: mol_type = other DNA
atgtggaactccctagaagcgagatccccgaaatctagatgttgttctactggaccgtgggaagccgtacaagaaaatctactatgggcctctctactagcaccaggtggtgcaacaatttttccagatccgtggctattgaaggcctcgccatcttctttaccgcatctacacagaacatccccgtgtgcttgtctatgtccgaactttccgttctacaaggacaccgtactactacatcaaggaccaagaggtggtggaaaaggtggaatgtgtgcactacaaggaagaggtgctagtccagctggtgctggattattccattggacaatgtccccgttcctactaggatctctatacggacacatccacaacgaagctgtcggaggtggaaaaggtggaatggctatgggacaaagaacaccatctctagcggagctaagaaagtcctctgcgacattcctaacctgtaacttgggagcgcaagcggagaaaagatctagagcacctggaaagctaacctatgtctccaccatagtactagatgcgccggtcacaaagctagaacagggactagtaatgaagagatacaagatcgtcacccagggattcgactacacctctgtagaatctggtggtggtggaaccggtgtcctaattagaagaggagatctagtcaccgatatggtcgcgtgtagaatcaagacattcagaggacatacagagaaggcggcgatctgcaagacaagaaaagaatcttctgcggaaacctctccggcggactctctaatcttagattttcagccgctacagctaatgtcctccttctctacattggcctcgctagacaaaggtggtggaatggcgaaaacgaagggatccttgtccgtattcagagaactacatccagctgcggctttcgatagagcggtacatttcctattcctagagctatggctaccggaaccgatgctatcttctagtccaccatcttctacagcgccgctattaggatctgaaccgctaagacattgggaagcgagtctatctagaggtggtggtggaatgtggaacttcttcagaagagagctaacctccaacggattccccgagaacttctctttagatgtaccggcgaacacctacaacgcgctaaagtctagattgtgtgatccgaacgcggaccatacttcttgtccatctccgtgttctttacatgctgctggtgctttacctggaacgggtagacaaagatggagagtagaactagcgcacctagcggacagaaagctatccctaagagatgtctccagactaagacaaggtggagagagaagatctggaatcgcggtcaaagtagtcagaggtggtgcaggatttgctgcgagattacagggatctgtaaccttggtacagcaaggatggttcttcccaagacttggaggatgtcaagcttggtggagaatgggagctgtagtttggtgcggagaactattgacatgtacatcttga

SEQ ID NO: 82 - DNA, 1830 bp, synthetic construct
  misc_feature 1..1830: note = cDNA sequence encoding CLT Antigen Fusion Protein 3
  source 1..1830: mol_type = other DNA
atgtggaatttcttccggcgcgagctgaccagcaacggcttccctgagaacttcagcctggacgtgcccgccaacacctacaatgccctgaagtccagactgtgcgaccccaacgccgatcacaccagctgtccatctccatgttctctgcatgccgctggcgctctgcctggaacaggcagacaaagatggcgcgtggaactggcccatctggccgatagaaagctgagcctgagggatgtgtcccggctgagacaaggcggcgagagaagatctggaatcgccgtgaaggtcgtcagaggcggagctggatttgccgctagactgcagggaagcgtgaccctggttcagcaaggctggttcttccctagactcggcggttgtcaagcctggtggcgaatgggagctgttgtttggtgcggcgagctgctgacatgtacaagcggcggaaaaaccggcgtgctgatcagaagaggcgacctggtcacagacatggtggcctgcagaatcaagaccttcagaggccacacagagaaggccgccatctgcaagacccggaaagagtctagcgccgagacaagccctgccgactctctgatcctggacttccagcctctgcagctgatgagcagctttagcacactggctagcctggacaaaggcggcggaatgaacacccctaacatcgtgtccctgagagcccaccagcctgaagtgggaatcatccctagcgtgctgctgatgcggcccctgagaatcaaaggcgtgttccaccacattcacagccctctgcacggcgagaatcagggcttcaccttgtgtctgcaaggcgcccctcctagcagttctgttggtggcaaaggcggaatggccatgggccagagaacaccatctctggccgagctgagaaagagcagcgccaccttcctgacctgtaacctgggagcccaggccgagaagagatctagagcccctggcaagctgacctacgtgtccaccattgtgctggacgcccctgtgaccaagctcgaacagggactcgtgatgaagcggtacaagatcgtgacccagggcttcgactacaccagcgtggaatctaaaggtggcatggccaagaccaagggcagcctgtccgtgttcagagagctgcatcctgccgccgctttcgatagagccgtgcacttcctgtttctggaactgtggctgcccgagcctatgctgtctagcagccctccatctagcacagccccactgctgggatctgagcctctgagacactgggaagccagcctgagcagaaaaggcggcctgcctagaacacccagacctgacctgattctgctgcagctgctgcctgctggactgagacaactgctgcagacaagcggccctgacaacgagcagcctatcgagcaggacctgatctgcaatgtgtgcggaggcggctggaacagcctggaagccagatctcctaagagccggtgctgtagcacaggcccttgggaagctgtgcaagagaatctgctgtgggcctctctgcttgctcctggcggagccaccatctttccagatccttggctgctgaaggctagcccctctagcctgcctcatctgcacagaacaagcccctgcgcctgcctgtgtcctaacttcccattctacaaggacacagtgctgctgcatcagggccctcgaggtggcggaaaaggtggaatgtgtgcccttcaaggcagaggcgcttctcctgctggtgccggactgttccactggacaatgagcccatttctgctggggagcctgtacggccacatccacaatgaagccgtctga

SEQ ID NO: 83 - DNA, 1854 bp, synthetic construct
  misc_feature 1..1854: note = cDNA sequence encoding CLT Antigen Fusion Protein 4
  source 1..1854: mol_type = other DNA
atgtggaactccctagaagcgagatccccgaaatctagatgttgttctactggaccgtgggaagccgtacaagaaaatctactatgggcctctctactagcaccaggtggtgcaacaatttttccagatccgtggctattgaaggcctcgccatcttctttaccgcatctacacagaacatccccgtgtgcttgtctatgtccgaactttccgttctacaaggacaccgtactactacatcaaggaccaagaggtggtggtggaatgaatactccgaacatcgtatctctaagagcgcatcaaccggaagttggaattatcccgtccgtcctactaatgagaccgctaagaatcaagggagtgttccaccatatccactctccactacacggagaaaaccagggattcaccctatgtttacaaggtgctccaccgtcctctagtgttggaggtggaaaaggtggaatgtggaatttcttcagaagagagctaacctccaacggattccccgagaacttctctttagatgtaccggcgaacacctacaacgcgctaaagtctagattgtgtgatccgaacgcggaccatacttcttgtccatctccatgttctctacatgctgctggtgctttacctggaactggtagacaaagatggagagtagaactagcgcacctagcggacagaaagctatccctaagagatgtctccagactaagacaaggtggagagagaagatctggaatcgcggtcaaagtagttagaggtggtgcaggatttgcggcgagattacaaggatctgtaaccctagtacagcaaggatggttcttcccaagacttggaggatgtcaagcttggtggagaatgggagctgtagtttggtgcggagaactactaacatgtacctctggtggtggtggaatgctaccaagaacaccaagaccggacctaatcctactacaactactaccagcgggattgagacagctactacaaacatctggaccggataacgaacagccgatcgaacaggatctaatctgtaacgtatgcggaggtggtggaatggcgaaaacaaagggatccttgtccgtgttcagagaactacatccagctgcggcttttgatagagcggtacacttcctattcctagagctatggctaccggaaccgatgttatcttcttccccaccatcttctacagcgccgttattaggatctgaaccgttgagacattgggaagcgagtctatctagaggtggtggtggaatgtgtgcgttacaaggaagaggtgctagtccagctggtgccggattatttcattggacaatgtccccgttcctactaggatctctatacggacacatccacaacgaagctgtcggtggtggaaaaggtggaatggctatgggacaaagaacaccatctctagcggagctaagaaagtcctctgcgacattcctaacctgtaacttgggagcgcaagcggagaaaagatctagagcacctggaaagctaacctatgtctccaccatagtactagatgcgccggtcacaaagctagaacagggactagtaatgaagagatacaagatcgtcacgcagggattcgactacacctctgtagaatccggtggtggtggtacaggtgtcctaattagaagaggagatctagtcaccgatatggtcgcgtgtagaatcaagacattcagaggacatacagagaaggcggcgatctgcaagacaagaaaagaatcttctgcggaaacctctccggcggactctctaatcttagattttcagccgctacagctaatgtcctccttctctactttggcctccttggataagtga

SEQ ID NO: 84 - skipped

SEQ ID NO: 85 - protein, 15 aa, synthetic construct
  REGION 1..15: note = TCR VB CDR3 AA sequence
CASSLTGGYTGELFF

SEQ ID NO: 86 - protein, 13 aa, synthetic construct
  REGION 1..13: note = TCR VB CDR3 AA sequence
CASNKLGYQPQHF

SEQ ID NO: 87 - protein, 13 aa, synthetic construct
  REGION 1..13: note = TCR VB CDR3 AA sequence
CASSLLENQPQHF

(19) United States
(12) Patent Application Publication
(10) Pub. No.: US 2023/0167163 A1
(43) Pub. Date: Jun. 1, 2023

(54) FUSION PROTEINS OF CTL ANTIGENS FOR TREATING MELANOMA

(71) Applicants: The Francis Crick Institute Limited, London Greater London (GB); Enara Bio Limited, Oxford (GB)

(72) Inventors: George KASSIOTIS, London (GB); George YOUNG, London (GB); Jan ATTIG, London (GB); Ambrosius SNIJDERS, London (GB); David PERKINS, London (GB); Fabio MARINO, Oxford (GB); Ray JUPP, Oxford (GB); Magdalena VON ESSEN, Oxford (GB); Peter MASON, Oxford (GB); Nicola TERNETTE, Oxford (GB)

(21) Appl. No.: 18/046,675

(22) Filed: Oct. 14, 2022

Related U.S. Application Data
(63) Continuation of application No. PCT/US2021/028029, filed on Apr. 19, 2021.

(30) Foreign Application Priority Data
Apr. 17, 2020 (GB) 20170174.5

(51) Int. Cl. C07K 14/47; A61K 39/39; A61P 35/00; A61P 17/00; A61K 39/00

(52) U.S. Cl. CPC C07K 14/4748; A61K 39/39; A61P 35/00; A61P 17/00; A61K 39/00119; C07K 2319/00; A61K 2039/876

(57) ABSTRACT

There are disclosed inter alia fusion proteins and nucleic acids encoding said fusion proteins which are useful in the treatment and prevention of cancer, particularly melanoma, especially cutaneous melanoma and uveal melanoma.

### RELATED APPLICATIONS

This application is a continuation of International Application No. PCT/US2021/028029, filed on Apr. 19, 2021, which claims the benefit of United Kingdom Application No. 20170174.5, filed Apr. 17, 2020, the entire disclosures of which are hereby incorporated herein by reference in their entirety.

### SEQUENCE LISTING

The instant application contains a sequence listing which has been submitted electronically in ASCII ST.26 format and is hereby incorporated by reference in its entirety (said ASCII, ST.26 copy, created on Oct. 14, 2022, is named 193817_SL.xml and is 107353 bytes in size).

### FIELD OF THE INVENTION

The present invention relates to fusion proteins and their corresponding polynucleotides for use in the treatment or prevention of cancer, in particular for use in treating or preventing melanoma (e.g. cutaneous melanoma or uveal melanoma). The present invention further relates inter alia to pharmaceutical and immunogenic compositions comprising said fusion proteins or nucleic acids, medical use of said pharmaceutical and immunogenic compositions and methods of treatment comprising administering said pharmaceutical and immunogenic compositions.

### BACKGROUND OF THE INVENTION

As part of normal immunosurveillance for pathogenic microbes, all cells degrade intracellular proteins to produce peptides that are loaded onto Major Histocompatibility Complex (MHC) Class I molecules that are expressed on the surface of all cells. Most of these peptides, which are derived from the host cell, are recognized as self, and remain invisible to the adaptive immune system. However, peptides that are foreign (non-self), are capable of stimulating the expansion of naïve CD8+ T-cells that encode a T-cell receptor (TCR) that tightly binds the MHC I-peptide complex. This expanded T-cell population can produce effector CD8+ T-cells (including cytotoxic T-lymphocytes—CTLs) that can eliminate the foreign antigen-tagged cells, as well as memory CD8+ T-cells that can be re-amplified when the foreign antigen-tagged cells appear later in the animal's life.

MHC Class II molecules, whose expression is normally limited to professional antigen-presenting cells (APCs) such as dendritic cells (DCs), are usually loaded with peptides which have been internalised from the extracellular environment. Binding of a complementary TCR from a naïve CD4+ T-cell to the MHC II-peptide complex, in the presence of various factors, including T-cell adhesion molecules (CD54, CD48) and co-stimulatory molecules (CD40, CD80, CD86), induces the maturation of CD4+ T-cells into effector cells (e.g., T_{H}1, T_{H}2, T_{H}17, T_{FH}, T_{reg} cells). These effector CD4+ T-cells can promote B-cell differentiation to antibody-secreting plasma cells as well as facilitate the differentiation of antigen-specific CD8+ CTLs, thereby helping induce the adaptive immune response to foreign antigens, that include both short-term effector functions and longer-term immunological memory. DCs can perform the process of cross-presentation of peptide antigens by delivering exogenously-derived antigens (such as a peptide or protein released from a pathogen or a tumor cell) onto their MHC I molecules, contributing to the generation of immunological memory by providing an alternative pathway to stimulating the expansion of naïve CD8+ T-cells.

Immunological memory (specifically antigen-specific B cells/antibodies and antigen-specific CTLs) are critical players in controlling microbial infections, and immunological memory has been exploited to develop numerous vaccines that prevent the diseases caused by important pathogenic microbes. Immunological memory is also known to play a key role in controlling tumor formation, but very few efficacious cancer vaccines have been developed.

Cancer is the second leading cause of morbidity, accounting for nearly 1 in 6 of all deaths globally. Of the 8.8 million deaths caused by cancer in 2015, the cancers which claimed the most lives were from lung (1.69 million), liver (788,000), colorectal (774,000), stomach (754,000) and breast (571,000) carcinomas. The economic impact of cancer in 2010 was estimated to be USD1.16 Trillion, and the number of new cases is expected to rise by approximately 70% over the next two decades (World Health Organisation Cancer Facts 2017).

Current therapies for cutaneous melanoma are varied and are highly dependent on the location of the tumor and stage of the disease. The main treatment for a non-metastatic melanoma is surgery to remove the tumor and surrounding tissue. Later stage melanomas may require treatment comprising lymph node dissection, radiotherapy, or chemotherapy. Immune checkpoint blockade strategies, including the use of antibodies targeting negative immune regulators such PD-1/PD-L1 and CTLA4, have recently revolutionised treatments to a variety of malignancies, including melanoma (Ribas, A., & Wolchok, J. D. (2018) Science, 359:1350-1355.). The extraordinary value of checkpoint blockade therapies, and the well-recognized association of their clinical benefit with patient's adaptive immune responses (specifically T-cell based immune responses) to their own cancer antigens has re-invigorated the search for effective cancer vaccines, vaccine modalities, and cancer vaccine antigens.

Human endogenous retroviruses (HERVs) are remnants of ancestral germ line integrations of exogenous infectious retroviruses. HERVs belong to the group of endogenous retroelements that are characterised by the presence of Long Terminal Repeats (LTRs) flanking the viral genome. This group also includes the Mammalian apparent LTR Retrotransposons (MaLRs) and are therefore collectively known as LTR elements (here referred to collectively as ERV to mean all LTR elements). ERVs constitute a considerable proportion of the mammalian genome (8%), and can be grouped into approximately 100 families based on sequence homology. Many ERV sequences encode defective proviruses which share the prototypical retroviral genomic structure consisting of gag, pro, pol and env genes flanked by LTRs. Some intact ERV ORFs produce retroviral proteins which share features with proteins encoded by exogenous infectious retroviruses such as HIV-1. Such proteins may serve as antigens to induce a potent immune response (Hurst & Magiorkinis, 2015, J. Gen. Virol 96:1207-1218), suggesting that polypeptides encoded by ERVs can escape T and B-cell receptor selection processes and central and peripheral tolerance. Immune reactivity to ERV products may occur spontaneously in infection or cancer, and ERV products have been implicated as a cause of some autoimmune diseases (Kassiotis & Stoye, 2016, Nat. Rev. Immunol. 16:207-219).

Due to the accumulation of mutations and recombination events during evolution, most ERV-derived sequences have lost functional open reading frames for some or all of their genes and therefore their ability to produce infectious virus. However, these ERV elements are maintained in germline DNA like other genes and still have the potential to produce proteins from at least some of their genes. Indeed, HERV-encoded proteins have been detected in a variety of human cancers. For example, splice variants of the HERV-K env gene, Rec and Np9, are found exclusively in malignant testicular germ cells and not in healthy cells (Ruprecht et. al, 2008, Cell Mol Life Sci 65:3366-3382). Increased levels of HERV transcripts have also been observed in cancers such as those of the prostate, as compared to healthy tissue (Wang-Johanning, 2003, Cancer 98:187-197; Andersson et al., 1998, Int. J. Oncol, 12:309-313). Additionally, overexpression of HERV-E and HERV-H has been demonstrated to be immunosuppressive, which could also contribute to the development of cancer (Mangeney et al., 2001, J. Gen. Virol. 82:2515-2518). However, the exact mechanism(s) by which HERVs could contribute to the development or pathogenicity of cancer remains unknown.

In addition to deregulating the expression of surrounding neighbouring host genes, the activity and transposition of ERV regulatory elements to new genomic sites may lead to the production of novel transcripts, some of which may have oncogenic properties (Babaian & Mager, Mob. DNA, 2016, Lock et al., PNAS, 2014, 111:3534-3543).

A wide range of vaccine modalities are known. One well-described approach involves directly delivering an antigenic polypeptide to a subject with a view to raising an immune response (including B- and T-cell responses) and stimulating immunological memory. Alternatively, a polynucleotide may be administered to the subject by means of a vector such that the polynucleotide-encoded immunogenic polypeptide is expressed in vivo. The use of viral vectors, for example adenovirus vectors, has been well explored for the delivery of antigens in both prophylactic vaccination and therapeutic treatment strategies against cancer (Wold et al. Current Gene Therapy, 2013, Adenovirus Vectors for Gene Therapy, Vaccination and Cancer Gene Therapy, 13:421-433). Immunogenic peptides, polypeptides, or polynucleotides encoding them, can also be used to load patient-derived antigen presenting cells (APCs), that can then be infused into the subject as a vaccine that elicits a therapeutic or prophylactic immune response. An example of this approach is Provenge, which is presently the only FDA-approved anti-cancer vaccine.

Cancer antigens, may also be exploited in the treatment and prevention of cancer by using them to create a variety of non-vaccine therapeutic modalities. These therapies fall into two different classes: 1) antigen-binding biologics, 2) adoptive cell therapies.

Antigen-binding biologics typically consist of multivalent engineered polypeptides that recognize antigen-decorated cancer cells and facilitate their destruction. The antigen-binding components of these biologics may consist of TCR-based biologicals, including, but not limited to TCRs, high-affinity TCRs, and TCR mimetics produced by various technologies (including those based on monoclonal antibody technologies). Cytolytic moieties of these types of multivalent biologics may consist of cytotoxic chemicals, biological toxins, targeting motifs and/or immune stimulating motifs that facilitate targeting and activation of immune cells, any of which facilitate the therapeutic destruction of tumor cells.

Adoptive cell therapies may be based on a patient's own T-cells that are removed and stimulated ex vivo with vaccine antigen preparations (cultivated with T-cells in the presence or absence of other factors, including cellular and acellular components) (Yossef et al., JCI Insight. 2018 Oct. 4; 3(19). pii: 122467. doi: 10.1172/jci.insight.122467). Alternatively, adoptive cell therapies can be based on cells (including patient- or non-patient-derived cells) that have been deliberately engineered to express antigen-binding polypeptides that recognize cancer antigens. These antigen-binding polypeptides fall into the same classes as those described above for antigen-binding biologics. Thus, lymphocytes (autologous or non-autologous), that have been genetically manipulated to express cancer antigen-binding polypeptides can be administered to a patient as adoptive cell therapies to treat their cancer.

Use of ERV-derived antigens in raising an effective immune response to cancer has shown promising results in promoting tumor regression and a more favourable prognosis in murine models of cancer (Kershaw et al., 2001, Cancer Res. 61:7920-7924; Slansky et al., 2000, Immunity 13:529-538). Thus, HERV antigen-centric immunotherapy trials have been contemplated in humans (Sacha et al., 2012, J. Immunol 189:1467-1479), although progress has been restricted, in part, due to a severe limitation of identified tumor-specific ERV antigens.

WO 2005/099750 identifies anchored sequences in existing vaccines against infectious pathogens, which are common in raising cross-reactive immune responses against the HERV-K Mel tumor antigen and confers protection to melanoma.

WO 00/06598 relates to the identification of HERV-AVL3-B tumor associated genes which are preferentially expressed in melanomas, and methods and products for diagnosing and treating conditions characterised by expression of said genes.

WO 2006/119527 provides antigenic polypeptides derived from the melanoma-associated endogenous retrovirus (MERV), and their use for the detection and diagnosis of melanoma as well as prognosis of the disease. The use of antigenic polypeptides as anticancer vaccines is also disclosed.

WO 2007/137279 discloses methods and compositions for detecting, preventing and treating HERV-K+ cancers, for example with use of a HERV-K+ binding antibody to prevent or inhibit cancer cell proliferation.

WO 2006/103562 discloses a method for treating or preventing cancers in which the immunosuppressive Np9 protein from the env gene of HERV-K is expressed. The invention also relates to pharmaceutical compositions comprising nucleic acid or antibodies capable of inhibiting the activity of said protein, or immunogen or vaccinal composition capable of inducing an immune response directed against said protein.

WO 2007/109583 provides compositions and methods for preventing or treating neoplastic disease in a mammalian subject, by providing a composition comprising an enriched immune cell population reactive to a HERV-E antigen on a tumor cell.

Humer J, et al., 2006, Canc. Res., 66:1658-63 identifies a melanoma marker derived from melanoma-associated endogenous retroviruses.

There is a need to identify novel fusion proteins comprising HERV-associated antigenic sequences which can be used in immunotherapy of cancer, particularly melanoma, especially cutaneous and uveal melanoma.

### SUMMARY OF THE INVENTION

The inventors have surprisingly discovered certain RNA transcripts which comprise LTR elements or are derived from genomic sequences adjacent to LTR elements which are found at high levels in cutaneous melanoma cells, but are undetectable or found at very low levels in normal, healthy tissues (see Example 1). Such transcripts are herein referred to as cancer-specific LTR-element spanning transcripts (CLTs). Further, the inventors have shown that a subset of the potential polypeptide sequences (i.e., open reading frames (ORFs)) encoded by these CLTs are translated in cancer cells, processed by components of the antigen-processing apparatus, and presented on the surface of cells found in tumor tissue in association with the class I and class II major histocompatibility complex (MHC Class I, and MHC Class II) and class I and class II human leukocyte antigen (HLA Class I, HLA Class II) molecules (see Example 2). These findings demonstrate that these polypeptides (herein referred to as CLT antigens) are, ipso facto, antigenic. Thus, cancer cell presentation of CLT antigens is expected to render these cells susceptible to elimination by T-cells that bear cognate T-cell receptors (TCRs) for the CLT antigens, and CLT antigen-based vaccination methods/regimens that amplify T-cells bearing these cognate TCRs are expected to elicit immune responses against cancer cells (and tumors containing them), particularly melanoma particularly cutaneous melanoma tumors. T-cells from melanoma subjects are indeed reactive to peptides derived from CLT antigens disclosed herein and amplify T-cells and amplify T-cell receptor sequences (see Example 3). The inventors have confirmed that T-cells specific for CLT antigens have not been deleted from normal subject's T-cell repertoire by central tolerance (see Example 4). The presence and killing activity of CLT antigen specific T-cells in ex vivo cultures of healthy donor T-cells has been determined (see Example 5). Further, qRT-PCR and RNA Scope studies have confirmed that CLTs are specifically expressed in RNA extracted from melanoma cell lines or melanoma tumor tissue as compared to non-melanoma cells lines or tissues (see Example 6). The inventors have also produced fusion proteins comprising unique CLT antigens for vaccine delivery (Example 8).

The inventors have also surprisingly discovered that certain CLT antigen-encoding CLTs as well as being overexpressed in cutaneous melanoma are also overexpressed in uveal melanoma. The CLT antigen polypeptide sequences encoded by these CLTs and fusion proteins containing them are expected to elicit immune responses against uveal melanoma cells and tumors containing them.

The CLTs and the CLT antigens are not canonical sequences which can be readily derived from known tumor genome sequences found in the cancer genome atlas. The CLTs are transcripts resulting from complex transcription and splicing events driven by transcription control sequences of ERV origin. Since the CLTs are expressed at high level and since CLT antigen polypeptide sequences are not sequences of normal human proteins, it is expected that they will be capable of eliciting strong, specific immune responses (as indeed has been established—see Examples 3-5 and 10) and are thus suitable for therapeutic use in a cancer immunotherapy setting.

The CLT antigens discovered in the highly expressed transcripts that characterize tumor cells, which were previously not known to exist and produce protein products in man and to stimulate immune responses, can be used in several formats. For example, fusion proteins that are the subject of the present invention comprising CLT antigen polypeptides can be directly delivered to a subject as a vaccine that elicits a therapeutic or prophylactic immune response to tumor cells. Further, nucleic acids encoding for fusion proteins of the invention, where the nucleic acid which encodes the CLT antigens may be codon optimised to enhance the expression of their encoded CLT antigens, can be directly administered or else inserted into vectors for delivery in vivo to produce the encoded protein products in a subject as a vaccine that elicits a therapeutic or prophylactic immune response to tumor cells. These and other applications are described in greater detail below.
Thus, the invention provides inter alia a fusion protein comprising six antigenic polypeptides (a) to (f), wherein the antigenic polypeptides (a) to (f) have the amino acid sequences:
(a) SEQ ID NO: 1 or a variant thereof, or an immunogenic fragment of SEQ ID NO: 1 or a variant thereof;
(b) SEQ ID NO: 2 or a variant thereof or an immunogenic fragment of SEQ ID NO: 2 or a variant thereof;
(c) SEQ ID NO: 6 or a variant thereof, or an immunogenic fragment of SEQ ID NO: 6 or a variant thereof;
(d) SEQ ID NO: 7 or a variant thereof, or an immunogenic fragment of SEQ ID NO: 7 or a variant thereof;
(e) SEQ ID NO: 4 or a variant thereof, or an immunogenic fragment of SEQ ID NO: 4 or a variant thereof; and
(f) SEQ ID NO: 8 or a variant thereof, or an immunogenic fragment of SEQ ID NO: 8 or a variant thereof.
(hereinafter referred to as “fusion proteins of the invention”).

The invention also provides a nucleic acid molecule which encodes a fusion protein of the invention (hereinafter referred to as “nucleic acids of the invention”).

The fusion proteins of the invention and the nucleic acids of the invention, as well as related aspects of the invention, are expected to be useful in a range of embodiments in cancer immunotherapy and prophylaxis, particularly immunotherapy and prophylaxis of melanoma, as discussed in more detail below.

### DESCRIPTION OF THE FIGURES

Each of FIGS. 1-38 shows an extracted MS/MS spectrum (with assigned fragment ions) of a peptide obtained from a tumor sample of a patient and either a bottom panel showing a rendering of the spectrum indicating the positions of the linear peptide sequences that have been mapped to the fragment ions or similar data shown in tabular form.

FIG. 1. Spectra for the peptide of SEQ ID NO. 9 obtained from a tumor sample of patient Mel-3.

FIG. 2. Spectra for the peptide of SEQ ID NO. 10 obtained from a tumor sample of patient Mel-3.

FIG. 3. Spectra for the peptide of SEQ ID NO. 10 obtained from a tumor sample of patient Mel-3.

FIG. 4. Spectra for the peptide of SEQ ID NO. 10 obtained from a tumor sample of patient 2MT3.

FIG. 5. Spectra for the peptide of SEQ ID NO. 11 obtained from a tumor sample of patient Mel-5.

FIG. 6. Spectra for the peptide of SEQ ID NO. 11 obtained from a tumor sample of patient Mel-16.

FIG. 7. Spectra for the peptide of SEQ ID NO. 11 obtained from a tumor sample of patient Mel-16.

FIG. 8. Spectra for the peptide of SEQ ID NO. 11 obtained from a tumor sample of patient 2MT3.

FIG. 9. Spectra for the peptide of SEQ ID NO. 11 obtained from a tumor sample of patient 2MT10.

FIG. 10 Spectra for the peptide of SEQ ID NO. 12 obtained from a tumor sample of patient Mel-5.

FIG. 11. Spectra for the peptide of SEQ ID NO. 18 obtained from a tumor sample of patient Mel-26.

FIG. 12. Spectra for the peptide of SEQ ID NO. 19 obtained from a tumor sample of patient Mel-20.

FIG. 13. Spectra for the peptide of SEQ ID NO. 19 obtained from a tumor sample of patient Mel-20.

FIG. 14. Spectra for the peptide of SEQ ID NO. 19 obtained from a tumor sample of patient 2MT4.

FIG. 15. Spectra for the peptide of SEQ ID NO. 31 obtained from a tumor sample of patient Mel-35.

FIG. 16. Spectra for the peptide of SEQ ID NO. 31 obtained from a tumor sample of patient 2MT3.

FIG. 17. Spectra for the peptide of SEQ ID NO. 32 obtained from a tumor sample of patient 1MT1.

FIG. 18. Spectra for the peptide of SEQ ID NO. 36 obtained from a tumor sample of patient Mel-3.

FIG. 19. Spectra for the peptide of SEQ ID NO. 36 obtained from a tumor sample of patient Mel-3.

FIG. 20. Spectra for the peptide of SEQ ID NO. 36 obtained from a tumor sample of patient 2MT3.

FIG. 21. Spectra for the peptide of SEQ ID NO. 36 obtained from a tumor sample of patient 2MT1.

FIG. 22. Spectra for the peptide of SEQ ID NO. 37 obtained from a tumor sample of patient Mel-40.

FIG. 23. Spectra for the peptide of SEQ ID NO. 37 obtained from a tumor sample of patient Mel-41.

FIG. 24. Spectra for the peptide of SEQ ID NO. 37 obtained from a tumor sample of patient 2MT3.

FIG. 25. Spectra for the peptide of SEQ ID NO. 38 obtained from a tumor sample of patient Mel-27.

FIG. 26. Spectra for the peptide of SEQ ID NO. 38 obtained from a tumor sample of patient Mel-39.

FIG. 27. Spectra for the peptide of SEQ ID NO. 39 obtained from a tumor sample of patient 2MT12.

FIG. 28. Spectra for the peptide of SEQ ID NO. 45 obtained from a tumor sample of patient Mel-29.

FIG. 29. Spectra for the peptide of SEQ ID NO. 48 obtained from a tumor sample of patient Mel-41.

FIG. 30. Spectra for the peptide of SEQ ID NO. 49 obtained from a tumor sample of patient Mel-41.

FIG. 31. Spectra for the peptide of SEQ ID NO. 50 obtained from a tumor sample of patient Mel-41.

FIG. 32. Spectra for the peptide of SEQ ID NO. 51 obtained from a tumor sample of patient Mel-41.

FIG. 33. Spectra for the peptide of SEQ ID NO. 52 obtained from a tumor sample of patient Mel-21.

FIG. 34. Spectra for the peptide of SEQ ID NO. 52 obtained from a tumor sample of patient 2MT3.

FIG. 35. Spectra for the peptide of SEQ ID NO. 53 obtained from a tumor sample of patient Mel-27.

FIG. 36. Spectra for the peptide of SEQ ID NO. 54 obtained from a tumor sample of patient Mel-27.

FIG. 37. Spectra for the peptide of SEQ ID NO. 54 obtained from a tumor sample of patient 2MT4.

Each of FIGS. 38-53 shows an alignment of a native MS/MS spectrum of a peptide obtained from a patient tumor sample (upper) to the native spectrum of a synthetic peptide corresponding to the same sequence (lower).

FIG. 38 shows a mass spectrometry spectrum of a peptide fragment from immunopeptidomic analysis of patient 2MT3 attributed to SEQ ID NO. 10.

FIG. 39 shows a mass spectrometry spectrum of a peptide fragment from immunopeptidomic analysis of patient 2MT3 attributed to SEQ ID NO. 11.

FIG. 40 shows a mass spectrometry spectrum of a peptide fragment from immunopeptidomic analysis of patient 2MT4 attributed to SEQ ID NO. 19.

FIG. 41 shows a mass spectrometry spectrum of a peptide fragment from immunopeptidomic analysis of patient 2MT3 attributed to SEQ ID NO. 31.

FIG. 42 shows a mass spectrometry spectrum of a peptide fragment from immunopeptidomic analysis of patient 1MT1 attributed to SEQ ID NO. 32.

FIG. 43 shows a mass spectrometry spectrum of a peptide fragment from immunopeptidomic analysis of patient 2MT3 attributed to SEQ ID NO. 36.

FIG. 44 shows a mass spectrometry spectrum of a peptide fragment from immunopeptidomic analysis of patient 2MT3 attributed to SEQ ID NO. 37.

FIG. 45 shows a mass spectrometry spectrum of a peptide fragment from immunopeptidomic analysis of patient 2MT12 attributed to SEQ ID NO. 39.

FIG. 46 shows a mass spectrometry spectrum of a peptide fragment from immunopeptidomic analysis of patient Mel-29 attributed to SEQ ID NO. 45.

FIG. 47 shows a mass spectrometry spectrum of a peptide fragment from immunopeptidomic analysis of patient Mel-41 attributed to SEQ ID NO. 48.

FIG. 48 shows a mass spectrometry spectrum of a peptide fragment from immunopeptidomic analysis of patient Mel-41 attributed to SEQ ID NO. 49.

FIG. 49 shows a mass spectrometry spectrum of a peptide fragment from immunopeptidomic analysis of patient Mel-41 attributed to SEQ ID NO. 50.

FIG. 50 shows a mass spectrometry spectrum of a peptide fragment from immunopeptidomic analysis of patient Mel-41 attributed to SEQ ID NO. 51.

FIG. 51 shows a mass spectrometry spectrum of a peptide fragment from immunopeptidomic analysis of patient Mel-21 attributed to SEQ ID NO. 52.

FIG. 52 shows a mass spectrometry spectrum of a peptide fragment from immunopeptidomic analysis of patient Mel-27 attributed to SEQ ID NO. 53.

FIG. 53 shows a mass spectrometry spectrum of a peptide fragment from immunopeptidomic analysis of patient Mel-27 attributed to SEQ ID NO. 54.

FIG. 54 panels A to C shows tumor antigen-specific T-cell amplification from patient PBMC cultures in response to cultivation with specific tumor antigen-derived peptides.

FIG. 55 panels A to D provides a summary of CLT Antigen-derived peptides (SEQ ID NO. 11, 13-15, 19-29, 33-35, 40-42) that were capable of amplifying specific TCR-bearing T-cells from melanoma patient PBMCs.

FIG. 56 shows CD8 T-cell responses from a normal blood donor to a HLA-A*02:01-restricted peptide (SEQ ID NO. 16) from CLT Antigen 1.

FIG. 57 shows CD8 T-cell responses from a normal blood donor to HLA-A*02:01-restricted peptide (SEQ ID NO. 30) from CLT Antigen 2.

FIG. 58 shows CD8 T-cell responses from a normal blood donor to HLA-A*02:01-restricted peptide (SEQ ID NO. 43) from CLT Antigen 4.

FIG. 59 shows CD8 T-cell responses from a normal blood donor to HLA-A*03:01-restricted peptide (SEQ ID NO. 47) from CLT Antigen 5.

FIG. 60 shows CD8 T-cell responses from a normal blood donor to HLA-B*07:02-restricted peptide (SEQ ID NO. 50) from CLT Antigen 6.

FIG. 61 shows CD8 T-cell responses from a normal blood donor to HLA-A*03:01-restricted peptide (SEQ ID NO. 52) from CLT Antigen 7.

FIG. 62 shows CD8 T-cell responses from a normal blood donor to HLA-A*02:01-restricted peptide (SEQ ID NO. 55) from CLT Antigen 8.

FIG. 63 panels A to D shows responsiveness to HLA-B*07:02 restricted peptides (SEQ ID NO. 17 and 44) from CLT Antigen 1 and CLT Antigen 4 respectively in memory CD45RO-positive CD8 T-cells as compared with naïve CD45RO-negative CD8 T-cells from the same donor.

FIG. 64 shows expanded, pentamer-sorted CD8 T-cells killing C1RB7-target cells pulsed with a peptide (SEQ ID NO. 44) derived from CLT Antigen 4.

FIG. 65 shows expanded, pentamer-sorted CD8 T-cells killing of CaSki cells transfected with the open reading frame of CLT Antigen 8 (SEQ ID NO. 8).

FIG. 66 panels A to G shows qRT-PCR assay results to verify the transcription of the CLT encoding CLT Antigen 1 (SEQ ID NO. 56), the CLT encoding CLT Antigen 2 (SEQ ID NO. 57), the CLT encoding CLT Antigen 3 and 4 (SEQ ID NO. 58), the CLT encoding CLT Antigen 5 (SEQ ID NO. 59), the CLT encoding CLT Antigen 6 (SEQ ID NO. 60), the CLT encoding CLT Antigen 7 (SEQ ID NO. 61) and the CLT encoding CLT Antigen 8 (SEQ ID NO. 62) in melanoma cancer cell lines or primary tissue samples.

FIG. 67 shows schematically the construction of CLT Antigen Fusion Protein 1 (SEQ ID NO. 76), the linker sequences between CLT Antigens and likely HLA binding of linker-derived epitopes. FP=fusion protein.

FIG. 68 shows schematically the construction of CLT Antigen Fusion Protein 2 (SEQ ID NO. 77), the linker sequences between CLT Antigens and likely HLA binding of linker-derived epitopes. FP=fusion protein.

FIG. 69 shows schematically the construction of CLT Antigen Fusion Protein 3 (SEQ ID NO. 78), the linker sequences between CLT Antigens and likely HLA binding of linker-derived epitopes. FP=fusion protein.

FIG. 70 shows schematically the construction of CLT Antigen Fusion Protein 4 (SEQ ID NO. 79), the linker sequences between CLT Antigens and likely HLA binding of linker-derived epitopes. FP=fusion protein.

FIG. 71 provides a schematic explanation of the murine immunogenicity data supporting CLT Antigen Fusion Protein 1 (SEQ ID NO. 76) and CLT Antigen Fusion Protein 2 (SEQ ID NO. 77).

### DESCRIPTION OF THE SEQUENCES

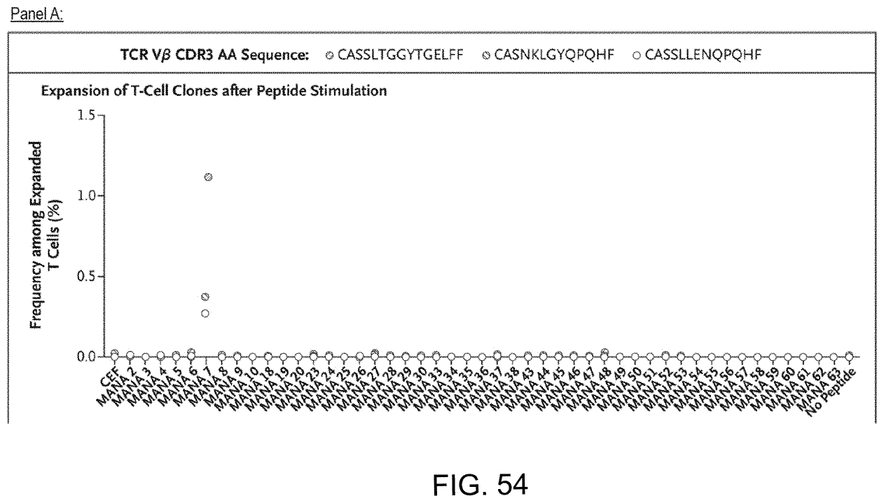

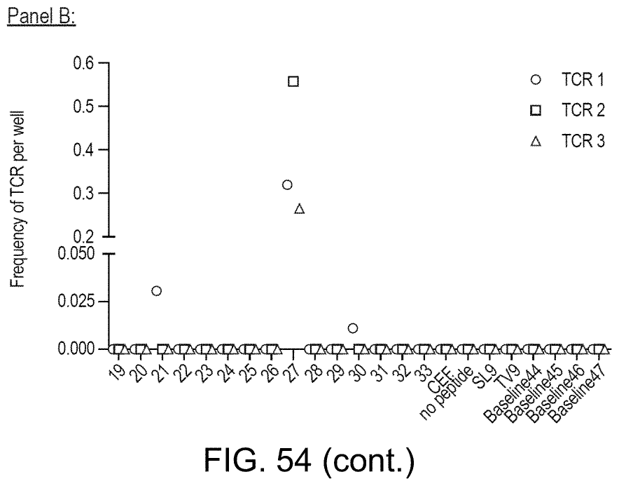

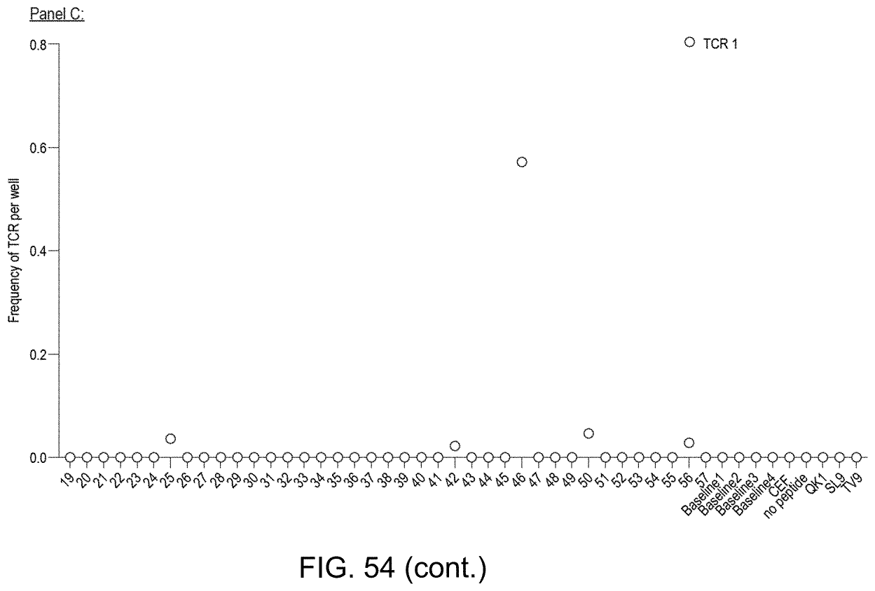

SEQ ID NO. 1 is the polypeptide sequence of CLT Antigen 1
SEQ ID NO. 2 is the polypeptide sequence of CLT Antigen 2
SEQ ID NO. 3 is the polypeptide sequence of CLT Antigen 3
SEQ ID NO. 4 is the polypeptide sequence of CLT Antigen 4
SEQ ID NO. 5 is the polypeptide sequence of CLT Antigen 5
SEQ ID NO. 6 is the polypeptide sequence of CLT Antigen 6
SEQ ID NO. 7 is the polypeptide sequence of CLT Antigen 7
SEQ ID NO. 8 is the polypeptide sequence of CLT Antigen 8
SEQ ID NOs. 9-17 are peptide sequences derived from CLT Antigen 1
SEQ ID NOs. 18-30 are peptide sequences derived from CLT Antigen 2
SEQ ID NOs. 31-35 are peptide sequences derived from CLT Antigen 3
SEQ ID NOs. 36-44 are peptide sequences derived from CLT Antigen 4
SEQ ID NOs. 45-47 are peptide sequences derived from CLT Antigen 5
SEQ ID NOs. 48-51 are peptide sequences derived from CLT Antigen 6
SEQ ID NOs. 52 is a peptide sequence derived from CLT Antigen 7
SEQ ID NOs. 53-55 are peptide sequences derived from CLT Antigen 8
SEQ ID NO. 56 is the cDNA sequence of the CLT encoding CLT Antigen 1
SEQ ID NO. 57 is the cDNA sequence of the CLT encoding CLT Antigen 2
SEQ ID NO. 58 is the cDNA sequence of the CLT encoding CLT Antigens 3 and 4
SEQ ID NO. 59 is the cDNA sequence of the CLT encoding CLT Antigen 5
SEQ ID NO. 60 is the cDNA sequence of the CLT encoding CLT Antigen 6
SEQ ID NO. 61 is the cDNA sequence of the CLT encoding CLT Antigens 7
SEQ ID NO. 62 is the cDNA sequence of the CLT encoding CLT Antigen 8
SEQ ID NO. 63 is a cDNA sequence encoding CLT Antigen 1
SEQ ID NO. 64 is a cDNA sequence encoding CLT Antigen 2
SEQ ID NO. 65 is a cDNA sequence encoding CLT Antigen 3
SEQ ID NO. 66 is a cDNA sequence encoding CLT Antigen 4
SEQ ID NO. 67 is a cDNA sequence encoding CLT Antigen 5
SEQ ID NO. 68 is a cDNA sequence encoding CLT Antigen 6
SEQ ID NO. 69 is a cDNA sequence encoding CLT Antigen 7
SEQ ID NO. 70 is a cDNA sequence encoding CLT Antigen 8
SEQ ID NOs. 71-75 are linker sequences used to construct CLT Antigen Fusion Proteins
SEQ ID NO. 76 is the polypeptide sequence of CLT Antigen Fusion Protein 1
SEQ ID NO. 77 is the polypeptide sequence of CLT Antigen Fusion Protein 2
SEQ ID NO. 78 is the polypeptide sequence of CLT Antigen Fusion Protein 3
SEQ ID NO. 79 is the polypeptide sequence of CLT Antigen Fusion Protein 4
SEQ ID NO. 80 is a cDNA sequence encoding CLT Antigen Fusion Protein 1
SEQ ID NO. 81 is a cDNA sequence encoding CLT Antigen Fusion Protein 2
SEQ ID NO. 82 is a cDNA sequence encoding CLT Antigen Fusion Protein 3
SEQ ID NO. 83 is a cDNA sequence encoding CLT Antigen Fusion Protein 4
SEQ ID NO. 84 is a linker sequence used to construct CLT Antigen Fusion Proteins
SEQ ID NOs: 85-87 are TCR VB CDR3 AA sequences shown in FIG. 54

### DETAILED DESCRIPTION OF THE INVENTION

### Fusion Proteins

The term “fusion protein” refers to any protein comprising at least two polypeptides that are joined together by peptide bonds, through protein synthesis. The fusion protein may be created through the joining of two or more genes that encode for separate polypeptides that have been joined so that they are transcribed and translated as a single unit producing a single protein.

The invention provides a fusion protein comprising at least six polypeptides where each polypeptide is fused to a second or further polypeptide, by creating nucleic acid constructs that fuse together the sequences encoding the individual polypeptides. Fusion proteins of the invention are expected to have the utilities described herein and may have the advantage of superior immunogenic or vaccine activity or prophylactic or therapeutic effect (including increasing the breadth and depth of responses) as compared with the individual component polypeptides, and may be especially valuable in an outbred population. Fusion proteins of the invention may also provide the benefit of increasing the efficiency of construction and manufacture of vaccine antigens and/or vectored vaccines (including nucleic acid vaccines).

Thus, the invention provides a fusion protein comprising six antigenic polypeptides (a) to (f), wherein the antigenic polypeptides (a) to (f) have the amino acid sequences:
(a) SEQ ID NO: 1 or a variant thereof, or an immunogenic fragment of SEQ ID NO: 1 or a variant thereof;
(b) SEQ ID NO: 2 or a variant thereof or an immunogenic fragment of SEQ ID NO: 2 or a variant thereof;
(c) SEQ ID NO: 6 or a variant thereof, or an immunogenic fragment of SEQ ID NO: 6 or a variant thereof;
(d) SEQ ID NO: 7 or a variant thereof, or an immunogenic fragment of SEQ ID NO: 7 or a variant thereof;
(e) SEQ ID NO: 4 or a variant thereof, or an immunogenic fragment of SEQ ID NO: 4 or a variant thereof; and
(f) SEQ ID NO: 8 or a variant thereof, or an immunogenic fragment of SEQ ID NO: 8 or a variant thereof.

The fusion proteins of the invention may further comprise one or more additional antigenic polypeptides selected from antigenic polypeptides (g) and (h), wherein the antigenic polypeptides (g) and (h) have amino acid sequences:
(g) SEQ ID NO: 3 or a variant thereof, or an immunogenic fragment of SEQ ID NO: 3 or a variant thereof; and
(h) SEQ ID NO: 5 or a variant thereof, or an immunogenic fragment of SEQ ID NO: 5 or a variant thereof.

In one embodiment, the fusion polypeptide comprises six antigenic polypeptides (a) to (f). In one embodiment, the fusion polypeptide comprises eight antigenic polypeptides (a) to (h). In one embodiment, the fusion polypeptide comprises seven antigenic polypeptides (a) to (g). In one embodiment, the fusion polypeptide comprises seven antigenic polypeptides (a) to (f) and (h).

One or more of the antigenic polypeptides (a) to (f) (e.g. one, two, three, four, five or all six of the antigenic polypeptides (a) to (f)) may comprise or consist of a sequence lacking an N-terminal methionine amino acid residue. For example, antigenic polypeptide (a) may have the sequence of SEQ ID NO: 1 with the N-terminal methionine amino acid removed and/or antigenic polypeptide (b) may have the sequence of SEQ ID NO: 2 with the N-terminal methionine amino acid removed and/or antigenic polypeptide (c) may have the sequence of SEQ ID NO: 3 with the N-terminal methionine amino acid removed and/or antigenic polypeptide (d) may have the sequence of SEQ ID NO: 4 with the N-terminal methionine amino acid removed and/or antigenic polypeptide (e) may have the sequence of SEQ ID NO: 5 with the N-terminal methionine amino acid removed and/or antigenic polypeptide (f) may have the sequence of SEQ ID NO: 6 with the N-terminal methionine amino acid removed. Where present, one or more (e.g. either one or both) of the antigenic polypeptides (g) and (h), may comprise or consist of a sequence lacking an N-terminal methionine amino acid residue. For example, antigenic polypeptide (g) may have the sequence of SEQ ID NO: 7 with the N-terminal methionine amino acid removed and/or antigenic polypeptide (h) may have the sequence of SEQ ID NO: 8 with the N-terminal methionine amino acid removed.

Thus, the invention provides a fusion protein comprising six antigenic polypeptides (a) to (f), wherein the antigenic polypeptides (a) to (f) have the amino acid sequences:
(a) SEQ ID NO: 1 with or without the N-terminal methionine residue or a variant thereof, or an immunogenic fragment of SEQ ID NO: 1 or a variant thereof;
(b) SEQ ID NO: 2 with or without the N-terminal methionine residue or a variant thereof, or an immunogenic fragment of SEQ ID NO: 2 or a variant thereof;
(c) SEQ ID NO: 6 with or without the N-terminal methionine residue or a variant thereof, or an immunogenic fragment of SEQ ID NO: 6 or a variant thereof;
(d) SEQ ID NO: 7 with or without the N-terminal methionine residue or a variant thereof, or an immunogenic fragment of SEQ ID NO: 7 or a variant thereof;
(e) SEQ ID NO: 4 with or without the N-terminal methionine residue or a variant thereof, or an immunogenic fragment of SEQ ID NO: 4 or a variant thereof;
(f) SEQ ID NO: 8 with or without the N-terminal methionine residue or a variant thereof, or an immunogenic fragment of SEQ ID NO: 8 or a variant thereof.

The fusion proteins of the invention may further comprise one or more additional antigenic polypeptides selected from antigenic polypeptides (g) and (h), wherein the antigenic polypeptides (g) and (h) have amino acid sequences:
(g) SEQ ID NO: 3 with or without the N-terminal methionine residue or a variant thereof, or an immunogenic fragment of SEQ ID NO: 3 or a variant thereof; and
(h) SEQ ID NO: 5 with or without the N-terminal methionine residue or a variant thereof, or an immunogenic fragment of SEQ ID NO: 5 or a variant thereof.

In an embodiment, the fusion proteins of the invention comprise an antigenic polypeptide having the amino acid sequence of SEQ ID NO: 2 minus the N-terminal methionine residue. In an embodiment, the fusion proteins of the invention comprise an antigenic polypeptide having the amino acid sequence of SEQ ID NO: 6 minus the N-terminal methionine residue. In an embodiment, the fusion proteins of the invention comprise an antigenic polypeptide having the amino acid sequence of SEQ ID NO: 5 minus the N-terminal methionine residue. In an embodiment, the fusion proteins of the invention comprise an antigenic polypeptide having the amino acid sequence of SEQ ID NO: 2 minus the N-terminal methionine residue, an antigenic polypeptide having the amino acid sequence of SEQ ID NO: 6 minus the N-terminal methionine residue, and an antigenic polypeptide having the amino acid sequence of SEQ ID NO: 5 minus the N-terminal methionine residue.

Suitably, the fusion protein of the invention comprises six antigenic polypeptides (a) to (f) wherein the antigenic polypeptides (a) to (f) have the amino acid sequences:
(a) SEQ ID NO: 1;
(b) SEQ ID NO: 2 minus the N-terminal methionine residue;
(c) SEQ ID NO: 6;
(d) SEQ ID NO: 7;
(e) SEQ ID NO: 4; and
(f) SEQ ID NO: 8.

Suitably, the fusion protein of the invention comprises eight antigenic polypeptides (a) to (h) wherein the antigenic polypeptides (a) to (h) have the amino acid sequences:
(a) SEQ ID NO: 1;
(b) SEQ ID NO: 2 minus the N-terminal methionine residue;
(c) SEQ ID NO: 6 minus the N-terminal methionine residue;
(d) SEQ ID NO: 7;
(e) SEQ ID NO: 4;
(f) SEQ ID NO: 8;
(g) SEQ ID NO: 3; and
(h) SEQ ID NO: 5 minus the N-terminal methionine residue.

Suitably, the fusion protein of the invention comprises eight antigenic polypeptides (a) to (h) wherein the antigenic polypeptides (a) to (h) have the amino acid sequences:
(a) SEQ ID NO: 1;
(b) SEQ ID NO: 2 minus the N-terminal methionine residue;
(c) SEQ ID NO: 6;
(d) SEQ ID NO: 7;
(e) SEQ ID NO: 4;
(f) SEQ ID NO: 8;
(g) SEQ ID NO: 3; and
(h) SEQ ID NO: 5.

The antigenic polypeptides of the fusion protein of the present invention may be arranged in various sequential orders from the N terminus to the C terminus. The design and order of the polypeptides in the fusion proteins of the invention are described in Example 8. In particular, the order of the polypeptides in the fusion protein is important because such an order can in some cases lead to superior processing and presentation of desirable immunogenic peptide regions of a polypeptide and in other cases is necessary for optimal fusion design to reduce the likelihood of unnatural immunogenic peptides, derived from the junctions between the natural cancer-specific CLT Antigens could be presented on surface displayed Class I HLA molecules during vaccination, thus eliciting undesirable T cell responses.

The fusion proteins of the invention provide for a strong antigenic response to the component CLT Antigens, see Examples 9 & 10, and are expected to elicit minimal antigenic responses to their junction regions, see Example 8.

In one embodiment, when the fusion protein comprises six antigenic polypeptides (a) to (f), the six antigenic polypeptides are arranged in the order from N to C of (a), (b), (c), (d), (e) and (f).

In one suitable embodiment, the six antigenic polypeptides have the sequences of SEQ ID NOs. 1-2, 4, 6-8 and are arranged in the order from N to C of SEQ ID NO: 1, SEQ ID NO: 2, SEQ ID NO: 6, SEQ ID NO: 7, SEQ ID NO: 4 and SEQ ID NO: 8. A corresponding sequence in which the N-terminal methionine is omitted may optionally be used as explained above. Thus, suitably, SEQ ID NO: 1 is present at the N terminus and SEQ ID NO: 8 is present at the C terminus. Suitably the N-terminal methionine of SEQ ID NO: 2 is omitted. In an embodiment of the invention, the fusion protein has the sequence of SEQ ID NO: 76.

In another embodiment, when the fusion protein comprises six antigenic polypeptides (a) to (f), the six antigenic polypeptides are arranged in the order from N to C of (c), (f), (d), (b), (e) and (a).

In one suitable embodiment, the six antigenic polypeptides have the sequences of SEQ ID NOs. 1-2, 4, 6-8 and are arranged in the order from N to C of SEQ ID NO: 6, SEQ ID NO: 8, SEQ ID NO: 7, SEQ ID NO: 2, SEQ ID NO: 4 and SEQ ID NO: 1. A corresponding sequence in which the N-terminal methionine is omitted may optionally be used as explained above. Thus, suitably, SEQ ID NO: 6 is present at the N terminus and SEQ ID NO: 1 is present at the C terminus. Suitably the N-terminal methionine of SEQ ID NO: 2 is omitted. In an embodiment of the invention, the fusion protein has the sequence of SEQ ID NO: 77.

In another embodiment, when the fusion protein comprises eight antigenic polypeptides (a) to (h), the eight antigenic polypeptides are arranged in the order from N to C of (a), (b), (g), (d), (e), (h), (c) and (f).

In one suitable embodiment, the eight antigenic polypeptides have the sequences of SEQ ID NOs. 1-8 and are arranged in the order from N to C of SEQ ID NO: 1, SEQ ID NO: 2, SEQ ID NO: 3, SEQ ID NO: 7, SEQ ID NO: 4, SEQ ID NO: 5, SEQ ID NO: 6 and SEQ ID NO: 8. A corresponding sequence in which the N-terminal methionine is omitted may optionally be used as explained above. Suitably, SEQ ID NO: 1 present at the N-terminal and SEQ ID NO: 8 is present at the C terminus. Suitably the N-terminal methionine of SEQ ID NO: 2 is omitted. Suitably the N-terminal methionine of SEQ ID NO: 6 is omitted. Suitably the N-terminal methionine of SEQ ID NO: 5 is omitted. In an embodiment of the invention, the fusion protein has the sequence of SEQ ID NO: 78.

In another embodiment, when the fusion protein comprises eight antigenic polypeptides (a) to (h), the eight antigenic polypeptides are arranged in the order from N to C of (c), (g), (a), (h), (e), (f), (d) and (b).

In one suitable embodiment, the eight antigenic polypeptides have the sequences of SEQ ID NOs. 1-8 and are arranged in the order from N to C of SEQ ID NO: 6, SEQ ID NO: 3, SEQ ID NO: 1, SEQ ID NO: 5, SEQ ID NO: 4, SEQ ID NO: 8, SEQ ID NO: 7 and SEQ ID NO: 2. A corresponding sequence in which the N-terminal methionine is omitted may optionally be used as explained above. Suitably, SEQ ID NO: 6 is present at the N-terminal and SEQ ID NO: 2 is present at the C terminus. Suitably the N-terminal methionine of SEQ ID NO: 2 is omitted. In an embodiment of the invention, the fusion protein has the sequence of SEQ ID NO: 79.

Fusion proteins of the invention may be fused to a second or further polypeptide selected from (i) other polypeptides which are melanoma associated antigens; (ii) polypeptide sequences which are capable of enhancing an immune response (i.e. immunostimulant sequences); and (iii) polypeptide sequences, e.g. comprising universal CD4 helper epitopes, which are capable of providing strong CD4+ help to increase CD8+ T cell responses to antigen epitopes.

The invention also provides nucleic acids encoding the aforementioned fusion polypeptides and other aspects of the invention (vectors, compositions, cells etc) mutatis mutandis as for the polypeptides of the invention.

### Polypeptides

The terms “protein”, “polypeptide” and “peptide” are used interchangeably herein and refer to any peptide-linked chain of amino acids, regardless of length, co-translational or post-translational modification.

The term “amino acid” refers to any one of the naturally occurring amino acids, as well as amino acid analogs and amino acid mimetics that function in a manner which is similar to the naturally occurring amino acids. Naturally occurring amino acids are those 20 L-amino acids encoded by the genetic code, as well as those amino acids that are later modified, e.g., hydroxyproline, γ-carboxyglutamate, and O-phosphoserine. The term “amino acid analogue” refers to a compound that has the same basic chemical structure as a naturally occurring amino acid, i.e., an a carbon that is bound to a hydrogen, a carboxyl group, an amino group, and an R group but has a modified R group or a modified peptide backbone as compared with a natural amino acid. Examples include homoserine, norleucine, methionine sulfoxide, methionine methyl sulfonium and norleucine. Amino acid mimetics refers to chemical compounds that have a structure that is different from the general chemical structure of an amino acid, but that functions in a manner similar to a naturally occurring amino acid. Suitably an amino acid is a naturally occurring amino acid or an amino acid analogue, especially a naturally occurring amino acid and in particular one of those 20 L-amino acids encoded by the genetic code.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In general, variants of polypeptide sequences of the fusion proteins of the invention include sequences having a high degree of sequence identity thereto. For example variants suitably have at least about 80% identity, more preferably at least about 85% identity and most preferably at least about 90% identity (such as at least about 95%, at least about 98% or at least about 99%) to the associated reference sequence over their whole length.

Suitably the variant is an immunogenic variant. A variant is considered to be an immunogenic variant where it elicits a response which is at least 20%, suitably at least 50% and especially at least 75% (such as at least 90%) of the activity of the reference sequence (i.e. the sequence of which the variant is a variant) e.g., in an in vitro restimulation assay of PBMC or whole blood with the polypeptide as antigen (e.g., restimulation for a period of between several hours to up to 1 year, such as up to 6 months, 1 day to 1 month or 1 to 2 weeks), that measures the activation of the cells via lymphoproliferation (e.g., T-cell proliferation), production of cytokines (e.g., IFN-gamma) in the supernatant of culture (measured by ELISA etc.) or characterisation of T-cell responses by intra and extracellular staining (e.g., using antibodies specific to immune markers, such as CD3, CD4, CD8, IL2, TNF-alpha, IFNg, Type 1 IFN, CD40L, CD69 etc.) followed by analysis with a flow cytometer.

The variant may, for example, be a conservatively modified variant. A “conservatively modified variant” is one where the alteration(s) results in the substitution of an amino acid with a functionally similar amino acid or the substitution/deletion/addition of residues which do not substantially impact the biological function of the variant. Typically, such biological function of the variants will be to induce an immune response against a melanoma e.g. a cutaneous melanoma cancer antigen.

Conservative substitution tables providing functionally similar amino acids are well known in the art. Variants can include homologues of polypeptides found in other species.

Fusion proteins of the invention may comprise a polypeptide having a variant sequence that contains a number of substitutions, for example, conservative substitutions (for example, 1-25, such as 1-10, in particular 1-5, and especially 1 amino acid residue(s) may be altered) when compared to the reference sequence. The number of substitutions, for example, conservative substitutions, may be up to 20% e.g., up to 10% e.g., up to 5% e.g., up to 1% of the number of residues of the reference sequence. In general, conservative substitutions will fall within one of the amino-acid groupings specified below, though in some circumstances other substitutions may be possible without substantially affecting the immunogenic properties of the antigen. The following eight groups each contain amino acids that are typically conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins 1984).

Suitably such substitutions do not alter the immunological structure of an epitope (e.g., they do not occur within the epitope region as mapped in the primary sequence), and do not therefore have a significant impact on the immunogenic properties of the antigen.

Polypeptide variants also include those wherein additional amino acids are inserted compared to the reference sequence, for example, such insertions may occur at 1-10 locations (such as 1-5 locations, suitably 1 or 2 locations, in particular 1 location) and may, for example, involve the addition of 50 or fewer amino acids at each location (such as 20 or fewer, in particular 10 or fewer, especially 5 or fewer). Suitably such insertions do not occur in the region of an epitope, and do not therefore have a significant impact on the immunogenic properties of the antigen. One example of insertions includes a short stretch of histidine residues (e.g., 2-6 residues) to aid expression and/or purification of the antigen in question.

Polypeptide variants include those wherein amino acids have been deleted compared to the reference sequence, for example, such deletions may occur at 1-10 locations (such as 1-5 locations, suitably 1 or 2 locations, in particular 1 location) and may, for example, involve the deletion of 50 or fewer amino acids at each location (such as 20 or fewer, in particular 10 or fewer, especially 5 or fewer). Suitably such deletions do not occur in the region of an epitope, and do not therefore have a significant impact on the immunogenic properties of the antigen.

The skilled person will recognise that a particular protein variant may comprise substitutions, deletions and additions (or any combination thereof). For example, substitutions/deletions/additions might enhance (or have neutral effects) on binding to desired patient HLA molecules, potentially increasing immunogenicity (or leaving immunogenicity unchanged).

Immunogenic fragments of the polypeptides of the fusion proteins according to the present invention will typically comprise at least 9 contiguous amino acids from the full-length polypeptide sequence (e.g., at least 9 or 10), such as at least 12 contiguous amino acids (e.g., at least 15 or at least 20 contiguous amino acids), in particular at least 50 contiguous amino acids, such as at least 100 contiguous amino acids (for example at least 200 contiguous amino acids) depending on the length of the CLT antigen. Suitably the immunogenic fragments will be at least 10%, such as at least 20%, such as at least 50%, such as at least 70% or at least 80% of the length of the full-length polypeptide sequence.

Immunogenic fragments typically comprise at least one epitope. Epitopes include B cell and T-cell epitopes and suitably immunogenic fragments comprise at least one T-cell epitope such as a CD4+ or a CD8+ T-cell epitope.

T-cell epitopes are short contiguous stretches of amino acids which are recognised by T-cells (e.g., CD4+ or CD8+ T-cells) when bound to HLA molecules. Identification of T-cell epitopes may be achieved through epitope mapping experiments which are well known to the person skilled in the art (see, for example, Paul, Fundamental Immunology, 3rd ed., 243-247 (1993); Beißbarth et al., 2005, Bioinformatics, 21(Suppl. 1):i29-i37).

As a result of the crucial involvement of the T-cell response in cancer, it is readily apparent that fragments of the full-length polypeptides of SEQ ID NOs. 1-8 which contain at least one T-cell epitope may be immunogenic and may contribute to immunoprotection.

It will be understood that in a diverse outbred population, such as humans, different HLA types mean that specific epitopes may not be recognised by all members of the population. Consequently, to maximise the level of recognition and scale of immune response to a polypeptide, it is generally desirable that an immunogenic fragment contains a plurality of the epitopes from the full-length sequence (suitably all epitopes within a CLT antigen).

Particular fragments of the antigenic polypeptides of SEQ ID NOs. 1-8 which may be of use include those containing at least one CD8+ T-cell epitope, suitably at least two CD8+ T-cell epitopes and especially all CD8+ T-cell epitopes, particularly those associated with a plurality of HLA alleles, e.g., those associated with 2, 3, 4, 5 or more alleles). Particular fragments of the antigenic polypeptides of SEQ ID NOs. 1-8 which may be of use include those containing at least one CD4+ T-cell epitope, suitably at least two CD4+ T-cell epitopes and especially all CD4+ T-cell epitopes (particularly those associated with a plurality of HLA alleles, e.g., those associated with 2, 3, 4, 5 or more alleles). However, a person skilled in design of vaccines could combine exogenous CD4+ T-cell epitopes with CD8+ T-cells epitopes and achieve desired responses to the CD8+ T-cell epitopes.

Where an individual fragment of the full-length polypeptide is used, such a fragment is considered to be immunogenic where it elicits a response which is at least 20%, suitably at least 50% and especially at least 75% (such as at least 90%) of the activity of the reference sequence (i.e., the sequence of which the fragment is a fragment) e.g., activity in an in vitro restimulation assay of PBMC or whole blood with the polypeptide as antigen (e.g., restimulation for a period of between several hours to up to 1 year, such as up to 6 months, 1 day to 1 month or 1 to 2 weeks,) that measures the activation of the cells via lymphoproliferation (e.g., T-cell proliferation), production of cytokines (e.g., IFN-gamma) in the supernatant of culture (measured by ELISA etc.) or characterisation of T-cell responses by intra and extracellular staining (e.g., using antibodies specific to immune markers, such as CD3, CD4, CD8, IL2, TNF-alpha, IFN-gamma, Type 1 IFN, CD40L, CD69 etc.) followed by analysis with a flow cytometer.

In some circumstances a plurality of fragments of the full-length polypeptide (which may or may not be overlapping and may or may not cover the entirety of the full-length sequence) may be used to obtain an equivalent biological response to the full-length sequence itself. For example, at least two immunogenic fragments (such as three, four or five) as described above, which in combination provide at least 50%, suitably at least 75% and especially at least 90% activity of the reference sequence in an in vitro restimulation assay of PBMC or whole blood (e.g., a T-cell proliferation and/or IFN-gamma production assay).

Example immunogenic fragments of antigenic polypeptides of SEQ ID NOs. 1-8, and thus example component peptides of fusion proteins of the invention, include polypeptides which comprise or consist of the sequences of SEQ ID NOs. 9-55. The sequences of SEQ ID NOs. 9-12, 18-19, 30, 31-32 and 37-39, 45, 48-54 were identified as being bound to HLA Class I molecules from immunopeptidomic analysis (see Example 2). The sequences of SEQ ID NOs 13-17, 20-29, 33-35, 40-44 were predicted by NetMHC software as being bound to HLA Class I molecules and were used in immunological validation assays (see Examples 3, 4 and 5).

The antigenic polypeptide component (a) of the fusion protein may comprise or consist of SEQ ID NO. 1 or a variant thereof, or an immunogenic fragment of SEQ ID NO: 1 or a variant thereof. Exemplary fragments comprise or consist of any one of SEQ ID NOs. 9-12. Further exemplary fragments comprise two, three or four of SEQ ID NOs. 9-12. Further exemplary fragments comprise or consist of any one of SEQ ID NOs. 13-17. Further exemplary fragments comprise all of SEQ ID NOs. 9-17 (allowance being taken for possible sequence overlap so that any overlapping sequence does not need to be present more than once).

The antigenic polypeptide component (b) of the fusion protein may comprise or consist of SEQ ID NO. 2 or a variant thereof, or an immunogenic fragment of SEQ ID NO: 2 or a variant thereof. Exemplary fragments comprise or consist of SEQ ID NO. 18 or SEQ ID NO. 19. Further exemplary fragments comprise SEQ ID NO. 18 and SEQ ID NO. 19. Further exemplary fragments comprise or consist of any one of SEQ ID NOs. 20-30. Further exemplary fragments comprise all of SEQ ID NOs. 18-30 (allowance being taken for possible sequence overlap so that any overlapping sequence does not need to be present more than once).

The antigenic polypeptide component (c) of the fusion protein may comprise or consist of SEQ ID NO. 6 or a variant thereof, or an immunogenic fragment of SEQ ID NO: 6 or a variant thereof. Exemplary fragments comprise or consist of SEQ ID NO. 48-51.

The antigenic polypeptide component (d) of the fusion protein may comprise or consist of SEQ ID NO. 7 or a variant thereof, or an immunogenic fragment of SEQ ID NO: 7 or a variant thereof. Exemplary fragments comprise or consist of SEQ ID NO. 52.

The antigenic polypeptide component (e) of the fusion protein may comprise or consist of SEQ ID NO. 4 or a variant thereof, or an immunogenic fragment of SEQ ID NO: 4 or a variant thereof. Exemplary fragments comprise or consist of SEQ ID NO. 36. Further exemplary fragments comprise or consist of SEQ ID NO. 37 or SEQ ID NO. 38. Further exemplary fragments comprise or consist of SEQ ID NO. 39. Further exemplary fragments comprise or consist of any one of SEQ ID NOs. 40-44. Further exemplary fragments comprise SEQ ID NO. 36 and either SEQ ID NO. 37 or SEQ ID NO. 38. Further exemplary fragments comprise SEQ ID NO. 39 and either SEQ ID NO. 37 or SEQ ID NO. 38. Further exemplary fragments comprise all of SEQ ID NOs. 36-44 (allowance being taken for possible sequence overlap so that any overlapping sequence does not need to be present more than once).

The antigenic polypeptide component (f) of the fusion protein may comprise or consist of SEQ ID NO. 8 or a variant thereof, or an immunogenic fragment of SEQ ID NO: 8 or a variant thereof. Exemplary fragments comprise or consist of SEQ ID NO. 53-55.

The antigenic polypeptide component (g) of the fusion protein may comprise or consist of SEQ ID NO. 3 or a variant thereof, or an immunogenic fragment of SEQ ID NO: 3 or a variant thereof. Exemplary fragments comprise or consist of SEQ ID NO. 31. Further exemplary fragments comprise SEQ ID NO. 31. Further exemplary fragments comprise or consist of any one of SEQ ID NOs. 32-35. Further exemplary fragments comprise SEQ ID NO. 31 and SEQ ID NO. 32. Further exemplary fragments comprise all of SEQ ID NOs. 31-35 (allowance being taken for possible sequence overlap so that any overlapping sequence does not need to be present more than once).

The antigenic polypeptide component (h) of the fusion protein may comprise or consist of SEQ ID NO. 5 or a variant thereof, or an immunogenic fragment of SEQ ID NO: 5 or a variant thereof. Exemplary fragments comprise or consist of any one of SEQ ID NOs. 45-47.

### Linkers

The invention provides for fusion proteins wherein the antigenic polypeptides of the fusion proteins are joined together by one or more peptide linkers. In one embodiment of the invention, the antigenic polypeptides of the fusion protein of the present invention are joined together by one or more linkers (e.g. two, three, four, five, six or seven linkers). A linker may separate each of the antigenic polypeptides of the fusion protein. The linkers may be ‘internal’, i.e. the linkers are not present at the N terminus of the first polypeptide and the C terminus of the last polypeptide of the fusion protein. In one embodiment of the invention, the one or more linkers are positioned between antigenic polypeptides (a) and (b), (b) and (c), (c) and (d), (d) and (e), (e) and (f). In another embodiment of the invention, the one or more linkers are positioned between antigenic polypeptides (c) and (f), (f) and (d), (d) and (b), (b) and (e), (e) and (a). In a further embodiment of the invention, the linkers are positioned between antigenic polypeptides (a) and (b), (b) and (g), (g) and (d), (d) and (e), (e) and (h), (h) and (c), (c) and (f). In yet a further embodiment of the invention the linkers are positioned between antigenic polypeptides (c) and (g), (g) and (a), (a) and (h), (h) and (e), (e) and (f), (f) and (d), (d) and (b).

The linker may refer to the cDNA encoding the linker peptide sequence, or the encoded peptide. The linkers are placed between the individual antigens of each fusion protein of the invention by creating a single construct in which the linker sequence is inserted between the C terminus of one antigenic polypeptide and the N terminus of the following antigenic polypeptide, thereby linking the antigenic polypeptides of the fusion protein together. The individual linkers used in a fusion protein may have the same sequence or they may have different sequences. In one embodiment of the invention, the linkers are selected from the peptide linkers having the sequences of SEQ ID NOs: 71-75 and 84.

In an embodiment of the invention, the fusion protein comprises or consists of a sequence selected from SEQ ID NOs: 76-79.

The linkers of the present invention are glycine based linkers, which may also include lysines, in a connector of 3 to 6 amino acids in length (see of SEQ ID NOs: 71-75 and 84). The linkers of the present invention reduce the risk of introducing unwanted immunogenic epitopes which contain the linker itself; they also prevent the unwanted epitopes created by direct fusion of the individual antigenic polypeptides.

The fusion proteins of the present invention may be created through the joining of six or more genes (e.g. six, seven, eight, nine, ten, eleven, twelve, thirteen, fourteen, fifteen) that encode for separate antigenic polypeptides and cDNAs that encode linkers that have been joined so that the resulting open reading frames are transcribed and translated as a single unit producing a single protein. Nucleic acids encoding the fusion proteins of the present invention may comprise or consist of a sequence selected from SEQ ID NOs: 80-83.

### Nucleic Acids

The invention provides an isolated nucleic acid encoding the fusion proteins of the invention (referred to as a nucleic acid of the invention).

The terms “nucleic acid” and “polynucleotide” are used interchangeably herein and refer to a polymeric macromolecule made from nucleotide monomers particularly deoxyribonucleotide or ribonucleotide monomers. The term encompasses nucleic acids containing known nucleotide analogs or modified backbone residues or linkages, which are naturally occurring and non-naturally occurring, which have similar properties as the reference nucleic acid, and which are intended to be metabolized in a manner similar to the reference nucleotides or are intended to have extended half-life in the system. Examples of such analogs include, without limitation, phosphorothioates, phosphoramidates, methyl phosphonates, chiral-methyl phosphonates, 2-O-methyl ribonucleotides, peptide-nucleic acids (PNAs). Suitably the term “nucleic acid” refers to naturally occurring polymers of deoxyribonucleotide or ribonucleotide monomers. Suitably the nucleic acid molecules of the invention are recombinant. Recombinant means that the nucleic acid molecule is the product of at least one of cloning, restriction or ligation steps, or other procedures that result in a nucleic acid molecule that is distinct from a nucleic acid molecule found in nature (e.g., in the case of cDNA). In an embodiment the nucleic acid of the invention is an artificial nucleic acid sequence (e.g., a cDNA sequence or nucleic acid sequence with non-naturally occurring codon usage). In one embodiment, the nucleic acids of the invention are DNA. Alternatively, the nucleic acids of the invention are RNA.

DNA (deoxyribonucleic acid) and RNA (ribonucleic acid) refer to nucleic acid molecules having a backbone of sugar moieties which are deoxyribosyl and ribosyl moieties respectively. The sugar moieties may be linked to bases which are the 4 natural bases (adenine (A), guanine (G), cytosine (C) and thymine (T) in DNA and adenine (A), guanine (G), cytosine (C) and uracil (U) in RNA). As used herein, a “corresponding RNA” is an RNA having the same sequence as a reference DNA but for the substitution of thymine (T) in the DNA with uracil (U) in the RNA. The sugar moieties may also be linked to unnatural bases such as inosine, xanthosine, 7-methylguanosine, dihydrouridine and 5-methylcytidine. Natural phosphodiester linkages between sugar (deoxyribosyl/ribosyl) moieties may optionally be replaced with phosphorothioates linkages. Suitably nucleic acids of the invention consist of the natural bases attached to a deoxyribosyl or ribosyl sugar backbone with phosphodiester linkages between the sugar moieties.

In an embodiment the nucleic acid of the invention is a DNA. For example the nucleic acid comprises or consists of a sequence selected from SEQ ID NOs. 56-62 and 63-70. Also provided is a nucleic acid which comprises or consists of a variant of sequence selected from SEQ ID NOs. 56-62 or 63-70 which variant encodes the same amino acid sequence but has a different nucleic acid based on the degeneracy of the genetic code.

Thus, due to the degeneracy of the genetic code, a large number of different, but functionally identical nucleic acids can encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations lead to “silent” (sometimes referred to as “degenerate” or “synonymous”) variants, which are one species of conservatively modified variations. Every nucleic acid sequence disclosed herein which encodes a polypeptide also enables every possible silent variation of the nucleic acid. One of skill will recognise that each codon in a nucleic acid (except AUG, which is ordinarily the only codon for methionine, and UGG, which is ordinarily the only codon for tryptophan) can be modified to yield a functionally identical molecule. Accordingly, each silent variation of a nucleic acid that encodes a polypeptide is implicit in each described sequence and is provided as an aspect of the invention.

Degenerate codon substitutions may also be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; Rossolini et al., 1994, Mol. Cell. Probes 8:91-98).

A nucleic acid of the invention which comprises or consists of a sequence selected from SEQ ID NOs. 56-62 and 63-70 may contain a number of silent variations (for example, 1-50, such as 1-25, in particular 1-5, and especially 1 codon(s) may be altered) when compared to the reference sequence.

In an embodiment the nucleic acid of the invention is an RNA. RNA sequences are provided which correspond to a DNA sequence provided herein and have a ribonucleotide backbone instead of a deoxyribonucleotide backbone and have the sidechain base uracil (U) in place of thymine (T).

Thus a nucleic acid of the invention comprises or consists of the RNA equivalent of a cDNA sequence selected from SEQ ID NOs. 56-62 and 63-70 and may contain a number of silent variations (for example, 1-50, such as 1-25, in particular 1-5, and especially 1 codon(s) may be altered) when compared to the reference sequence. By “RNA equivalent” is meant an RNA sequence which contains the same genetic information as the reference cDNA sequence (i.e. contains the same codons with a ribonucleotide backbone instead of a deoxyribonucleotide backbone and having the sidechain base uracil (U) in place of thymine (T)).

The invention also comprises sequences which are complementary to the aforementioned cDNA and RNA sequences.

In an embodiment, the nucleic acids of the invention are codon optimised for expression in a human host cell.

The nucleic acids of the invention are capable of being transcribed and translated into fusion proteins of the invention in the case of DNA nucleic acids, and translated into fusion proteins of the invention in the case of RNA nucleic acids.

### Polypeptides and Nucleic Acids

Suitably, the nucleic acids used in the present invention are isolated. An “isolated” nucleic acid is one that is removed from its original environment. For example, a naturally-occurring nucleic acid is isolated if it is separated from some or all of the coexisting materials in the natural system. A nucleic acid is considered to be isolated if, for example, it is cloned into a vector that is not a part of its natural environment.

“Naturally occurring” when used with reference to a polypeptide or nucleic acid sequence means a sequence found in nature and not synthetically modified.

“Artificial” when used with reference to a polypeptide or nucleic acid sequence means a sequence not found in nature which is, for example, a synthetic modification of a natural sequence, or contains an unnatural sequence.

The term “heterologous” when used with reference to the relationship of one nucleic acid or polypeptide to another nucleic acid or polypeptide indicates that the two or more sequences are not found in the same relationship to each other in nature. A “heterologous” sequence can also mean a sequence which is not isolated from, derived from, or based upon a naturally occurring nucleic acid or polypeptide sequence found in the host organism.

As noted above, fusion proteins of the invention may comprise a polypeptide having a variant sequence, preferably having at least about 80% identity, more preferably at least about 85% identity and most preferably at least about 90% identity (such as at least about 95%, at least about 98% or at least about 99%) to the associated reference sequence over their whole length.

For the purposes of comparing two closely-related polypeptide or polynucleotide sequences, the “% sequence identity” between a first sequence and a second sequence may be calculated. Polypeptide sequences are said to be the same as or identical to other polypeptide sequences, if they share 100% sequence identity over their entire length. Residues in sequences are numbered from left to right, i.e. from N- to C-terminus for polypeptides. The terms “identical” or percentage “identity”, in the context of two or more polypeptide sequences, refer to two or more sequences or sub-sequences that are the same or have a specified percentage of amino acid residues that are the same (i.e., 70% identity, optionally 75%, 80%, 85%, 90%, 95%, 98% or 99% A identity over a specified region), when compared and aligned for maximum correspondence over a comparison window. Suitably, the comparison is performed over a window corresponding to the entire length of the reference sequence.

For sequence comparison, one sequence acts as the reference sequence, to which the test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percentage sequence identities for the test sequences relative to the reference sequence, based on the program parameters.

A “comparison window”, as used herein, refers to a segment in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerised implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, 1987, J. Mol. Evol. 35:351-360. The method used is similar to the method described by Higgins & Sharp, 1989, CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., 1984, Nuc. Acids Res. 12:387-395).

Another example of algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. Software for performing BLAST analyses is publicly available through the National Center for Biotechnology Information (website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance.

A “difference” between sequences refers to an insertion, deletion or substitution of a single residue in a position of the second sequence, compared to the first sequence. Two sequences can contain one, two or more such differences. Insertions, deletions or substitutions in a second sequence which is otherwise identical (100% sequence identity) to a first sequence result in reduced % sequence identity. For example, if the identical sequences are 9 residues long, one substitution in the second sequence results in a sequence identity of 88.9%. If the identical sequences are 17 amino acid residues long, two substitutions in the second sequence results in a sequence identity of 88.2%.

Alternatively, for the purposes of comparing a first, reference sequence to a second, comparison sequence, the number of additions, substitutions and/or deletions made to the first sequence to produce the second sequence may be ascertained. An addition is the addition of one residue into the first sequence (including addition at either terminus of the first sequence). A substitution is the substitution of one residue in the first sequence with one different residue. A deletion is the deletion of one residue from the first sequence (including deletion at either terminus of the first sequence).

### Production of Fusion Proteins of the Invention

Fusion proteins of the invention can be obtained and manipulated using the techniques disclosed for example in Green and Sambrook 2012 Molecular Cloning: A Laboratory Manual 4th Edition Cold Spring Harbour Laboratory Press. In particular, artificial gene synthesis may be used to produce polynucleotides (Nambiar et al., 1984, Science, 223:1299-1301, Sakamar and Khorana, 1988, Nucl. Acids Res., 14:6361-6372, Wells et al., 1985, Gene, 34:315-323 and Grundstrom et al., 1985, Nucl. Acids Res., 13:3305-3316) followed by expression in a suitable organism to produce polypeptides. A gene encoding a polypeptide of the fusion proteins of the invention can be synthetically produced by, for example, solid-phase DNA synthesis. Entire genes may be synthesized de novo, without the need for precursor template DNA. To obtain the desired oligonucleotide, the building blocks are sequentially coupled to the growing oligonucleotide chain in the order required by the sequence of the product. Upon the completion of the chain assembly, the product is released from the solid phase to solution, deprotected, and collected. Products can be isolated by high-performance liquid chromatography (HPLC) to obtain the desired oligonucleotides in high purity (Verma and Eckstein, 1998, Annu. Rev. Biochem. 67:99-134). These relatively short segments are readily assembled by using a variety of gene amplification methods (Methods Mol Biol., 2012; 834:93-109) into longer DNA molecules, suitable for use in innumerable recombinant DNA-based expression systems. In the context of this invention one skilled in the art would understand that the polynucleotide sequences encoding the polypeptide antigens of the fusion proteins described in this invention could be readily used in a variety of vaccine production systems, including, for example, viral vectors.

For the purposes of production of fusion proteins of the invention in a microbiological host (e.g., bacterial or fungal), nucleic acids of the invention will comprise suitable regulatory and control sequences (including promoters, termination signals etc) and sequences to promote polypeptide secretion suitable for protein production in the host. Similarly, fusion proteins of the invention could be produced by transducing cultures of eukaryotic cells (e.g., Chinese hamster ovary cells or drosophila S2 cells) with nucleic acids of the invention which have been combined with suitable regulatory and control sequences (including promoters, termination signals etc) and sequences to promote polypeptide secretion suitable for protein production in these cells.

Improved isolation of the fusion proteins of the invention produced by recombinant means may optionally be facilitated through the addition of a stretch of histidine residues (commonly known as a His-tag) towards one end of the protein.

Fusion proteins may also be produced synthetically.

### Vectors

In additional embodiments, genetic constructs comprising one or more of the nucleic acids of the invention are introduced into cells in vivo such that fusion proteins of the invention are produced in vivo eliciting an immune response. The nucleic acid (e.g., DNA) may be present within any of a variety of delivery systems known to those of ordinary skill in the art, including nucleic acid expression systems, bacteria and some viral expression systems. Numerous gene delivery techniques are well known in the art, such as those described by Rolland, 1998, Crit. Rev. Therap. Drug Carrier Systems 15:143-198, and references cited therein. Several of these approaches are outlined below for the purpose of illustration.

Accordingly, there is provided a vector (also referred to herein as a ‘DNA expression construct’ or ‘construct’) comprising a nucleic acid molecule of the invention.

Suitably, the vector comprises nucleic acid encoding regulatory elements (such as a suitable promoter and terminating signal) suitable for permitting transcription of a translationally active RNA molecule in a human host cell. A “translationally active RNA molecule” is an RNA molecule capable of being translated into a protein by a human cell's translation apparatus.

Accordingly, there is provided a vector comprising a nucleic acid of the invention (herein after a “vector of the invention”).

In particular, the vector may be a viral vector. The viral vector may be an adenovirus, adeno-associated virus (AAV) (e.g., AAV type 5 and type 2), alphavirus (e.g., Venezuelan equine encephalitis virus (VEEV), Sindbis virus (SIN), Semliki Forest virus (SFV)), herpes virus, arenavirus (e.g., lymphocytic choriomeningitis virus (LCMV)), measles virus, poxvirus (such as modified vaccinia Ankara (MVA)), paramyxovirus, lentivirus, or rhabdovirus (such as vesicular stomatitis virus (VSV)) vector i.e. the vector may be derived from any of the aforementioned viruses. In one embodiment of the invention, the viral vector is an adenovirus. In another embodiment of the invention, the viral vector is a pox virus, e.g. MVA.

Adenoviruses are particularly suitable for use as a gene transfer vector because of its mid-sized genome, ease of manipulation, high titre, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP is particularly efficient during the late phase of infection, and all the mRNAs transcribed from this promoter possess a 5c-tripartite leader (TPL) sequence which makes them preferred mRNAs for translation. Replication-deficient adenovirus, which are created by from viral genomes that are deleted for one or more of the early genes are particularly useful, since they have limited replication and less possibility of pathogenic spread within a vaccinated host and to contacts of the vaccinated host.

### Other Polynucleotide Delivery

The expression construct comprising one or more polynucleotide sequences may simply consist of naked recombinant DNA plasmids. See Ulmer et al., 1993, Science 259:1745-1749 and reviewed by Cohen, 1993, Science 259:1691-1692. Transfer of the construct may be performed, for example, by any method which physically or chemically permeabilises the cell membrane. This is particularly applicable for transfer in vitro but it may be applied to in vivo use as well. It is envisioned that DNA encoding a gene of interest may also be transferred in a similar manner in vivo and express the gene product. Multiple delivery systems have been used to deliver DNA molecules into animal models and into man. Some products based on this technology have been licensed for use in animals, and others are in phase 2 and 3 clinical trials in man.

### RNA Delivery

The expression construct comprising one or more polynucleotide sequences may consist of naked, recombinant DNA-derived RNA molecules (Ulmer et al., 2012, Vaccine 30:4414-4418). As for DNA-based expression constructs, a variety of methods can be utilized to introduce RNA molecules into cells in vitro or in vivo. The RNA-based constructs can be designed to mimic simple messenger RNA (mRNA) molecules, such that the introduced biological molecule is directly translated by the host cell's translation machinery to produce its encoded polypeptide in the cells to which it has been introduced. Alternatively, RNA molecules may be designed in a manner that allows them to self-amplify within cells they are introduced into, by incorporating into their structure genes for viral RNA-dependent RNA polymerases. Thus, these types of RNA molecules, known as self-amplifying mRNA (SAM™) molecules (Geall et al. 2012, PNAS, 109:14604-14609), share properties with some RNA-based viral vectors. Either mRNA-based or SAM™ RNAs may be further modified (e.g., by alteration of their sequences, or by use of modified nucleotides) to enhance stability and translation (Schlake et al., RNA Biology, 9: 1319-1330), and both types of RNAs may be formulated (e.g., in emulsions (Brito et al., Molecular Therapy, 2014 22:2118-2129) or lipid nanoparticles (Kranz et al., 2006, Nature, 534:396-401)) to facilitate stability and/or entry into cells in vitro or in vivo. Myriad formulations of modified (and non-modified) RNAs have been tested as vaccines in animal models and in man, and multiple RNA-based vaccines are being used in ongoing clinical trials.

### Pharmaceutical Compositions

The fusion proteins, nucleic acids and vectors of the invention may be formulated for delivery in pharmaceutical compositions such as immunogenic compositions and vaccine compositions (all hereinafter “compositions of the invention”). Compositions of the invention suitably comprise a fusion protein, nucleic acid or vector of the invention together with a pharmaceutically acceptable carrier.

Thus, in an embodiment, there is provided an immunogenic pharmaceutical composition comprising a fusion protein, nucleic acid or vector of the invention together with a pharmaceutically acceptable carrier.

In another embodiment there is provided a vaccine composition comprising a fusion protein, nucleic acid or vector of the invention together with a pharmaceutically acceptable carrier. Preparation of pharmaceutical compositions is generally described in, for example, Powell & Newman, eds., Vaccine Design (the subunit and adjuvant approach), 1995. Compositions of the invention may also contain other compounds, which may be biologically active or inactive. Suitably, the composition of the invention is a sterile composition suitable for parenteral administration.

In certain preferred embodiments of the present invention, pharmaceutical compositions of the invention are provided which comprise one or more (e.g. one) fusion proteins of the invention in combination with a pharmaceutically acceptable carrier.

In certain preferred embodiments of the present invention, compositions of the invention are provided which comprise one or more (e.g. one) nucleic acids encoding a fusion protein of the invention or one or more (e.g., one) vectors of the invention in combination with a pharmaceutically acceptable carrier.

The compositions of the invention may comprise one or more (e.g., one) polynucleotide and one or more (e.g., one) fusion protein components. Alternatively, the compositions may comprise one or more (e.g., one) vector and one or more (e.g., one) fusion protein components. Alternatively, the compositions may comprise one or more (e.g., one) vector and one or more (e.g., one) polynucleotide components. Such compositions may provide for an enhanced immune response.

### Pharmaceutically Acceptable Salts

It will be apparent that a composition of the invention may contain pharmaceutically acceptable salts of the nucleic acids or fusion proteins provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

### Pharmaceutically Acceptable Carriers

While many pharmaceutically acceptable carriers known to those of ordinary skill in the art may be employed in the compositions of the invention, the optimal type of carrier used will vary depending on the mode of administration. Compositions of the present invention may be formulated for any appropriate manner of administration, including for example, parenteral, topical, oral, nasal, intravenous, intracranial, intraperitoneal, subcutaneous or intramuscular administration, preferably parenteral e.g., intramuscular, subcutaneous or intravenous administration. For parenteral administration, the carrier preferably comprises water and may contain buffers for pH control, stabilising agents e.g., surfactants and amino acids and tonicity modifying agents e.g., salts and sugars. If the composition is intended to be provided in lyophilised form for dilution at the point of use, the formulation may contain a lyoprotectant e.g., sugars such as trehalose. For oral administration, any of the above carriers or a solid carrier, such as mannitol, lactose, starch, magnesium stearate, sodium saccharine, talcum, cellulose, glucose, sucrose, and magnesium carbonate, may be employed.

Thus, compositions of the invention may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, solutes that render the formulation isotonic, hypotonic or weakly hypertonic with the blood of a recipient, suspending agents, thickening agents and/or preservatives. Alternatively, compositions of the invention may be formulated as a lyophilizate.

### Immunostimulants

Compositions of the invention may also comprise one or more immunostimulants. An immunostimulant may be any substance that enhances or potentiates an immune response (antibody and/or cell-mediated) to an exogenous antigen. Examples of immunostimulants, which are often referred to as adjuvants in the context of vaccine formulations, include aluminium salts such as aluminium hydroxide gel (alum) or aluminium phosphate, saponins including QS21, immunostimulatory oligonucleotides such as CPG, oil-in-water emulsion (e.g., where the oil is squalene), aminoalkyl glucosaminide 4-phosphates, lipopolysaccharide or a derivative thereof e.g., 3-de-O-acylated monophosphoryl lipid A (3D-MPL®) and other TLR4 ligands, TLR7 ligands, TLR8 ligands, TLR9 ligands, IL-12 and interferons. Thus, suitably the one or more immunostimulants of the composition of the invention are selected from aluminium salts, saponins, immunostimulatory oligonucleotides, oil-in-water emulsions, aminoalkyl glucosaminide 4-phosphates, lipopolysaccharides and derivatives thereof and other TLR4 ligands, TLR7 ligands, TLR8 ligands and TLR9 ligands. Immunostimulants may also include monoclonal antibodies which specifically interact with other immune components, for example monoclonal antibodies that block the interaction of immune checkpoint receptors, including PD-1 and CTLA4.

In the case of recombinant-nucleic acid methods of delivery (e.g., DNA, RNA, viral vectors), the genes encoding protein-based immunostimulants may be readily delivered along with the genes encoding fusion proteins of the invention.

### Sustained Release

The compositions described herein may be administered as part of a sustained-release formulation (i.e., a formulation such as a capsule, sponge, patch or gel (composed of polysaccharides, for example)) that effects a slow/sustained release of compound following administration.

### Storage and Packaging

Compositions of the invention may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a composition of the invention may be stored in a freeze-dried condition requiring only the addition of a sterile liquid carrier (such as water or saline for injection) immediately prior to use.

### Dosage

The amount of nucleic acid, fusion protein or vector in each composition of the invention may be prepared in such a way that a suitable dosage for therapeutic or prophylactic use will be obtained. Factors such as solubility, bioavailability, biological half-life, route of administration, product shelf life, as well as other pharmacological considerations will be contemplated by one skilled in the art of preparing such compositions, and as such, a variety of dosages and treatment regimens may be desirable.

Typically, compositions comprising a therapeutically or prophylactically effective amount deliver about 0.1 ug to about 1000 ug of fusion protein of the invention per administration, more typically about 2.5 ug to about 100 ug of fusion protein per administration. If delivered in the form of short, synthetic long peptides, doses could range from 1 to 200 ug/peptide/dose. In respect of polynucleotide compositions, these typically deliver about 10 ug to about 20 mg of the nucleic acid of the invention per administration, more typically about 0.1 mg to about 10 mg of the nucleic acid of the invention per administration.

### Diseases to be Treated or Prevented

As noted elsewhere, SEQ ID NOs. 1-8 are polypeptide sequences corresponding to CLT antigens of the fusion proteins of the invention which are over-expressed in cutaneous melanoma.

In one embodiment, the invention provides a fusion protein, nucleic acid, vector or composition of the invention for use in medicine.

Further aspects of the invention relate to a method of raising an immune response in a human which comprises administering to said human the fusion protein, nucleic acid, vector or composition of the invention.

The present invention also provides a fusion protein, nucleic acid, vector or composition of the invention for use in raising an immune response in a human.

The use of the fusion protein, nucleic acid, vector or composition in raising an immune response in a human against a cancer depends on corresponding antigenic sequences (or one or more of them) being expressed by the cancer. Thus, there is a relationship between the design of the fusion protein, nucleic acid, vector or composition and the antigenic sequences that the cancer expresses or is likely to express. Suitably the immune response is raised against a cancer expressing a corresponding sequence selected from (a) to (f), optionally (g) and (h) or variant or immunogenic fragment thereof. In this context, “corresponding” means that if the tumor expresses (or is likely to express), say, SEQ ID NO. A (A being one of SEQ ID NOs. 1-8) or a variant or immunogenic fragment thereof, then the fusion protein, nucleic acid, vector or composition of the invention and medicaments involving these will include SEQ ID NO. A or a variant or immunogenic fragment thereof. The inclusion of a number of antigen sequences potentially makes possible a greater immune response against a cancer or an immune response against cancer in a wider range of patients.

Suitably the immune response comprises CD8+ T-cell, a CD4+ T-cell and/or an antibody response, particularly CD8+ cytolytic T-cell response and a CD4+ helper T-cell response.

Suitably the immune response is raised against a tumor, particularly one expressing a sequence selected from (a) to (f), optionally (g) and (h) or variant or immunogenic fragment thereof.

In a preferred embodiment, the tumor is a melanoma tumor e.g. a cutaneous melanoma tumor.

The tumor may be a primary tumor or a metastatic tumor.

Further aspects of the invention relate to a method of treating a human patient suffering from cancer wherein the cells of the cancer express a sequence selected from SEQ ID NOs. 1-8 and immunogenic fragments and variants of any one thereof, or of preventing a human from suffering from cancer which cancer would express a sequence selected from SEQ ID NOs. 1-8 and immunogenic fragments and variants of any one thereof, which method comprises administering to said human a fusion protein, nucleic acid, vector or composition of the invention.

The present invention also provides a fusion protein, nucleic acid, vector or composition of the invention for use in treating or preventing cancer in a human, wherein the cells of the cancer express a corresponding sequence selected from SEQ ID NOs. 1-8 and immunogenic fragments of any one thereof.

The present invention also provides a method of treating a human suffering from cancer, comprising the steps of: (a) determining if the cells of said cancer express a polypeptide sequence selected from antigenic polypeptides (a) to (h) or a nucleic acid encoding said antigenic polypeptide or variant or immunogenic fragment thereof; and if so, (b) administering to said human a corresponding fusion protein, nucleic acid, vector, composition according to the invention.

Transcripts corresponding to SEQ ID NOs. 14 and 20 were also overexpressed in uveal melanoma. Consequently, in an alternative embodiment, the tumor is a uveal melanoma tumor and/or the tumor expresses a sequence selected from SEQ ID NOs. 1, 3 and 4. Thus, fusion proteins of the present invention may therefore be indicated in subjects having uveal cancer.

The words “prevention” and “prophylaxis” are used interchangeably herein.

### Treatment and Vaccination Regimes

A therapeutic regimen may involve either simultaneous (such as co-administration) or sequential (such as a prime-boost) delivery of (i) a fusion protein, nucleic acid or vector of the invention with (ii) one or more further fusion proteins, nucleic acids or vectors of the invention and/or (iii) a further component such as a variety of other therapeutically useful compounds or molecules such as antigenic proteins optionally simultaneously administered with adjuvant. Examples of co-administration include homo-lateral co-administration and contra-lateral co-administration. “Simultaneous” administration suitably refers to all components being delivered during the same round of treatment. Suitably all components are administered at the same time (such as simultaneous administration of both DNA and protein), however, one component could be administered within a few minutes (for example, at the same medical appointment or doctor's visit) or within a few hours.

A “priming” or first administration of a fusion protein, nucleic acid or vector of the invention may be followed by one or more “boosting” or subsequent administrations of a fusion protein, nucleic acid or vector of the invention (“prime and boost” method). The fusion protein, nucleic acid or vector of the invention may be used in a prime-boost vaccination regimen. Both the prime and boost may be a fusion protein of the invention, the same fusion protein of the invention in each case. Both the prime and boost may be a fusion protein of the invention, where different fusion proteins of the invention are used in each case. Both the prime and boost may be a nucleic acid or vector of the invention, the same nucleic acid or vector of the invention in each case. Both the prime and boost may be a nucleic acid or vector of the invention, where different nucleic acids or vectors of the invention are used in each case. Alternatively, the prime may be performed using a nucleic acid or vector of the invention and the boost performed using a fusion protein of the invention or the prime may be performed using a fusion protein of the invention and the boost performed using a nucleic acid or vector of the invention. Usually the first or “priming” administration and the second or “boosting” administration are given about 1-12 weeks later, or up to 4-6 months later. Subsequent “booster” administrations may be given as frequently as every 1-6 weeks or may be given much later (up to years later).

Suitably, a prime fusion protein comprises six antigenic polypeptides (a) to (f) wherein the antigenic polypeptides (a) to (f) are arranged in the order from N to C of (a), (b), (c), (d), (e) and (f) as exemplified by CLT Antigen Fusion Protein 1 (SEQ ID NO. 76). Suitably a boost fusion protein comprises six antigenic polypeptides (a) to (f) wherein the antigenic polypeptides (a) to (f) are arranged in the order from N to C of (c), (f), (d), (b), (e) and (a) as exemplified by CLT Antigen Fusion Protein 2 (SEQ ID NO. 77). Preferably, (a) is present at the N terminus and (f) is present at the C terminal of the prime and (c) is present at the N terminus and (a) is present at the C terminal of the boost.

More suitably, a prime fusion protein comprises eight antigenic polypeptides (a) to (h) wherein the antigenic polypeptides (a) to (h) are arranged in the order from N to C of (a), (b), (g), (d), (e), (h), (c) and (f) as exemplified by CLT Antigen Fusion Protein 3 (SEQ ID NO. 78). Suitably the boost fusion protein comprises eight antigenic polypeptides (a) to (h) wherein the antigenic polypeptides (a) to (h) are arranged in the order from N to C of (c), (g), (a), (h), (e), (f), (d) and (b) as exemplified by CLT Antigen Fusion Protein 4 (SEQ ID NO. 79). Preferably, (a) is present at the N terminus and (f) is present at the C terminal of the prime and (c) is present at the N terminus and (b) is present at the C terminal of the boost.

### Antigen Combinations

The fusion proteins, nucleic acids or vectors of the invention can be used in combination with one or more other antigenic polypeptides (or polynucleotides or vectors encoding them) which cause an immune response to be raised against melanoma e.g. cutaneous or uveal melanoma. These other antigenic polypeptides could be derived from diverse sources, they could include well-described melanoma-associated antigens, such as GPR143, PRAME, MAGE-A3 or pMel (gp100). Alternatively they could include other types of melanoma antigens, including patient-specific neoantigens (Lauss et al. (2017). Nature Communications, 8(1), 1738. http://doi.org/10.1038/s41467-017-01460-0), retained-intron neoantigens (Smart et al. (2018). Nature Biotechnology. http://doi.org/10.1038/nbt.4239), spliced variant neoantigens (Hoyos et al., Cancer Cell, 34(2), 181-183. http://doi.org/10.1016/j.ccell.2018.07.008; Kahles et al. (2018). Cancer Cell, 34(2), 211-224.e6. http://doi.org/10.1016/j.ccell.2018.07.001), melanoma antigens that fit within the category known as antigens encoding T-cell epitopes associated with impaired peptide processing (TIEPPs; Gigoux, M., & Wolchok, J. (2018). JEM, 215, 2233, Marijt et al. (2018). JEM 215, 2325), or to-be discovered neoantigens (including CLT antigens). In addition, the antigenic peptides from these various sources could also be combined with (i) non-specific immunostimulant/adjuvant species and/or (ii) an antigen, e.g. comprising universal CD4 helper epitopes, known to elicit strong CD4 helper T-cells (delivered as a polypeptides, or as polynucleotides or vectors encoding these CD4 antigens), to amplify the anti-melanoma-specific responses elicited by co-administered antigens.

Nucleic acids and vectors comprising them may be provided which encode the aforementioned proteins.

Different proteins, nucleic acids or vectors may be formulated in the same formulation or in separate formulations.

More generally, when two or more components are utilised in combination, the components could be presented, for example:
(1) as two or more individual antigenic polypeptide components;
(2) as a fusion protein comprising both (or further) polypeptide components;
(3) as one or more polypeptide and one or more polynucleotide component;
(4) as two or more individual polynucleotide components;
(5) as a single polynucleotide encoding two or more individual polypeptide components; or
(6) as a single polynucleotide encoding a fusion protein comprising both (or further) polypeptide components.
   For convenience, it is often desirable that when a number of components are present they are contained within a single fusion protein or a polynucleotide encoding a single fusion protein (see below). All components may be provided within a single fusion protein. Alternatively, all components may be provided as polynucleotides (e.g., a single polynucleotide, such as one encoding a single fusion protein).

### EXAMPLES

### Example 1—CLT Identification

The objective was to identify cancer-specific transcripts that entirely or partially consist of LTR elements.

As a first step, we de novo assembled a comprehensive pan-cancer transcriptome. To achieve this, RNA-sequencing reads from 768 patient samples, obtained from The Cancer Genome Atlas (TCGA) consortium to represent a wide variety of cancer types (24 gender-balanced samples from each of 32 cancer types (31 primary and 1 metastatic melanoma); Table S1), were used for genome-guided assembly. The gender-balanced samples (excluding gender-specific tissues) were adapter and quality (Q20) trimmed and length filtered (both reads of the pair ≥35 nucleotides) using cutadapt (v1.13) (Marcel M., 2011, EMBnet J., 17:3) and kmer-normalized (k=20) using khmer (v2.0) (Crusoe et al., 2015, F1000Res., 4:900) for maximum and minimum depths of 200 and 3, respectively. Reads were mapped to GRCh38 using STAR (2.5.2b) with settings identical to those used across TCGA and passed to Trinity (v2.2.0) (Trinity, Grabherr, M. G., et al., 2011, Nat. Biotechnol., 29:644-52) for a genome-guided assembly with inbuilt in silico depth normalization disabled. The majority of assembly processes were completed within 256 GB RAM on 32-core HPC nodes, with failed processes re-run using 1.5 TB RAM nodes. Resulting contigs were poly(A)-trimmed (trimpoly within SeqClean v110222) and entropy-filtered (≥0.7) to remove low-quality and artefactual contigs (bbduk within BBMap v36.2). Per cancer type, the original 24 samples were quasi-mapped to the cleaned assembly using Salmon (v0.8.2 or v0.9.2) (Patro, R., et al., 2017, Nat. Methods, 14:417-419), with contigs found expressed at <0.1 transcripts per million (TPM) being removed. Those remaining were mapped to GRCh38 using GMAP (v161107) (Wu et al., 2005, Bioinf., 21:1859-1875), and contigs not aligning with ≥85% identity over ≥85% of their length were removed from the assembly. Finally, assemblies for all cancer types together were flattened and merged into the longest continuous transcripts using gffread (Cufflinks v2.2.1) (Trapnell et al., 2010, Nat. Biotech., 28:511-515). As this assembly process was specifically designed to enable assessment of repetitive elements, monoexonic transcripts were retained, but flagged. Transcript assembly completeness and quality was assessed by comparison with GENCODE v24basic and MiTranscriptome1 (Iyer et al. 2015, Nat. Genet., 47: 199-208). We compiled the list of unique splice sites represented within GENCODE and tested if the splice site was present within the transcriptome assembly within a 2-nucleotide grace window. This process resulted in the identification of 1,001,931 transcripts, 771,006 of which were spliced and 230,925 monoexonic.

Separately, the assembled contigs were overlaid with a genomic repeat sequence annotation to identify transcripts that contain an LTR element. LTR and non-LTR elements were annotated as previously described (Attig et al., 2017, Front. In Microbiol., 8:2489). Briefly, hidden Markov models (HMMs) representing known Human repeat families (Dfam 2.0 library v150923) were used to annotate GRCh38 using RepeatMasker Open-3.0 (Smit, A., R. Hubley, and P. Green, http://www.repeatmasker.org, 1996-2010), configured with nhmmer (Wheeler et al., 2013, Bioinform., 29:2487-2489). HMM-based scanning increases the accuracy of annotation in comparison with BLAST-based methods (Hubley et al., 2016, Nuc. Acid. Res., 44:81-89). RepeatMasker annotates LTR and internal regions separately, thus tabular outputs were parsed to merge adjacent annotations for the same element. This process yielded 181,967 transcripts that contained one or more, complete or partial LTR element.

Transcripts per million (TPM) were estimated for all transcripts using Salmon and expression within each cancer type was compared with expression across 811 healthy tissue samples (healthy tissue-matched controls for all cancer types, where available, from TCGA and, separately from, GTEx (The Genotype-Tissue Expression Consortium, 2015, Science, 348:648-60). Transcripts were considered expressed in cancer if detected at more than 1 TPM in any sample and as cancer-specific if the following criteria were fulfilled: i, expressed in of the 24 samples of each cancer type; ii, expressed at <10 TPM in ≥90% of all healthy tissue samples; iii, expressed in the cancer type of interest ≥3×the median expression in any control tissue type; and iv, expressed in the cancer type of interest ≥3×the ≥90th percentile of the respective healthy tissue, where available.

The list of cancer-specific transcripts was then intersected with the list of transcripts containing complete or partial LTR elements to produce a list of 5,923 transcripts that fulfilled all criteria (referred to as CLTs for Cancer-specific LTR element-spanning Transcripts).

Further curation was carried out on 403 CLTs specifically expressed in melanoma to exclude potentially misassembled contigs and those corresponding to the assembly of cellular genes. Additional manual assessment was conducted to ensure that splicing patterns were supported by the original RNA-sequencing reads from melanoma. CLTs were additionally triaged such that those where the median expression in any GTEx normal tissue exceeded 1 TPM were discarded.

Within the 403 CLTs for cutaneous melanoma, 97 CLTs passed these filters.

### Example 2—Immunopeptidomic Analysis

Mass spectrometry (MS)-based immunopeptidomics analysis is a powerful technology that allows for the direct identification of specific peptides associated with HLA molecules (pHLA) and presented on the cell surface. The technique consists of affinity purification of the pHLA from biological samples such as cells or tissues by anti-HLA antibody capture. The isolated HLA molecules and bound peptides are then separated from each other and the eluted peptides are analyzed by nano-ultra performance liquid chromatography coupled to mass spectrometry (nUPLC-MS) (Freudenmann et al., 2018, Immunology 154(3):331-345). In the mass spectrometer, specific peptides of defined charge-to-mass ratio (m/z) are selected, isolated, fragmented, and then subjected to a second round of mass spectrometry (MS/MS) to reveal the m/z of the resulting fragment ions. The fragmentation spectra (MS/MS) can then be interrogated to precisely identify the amino acid sequence of the selected peptide that gave rise to the detected fragment ions.

MS/MS spectral interpretation and subsequent peptide sequence identification relies on the match between experimental data and theoretical spectra created from peptide sequences found in a reference database. Although it is possible to search MS data by using pre-defined lists corresponding to all open reading frames (ORFs) derived from the known transcriptome or even the entire genome (Nesvizhskii et al., 2014, Nat. Methods 11: 1114-1125), interrogating these very large sequence databases leads to very high false discovery rates (FDR) that limit the identification of presented peptides. Further technical issues (e.g., mass of leucine=mass of isoleucine), and theoretical issues (e.g., peptide splicing (Liepe, et al., 2016, Science 354(6310): 354-358)) increase the limitations associated with use of very large databases, such as those produced from the known transcriptome or entire genome. Thus, in practice, it is exceptionally difficult to perform accurate immunopeptidomics analyses to identify novel antigens without reference to a well-defined set of potential polypeptide sequences (Li, et al., 2016, BMC Genomics 17 (Suppl 13):1031).

Bassani-Sternberg et al. (Bassani-Sternberg et al., 2016, Nature Commun., 7: 13404; database link: https://www.ebi.ac.uk/pride/archive/projects/PXD004894) interrogated MS/MS data collected from HLA-bound peptide samples derived from 25 cutaneous melanoma patients against the polypeptide sequences reported for the entire human proteome. These analyses revealed tens of thousands of peptides that matched to known human proteins. As expected, these peptides included peptides found within multiple tumor-associated antigens (TAA), including PRAME, MAGEA3, and TRPM1 (melastatin).

The inventors procured frozen tumor tissue from 6 patients diagnosed with melanoma. Samples between 0.05-1 g were homogenized, the lysate was centrifugate at high speed and the cleared lysate was mixed with protein A (ProA) beads covalently linked to an anti-human HLA class I monoclonal antibody (W6/32). The mixture was incubated overnight at 4° C. to improve HLA Class I molecule binding to antibody (Ternette et al., 2018 Proteomics 18, 1700465). The HLA Class I-bound peptides were eluted from the antibody by using 10% acetic acid, and the peptides were then separated from other high molecular mass components using reversed-phase column chromatography (Ternette et al., 2018). The purified, eluted peptides were subjected to nUPLC-MS, and specific peptides of defined charge-to-mass ratio (m/z) were selected within the mass spectrometer, isolated, fragmented, and subjected to a second round of mass spectrometry (MS/MS) to reveal the m/z of the resulting fragment ions (Ternette et al., 2018), producing an MS/MS dataset corresponding to the immunopeptidome for each of these tumor samples.

By applying detailed knowledge of immunopeptidomics evaluation, the inventors interrogated the spectra from the PXD004894 HLA Class I dataset for 25 melanoma patients (Bassani-Sternberg et al., 2016) and the spectra of the HLA-Class I dataset for the 6 melanoma patients prepared by the inventors with the CLT-derived ORFs (of Example 1). Three types of analyses were conducted:
Analysis A: Predicted ORFs of greater than 23 amino acid residues from a subset of approximately 1 dozen CLTs derived from those identified in Example 1 were concatenated into a single polypeptide file for each CLT, and these concatenated ORF polypeptides were interrogated against the PXD004894 HLA Class I dataset for 25 melanoma patients alongside all polypeptides found in the human proteome (UniProt database) by using the PEAKS™ software (v8.5, Bioinformatics Solutions Inc)
Analysis B: Polypeptide files consisting of each of the predicted ORFs of greater than 23 amino acid residues from a subset of approximately 1 dozen CLTs derived from those identified in Example 1 were interrogated against the PXD004894 HLA Class I dataset for 25 melanoma patients alongside all the polypeptides found in the human proteome (UniProt and masDB databases) by using the Mascot software
Analysis C: All predicted ORFs derived from the 97 CLTs identified in Example 1 of 10 or more amino acid residues in length, were interrogated against the PXD004894 HLA Class I dataset for 25 melanoma patients and the inventors' HLA Class I dataset for 6 melanoma patients alongside all the polypeptides found in the human proteome (UniProt) using PEAKS™ software (v8.5 and vX, Bioinformatics Solutions Inc)

Since the majority of Class I HLA-bound peptides found in cells are derived from constitutively expressed proteins, the simultaneous interrogation of these databases with the UniProt proteome helps to ensure that assignments of our CLT ORF sequences to MS/MS spectra are correct. The PEAKS software, like other MS/MS interrogation software, assigns a probability value (−10IgP; see Table 1) to each spectral assignment to quantify the assignment.

The results of these studies identified >50 individual peptides that were associated with the HLA Class I molecules immunoprecipitated from tumor samples from the 25 patients examined by Bassani-Sternberg et al. and the 6 melanoma patient samples in the inventors' dataset, that corresponded to the amino acid sequence of CLT-derived ORFs, and did not correspond to polypeptide sequences present within the known human proteome (UniProt and/or masDB).

Further manual review of the peptide spectra assigned by the PEAKS software was used to confirm assignment of spectra to peptides that were mapped to 8 CLT-derived ORFs, and thus defined as CLT antigens (Table 1; SEQ ID NOs. 1-8).

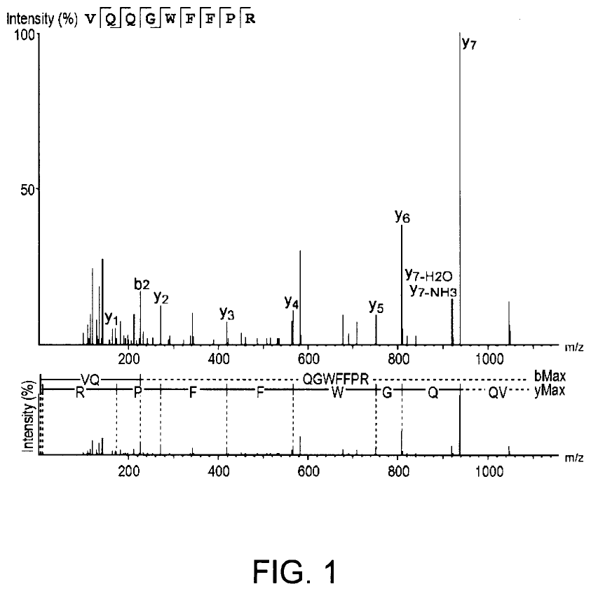

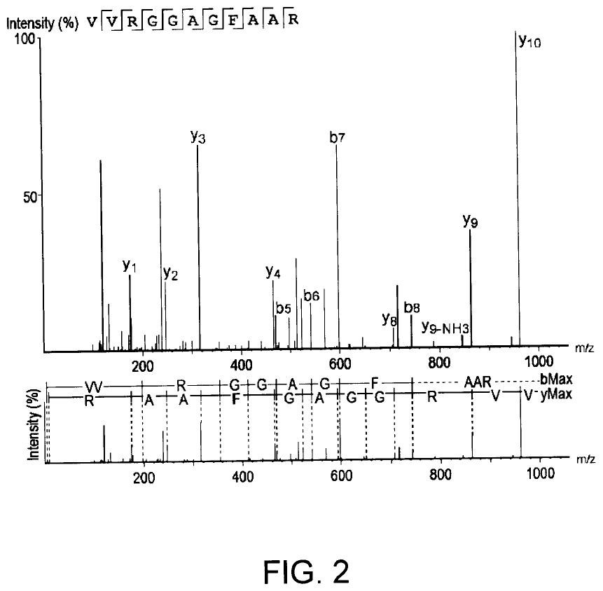

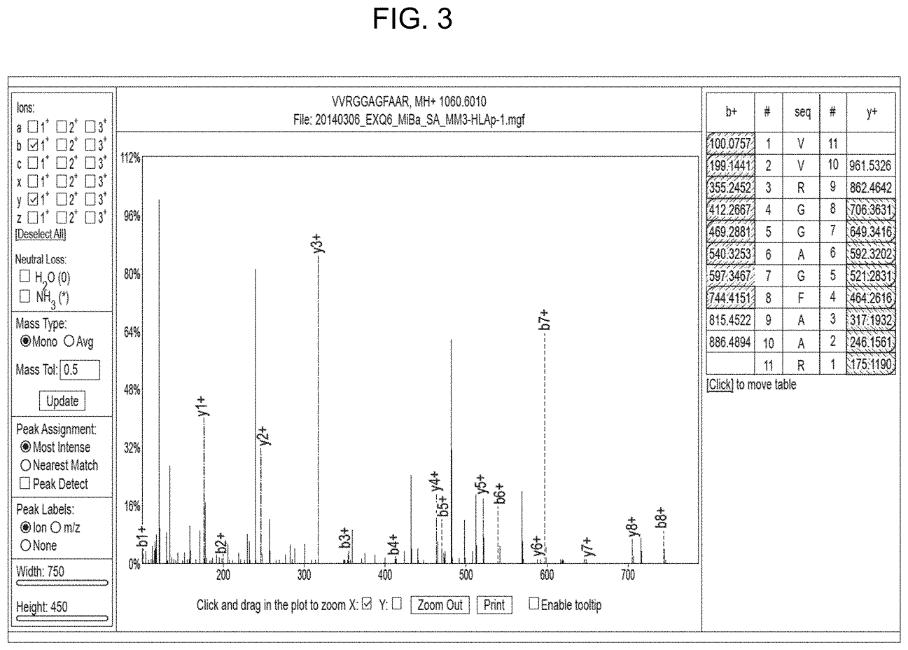

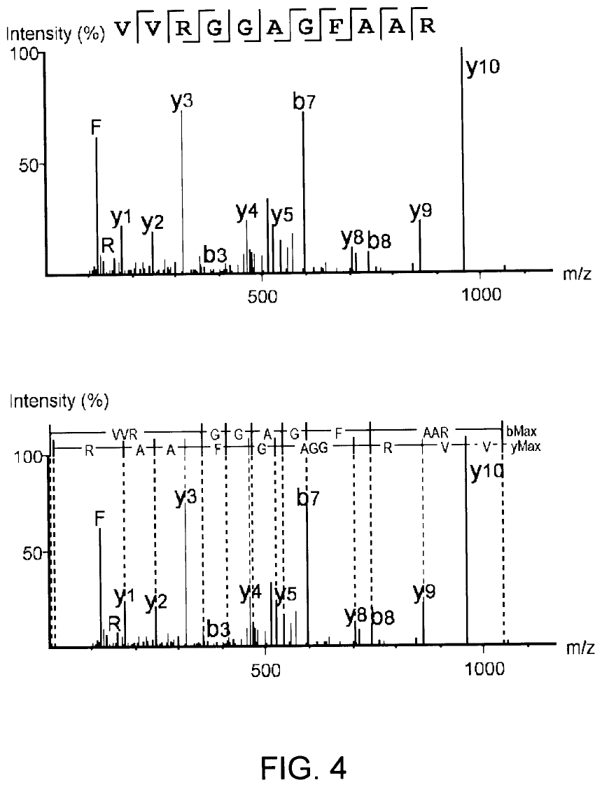

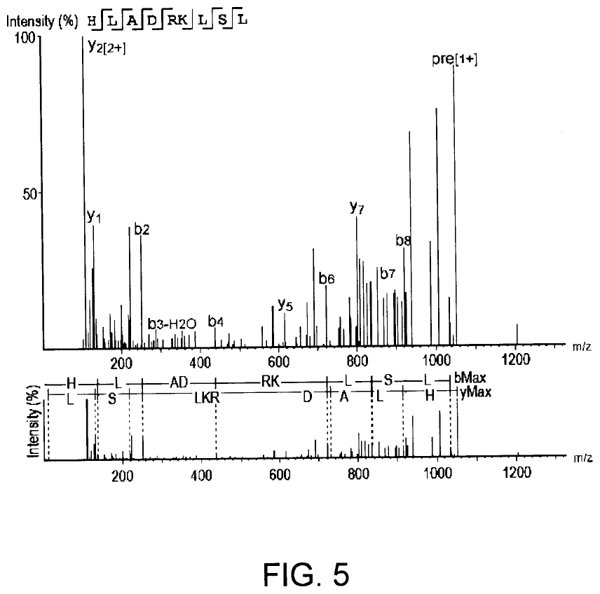

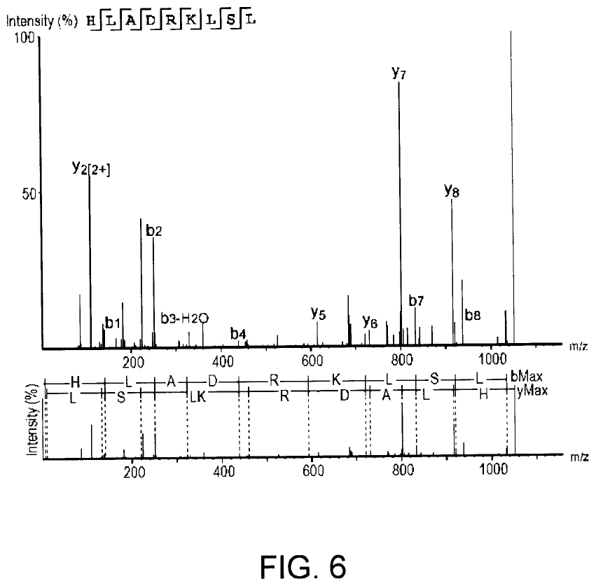

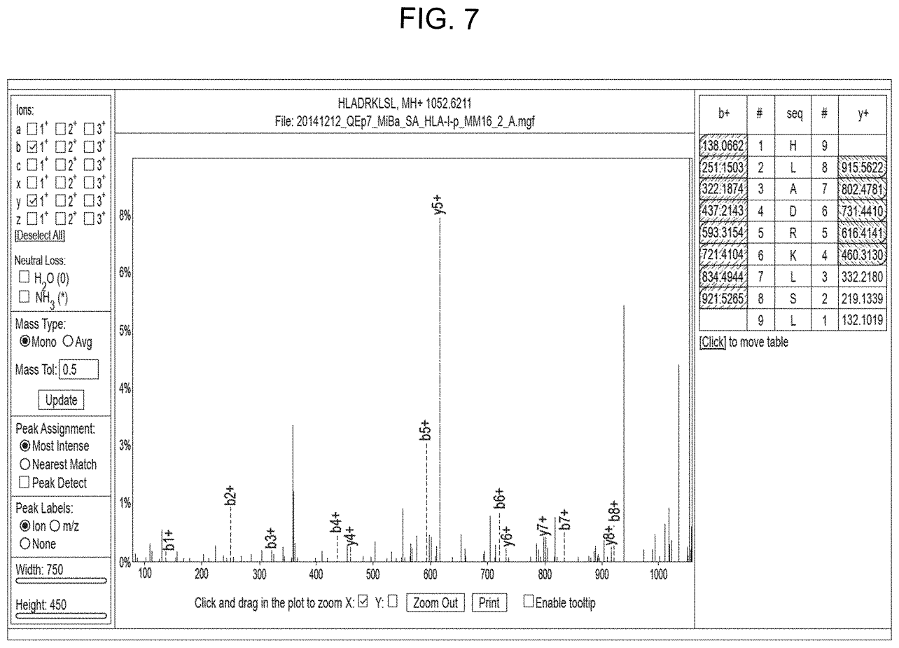

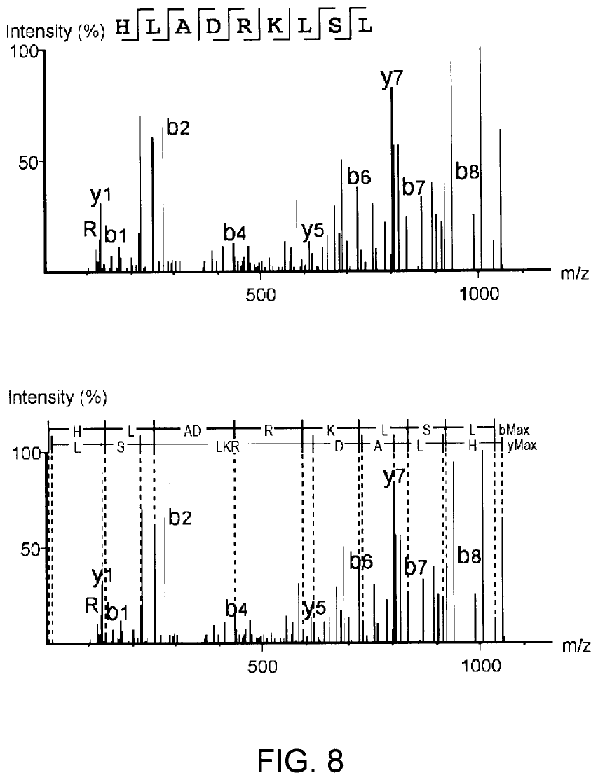

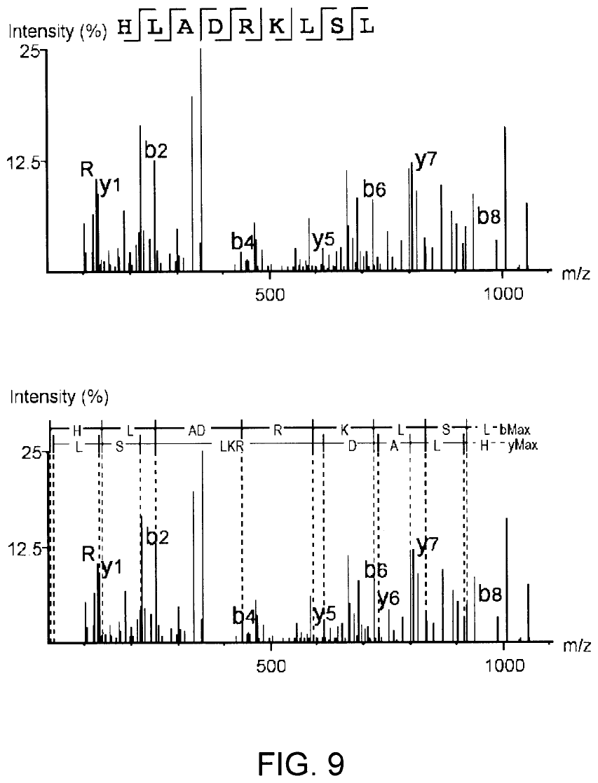

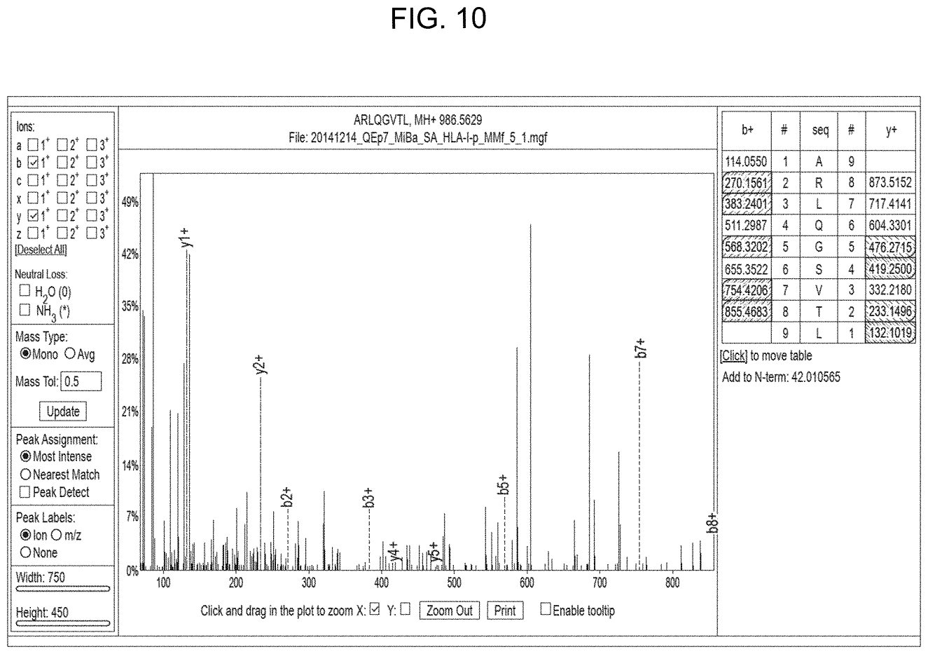

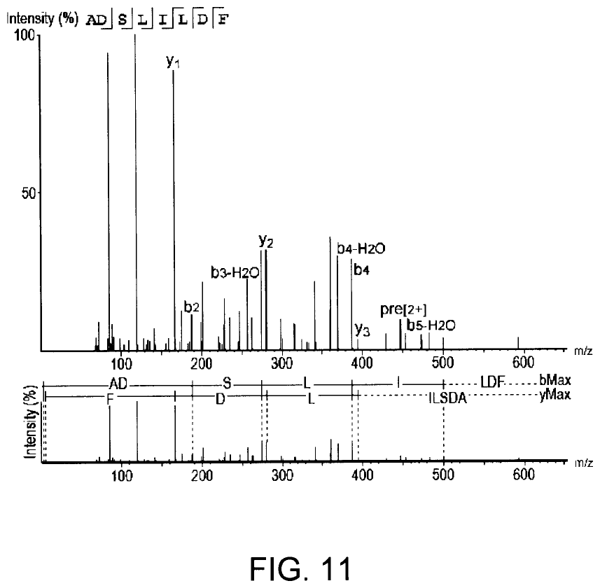

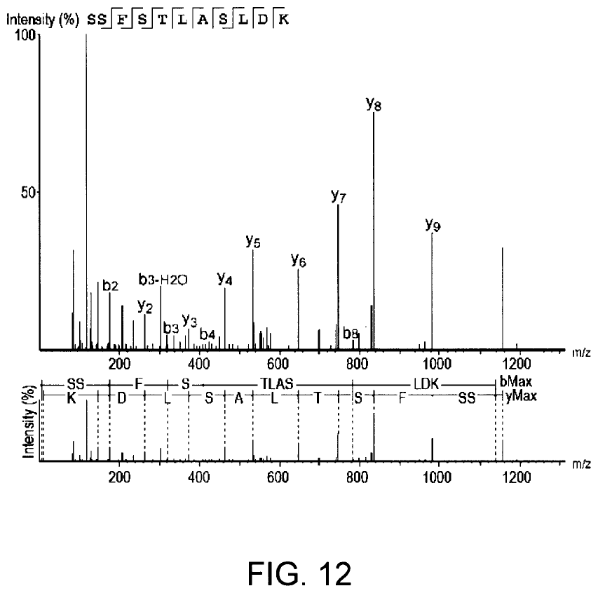

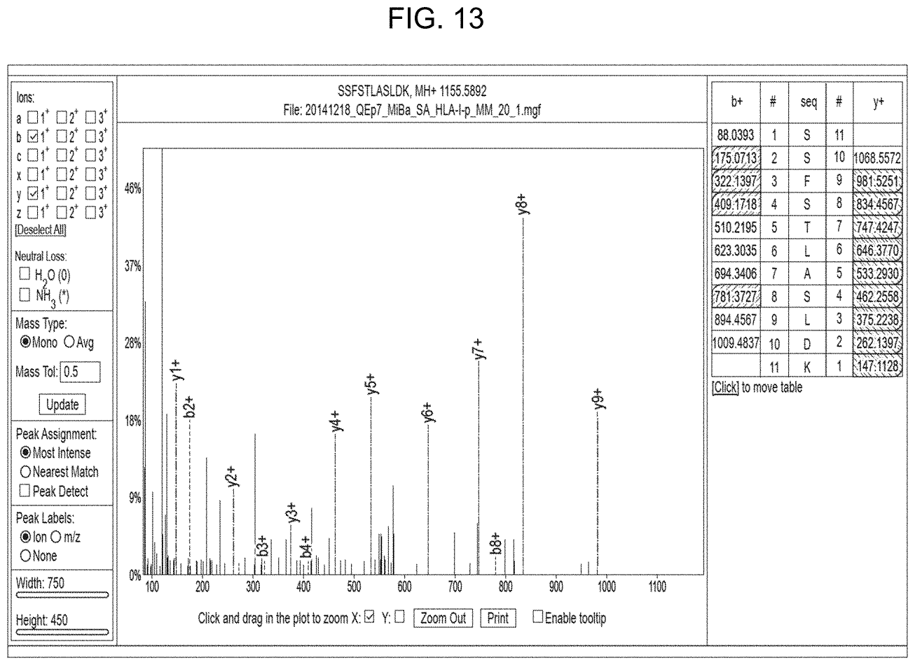

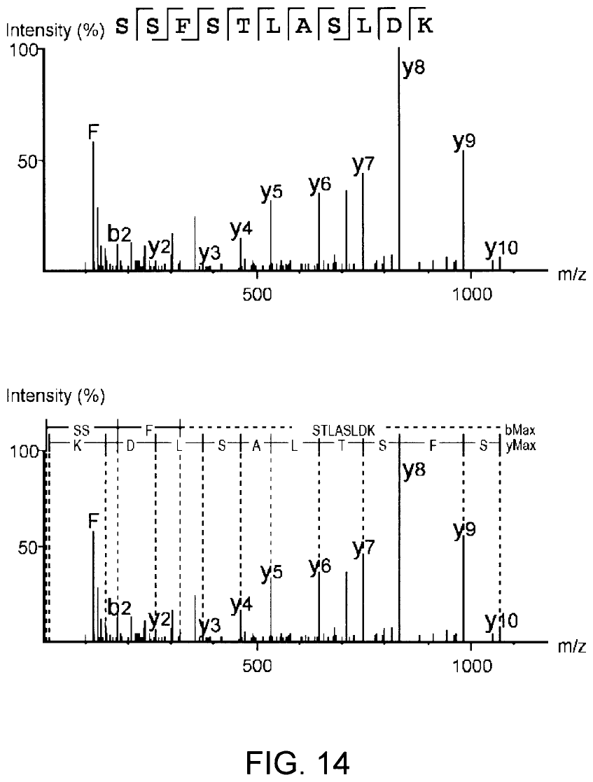

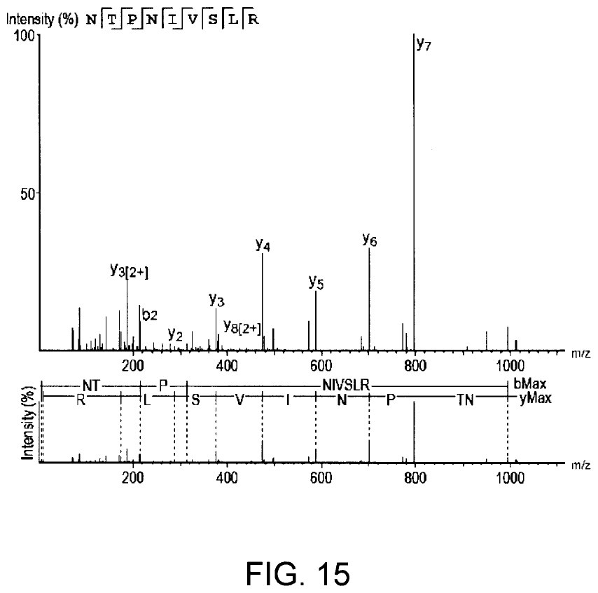

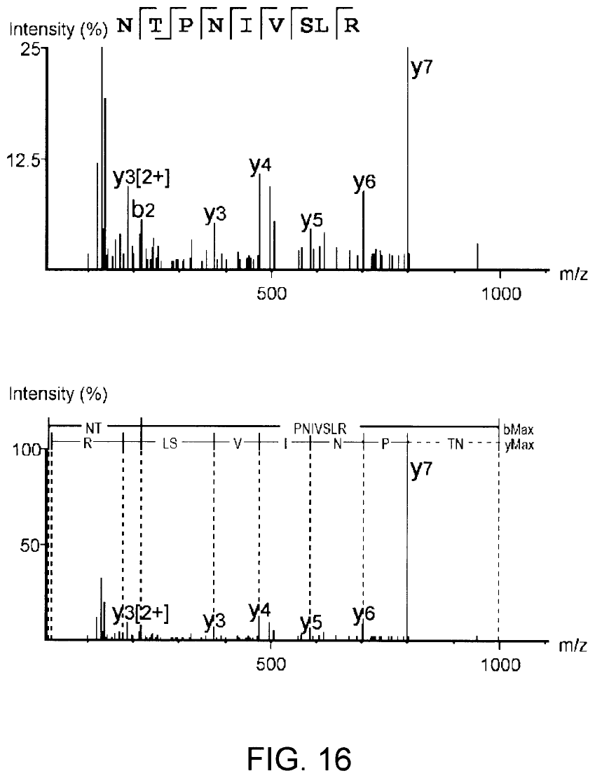

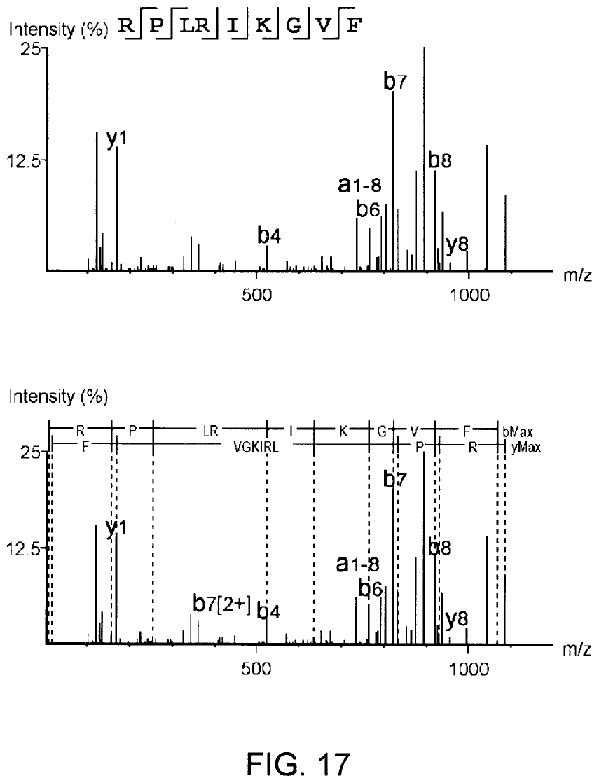

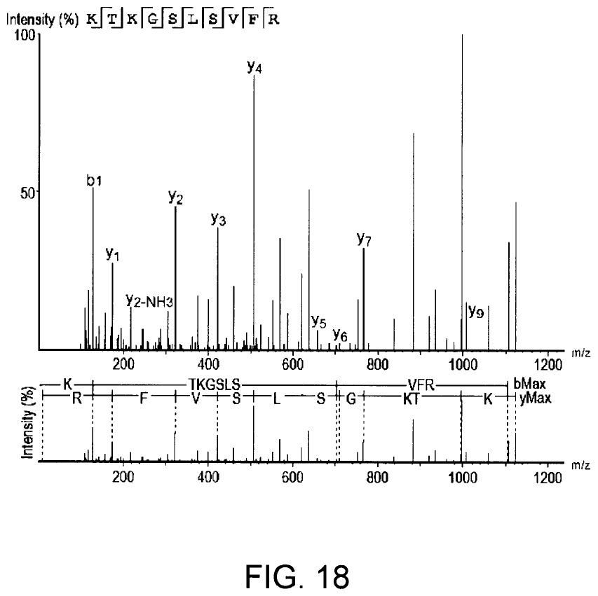

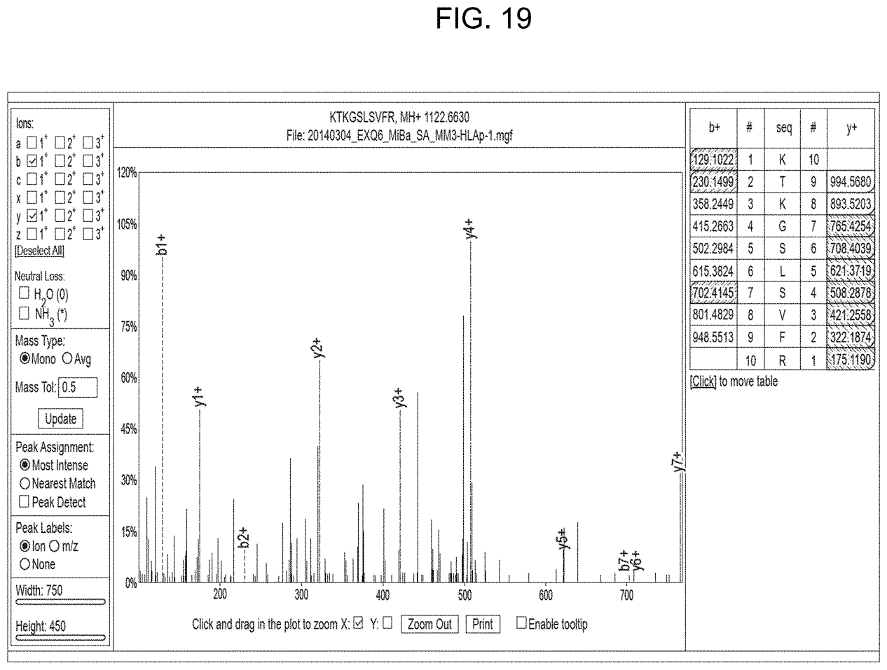

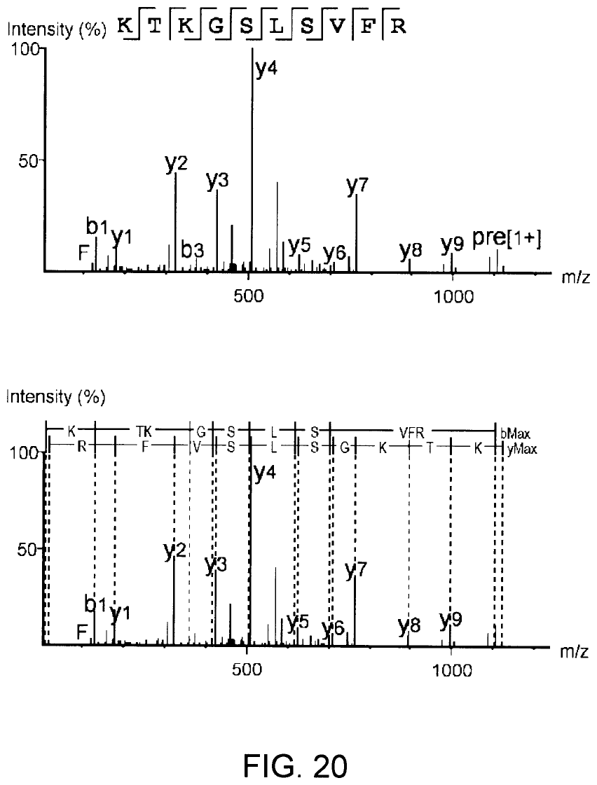

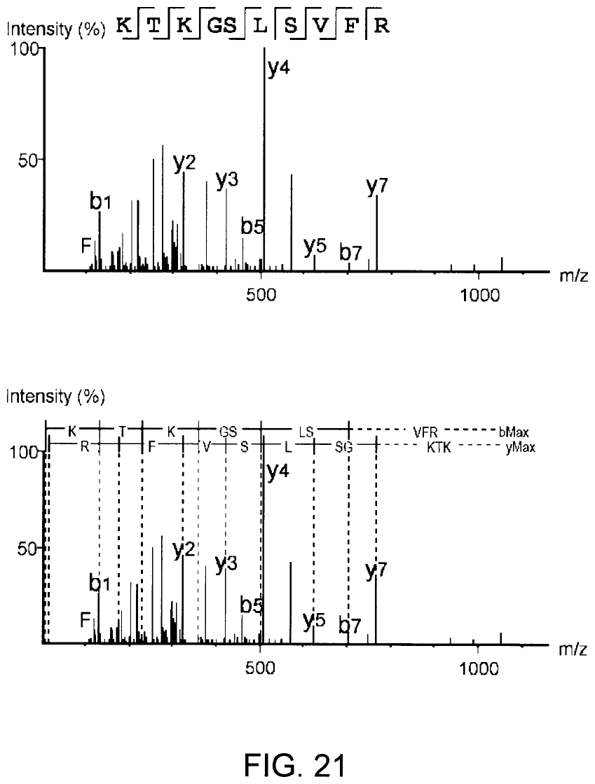

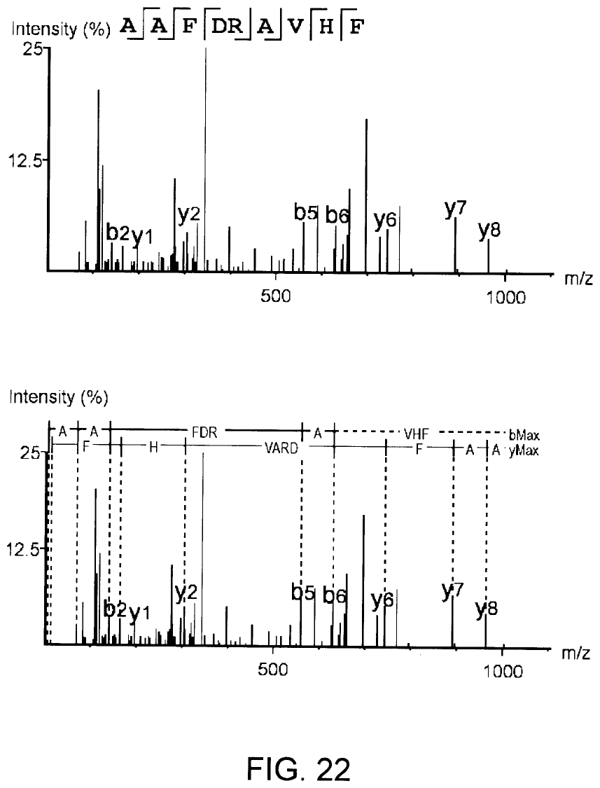

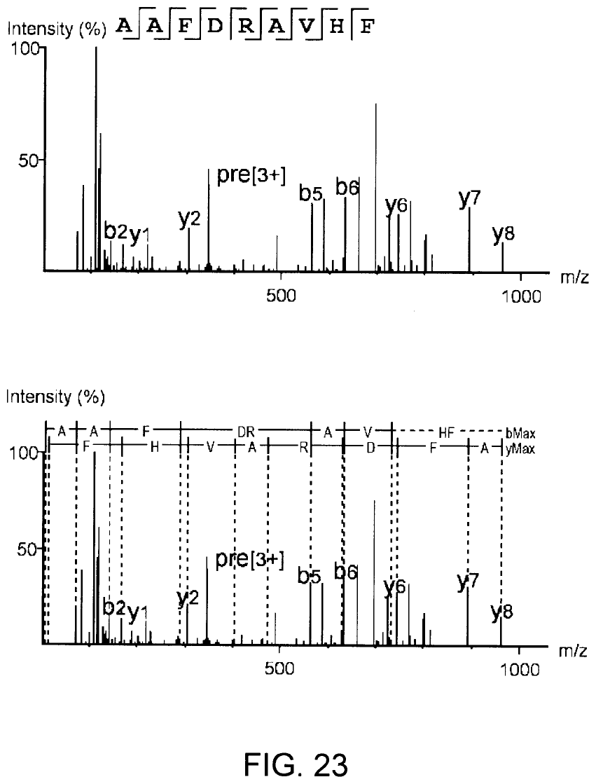

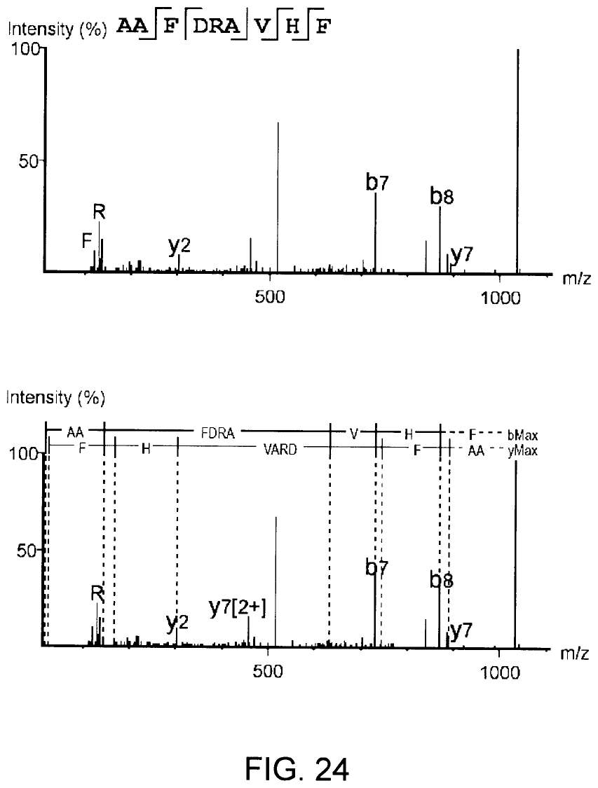

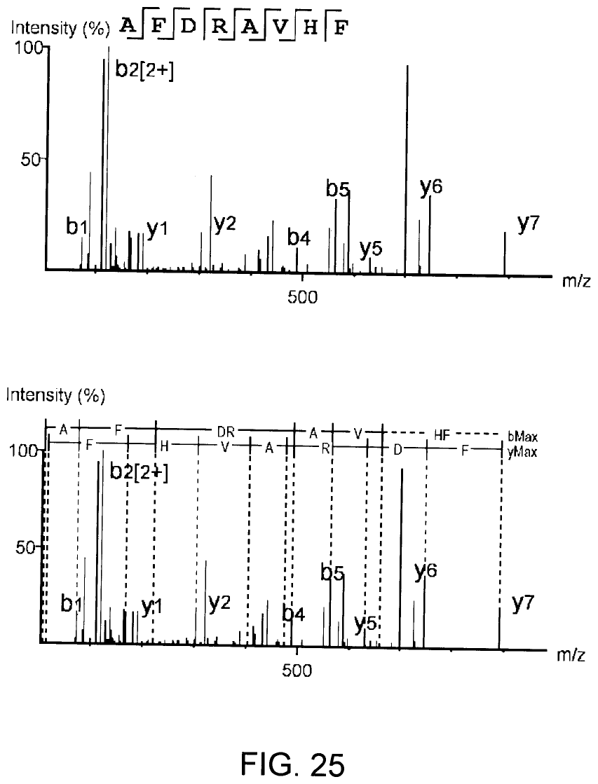

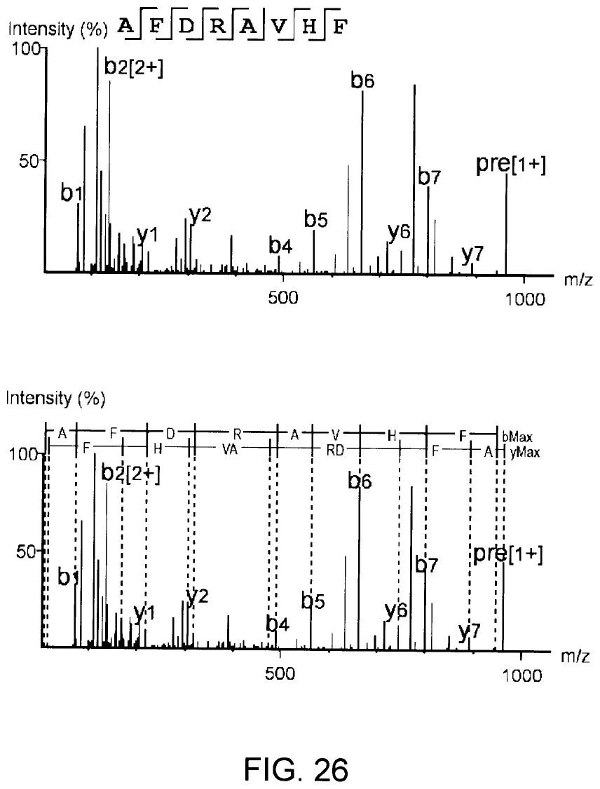

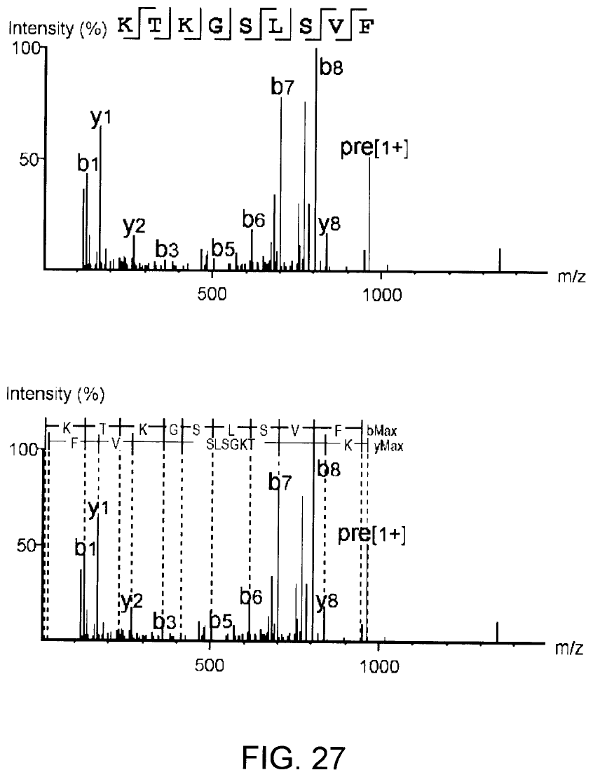

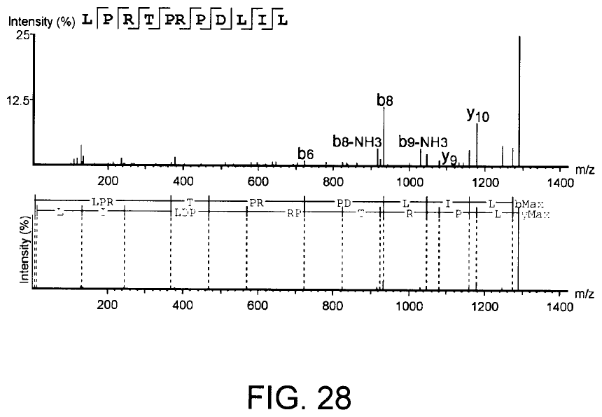

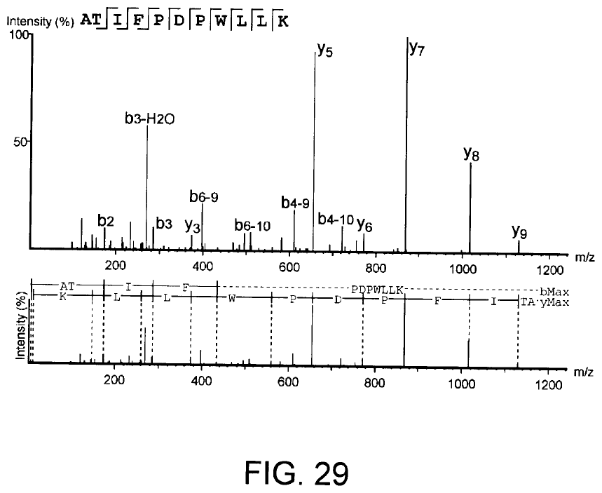

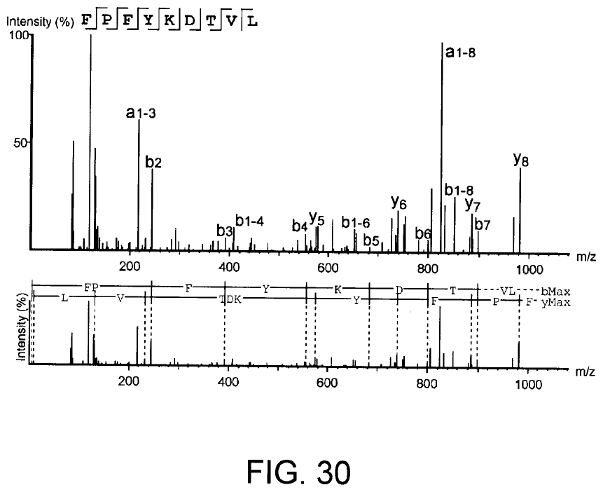

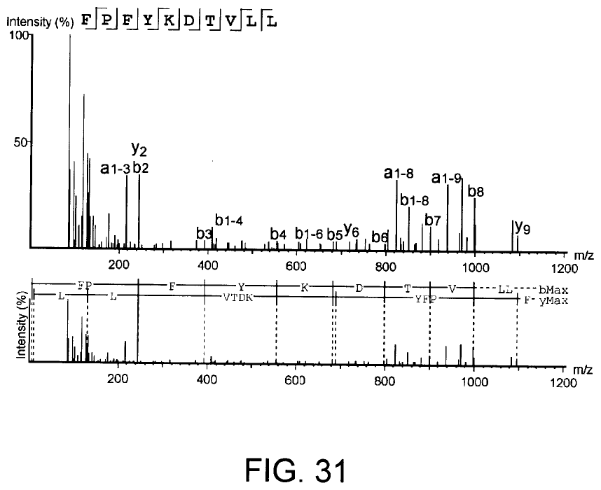

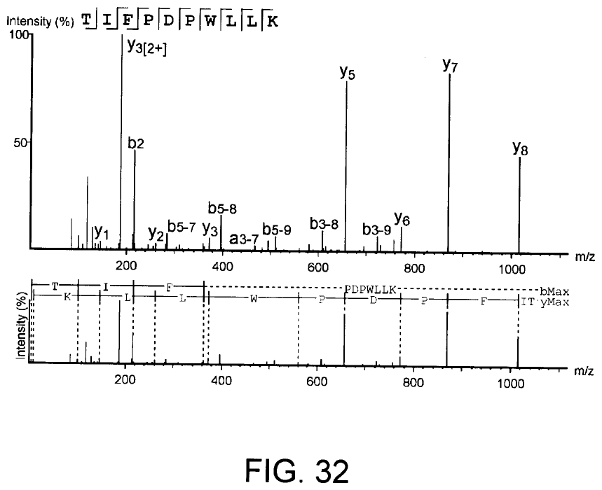

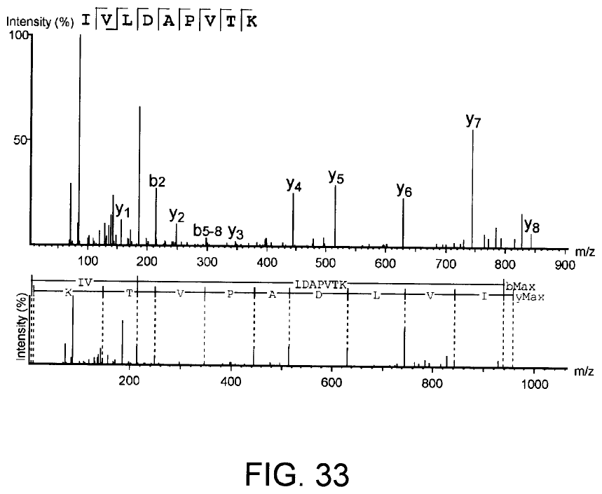

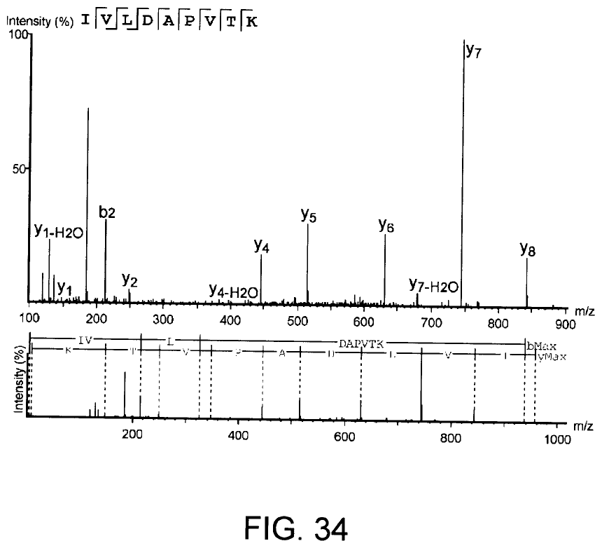

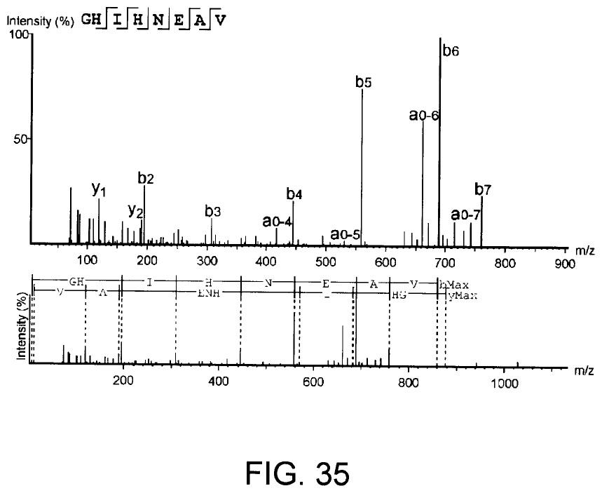

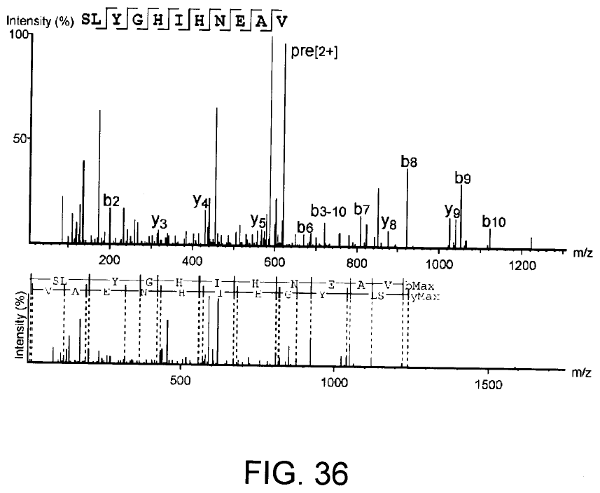

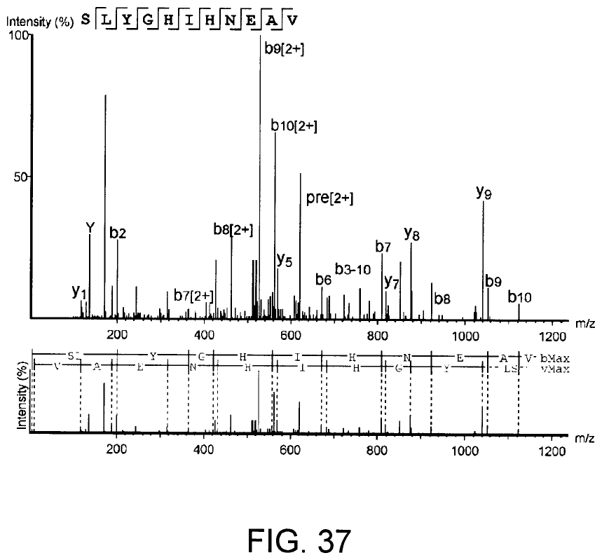

The detection of these peptides associated with the HLA Class I molecules confirms, that the 8 ORFs from which they were derived, were first translated in melanoma tissues, processed through the HLA Class I pathway and finally presented to the immune system in a complex with HLA Class I molecules. Table 1 shows the properties of the peptides found in the CLT antigens. FIGS. 1-37 show representative MS/MS spectra from each of the peptides shown in Table 1. The figures show fragment spectra for indicated peptide sequences as detected in individual patient SKCM tumors by nUPLC-MS² (images extracted by PEAKS™ software from the inventors' internal dataset or from Bassani-Sternberg et al. dataset stored in PRIDE). All fragments that have been detected are indicated in the peptide sequence above the spectrum and the most abundant fragment ions are assigned in each spectrum. In FIGS. 1-2, 4-6, 8-9, 11-12, 14-37, the lower panel of the figures illustrates the peptide sequences assigned to the MS/MS spectrum, whereas similar data are shown in tabular form on the right side of FIGS. 3, 7, 10, 13 and 19. Fragment ions are annotated as follows: b: N-terminal fragment ion; y: C-terminal fragment ion; —H₂O: water loss; —NH₃: loss of ammonia; [2+]: doubly charged peptide ion; pre: unfragmented precursor peptide ion. Consistent with the high −10IgP scores assigned to the peptides in Table 1, these spectra contain numerous fragments that precisely match the sequences of the peptides (SEQ ID NOs. 9-12, 18-19, 31-32, 36-39, 45, 48-54) that we discovered in these analyses.

All of the peptides detected in association with HLA Class I molecules from Table 1 that were 9 amino acid residues or more in length were assessed to determine their predicted strength of binding to HLA Class I type A and B supertypes by using the NetMHCpan 4.0 prediction software (http://www.cbs.dtu.dk/services/NetMHCpan/). The results of these prediction studies showed that all of the 17 peptides (or 9-mers contained within each full sequence) were predicted to bind to at least one of the supertypes tested (see Table 2). Amongst these, many of the sequences were predicted to bind with high confidence (low % rank scores) to specific types within the HLA Class I supertypes examined. The fact that all of the detected peptides were expected to bind to the standard set of HLA types provides additional validation around their detection. Moreover, every peptide discovered in a tumor sample from the inventors' dataset was predicted by NetMHCpan 4.0 to bind to one of the HLA types we detected in the patient sample. HLA types were not reported by Bassani-Sternberg et al. (2016, Nature Commun., 7: 13404) for every patient associated with the peptides we discovered, but where this was reported, we found matches between the known and predicted HLA types.

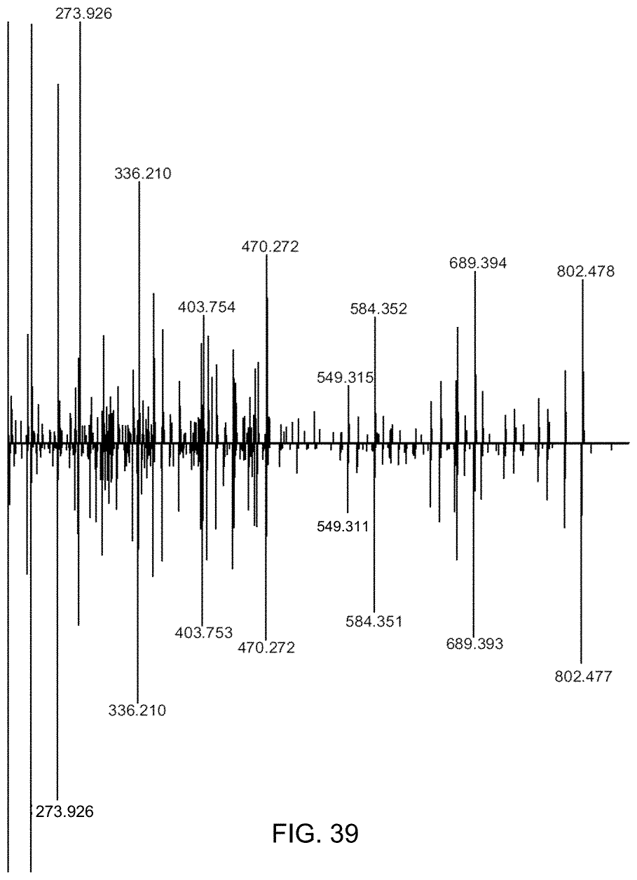

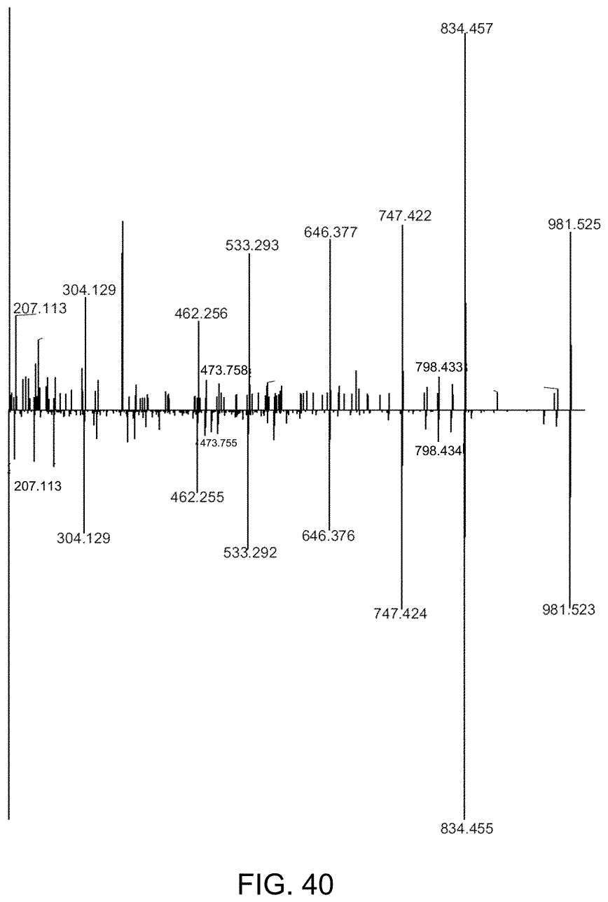

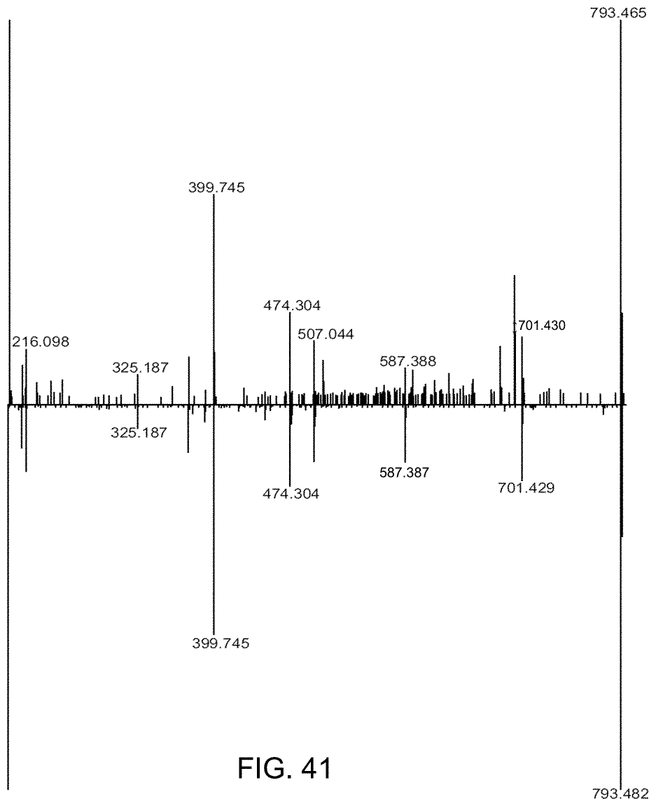

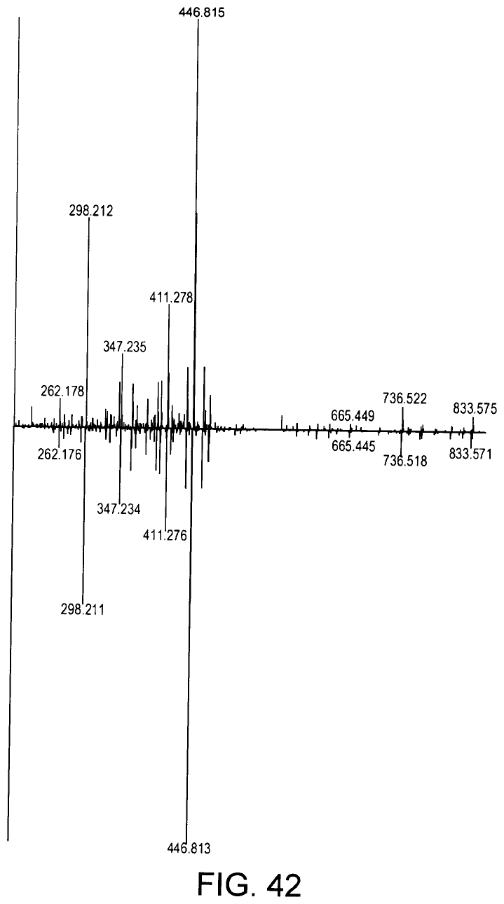

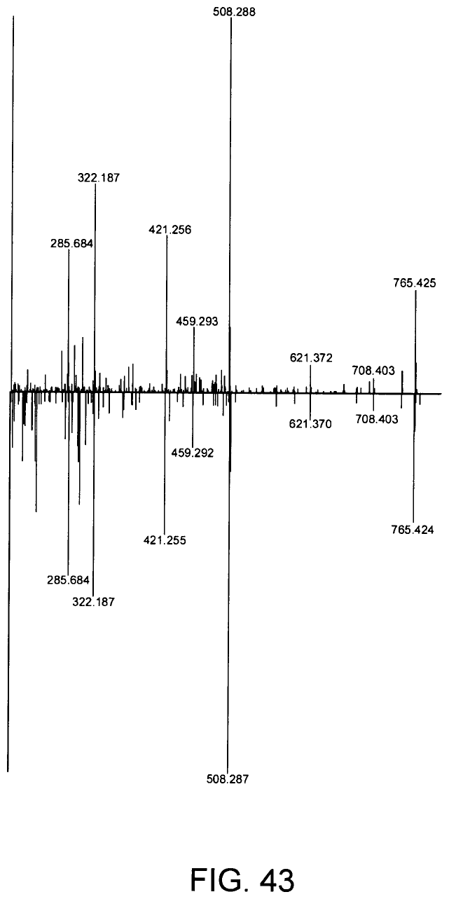

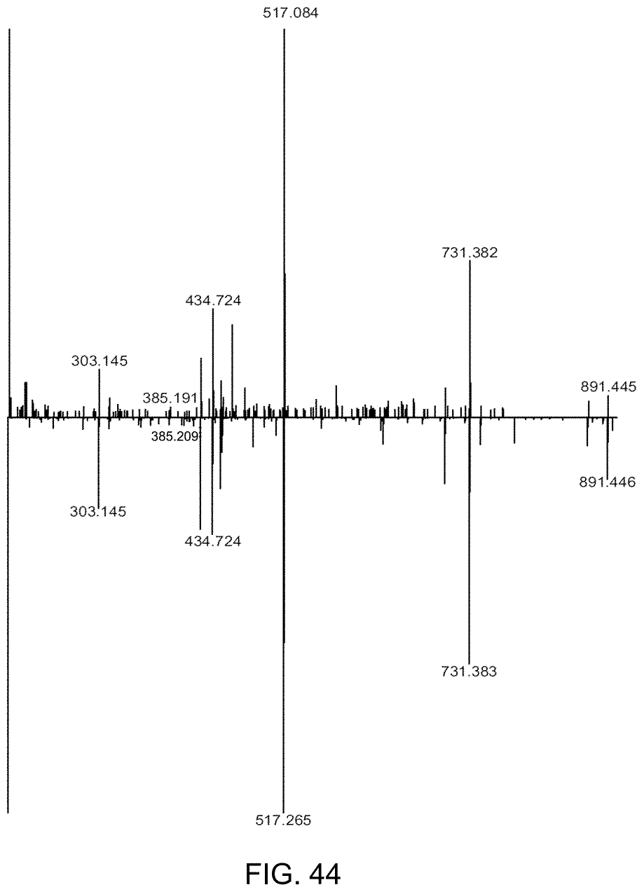

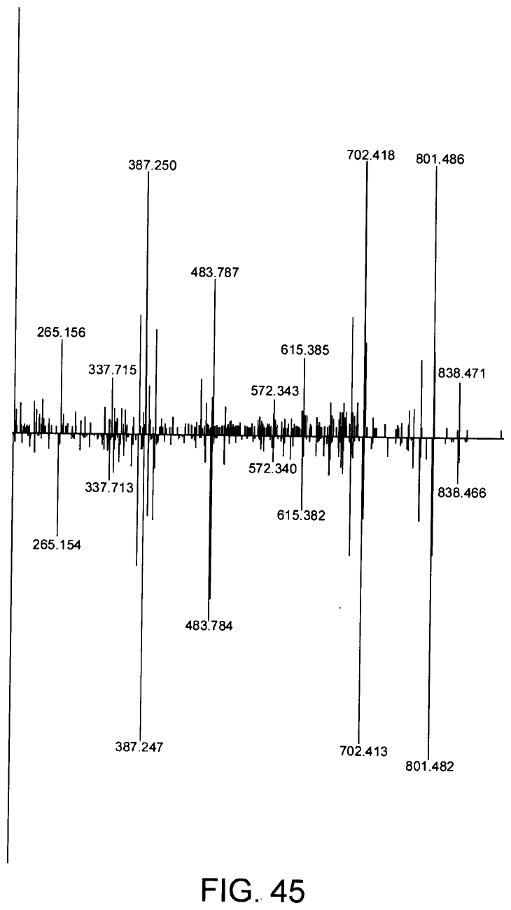

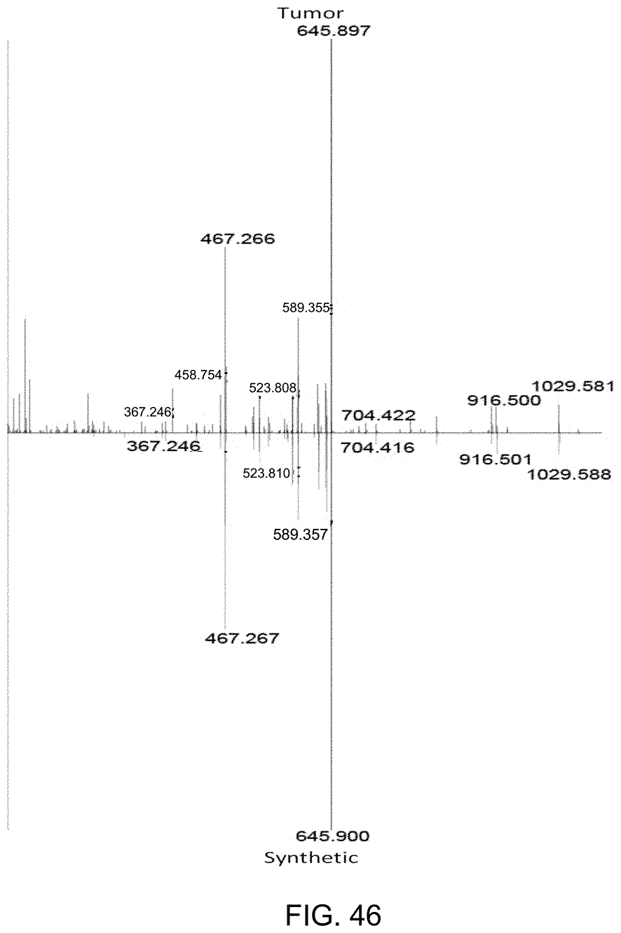

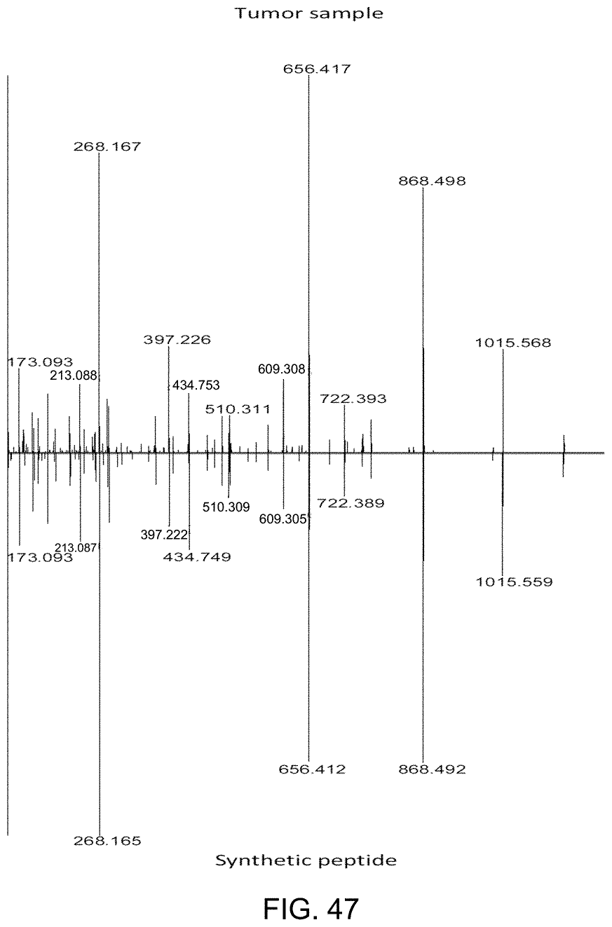

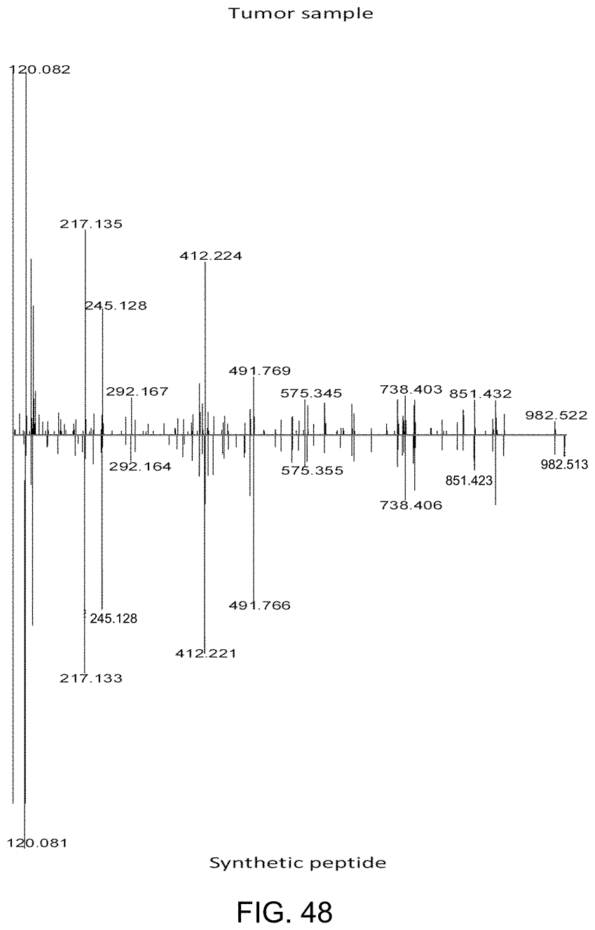

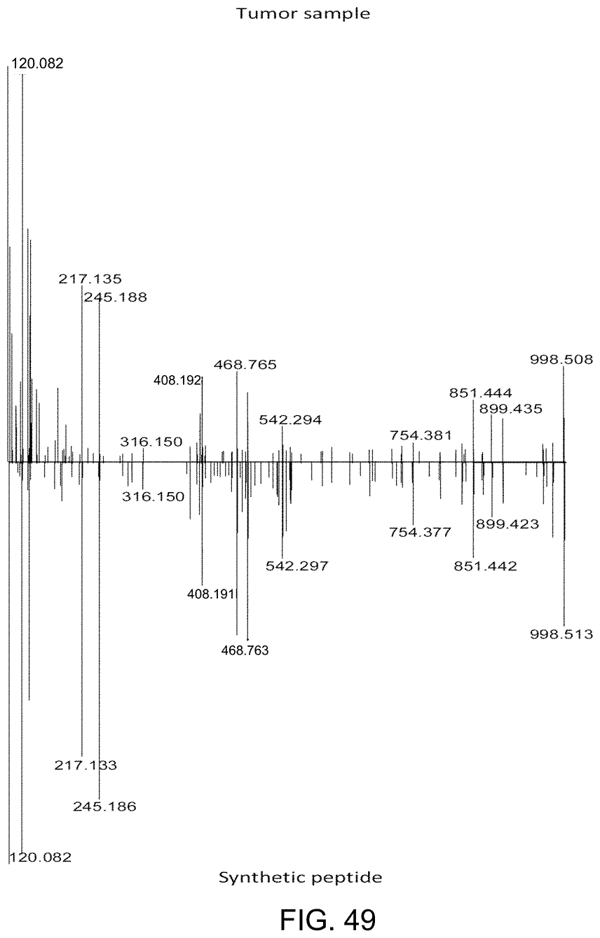

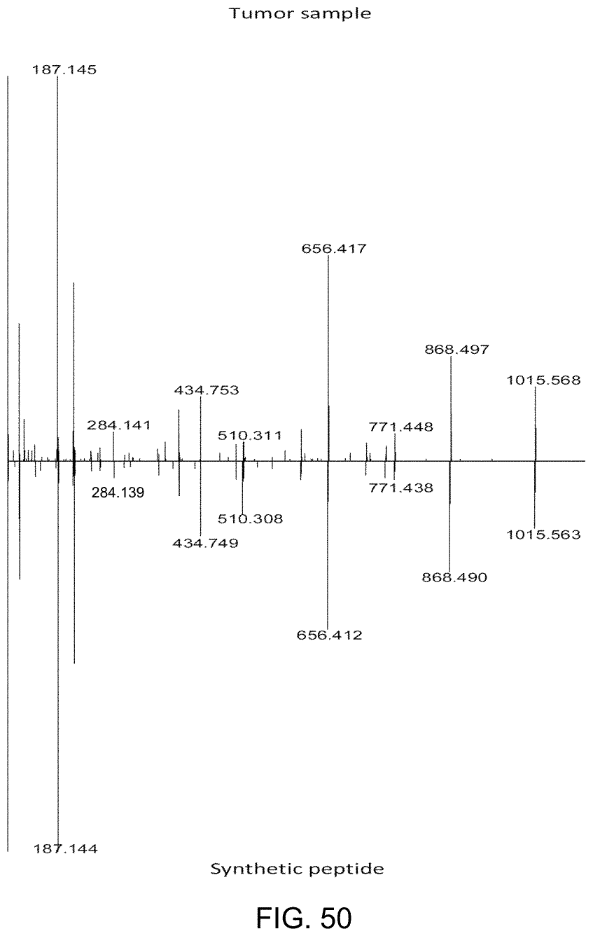

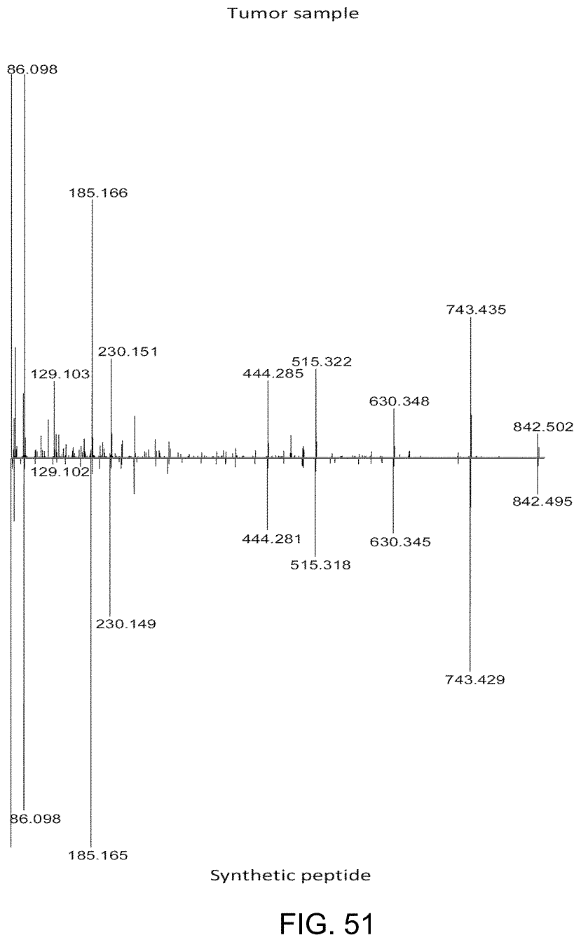

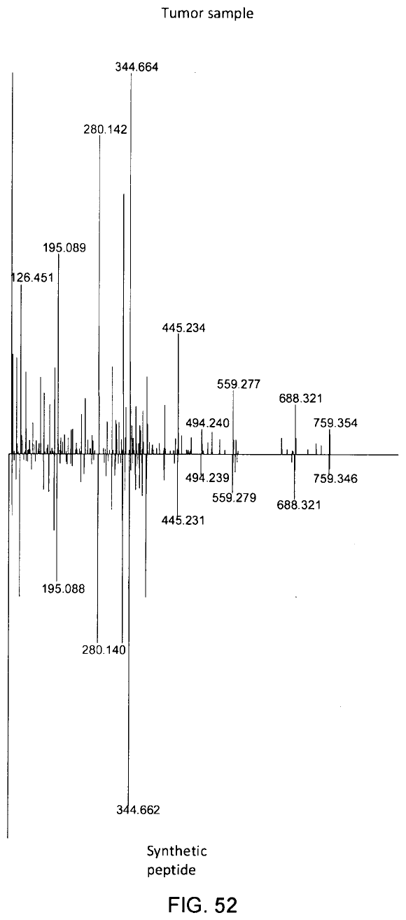

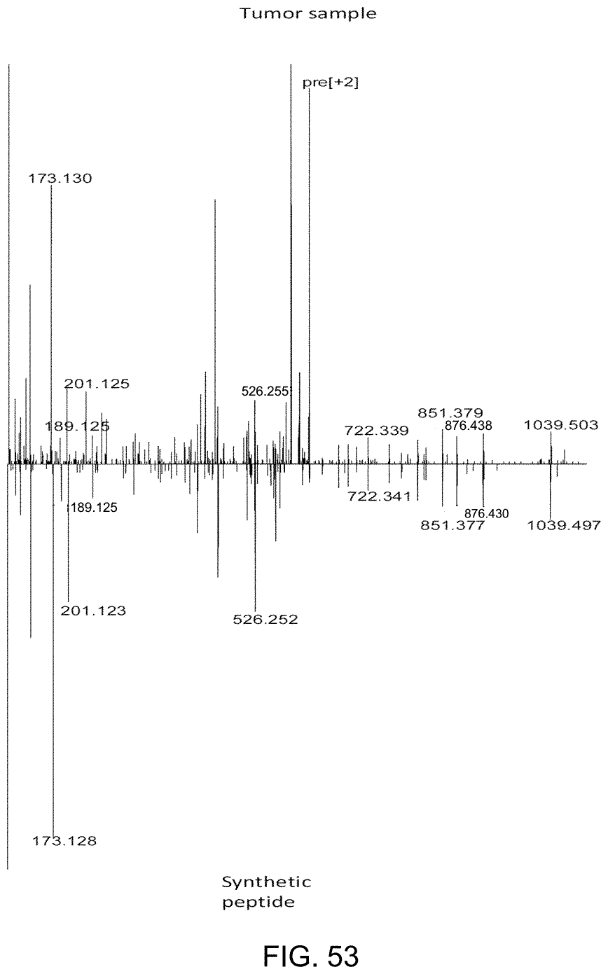

To provide further certainty of the assignment of tumor tissue-derived MS spectra to the peptide sequences that we discovered, peptides with these discovered sequences were synthesized and subjected to nUPLC-MS² using the same conditions applied to the tumor samples in the original study (Bassani-Sternberg et al., 2016, Nature Commun., 7: 13404; Inventors' data). Comparison of the spectra for selected peptides are shown in FIGS. 39-54. In each Figure the upper spectrum corresponds to the tumor sample (from the PRIDE database (Bassani-Sternberg et al., 2016, Nature Commun., 7: 13404; database link: https://www.ebi.ac.uk/pride/archive/projects/PXD004894 or in the inventors' database) and the lower spectrum corresponds to the synthetically produced peptide of the same sequence. Selected m/z values of detected ion fragments are shown above/below each fragment peak in these MS/MS spectra. These Figures reveal a precise alignment of fragments (tiny differences in the experimentally determined m/z values between tumor- and synthetic peptide-derived fragment ions being well within the m/z tolerances of <0.05 Daltons), confirming the veracity of the assignment of each of the tumor tissue-derived spectra to the CLT-encoded peptides.

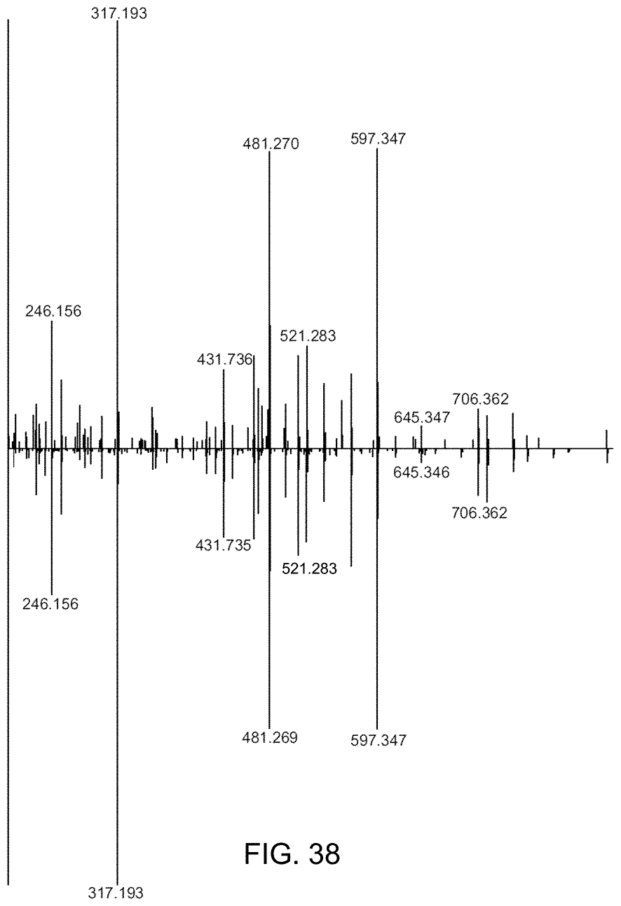

Taken together, the data shown in Tables 1 & 2 and FIGS. 1-53 supply exceptionally strong support for the translation, processing, and presentation of the corresponding CLT antigens in melanoma patients.

To further confirm the cancer-specificity of these CLTs, the inventors processed 37 normal tissue samples (10 normal skin, 9 normal lung and 18 normal breast tissue) and prepared for immunopeptidomic analysis. The inventors interrogated the spectra of the HLA-Class I dataset from these normal tissue samples, searching for all possible peptide sequences derived from the polypeptide sequences of CLT antigens 1, 2, 3, 4, 5, 6, 7 and 8, alongside all the polypeptides found in the human proteome (UniProt) using the Peaks™ software (V8.5 and X). No peptides derived from CLT antigen 1, 2, 3, 4, 5, 6, 7 or 8 were detected in the set of normal tissue samples (Table 3) providing additional evidence that the CLTs have cancer-specific expression.

In summary: the identification of immunopeptidomic peptides derived from the predicted ORFs, demonstrates that these CLTs are translated into polypeptides (SEQ ID NOs. 1-8; referred to as CLT antigens) in tumor tissue. These are then processed by the immune surveillance apparatus of the cells, and component peptides are loaded onto HLA Class I molecules, enabling the cell to be targeted for cytolysis by T cells that recognize the resulting peptide/HLA Class I complexes. Thus, these CLT antigens and their fragments are expected to be useful in a variety of therapeutic modalities for the treatment of melanoma in patients whose tumors express these antigens.

TABLE 1
List of peptides identified by immunopeptidomic analyses of melanoma tumor
samples, along with CLT antigen name and cross reference to SEQ ID NOs.

| | Peptide | CLT | CLT Ant. | | | | | | |
|---|---|---|---|---|---|---|---|---|---|
| Peptide | SEQ ID | Ant. | SEQ ID | Analysis | Patient | Peptide | −10lg | # of | |
| sequence¹ | No. | No. | NO. | type | #² | mass³ | P⁴ | spectra⁵ | Ppm⁶ |
| VQQGWFFPR | SEQ ID | 1 | SEQ ID | A | Mel-3 | 1163.59 | 15.54 | 1 | 9.4 |
| | NO. 9 | | NO. 1 | | | | | | |
| VVRGGAGFAAR | SEQ ID | 1 | SEQ ID | A | Mel-3 | 1059.59 | 49.12 | 8 | 5.1 |
| | NO. 10 | | NO. 1 | | | | | | |
| VVRGGAGFAAR | SEQ ID | 1 | SEQ ID | B | Mel-3 | 1059.59 | na | 1 | na |
| | NO. 10 | | NO. 1 | | | | | | |
| VVRGGAGFAAR | SEQ ID | 1 | SEQ ID | C | 2MT3 | 1059.594 | 42.49 | 6 | −0.9 |
| | NO. 10 | | NO. 1 | | | | | | |
| HLADRKLSL | SEQ ID | 1 | SEQ ID | A | Mel-5 | 1051.61 | 20.75 | 2 | −1.1 |
| | NO. 11 | | NO. 1 | | | | | | |
| HLADRKLSL | SEQ ID | 1 | SEQ ID | A | Mel-16 | 1051.61 | 33.22 | 30 | 5.1 |
| | NO. 11 | | NO. 1 | | | | | | |
| HLADRKLSL | SEQ ID | 1 | SEQ ID | B | Mel-16 | 1051.61 | — | 3 | |
| | NO. 11 | | NO. 1 | | | | | | |
| HLADRKLSL | SEQ ID | 1 | SEQ ID | C | 2MT3 | 1051.61 | 28.11 | 2 | −1.4 |
| | NO. 11 | | NO. 1 | | | | | | |
| HLADRKLSL | SEQ ID | 1 | SEQ ID | C | 2MT10 | 1051.61 | 21.21 | 1 | 3.4 |
| | NO. 11 | | NO. 1 | | | | | | |
| ARLQGSVTL | SEQ ID | 1 | SEQ ID | B | Mel-5 | 985.56 | na | 1 | na |
| | NO. 12 | | NO. 1 | | | | | | |
| ADSLILDF | SEQ ID | 2 | SEQ ID | A | Mel-26 | 892.45 | 16.68 | 1 | 0.9 |
| | NO. 18 | | NO. 2 | | | | | | |
| SSFSTLASLDK | SEQ ID | 2 | SEQ ID | A | Mel-20 | 1154.58 | 42.38 | 2 | −6.7 |
| | NO. 19 | | NO. 2 | | | | | | |
| SSFSTLASLDK | SEQ ID | 2 | SEQ ID | B | Mel-20 | 1154.58 | na | 2 | na |
| | NO. 19 | | NO. 2 | | | | | | |
| SSFSTLASLDK | SEQ ID | 2 | SEQ ID | C | 2MT4 | 1154.582 | 35.88 | 1 | 0.9 |
| | NO. 19 | | NO. 2 | | | | | | |
| NTPNIVSLR | SEQ ID | 3 | SEQ ID | A | Mel-35 | 1012.57 | 20.91 | 2 | 3.8 |
| | NO. 31 | | NO. 3 | | | | | | |
| NTPNIVSLR | SEQ ID | 3 | SEQ ID | C | 2MT3 | 1012.567 | 18.09 | 1 | −2.8 |
| | NO. 31 | | NO. 3 | | | | | | |
| RPLRIKGVF | SEQ ID | 3 | SEQ ID | C | 1MT1 | 1084.6869 | 26.28 | 7 | 0 |
| | NO. 32 | | NO. 3 | | | | | | |
| KTKGSLSVFR | SEQ ID | 4 | SEQ ID | A | Mel-3 | 1121.66 | 26.91 | 3 | 4.8 |
| | NO. 36 | | NO. 4 | | | | | | |
| KTKGSLSVFR | SEQ ID | 4 | SEQ ID | B | Mel-3 | 1121.66 | na | 2 | na |
| | NO. 36 | | NO. 4 | | | | | | |
| KTKGSLSVFR | SEQ ID | 4 | SEQ ID | C | 2MT3 | 1121.6556 | 40.46 | 6 | −0.7 |
| | NO. 36 | | NO. 4 | | | | | | |
| KTKGSLSVFR | SEQ ID | 4 | SEQ ID | C | 2MT1 | 1121.6556 | 35.73 | 2 | −0.7 |
| | NO. 36 | | NO. 4 | | | | | | |
| AAFDRAVHF | SEQ ID | 4 | SEQ ID | A | Mel-40 | 1032.51 | 17.24 | 1 | 0.3 |
| | NO. 37 | | NO. 4 | | | | | | |
| AAFDRAVHF | SEQ ID | 4 | SEQ ID | A | Mel-41 | 1032.514 | 26.11 | 3 | 7.8 |
| | NO. 37 | | NO. 4 | | | | | | |
| AAFDRAVHF | SEQ ID | 4 | SEQ ID | C | 2MT3 | 1032.514 | 18.73 | 1 | −1.3 |
| | NO. 37 | | NO. 4 | | | | | | |
| AFDRAVHF | SEQ ID | 4 | SEQ ID | A | Mel-27 | 961.4769 | 20.6 | 3 | 7.9 |
| | NO. 38 | | NO. 4 | | | | | | |
| AFDRAVHF | SEQ ID | 4 | SEQ ID | A | Mel-39 | 961.47 | 27.49 | 3 | 2.6 |
| | NO. 38 | | NO. 4 | | | | | | |
| KTKGSLSVF | SEQ ID | 4 | SEQ ID | C | 2MT12 | 965.555 | 27.84 | 1 | 1.2 |
| | NO. 39 | | NO. 4 | | | | | | |
| LPRTPRPDLIL | SEQ ID. | 5 | SEQ ID | C | Mel-8 | 1289.7819 | 22.53 | 2 | 2.3 |
| | NO. 45 | | NO. 5 | | | | | | |
| LPRTPRPDLIL | SEQ ID. | 5 | SEQ ID | C | Mel-16 | 1289.7819 | 28.23 | 5 | 7 |
| | NO. 45 | | NO. 5 | | | | | | |
| LPRTPRPDLIL | SEQ ID. | 5 | SEQ ID | C | Mel-29 | 1289.7819 | 28.79 | 1 | −1.4 |
| | NO. 45 | | NO. 5 | | | | | | |
| ATIFPDPWLLK | SEQ ID | 6 | SEQ ID | C | Mel-41 | 1299.7227 | 36.89 | 3 | 8.5 |
| | NO. 48 | | NO. 6 | | | | | | |
| FPFYKDTVL | SEQ ID | 6 | SEQ ID | C | Mel-41 | 1128.5854 | 31.32 | 5 | 8.3 |
| | NO. 49 | | NO. 6 | | | | | | |
| FPFYKDTVLL | SEQ ID | 6 | SEQ ID | C | Mel-41 | 1241.6696 | 29.36 | 1 | 9.2 |
| | NO. 50 | | NO. 6 | | | | | | |
| TIFPDPWLLK | SEQ ID | 6 | SEQ ID | C | Mel41 | 1228.6855 | 31.9 | 3 | 8.9 |
| | NO. 51 | | NO. 7 | | | | | | |
| IVLDAPVTK | SEQ ID | 7 | SEQ ID | C | Mel-21 | 954.575 | 19.95 | 1 | 8.7 |
| | NO. 52 | | NO. 7 | | | | | | |
| IVLDAPVTK | SEQ ID | 7 | SEQ ID | C | Mel-36 | 954.575 | 22.36 | 2 | 1.6 |
| | NO. 52 | | NO. 7 | | | | | | |
| IVLDAPVTK | SEQ ID | 7 | SEQ ID | C | Mel-39 | 954.575 | 20.11 | 2 | 1.3 |
| | NO. 52 | | NO. 7 | | | | | | |
| IVLDAPVTK | SEQ ID | 7 | SEQ ID | C | 2MT3 | 954.575 | 24.77 | 7 | 0.3 |
| | NO. 52 | | NO. 7 | | | | | | |
| GHIHNEAV | SEQ ID | 8 | SEQ ID | C | Mel-27 | 875.4249 | 21.32 | 2 | 7.9 |
| | NO. 53 | | NO. 8 | | | | | | |
| SLYGHIHNEAV | SEQ ID | 8 | SEQ ID | C | Mel-27 | 1238.6044 | 35.11 | 1 | 8.5 |
| | NO. 54 | | NO. 8 | | | | | | |
| SLYGHIHNEAV | SEQ ID | 8 | SEQ ID | C | 2MT4 | 1238.604 | 56.34 | 2 | 4.7 |
| | NO. 54 | | NO. 8 | | | | | | |

¹HLA Class I peptides identified by mass spectrometry.
²Bassani-Sternberg et al, 2016, Nature Comm., 7: 13404 (Mel-3, Mel-5, Mel-8, Mel1-6, Mel-21, Mel-27, Mel-29, Mel-30, Mel-36, Mel-39, Mel-41); Inventors' dataset (1MT1, 2MT1, 2MT3, 2MT4, 2MT10, 2MT12).
³Calculated peptide mass.
⁴PEAKS ™ program −10lgP values are shown for peptides for highest match for peptide/patients for which more than one spectral detection was obtained. Values are not available (na) for peptides identified via Analysis B performed using Mascot software.
⁵Number of spectra in which peptide was detected.
⁶Deviation between observed mass and calculated mass; selected ppm values are shown for peptides for which more than one spectrum was obtained. Values are not available (na) for peptides identified via Analysis B.

TABLE 2
Predicted NetMHCpan4.0 binding of Mass Spectrometry-identified peptides (length ≥
9 residues) to 18 HLA Class I Supertype Alleles (HLA-A01:01, HLA-A02:01, HLA-A03:01,
HLA-A11:01, HLA-A24:02, HLA-A25:01, HLA-A26:01, HLA-A68:01, HLA-B07:02, HLA-B08:01,
HLA-B15:01, HLA-B18:01, HLA-B27:05, HLA-B35:01, HLA-B35:03, HLA-B40:01, HLA-B40:02,
HLA-B51:01), along with CLT antigen name and cross reference to SEQ ID NOs.

| Predicted to | | Number | Number | Number | | | |
|---|---|---|---|---|---|---|---|
| bind 18 | | of alleles | of alleles | of alleles | Peptide | | |
| human HLA | | with a rank | with a rank | with a rank | SEQ | CLT | |
| Class I A & B | Peptide | score of | score of | score of | ID | Antigen | Patient |
| supertypes¹ | sequence | ≤5%² | ≤2%³ | ≤0.5%⁴ | NO | No. | #⁵ |
| YES | VQQGWFFPR | 3/18 | 0/18 | 0/18 | SEQ ID | 1 | Mel-3 |
| | | | | | NO. 9 | | |
| YES | VVRGGFAGFAAR | 4/18 | 0/18 | 0/18 | SEQ ID | 1 | Mel-3 |
| | | | | | NO. 10 | | 2MT3 |
| YES | HLADRKLSL | 10/18 | 7/18 | 2/18 | SEQ ID | 1 | Mel-5 |
| | | | | | NO. 11 | | Mel-16 |
| | | | | | | | 2MT3 |
| | | | | | | | 2MT10 |
| YES | ARLQGSVTL | 10/18 | 3/18 | 1/18 | SEQ ID | 1 | Mel-5 |
| | | | | | NO. 12 | | |
| YES | SSFSTLASLDK | 3/18 | 2/18 | 0/18 | SEQ ID | 2 | Mel-20 |
| | | | | | NO. 19 | | 2MT4 |
| YES | NTPNIVSLR | 6/18 | 3/18 | 1/18 | SEQ ID | 3 | Mel-35 |
| | | | | | NO. 31 | | 2MT3 |
| YES | RPLRIKGVF | 6/18 | 4/18 | 1/18 | SEQ ID | 3 | 2MT1 |
| | | | | | NO. 32 | | |
| YES | KTKGSLSVFR | 4/18 | 3/18 | 0/18 | SEQ ID | 4 | Mel-3 |
| | | | | | NO. 36 | | 2MT1 |
| | | | | | | | 2MT3 |
| YES | AAFDRAVHF | 18/18 | 10/18 | 5/18 | SEQ ID | 4 | Mel-40 |
| | | | | | NO. 37 | | Mel-41 |
| | | | | | | | 2MT3 |
| YES | KTKGSLSVF | 9/18 | 4/18 | 2/18 | SEQ ID | 4 | 2MT12 |
| | | | | | NO. 39 | | |
| YES | LPRTPRPDLIL | 4/18 | 1/18 | 1/18 | SEQ ID | 5 | Mel-8 |
| | | | | | NO. 45 | | Mel-16 |
| | | | | | | | Mel-29 |
| YES | ATIFPDPWLLK | 4/18 | 3/18 | 2/18 | SEQ ID | 6 | Mel-41 |
| | | | | | NO. 48 | | |
| YES | FPFYKDTVL | 10/18 | 7/18 | 5/18 | SEQ ID | 6 | Mel-41 |
| | | | | | NO. 49 | | |
| YES | FPFYKDTVLL | 8/18 | 6/18 | 4/18 | SEQ ID | 6 | Mel-41 |
| | | | | | NO. 50 | | |
| YES | TIFPDPWLLK | 6/18 | 3/18 | 3/18 | SEQ ID | 6 | Mel-41 |
| | | | | | NO. 51 | | |
| YES | IVLDAPVTK | 3/18 | 3/18 | 3/18 | SEQ ID | 7 | Mel-21, |
| | | | | | NO. 52 | | Mel-34, |
| | | | | | | | Mel-39 |
| YES | SLYGHIHNEAV | 1/18 | 1/18 | 1/18 | SEQ ID | 8 | Mel-27 |
| | | | | | NO. 54 | | |

¹Predicted binding to interrogated HLA Class I supertypes at a Rank score of ≤5.0% score.
²Number of the 18 HLA Class I supertypes that were predicted to bind with a rank score of ≤5.0%.
³Number of the 18 HLA Class I supertypes that were predicted to bind with a rank score of ≤2.0%.
⁴Number of the 18 HLA Class I supertypes that were predicted to bind with a rank score of ≤0.5%.
⁵Bassani-Sternberg et al, 2016, Nature Comm., 7: 13404 (Mel-3, Mel-8, Mel-16, Mel-21, Mel-27, Mel-29, Mel-30, Mel-36, Mel-39, Mel-41); Inventors' dataset (1MT1, 2MT1, 2MT3, 2MT4, 2MT10, 2MT12).

TABLE 3
Number of peptides-derived from CLT Antigens
1 to 8 in a set of normal tissue samples.

| Antigen | Skin | Lung | Breast |
|---|---|---|---|
| CLT Antigen 1 | 0/10 | 0/9 | 0/18 |
| CLT Antigen 2 | 0/10 | 0/9 | 0/18 |
| CLT Antigen 3 | 0/10 | 0/9 | 0/18 |
| CLT Antigen 4 | 0/10 | 0/9 | 0/18 |
| CLT Antigen 5 | 0/10 | 0/9 | 0/18 |
| CLT Antigen 6 | 0/10 | 0/9 | 0/18 |
| CLT Antigen 7 | 0/10 | 0/9 | 0/18 |
| CLT Antigen 8 | 0/10 | 0/9 | 0/18 |

The results presented here in Examples 1 and 2 are in whole or part based upon data generated by the The Cancer Genome Atlas (TCGA) Research Network (http://cancergenome.nih.gov/); and the Genotype-Tissue Expression (GTEx) Project (supported by the Common Fund of the Office of the Director of the National Institutes of Health, and by NCI, NHGRI, NHLBI, NIDA, NIMH, and NINDS).

Example 3—HERVFEST

Functional expansion of specific T-cells (FEST) technology has been used to identify therapeutically relevant tumor-derived epitopes present in the “mutation-associated neoantigen” (MANA) repertoire found in tumor cells of cancer patients based on detection of patient T-cells that react to MANA epitopes (Anagnostou et al., Cancer Discovery 2017; Le et al., Science 2017; Forde et al., NEJM 2018; Danilova et al., Cancer Immunol. Res. 2018). Application of FEST technology to CLT antigens discovered by using the methods elucidated in Example 1 & 2 (Tables 1-3, FIGS. 1-53) can be used to identify therapeutically relevant T-cell responses to CLT antigens in cancer patients.

Like other assays (e.g., ELISPOT) to identify epitope-specific T-cells in a subject who has undergone immune exposure, “FEST” technologies derive their specificity by activating/expanding the cognate T-cells in ex vivo cultures that include antigen-presenting cells and suitable antigenic peptides. The technique differs from other immunological assays in that it utilizes next-generation sequencing of the T-cell receptor (TCR) DNA sequences present in these amplified cultures (specifically: TCRseq targeting the TCR-Vβ CDR3 region) to detect the specific TCRs that are expanded in the cells cultured with individual peptides from a panel of target peptides derived from an antigen (or antigens). Application of TCRseq to tumor tissues in the same patient can also be used to demonstrate if TCRs/T-cells detected in the ex vivo, peptide-stimulated cultures are also present within the tumor-infiltrating lymphocytes found in cancer tissues in situ. Thus, MANAFEST has proven to be a powerful technology for identifying MANA epitopes that are recognized by patient T-cells, permitting identification of functionally relevant MANA peptides among the multitude of mutant peptides found by whole-exome sequencing of normal and tumor tissues from cancer patients (Le et al., Science 2017; Forde et al., NEJM 2018; Danilova et al., Cancer Immunol. Res. 2018; Smith et al., J Immunother Cancer 2019).

Application of MANAFEST methodology (Danilova et al., Cancer Immunol. Res. 2018) to CLT antigens was performed as follows. The method, which we will refer to as HERVFEST, consists of the following steps: Step 1: Peptides predicted to contain epitopes that efficiently bind selected HLA Class I alleles were identified in CLT Antigens. Step 2: PBMCs from suitable melanoma patients were matched by HLA Class I type to the peptide library selected in step 1. Step 3: PBMCs from these patients were separated into T-cell and non T-cell fractions. Non T-cells were added back to the patient's T-cells, and then divided into 20-50 wells (containing 250,000 T-cells per culture) and propagated with various T-cell growth factors and individual CLT Antigen-derived synthetic peptides (selected in step 1/2) for 10 days. Step 4: TCRseq (sequencing of the TCR-Vβ CDR3 sequences) was performed on all wells, and TCR-Vβ CDR3 sequences that were amplified in the presence of individual CLT Antigen-derived peptides (but not amplified in the presence of control peptides or in the absence of peptide stimulation) were identified. The presence of amplified TCR-Vβ CDR3 sequences in individual wells of the assay thus identifies CLT Antigen-derived peptides that elicited an immune response in the melanoma patient. Step 5: TCRseq may also be performed on tumor samples to determine whether the T-cells bearing the CLT-Antigen amplified TCRs homed to patient tumors, providing additional evidence that T-cells bearing these TCRs recognize CLT Antigen-derived peptides within a patient's tumor.

HERVFEST assays were performed with peptides derived from CLT Antigens 1-4 (SEQ ID NOs 1-4). The panel of peptides (see step 1 above) used for these studies was based on NetMHC predictions of CLT Antigen-derived peptides that were predicted to strongly bind the 8 HLA Class I types commonly found in patient tumor samples available for our analyses. CLT Antigen-derived peptides that amplified one or more TCRs in these HERVFEST assays are provided in Table 4. Table 4 also indicates the HLA Class I type(s) of the CLT antigen peptides that were tested with each patient's PBMC-derived cultures. The HLA Class I type of the patients whose PBMCs were tested in these studies and amplified one or more TCRs in the assays, are shown in Table 5.

FIG. 54 panel A shows published data demonstrating TCR amplification with NSCLC patient-specific MANA peptides (Forde et al., NEJM 2018). The vertical axis shows the prevalence of each indicated TCR Vβ CDR3 AA Sequence for wells of cells cultivated in the presence of the MANA or control peptides listed on the horizontal axis. The amplification in the well containing MANA7 indicates the patient's T-cell repertoire include T-cells that are reactive to this peptide. Panels B and C of FIG. 54 show representative TCR amplification data from PBMCs from 2 melanoma patients that were incubated in the presence of the indicated CLT Antigen peptides and control peptides. As with Panel A, the specific amplifications observed in Panels B & C demonstrate that the T-cell repertoire of these melanoma patients includes T-cells that are reactive with specific CLT Antigen-derived peptides. Panel B shows the frequency of TCRs detected in the LMSSFSTLASL-stimulated well of PBMCs from melanoma patient 222B in all wells stimulated with the panel 15 Class I HLA-A*02 peptides from CLT Antigens 1, 2 & 4. Three TCR sequences were amplified. LMSSFSTLASL (SEQ ID NO. 23) is an HLA-A*02 binding peptide derived from CLT Antigen 2. Panel C shows the frequency of TCRs detected in the MVACRIKTFR-stimulated well of PBMCs from melanoma patient 224B in all wells stimulated with the panel of 15 Class I HLA-A*02 peptides from CLT Antigens 1, 2 & 4 and 24 Class I HLA-A*03 peptides from CLT Antigens 1, 2, 3, & 4. One TCR sequence was amplified. MVACRIKTFR (SEQ ID NO. 26) is an HLA-A*03 binding peptide derived from CLT Antigen 2.

The control peptides/conditions used in these experiments were as follows: CEF=mixture of CMV, EBV, and influenza peptides; SL9, TV9 and QK1=HIV-1 control peptides; no peptide=cultivation in absence of peptide; Baseline=T-cells before culture.

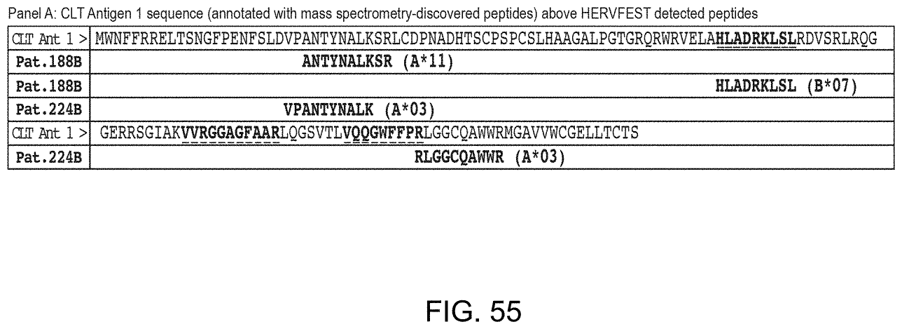

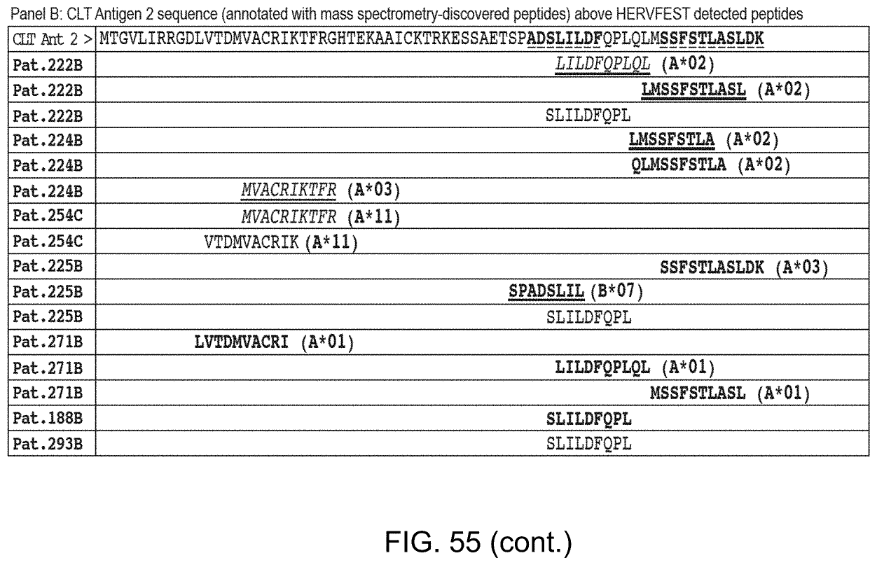

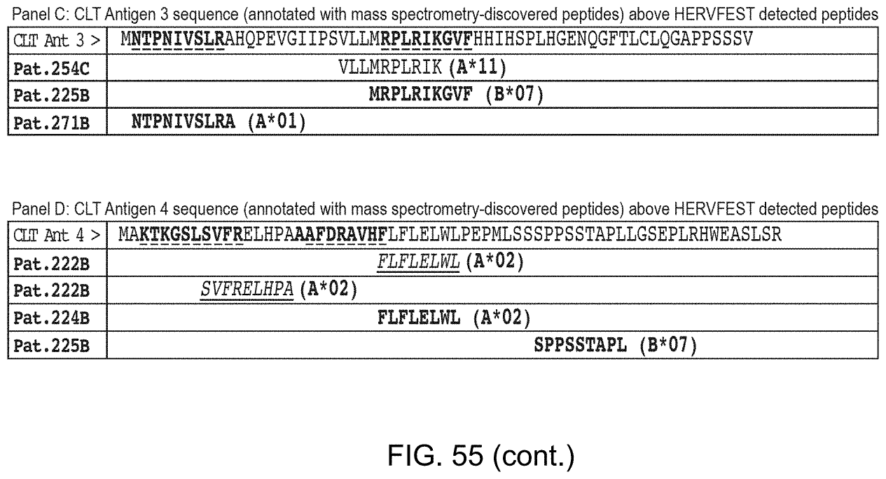

FIG. 55 shows a summary of all CLT Antigen peptides for CLT Antigens 1-4 which amplified one or more TCRs in studies completed with these patients. Each panel displays the amino acid sequences of CLT Antigens 1-4 overlaid with peptides detected by immunopeptidomic analyses (denoted by dashed underlined or bold text; see Example 2). Below these sequences, the HERVFEST-detected peptides (see FIG. 54) are displayed with the numeric identifier of the melanoma patient in which they were detected (Table 5) and the targeted HLA Class I type.

The properties of each HERVFEST detection are defined as follows:
Plain text: Significant amplification of a single TCR
Bold text: Significant amplification of multiple TCRs
Underlined italics text: Significant amplification of a single TCR which was detected in other wells
Underlined bold text: Significant amplification of multiple TCRs, at least one of which was detected in other wells

These results provide strong evidence that CLT Antigens 1-4 are present in melanoma patients and that peptides derived from these CLT antigens have elicited specific T-cell responses in these melanoma patients, confirming the value of these CLT antigens as targets for therapeutic interventions to treat melanoma.

TABLE 4
CLT Antigen-derived peptides that amplified
one or more TCRs in HERVFEST assays

| | | | HLA Class | |
|---|---|---|---|---|
| | | | I-type | |
| | | | for | |
| | | | peptide | |
| Reference | SEQ | | (predicted | HERVFEST |
| CLT | ID | Peptide | by | panel |
| Antigen | NO | sequence | NetMHC) | reference |
| 1 | SEQ ID | VPANTYNALK | HLA-A*03:01 | 37 |
| | NO. 13 | | | |
| 1 | SEQ ID | RLGGCQAWWR | HLA-A*03:01 | 41 |
| | NO. 14 | | | |
| 1 | SEQ ID | ANTYNALKSR | HLA-A*11:01 | 60 |
| | NO. 15 | | | |
| 1 | SEQ ID | HLADRKLSL | HLA-B*07:02 | 109 |
| | NO. 11 | | | |
| 2 | SEQ ID | LVTDMVACRI | HLA-A*01:01 | 8 |
| | NO. 20 | | | |
| 2 | SEQ ID | LILDFQPLQL | HLA-A*01:01 | 13 |
| | NO. 21 | | | |
| 2 | SEQ ID | MSSFSTLASL | HLA-A*01:01 | 15 |
| | NO. 22 | | | |
| 2 | SEQ ID | LILDFQPLQL | HLA-A*02:01 | 25 |
| | NO. 21 | | | |
| 2 | SEQ ID | LMSSFSTLASL | HLA-A*02:01 | 27 |
| | NO. 23 | | | |
| 2 | SEQ ID | LMSSFSTLA | HLA-A*02:01 | 23 |
| | NO. 24 | | | |
| 2 | SEQ ID | QLMSSFSTLA | HLA-A*02:01 | 26 |
| | NO. 25 | | | |
| 2 | SEQ ID | MVACRIKTFR | HLA-A*03:01 | 46 |
| | NO. 26 | | | |
| 2 | SEQ ID | SSFSTLASLDK | HLA-A*03:01 | 45 |
| | NO. 19 | | | |
| 2 | SEQ ID | MVACRIKTFR | HLA-A*11:01 | 67 |
| | NO. 26 | | | |
| 2 | SEQ ID | VTDMVACRIK | HLA-A*11:01 | 69 |
| | NO. 27 | | | |
| 2 | SEQ ID | SPADSLIL | HLA-B*07:02 | 119 |
| | NO. 28 | | | |
| 2 | SEQ ID | SLILDFQPL | HLA-A*02:01 | 20 |
| | NO. 29 | | | |
| 3 | SEQ ID | NTPNIVSLRA | HLA-A*01:01 | 18 |
| | NO. 33 | | | |
| 3 | SEQ ID | VLLMRPLRIK | HLA-A*11:01 | 78 |
| | NO. 34 | | | |
| 3 | SEQ ID | MRPLRIKGVF | HLA-B*07:02 | 124 |
| | NO. 35 | | | |
| 4 | SEQ ID | FLFLELWL | HLA-A*02:01 | 30 |
| | NO. 40 | | | |
| 4 | SEQ ID | SVFRELHPA | HLA-A*02:01 | 29 |
| | NO. 41 | | | |
| 4 | SEQ ID | SPPSSTAPL | HLA-B*07:02 | 132 |
| | NO. 42 | | | |

TABLE 5
Characteristics of the melanoma patient PBMCs used in HERVFEST assays

| | | | | | | | HLA(s) |
|---|---|---|---|---|---|---|---|
| | HLA | HLA | HLA | HLA | HLA | HLA | targeted in |
| | allele | allele | allele | allele | allele | allele | HERVFEST |
| Patient | A1 | A2 | B1 | B2 | C1 | C2 | studies |
| 204B | A*01:01 | A*25:01 | B*03:01 | B*16:01 | C*07:01 | C*12:03 | A*01 |
| 222B | A*02:01 | A*30:02 | B*44:02 | Not | C*05:01 | B*18:01 | A*02 |
| | | | | known | | | |
| 224B | A*02:01 | A*03:01 | B*35:01 | B*40:02 | C*04:01 | C*02:02 | A*02, A*03 |
| 293B | A*02:01 | A*30:01 | B*13:02 | B*44:02 | C*06:02 | C*05:01 | A*02 |
| 254C | A*11:01 | A*32:01 | B*15:01 | B*56:01/ | C*01:02 | C*03:03 | A*11 |
| | | | | 56:55 | | | |
| 188B | A*02:01 | A*11:01 | B8*7:02 | B*14:02 | C*07:02 | C*08:02 | A*11, B*07 |
| 271B | A*01:01 | A*01:01 | B*08:01 | B*08:01 | C*07:01 | C*07:01 | A*01 |
| 225B | A*02:01 | A*03:01 | B*07:02 | B*51:01 | C*07:02 | C*14:02 | A*03, B*07 |

### Example 4—Assays to Demonstrate High-Affinity T-Cells Specific for CLT Antigens have not been Deleted from Normal Subjects' T-Cell Repertoire

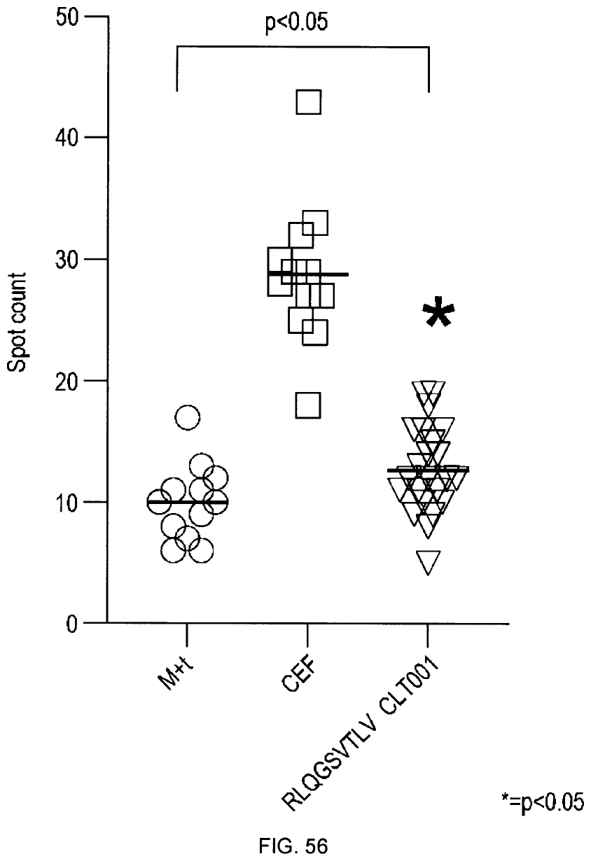

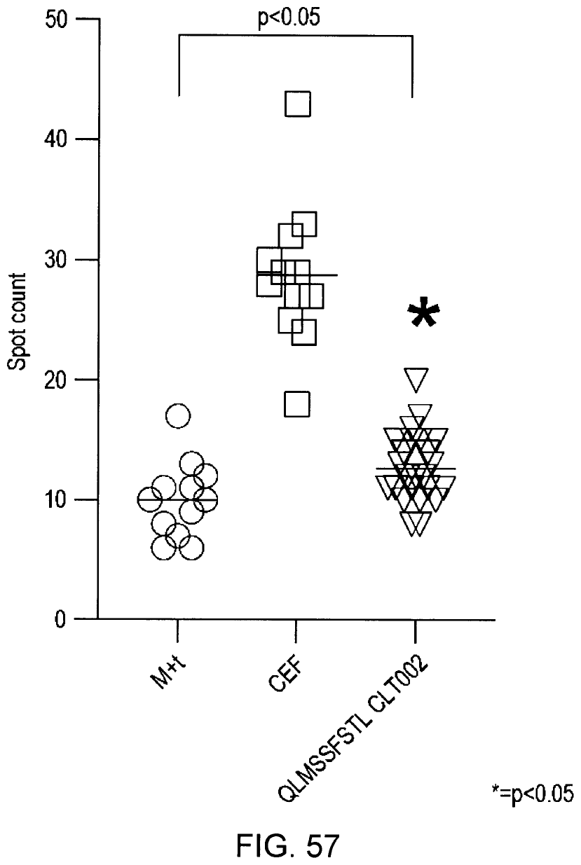

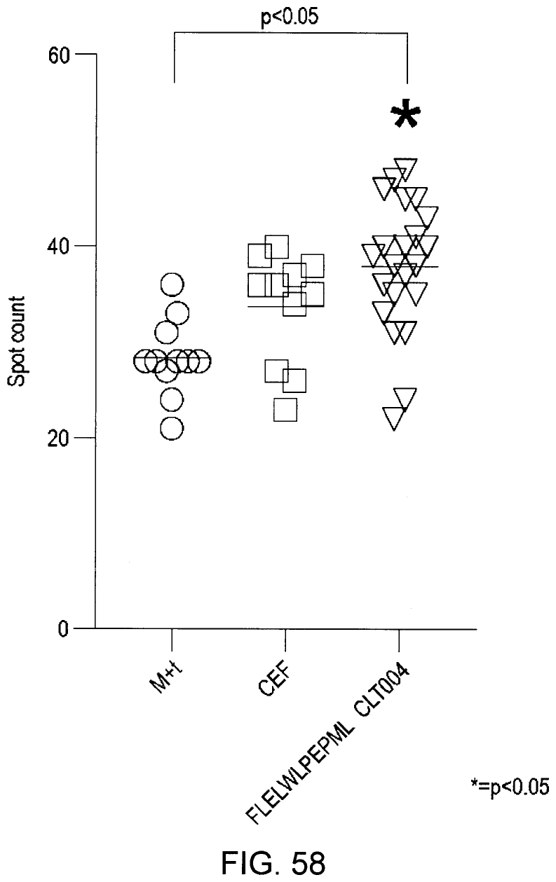

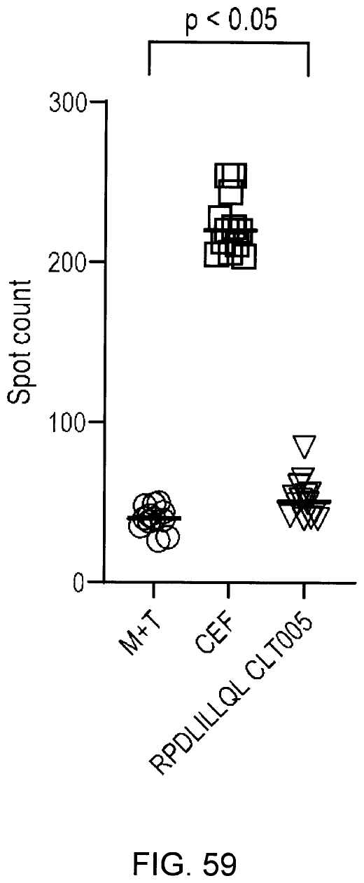

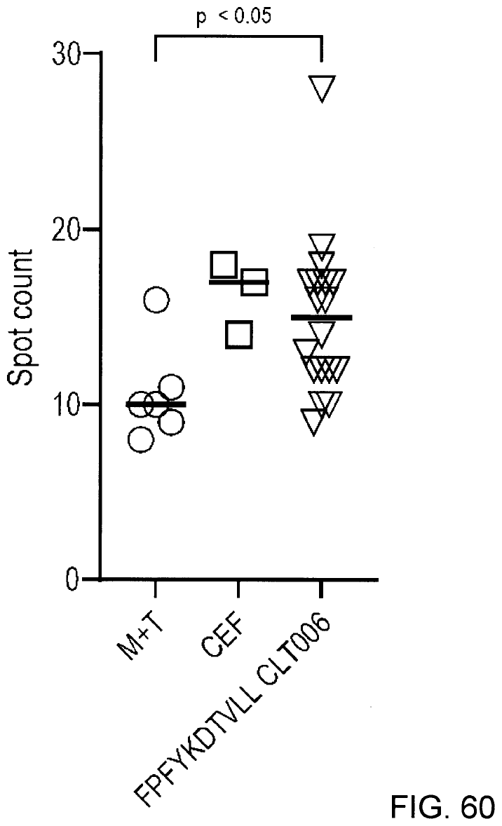

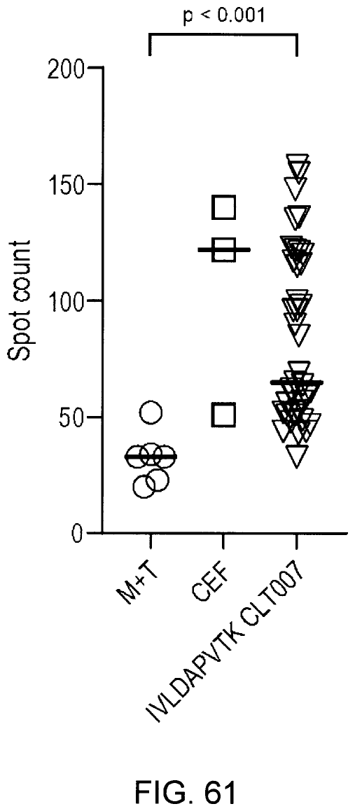

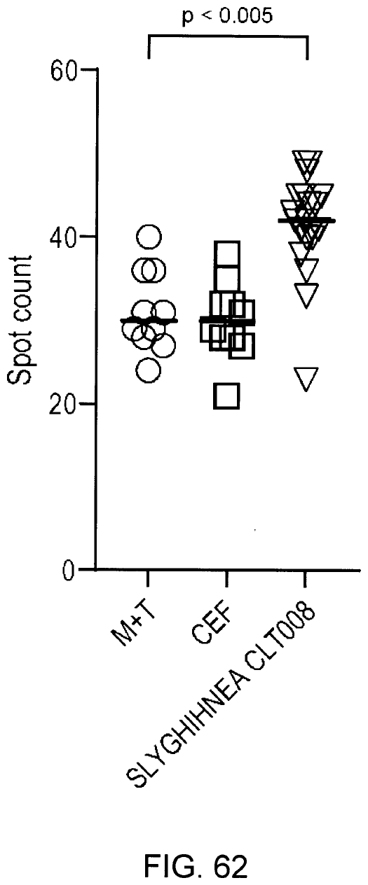

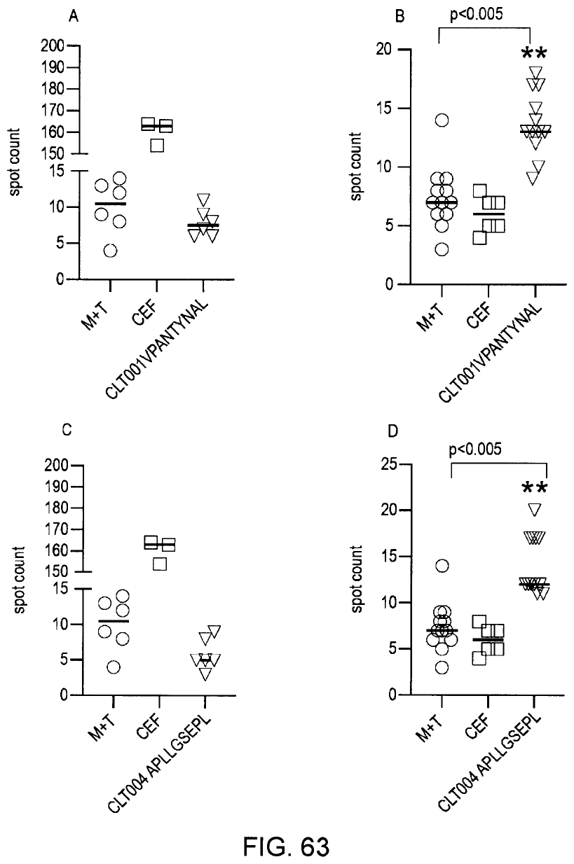

An ELISPOT assay may be used to show that CLT antigen-specific CD8 T-cells are present in the normal T-cell repertoire of healthy individuals, and thus have not been deleted by central tolerance due to the expression of cancer-specific CLT antigens in naïve and thymic tissues in these patients. This type of ELISPOT assay comprises multiple steps. Step 1: CD8 T-cells and CD14 monocytes can be isolated from the peripheral blood of normal blood donors, these cells are HLA Class I-typed to match the specific CLT antigens being tested. CD8 T-cells can be further sub-divided into naïve and memory sub-types using magnetically labelled antibodies to the memory marker CD45RO. Step 2: CD14 monocytes are pulsed with individual or pooled CLT antigen peptides for three hours prior to being co-cultured with CD8 T-cells for 14 days. Step 3: Expanded CD8 T-cells are isolated from these cultures and re-stimulated overnight with fresh monocytes pulsed with peptides. These peptides may include; individual CLT antigen peptides, irrelevant control peptides or peptides known to elicit a robust response to infectious (e.g., CMV, EBV, Flu, HCV) or self (e.g. MART-1) antigens. Re-stimulation is performed on anti-Interferon gamma (IFNγ) antibody-coated plates. The antibody captures any IFNγ secreted by the peptide-stimulated T-cells. Following overnight activation, the cells are washed from the plate and IFNγ captured on the plate is detected with further anti-IFNγ antibodies and standard colorimetric dyes. Where IFNγ-producing cells were originally on the plate, dark spots are left behind. Data derived from such assays includes spot count, median spot size and median spot intensity. These are measures of frequency of T-cells producing IFNγ and amount of IFNγ per cell. Additionally, a measure of the magnitude of the response to the CLT antigen can be derived from the stimulation index (SI) which is the specific response, measured in spot count or median spot size, divided by the background response to monocytes with no specific peptide. A metric of stimulation strength is derived by multiplying the stimulation index for spot number by the stimulation index for spot intensity. In this way, comparisons of the responses to CLT antigens and control antigens can be used to demonstrate that naïve subjects contain a robust repertoire of CLT antigen-reactive T-cells that can be expanded by vaccination with CLT antigen-based immunogenic formulations. Table 6 provides a list of CLT Antigen-derived peptides that induced significant CD8 T-cell responses from HLA-matched normal blood donors. The results are shown in FIGS. 56-63. Horizontal bars represent the mean of the data. M+t indicates the no peptide, negative control (monocytes and T cells). CEF indicates the positive control (a mixture of 23 CMV, EBV and influenza peptides). Statistical significance was measured with Kruskall Wallis test One-way Anova with correction for repeated measures with Dunns correction. FIG. 56 shows significant CD8 T-cell responses from a normal blood donor to HLA-A*02:01-restricted peptides from CLT Antigen 1 (CLT001 in the figure). The example shown in FIG. 57 demonstrates CD8 responses from a normal donor to a peptide derived from CLT Antigen 2 (CLT002 in the figure) also restricted by HLA-A*02:01. FIG. 58 shows significant CD8 T-cell responses from a normal blood donor to an HLA-A*02:01-restricted peptide from CLT Antigen 4 (CLT004 in the Figure). FIG. 59 shows significant CD8 T-cell responses from a normal blood donor to HLA-A*03:01-restricted peptide from CLT Antigen 5 (CLT005 in the Figure). FIG. 60 shows significant CD8 T-cell responses from a normal blood donor to an HLA-B*07:02-restricted peptide from CLT Antigen 6 (CLT006 in the Figure). FIG. 61 shows significant CD8 T-cell responses from a normal blood donor to an HLA-A*03:01-restricted peptide from CLT Antigen 7 (CLT007 in the Figure). FIG. 62 shows significant CD8 T-cell responses from a normal blood donor to an HLA-A*02:01-restricted peptide from CLT Antigen 8 (CLT008 in the Figure). FIG. 63 shows a lack of response to HLA-B*0702 restricted peptides from CLT Antigens 1 and 4 (CLT001 and CLT004 in the figure) in memory CD45RO-positive CD8 T-cells (panels A and C). By contrast, Naïve CD45RO-negative CD8 T-cells from the same donor respond significantly to peptides from both CLT001 and CLT004 (FIG. 63, panels B and D).

TABLE 6
CLT Antigen-derived peptides that induced
significant CD8 T-cell responses from
HLA-matched normal blood donors

| | | | HLA-type |
|---|---|---|---|
| Reference | | | for peptide |
| CLT | | Peptide | (predicted |
| Antigen | SEQ ID NO | sequence | by NetMHC) |
| 1 | SEQ ID. NO. 16 | RLQGSVTLV | HLA-A*02:01 |
| 1 | SEQ ID. NO. 17 | VPANTYNAL | HLA-B*07:02 |
| 2 | SEQ ID. NO. 30 | QLMSSFSTL | HLA-A*02:01 |
| 4 | SEQ ID. NO. 43 | FLELWLPEPM | HLA-A*02:01 |
| | | L | |
| 4 | SEQ ID. NO. 44 | APLLGSEPL | HLA-B*07:02 |
| 5 | SEQ ID. NO. 46 | TPRPDLILL | HLA-A*03:01 |
| 5 | SEQ ID. NO. 47 | RPDLILLQL | HLA-B*07:02 |
| 6 | SEQ ID. NO. 50 | FPFYKDTVLL | HLA-B*07:02 |
| 7 | SEQ ID. NO. 52 | IVLDAPVTK | HLA-A*03:01 |
| 8 | SEQ ID. NO. 55 | SLYGHIHNEA | HLA-A*02:01 |

### Example 5—Staining Reactive T-Cells with CLT Antigen Peptide Pentamers and Demonstration of their Killing of Peptide-Pulsed or CLT-Expressing Target Cells

The presence and activity of circulating CD8 T-cells specific for CLT antigens in healthy donors and melanoma patients can be measured by using HLA Class I/peptide-pentamer (“pentamer”) staining and/or in vitro killing assays. Thus, application of these methodologies to CLT antigens discovered using the methods elucidated in Examples 1 and 2 (Table 1-3, FIGS. 1-53) can be used to demonstrate the existence of therapeutically relevant T-cell responses to the CLT antigens in cancer patients.

For these studies, CD8 T-cells isolated from healthy donor or patient blood are expanded using various cultivation methods, for example anti-CD3 and anti-CD28 coated microscopic beads plus Interleukin-2. Expanded cells can then be stained for specific CLT antigen-reactivity of their T-cell receptors using CLT peptide pentamers, which consist of pentamers of HLA Class I molecules bound to the relevant CLT Antigen peptide in the peptide-binding groove of the HLA molecule. Binding is measured by detection with phycoerythrin or allophycocyanin-conjugated antibody fragments specific for the coiled-coil multimerisation domain of the pentamer structure. In addition to the pentamer stain, further surface markers can be interrogated such as the memory marker CD45RO and the lysosomal release marker CD107a. Association of pentamer positivity with specific surface markers can be used to infer both the number and phenotype (memory versus naive/stem) of the pentamer-reactive T-cell populations

Pentamer stained cells may also be sorted and purified using a fluorescence activated cell sorter (FACS). Sorted cells may then be further tested for their ability to kill target cells in in vitro killing assays. These assays comprise a CD8 T-cell population, and a fluorescently labelled target cell population. In this case, the CD8 population is either CLT antigen-specific or CD8 T-cells pentamer-sorted and specific for a positive-control antigen known to induce a strong killing response such as Mart-1. The target cells for these studies may include peptide-pulsed T2 cells which express HLA-A*02, peptide-pulsed C1R cells transfected with HLA-A*02,03, B*07, melanoma cells lines previously shown to express the CLTs/CLT antigens, patient tumor cells or cell lines such as CaSki transfected with the CLT open reading frames. Peptides used to pulse the T2 or C1R cells include CLT antigen peptides or positive control peptides. Target cell death is indicated by take up of 7AAD. In this way, as target cells are killed, by apoptosis mediated by CD8 T-cells, they gain red fluorescence. Thus, application of such killing assays to pentamer-sorted, CLT antigen-specific CD8 T-cells can be used to enumerate the cytotoxic activity of CLT-antigen-specific T-cells in ex vivo cultures of melanoma patient or healthy donor T-cells.

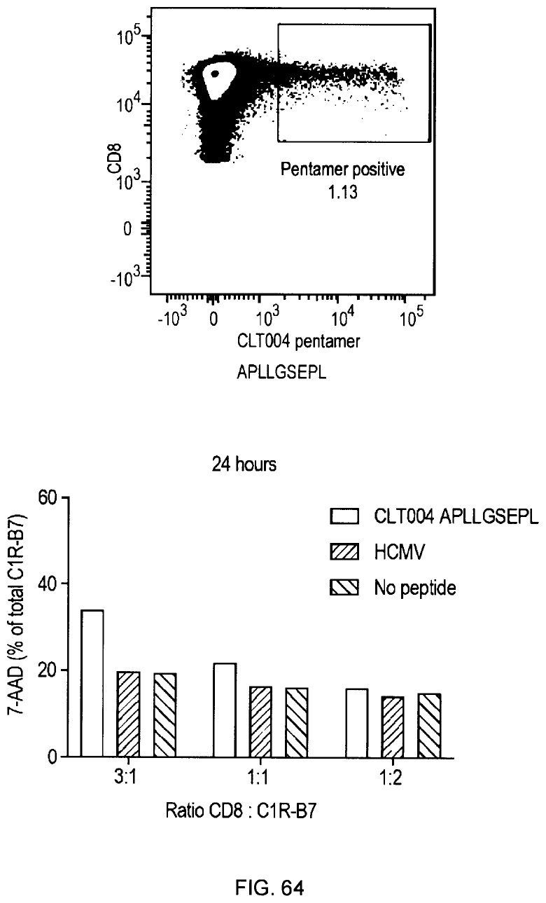

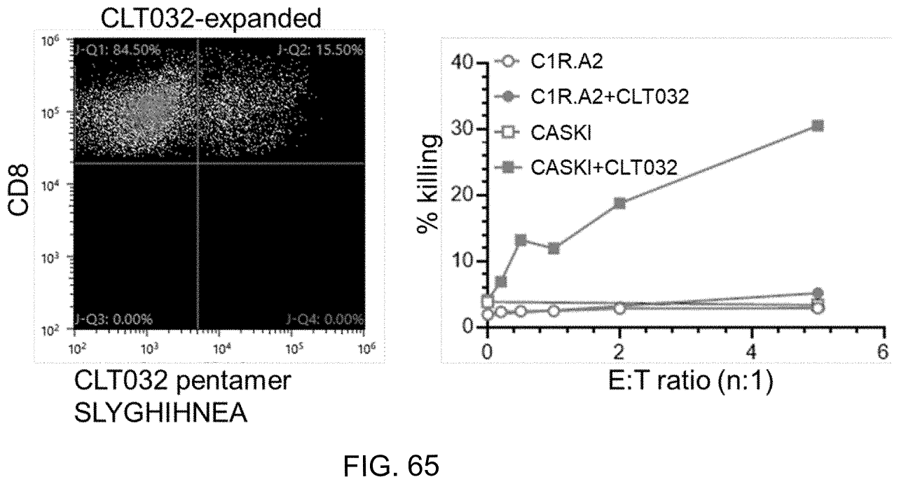

FIG. 64 shows HLA pentamer staining of healthy donor CD8 T-cells with a peptide-derived from CLT Antigen 4, peptide APPLGSEPL (top panel). The bottom panel shows antigen-specific killing of peptide pulsed C1R.B7 target cells by these CD8 T cells. The negative controls for the in vitro killing assay include an irrelevant peptide derived from human cytomegalovirus (HCMV) and no peptide. FIG. 65 shows HLA pentamer staining of healthy donor CD8 T-cells with peptides-derived from CLT Antigen 8, peptide SLYGHIHNEA following fluorescence activated cell sorting of pentamer positive cells and 14 days of expansion using anti-CD3 and anti-CD28 coated beads plus IL-2. The right-hand side panel shows very weak antigen-specific killing of peptide-pulsed A2 target cells by these CD8 T cells but effective antigen specific killing of CaSki cells transfected with the open reading frame of CLT Antigen 8. The negative controls for this in vitro killing assay include an irrelevant T2 cells with no peptide and untransfected CaSki cells.

### Example 6—Assays to Validate CLT Expression in Melanoma Cells

a) qRT-PCR Validation of CLT Expression in Melanoma Cell Lines

Quantitative real-time polymerase chain reaction (qRT-PCR) is a widespread technique to determine the amount of a particular transcript present in RNA extracted from a given biological sample. Specific nucleic acid primer sequences are designed against the transcript of interest, and the region between the primers is subsequently amplified through a series of thermocyle reactions and fluorescently quantified through the use of intercalating dyes (SYBR Green). Primer pairs were designed against the CLTs and assayed against RNA extracted from melanoma cell lines or primary patient tissue. Non-melanoma cell lines were utilised as negative controls. Melanoma cell lines used included COLO 829 (ATCC reference CRL-1974), MeWo (ATCC reference HTB-65), SH-4 (ATCC reference CRL-7724) and control cell lines HepG2 (hepatocellular carcinoma, ATCC reference HB-8065), Jurkat (T-cell leukemia, ATCC reference TIB152) and MCF7 (adenocarcinoma, ATCC reference HTB-22). Patient-derived melanoma tissue was obtained from 6 primary lesions and 6 metastases, all from patients with at least stage IIC disease. RNA was extracted from each sample and reverse transcribed into cDNA following standard procedures. qRT-PCR analysis with SYBR Green detection following standard techniques was performed with primers designed against two regions of each CLT, and reference genes. Relative quantification (RQ) was calculated as:

RQ=2[Ct(REFERENCE)−Ct(TARGET)].

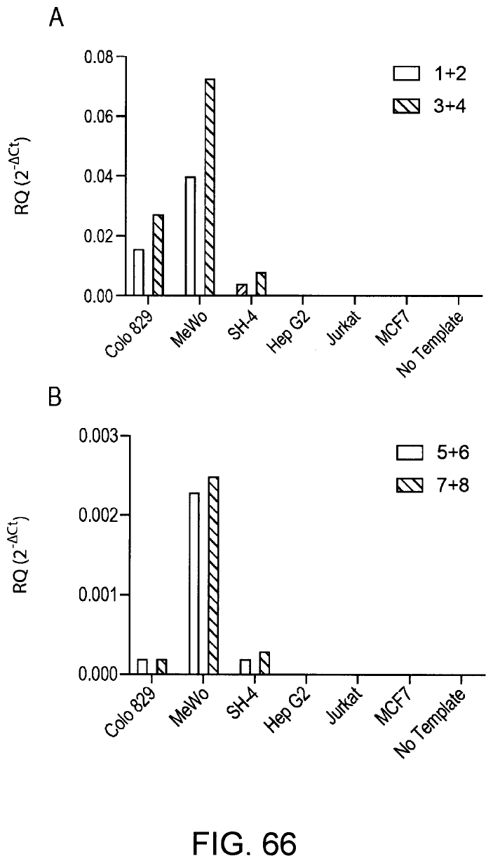

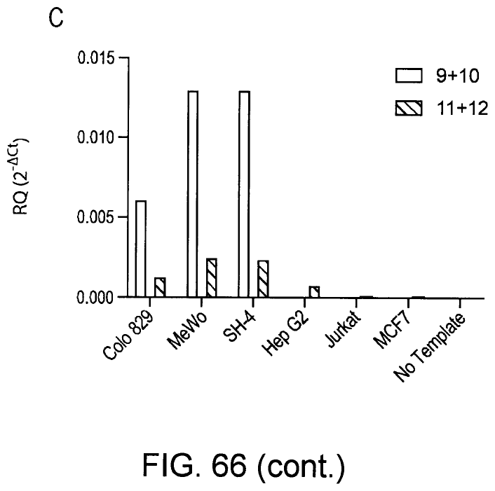

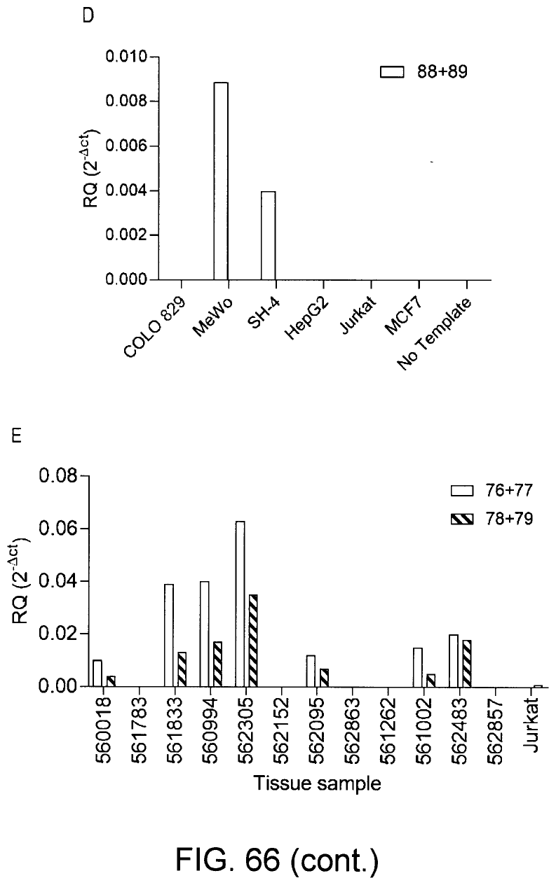

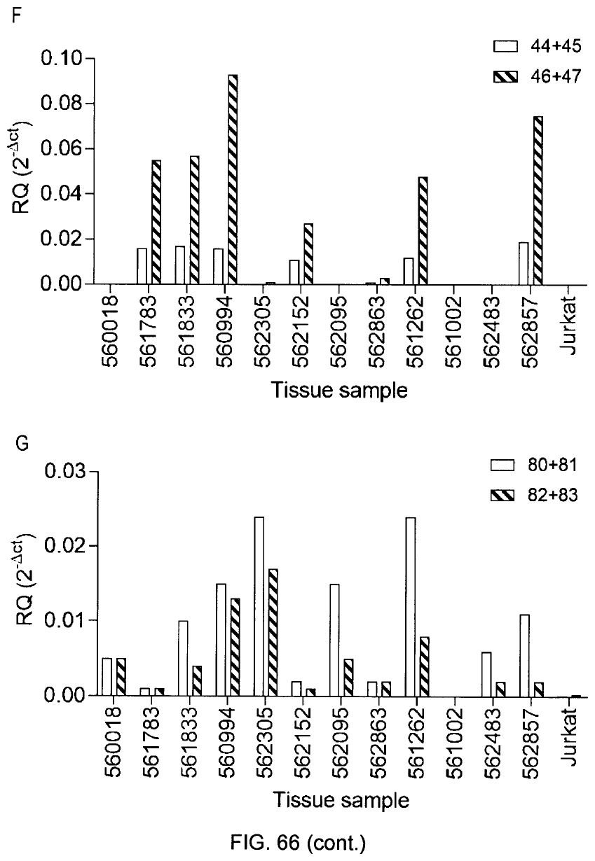

The results of these experiments are presented in FIG. 66. Panel A shows results from a qRT-PCR assay with two primer sets (1+2 and 3+4) targeting different regions of the CLT encoding CLT Antigen 1 (SEQ ID 56) on RNA extracted from three melanoma cell lines and four non-melanoma cell lines. Panel B shows results from qRT-PCR assay with two primer sets (5+6 and 7+8) targeting different regions of the CLT encoding CLT Antigen 2 (SEQ ID 57) on RNA extracted from three melanoma cell lines and four non-melanoma cell lines. Panel C shows results from qRT-PCR assay with two primer sets (9+10 AND 11+12) targeting different regions of the CLT encoding CLT Antigens 3/4 (SEQ ID 58) on RNA extracted from three melanoma cell lines and four non-melanoma cell lines. Panel D shows results from qRT-PCR assay with one primer set (88+89) targeting the CLT encoding CLT Antigen 5 (SEQ ID 59) on RNA extracted from three melanoma cell lines and four non-melanoma cell lines. Panel E shows results from qRT-PCR assay with two primer sets (76+77 AND 78+79) targeting different regions of the CLT encoding CLT Antigen 6 (SEQ ID 60) on RNA extracted from 12 melanoma tissue samples and one non-melanoma cell line. Panel F shows results from qRT-PCR assay with two primer sets (44+45 AND 46+47) targeting different regions of the CLT encoding CLT Antigen 7 (SEQ ID 61) on RNA extracted from 12 melanoma tissue samples and one non-melanoma cell line. Panel G shows results from qRT-PCR assay with two primer sets (80-81 AND 82-83) targeting different regions of the CLT encoding CLT Antigen 8 (SEQ ID 62) on RNA extracted from 12 melanoma tissue samples and one non-melanoma cell line. These results confirmed the specific expression of CLTs in RNA extracted from melanoma cell lines or tissue samples, compared to non-melanoma cell lines. Each CLT was detected in two or more cell lines or tissue samples analysed, with little to no expression detected in non-melanoma control cell lines.

b) RNAScope Validation of CLT Expression in Melanoma Cells In Situ

In situ hybridisation (ISH) methods of transcript expression analysis allow the presence and expression levels of a given transcript to be visualised within the histopathological context of a specimen. Traditional RNA ISH assays involve the recognition of native RNA molecules in situ with oligonucleotide probes specific to a short stretch of the desired RNA sequence, which are visualised through a signal produced by a combination of antibody or enzymatic-based colorimetric reactions. RNAScope is a recently developed in situ hybridization-based technique with more advanced probe chemistry ensuring specificity of the signal produced and allowing sensitive, single-molecule visualization of target transcripts (Wang et al 2012 J Mol Diagn. 14(1): 22-29). Positive staining for a transcript molecule appears as a small red dot in a given cell, with multiple dots indicative of multiple transcripts present.

RNAScope probes were designed against the CLTs and assayed on sections of 12 formalin-fixed, paraffin-embedded cutaneous melanoma tumor cores. Scoring of the expression signal was performed on representative images from each core as follows:
Estimated % cells with positive staining for the CLT probe, rounded up to the nearest 10
Estimated level of per cell expression across the given section as:
   0=no staining
   1=1-2 dots per cell
   2=2-6 dots per cell
   3=6-10 dots per cell
   4=>10 dots per cell

Expression of each of each CLT was detected across a number of different patient tumor cores, independently validating the discovery of CLTs from tumor-derived RNAseq data and confirming homogeneity of expression within tumor tissue across certain samples and also highlighting the presence of at least one CLT in each patient core analysed.

TABLE 10
Scoring of RNAScope in melanoma patient tissue cores

| | | CLT Antigen 1 | | CLT Antigen 2 | | CLT Antigen 3/4 | |
|---|---|---|---|---|---|---|---|
| | | (SEQ ID 56) | | (SEQ ID 57) | | (SEQ ID 58) | |
| | | % of | | % of | | % of | |
| | | (+)ve | | (+)ve | | (+)ve | |
| Tissue | Core | cells | Score | cells | Score | cells | Score |
| Melanoma | 61 | 10 | 1 | 80 | 4 | 10 | 1 |
| Melanoma | 62 | 80 | 2 | 80 | 2 | 100 | 4 |
| Melanoma | 63 | 100 | 4 | 0 | 0 | 100 | 4 |
| Melanoma | 64 | 0 | 0 | 0 | 0 | 100 | 3 |
| Melanoma | 65 | 70 | 3 | 0 | 0 | 80 | 4 |
| Melanoma | 66 | 100 | 3 | 50 | 2 | 90 | 3 |
| Melanoma | 67 | 100 | 3 | 100 | 4 | 50 | 2 |
| Melanoma | 68 | 0 | 0 | 0 | 0 | 100 | 3 |
| Melanoma | 69 | 90 | 3 | 100 | 4 | 100 | 4 |
| Melanoma | 70 | 0 | 0 | 0 | 0 | 70 | 3 |
| Melanoma | 71 | 0 | 0 | 0 | 0 | 80 | 4 |
| Melanoma | 72 | 100 | 3 | 100 | 4 | 60 | 2 |

| | | CLT Antigen 5 | | CLT Antigen 6 | | CLT Antigen 7 | | CLT Antigen 8 | |
|---|---|---|---|---|---|---|---|---|---|
| | | (SEQ ID 59) | | (SEQ ID 60) | | (SEQ ID 61) | | (SEQ ID 62) | |
| | | % of | | % of | | % of | | % of | |
| | | (+)ve | | (+)ve | | (+)ve | | (+)ve | |
| Tissue | Core | cells | Score | cells | Score | cells | Score | cells | Score |
| Melanoma | 61 | 30 | 2 | 0 | 0 | 0 | 0 | 10 | 1 |
| Melanoma | 62 | 20 | 1 | 90 | 3 | 20 | 1 | 10 | 1 |
| Melanoma | 63 | 10 | 1 | 100 | 4 | 0 | 0 | 80 | 2 |
| Melanoma | 64 | 70 | 2 | 50 | 1 | 0 | 0 | 40 | 2 |
| Melanoma | 65 | 10 | 1 | 50 | 2 | 30 | 2 | 50 | 2 |
| Melanoma | 66 | 80 | 3 | 100 | 4 | 50 | 1 | 50 | n/a |
| Melanoma | 67 | 100 | 4 | n/a | n/a | n/a | n/a | n/a | n/a |
| Melanoma | 68 | 30 | 2 | 40 | 1 | 0 | 0 | 50 | 1 |
| Melanoma | 69 | 10 | 1 | 80 | 3 | 80 | 1 | 50 | 1 |
| Melanoma | 70 | 10 | 1 | 10 | 1 | 20 | 1 | 10 | 1 |
| Melanoma | 71 | 50 | 3 | 0 | 0 | 0 | 0 | 10 | 1 |
| Melanoma | 72 | 90 | 3 | 100 | 3 | 0 | 0 | 10 | 1 |

### Example 7—Ex Vivo Stimulation of T Cells Using Pools of CLT Antigens or CLT Antigen Fusion Proteins

T cells from a healthy donor or patient with a given cancer, can be stimulated outside of the body (ex vivo) to activate T cell clones that recognise specified CLT Antigens, and subsequently rapidly expanded to generate large numbers of CLT-reactive T cells, where resultant anti-tumor activity might be anticipated. A number of steps are involved to employ this method.

A) Isolation of Relevant Patient Immune Cells

T cells from the donor (healthy or cancer patient) must be isolated but also autologous antigen presenting cells (APCs) may be required. The source of the immune cells can be obtained from peripheral blood through a blood draw or apheresis. Alternatively, T cells can be isolated from the tumor infiltrating lymphocytes (TILs) obtained from fresh biopsy or resection of a patient's tumor. APCs may be CD14-positive monocytes or alternatively dendritic cells (DCs) which would be derived from the monocyte fraction of the apheresis product. DCs can be generated by methods such as positive isolation via CD14 capture (for example, anti-CD14 antibodies conjugated to magnetic beads, where CD14-positive cells are labelled with the beads and captured on a magnetic column) or isolation via their adhesive properties, for example, adherence to tissue culture plastics by incubation of peripheral blood mononuclear cells (PBMCs) with cell culture dishes for a period of 4-48 hr to allow adherence of monocytes. DCs can be generated from the CD14-positive or adherent immune cell fractions by well-described methods utilising cytokines such as, but not limited to: GM-CSF, IL-4, TNFα, IL-1β, IL-6, Prostaglandin E2. Incubation with such cytokines over the course of 2-7 days allows differentiation of the CD14+ monocytes into DCs, typically that will have lost the expression of CD14 and upregulated expression of DC markers such as CD11c, high levels of MHC Class II, etc. The nature of the T cells for selection and/or stimulation could be the monocyte-depleted fraction of PBMC (in the case of apheresis origin of T cells), pan-T cell isolation using isolation techniques based on the expression of markers such as CD3, or presence or absence of markers of specific T cell subsets, for example but not limited to, CD4, CD8, CD45RO, CD45RA, CCR7, CD62L, CD27 etc.

B) Selection of CLT Antigen-Recognising T Cells

Methods can be employed to select T cells prior to stimulation with APCs. Such methods would include peptide-HLA (pHLA) multimer approaches such as tetramer, pentamer, dextramer or similar, to label T cells that express TCRs that recognise the given pHLA. Such pHLAs would be defined based on mass spectrometry (MS) experiments as described in Example 2, and/or peptides predicted to bind specific HLA allotypes based on prediction algorithms. The multimer could possess a tag, such as phycoerythrin (PE) which could be isolated using fluorescent activated sorting or via an anti-PE antibody conjugated to magnetic beads. Alternatively, an antibody to the tag could be directly conjugated to magnetic beads. To isolate different T cells that recognise different pHLAs from different CLT Antigens, multimers could be generated with the same or different tags, or different multimers could be conjugated to magnetic beads.

C) Stimulation of T Cells

In order to potentiate pre-existing (memory) or stimulate new (naïve) T cell responses from cancer patients to CLT Antigens, the patient's T cells can be exposed to APCs that are presenting peptides derived from CLT Antigens on the surface in the context of Class I and Class II HLA complexes. This could involve the introduction of multiple CLT Antigens (anticipated to be expressed by a patient's tumor) into the APCs, such as autologous DCs generated from the patient's apheresis product. Introduction of CLT Antigens could be through concatenated polypeptide delivery of multiple CLT Antigens, such as viral vector delivery, or as individual pooled CLT Antigens, such as mRNA-based methods of delivery. Methods of stabilized, mature, mRNA delivery to the APC (that is, transfection) could include classical reagents such as polyethylenimine (PEI) or calcium phosphate for nucleic acid delivery into cells. Alternatively, efficient transfection can be achieved using lipid-based reagents for transfection into APCs. These transfection reactions use synthetic, in vitro transcription reaction (IVT)-derived mRNAs formulated in lipid complexes such as such as lipid nanoparticles (LNP) or lipid-based lipoplexes (formed by simple mixing of mRNAs with lipid reagents). To create these mRNAs, recombinant DNA constructs containing the well-described promoter element for phage T7 DNA-dependent, RNA polymerase, followed by a cDNA encoding high-stability mRNA 5′UTR, a cDNA encoding a codon-optimized open reading frame (ORF) for a CLT Antigen, a cDNA encoding a high stability mRNA 3′UTR, a poly-A sequence of >20 nucleotides, and a unique restriction endonuclease site designed to release a functional poly-A tail, can be used as a template for in vitro transcription (IVT) of suitable CLT Antigen-encoding mRNAs. To create human APC-expressing IVT mRNA-encoding antigens, lipoplex methods similar to those described by Cafri et al., Nat. Comm. 2019 could be used. Briefly, APCs (monocytes or DCs) would be plated on tissue culture flasks to achieve confluence of 70-90%. A lipid-based transfection reagent (for example, Lipotectamine™ MessengerMAX™ or FuGENE® HD or similar) would be diluted appropriately in serum-free medium such as Opti-MEM™, mixed and incubated with mRNA encoding the CLT Antigen. This could be done with multiple CLT Antigen mRNAs for transfection of a combination of CLT Antigens to the APC. Incubation times of the mRNA with the lipid reagent would be short (5-10 minutes) and at room temperature. The resultant mRNA-lipid complex would be added to APCs and incubated at 37° C./5% CO₂ for 16-72 hours, depending on optimal timepoint for presentation of translated peptides from the CLT-encoding mRNA molecules.

Delivery of CLT Antigens to APCs with such methods described should result in the expression of CLT Antigen polypeptides in the cytoplasm of the APC, which in turn will result in cellular processing of peptide fragments from the polypeptides for presentation on Class I and Class II HLA molecules. When T cells (either selected as described in (b) or unselected T cells from apheresis or TIL sources) are co-cultured with APCs expressing CLT Antigen-derived peptide-HLA complexes at the cell surface, those T cells possessing TCRs that have specificity for a given pHLA will be stimulated by engaging with the pHLA complex in addition to co-stimulatory molecules and signals from the APC. This will result in activation, differentiation and proliferation of the engaged T cell. For example, following successful transfection of APCs with IVT mRNA-encoding CLT Antigens in a method as described above, autologous CD3+ isolated T cells would be co-cultured with the APCs at a ratio of excess T cell to APC, for example 10 T cells per 1 APC (10:1), in cytokine-containing medium (such as IL-6 and IL-12 or other cytokines supplemented in the basal media used). The cells would be co-cultured for as little as overnight or up to 1 week to stimulate T cells, but typically 18-48 hours after which the T cells could be subjected to enrichment prior to expansion, if required.

D) Enrichment of Stimulated T Cells

T cells that have been stimulated by APCs that are expressing CLT Antigens can be further enriched prior to an expansion step if required. Markers of T cell activation (such as CD137, CD107a, CD69, OX40 or other surface marker associated with an activated state) or T cell functional responses (for example, T cells secreting cytokines such as TNFα or IFNγ) could be selected for, to enrich the T cell population for those cells that might be CLT Antigen-specific. Such enrichment methods could include cell sorting by FACS or bead-based methods of capture, for example, using antibodies to CD137 or similar that are conjugated to magnetic beads. Multiple enrichment strategies could be employed, either in parallel (for example, cells double positive for CD137 and CD69) or sequentially (for example, selecting cells positive for CD137 and subsequently selecting CD137+ cells positive for CD69). Such a positive selection should remove those T cells that are likely not stimulated by the CLT Antigen-expressing APCs.

E) Rapid Expansion of Stimulated T Cells

Following stimulation of T cells with APCs that have had CLT Antigens introduced into them, bulk or enriched (see (d) above) T cells can be rapidly expanded to achieve numbers >10⁸ total cells, using methods based on those described in the literature, with potential modifications for optimisation (for example, Jin et al, J Immunother, 2012). Such methods utilise cytokines such as IL-2 and stimulatory antibodies such as anti-CD3 as well as potential irradiated autologous cells from PBMC (termed “feeder” cells). Alternatively stimulatory antibodies to CD3 and CD28 can be used to avoid the use of feeder cells. The process can be further automated or enhanced using specialized gas-permeable flasks (for example G-Rex flasks) or closed expansion system (for example WAVE bioreactor). Significant expansion of T cells (100-1000 fold) can be achieved in as little as 7-14 days, depending on the numbers of T cells at the start.

F) Testing of Expanded T Cells for Evidence of CLT Antigen Immunogenicity

To demonstrate that the ex vivo autologous stimulation process has expanded T cells that recognize CLT Antigen-expressing target cells (including tumor cells), multimers corresponding to specific CLT Antigen peptide-HLA (pHLA) complexes could be used to detect the presence of T cells with reactivity for a particular CLT Antigen pHLA. Multiple pHLAs from a combination of CLT Antigens could be used with different labels to demonstrate recognition of more than 1 CLT Antigen by the ex vivo-stimulated T cells.

Functional assays would also demonstrate the ability of the ex vivo-immunized T cells to respond to target cells presenting peptides from the CLT Antigens. This could be achieved through a variety of approaches. Firstly, cytokine release assays could be performed to test for T cell activation from co-cultivation of the ex-vivo stimulated T cells with the target cells (for example, IFNγ ELISpot assays). Alternatively, T-cell mediated killing of target cells could be measured with cytotoxicity assays such as FACS-based methods to assess cell death of target cells (e.g. by 7-AAD measurement) co-cultured with the T cells, or other methods such as those that monitor markers of apoptosis of target cells or measure impedance (electrical measure of cell viability) of adherent target cells plated onto specialized surfaces.

A variety of methods could be used to create target cells for such assays. For example appropriate human cells with HLAs that match APCs used in the ex vivo stimulation could be pulsed with peptides derived from CLT Antigens that are known to be presented on Class I HLA molecules (as deconvoluted from mass spectrometry experiments—see Example 2). Further, tumor cell lines matching the HLA type of the APCs could also be assessed. Finally, primary tumor cells (in particular tumor cells from the same patient donor from which the starting T cells and APCs used for the process were derived) could be assessed.

In conclusion, these methods can be used to demonstrate that human T cells are able to be “immunized” with CLT Antigens ex vivo, producing immunologically reactive/cytolytic T cells, confirming the likelihood that vaccination of cancer patients with one or more CLT Antigen would have therapeutic value in controlling cancer.

### Example 8—Methodology for CLT Antigen Fusion Protein Design

To facilitate delivery of a multi-polypeptide antigen mixture via a vectored vaccine, it is highly desirable to combine the genes for component antigens into a single ORF, resulting in the synthesis of an antigenic fusion protein. Further, rather than directly linking the component polypeptides together, these can be connected by peptide linker regions to: 1) reduce the potential risk of generating novel epitopes at fusion junctions that mimicked normal human proteins (increasing safety) and 2) ensure that CLT Antigen T cell epitopes bordering the fusions/linkers are processed in a manner that mimics their presentation when expressed from individual ORFs encoded by tumor tissues (increasing effectiveness). To accomplish linkage to facilitate the design of safe and effective fusion proteins, an algorithm was developed. Simple short linkers are used in this algorithm, since these would be superior for achieving the above-mentioned goals. To this end, multiple Gly-based linkers were selected, some of which also contained Lys residues to eliminate identity to normal human proteins and facilitate processing at the ends of the component CLT antigens (GGG, GGGG, KGG, GGKGG, GGGKGG, GGK; SEQ ID NO. 71-75, 84 respectively).

For the purposes of this example, the algorithm was applied to two different sets of CLT Antigens, CLT Antigens 1,2,4,6,7 and 8 (CLT Antigen Fusion Protein 1 and 2) and CLT Antigens 1-8 (CLT Antigen Fusion Protein 3 and 4).

To accomplish the needs described above, six criteria were considered in the design of each of four individual fusion protein (CLT Antigen Fusion Protein 1/CLT Antigen Fusion Protein 2 for a six-CLT Antigen vaccine regimen and CLT Antigen Fusion Protein 3/CLT Antigen Fusion Protein 4 for an eight-CLT Antigen vaccine regimen). These were applied one-by-one, and then repeated, as needed, in an iterative manner to ensure that the final fusion protein candidates satisfied all criteria.

First, fusion protein sequences were designed so that no 9-mer peptides containing any portion of a linker peptide could be identical to the human proteome, as determined by a blastp search performed by Standalone Blast ver2.9.0 (AltSchul et al, J. Mol. Biol. 1990). For completeness, this blastp search was performed against three proteome sub-databases extracted from the Ensemble database (www.ensembl.org); SwissProt Human proteome, Trembl Ensembl Human up000005640 proteome, and Trembl all human proteome (created 14 Aug. 2019).

Second, fusion protein sequences were designed so that no 9-mer peptide containing any portion of a linker peptide could be a strong predicted binder (rank 0.5) for an MHC class I supertype (see below) by NetMHCpan 4.0. (Andreatta & Nielsen, Bioinformatics 2016). For the generation of a fusion protein encoding CLT Antigens suitable for use in a melanoma therapy, MHC HLA class I types important for the target population of melanoma patients was the key driver, resulting in selection of the following supertypes: HLA-A*01:01, HLA-A*02:01, HLA-A*03:01, HLA-A*11:01, HLA-A*24:02, HLA-A*25:01, HLA-A*26:01, HLA-A*68:01, HLA-B07:02, HLA-B*08:01, HLA-B*18:01, HLA-B*27:05, HLA-B*35:01, HLA-B*35:03, HLA-B*40:01, HLA-B*40:02, HLA-B51:01, HLA-C*07:01, HLA-C*07:02.

Third, fusion protein sequences were designed so that CLT Antigens for which HLA-bound peptides (see Example 2) were found precisely aligned with their C-termini were prioritized for positioning at the C-terminus of the fusion protein designs to help ensure that the C-terminal anchor residues (normally released by a stop codon when expressed in tumor tissues) would be similarly produced in the context of a fusion protein. When C-terminal placement was not possible for such CLT Antigens, linker sequences were further optimized based on proteasomal cleavage site predictions made with the NetChop 3.1 Server (Nielsen et al., Immunogenetics 2005) to select linker sequences expected to produce the C-termini found in the authentic (stop-codon-generated) CTA antigen polypeptides.

Fourth, fusion protein sequences were designed so that all 9-mer peptide sequences containing any portion of a linker peptide that were predicted to be weak binders (rank score 2.0) for a selected MHC class I supertype (see above) were altered to eliminate or reduce binding by adjusting linkers or, in some cases, by removing the N-terminal methionines from component CLT antigens. The truncation of N-terminal methionines was also adopted as a strategy to eliminate 9-mers predicted to weakly bind selected MHC Class I molecules, since methionine is rarely found in the N-terminal position of MHC Class I bound peptides (Abelin et al., Immunity 2017; Alvarez et al., Molecular & Cellular Proteomics 2019). Elimination/reduction of peptide sequences predicted to be weak binders for high frequency HLA class I types (HLA-A*02:01, HLA-A*03:01, HLA-B*07:02) were prioritized for alteration. Where possible, the elimination/reduction of peptide sequences predicted to be weak binders to all other selected supertypes (see above) was achieved by using the same procedures.

Fifth, since one application of the fusion protein cassettes is in a prime/boost regimen, the pairs of the fusion protein designs used for this purpose were designed so that there were not allowed to directly repeat CLT antigen junctions to reduce epitope repetition.

Sixth, the pairs of fusion proteins were refined to remove predicted weak binding epitopes repeated between prime and boost constructs (implemented for CLT Antigen Fusion Protein 3 and 4). This was achieved by either excluding N-terminal methionine residues (see rationale described above for N-terminal methionine removal) or by the use of alternative linkers (see above).

Implementation of Design for CLT Antigens

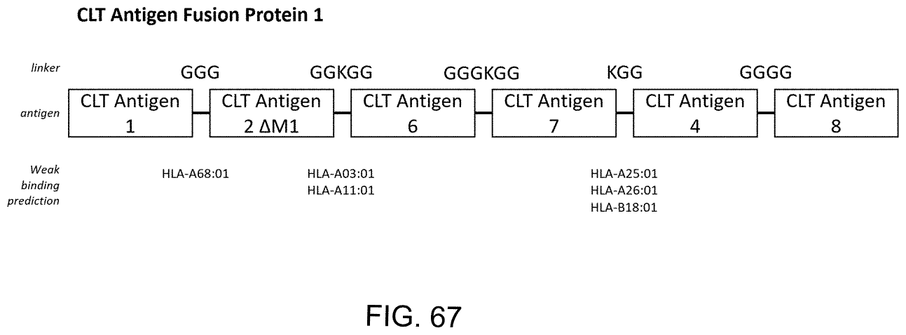

The result of the fusion protein design strategy described above was the four constructs (CLT Antigen Fusion Protein 1 (SEQ ID NO. 76), CLT Antigen Fusion Protein 2 (SEQ ID NO. 77), CLT Antigen Fusion Protein 3 (SEQ ID NO. 78), CLT Antigen Fusion Protein 4 (SEQ ID NO. 79)) shown in schematic form in FIGS. 67-70.

### Example 9—Fusion Protein Antigenicity

CLT Antigen Fusion Proteins designed as described in Example 8 are expected to be translated, proteolytically processed in the cytosol, and presented in association with HLA class I molecules on the cell surface. cDNA constructs encoding the fusion protein cassettes were transduced into human cells, and the HLA class I molecules were immunoprecipitated and subjected to MS analyses described in Example 2 for the discovery of the CLT antigens. This is done in order to demonstrate that the CLT Antigen fusion protein cassettes maintained similar antigen presentation properties of the component CLT Antigens previously identified in tumors tissues (shown Example 2).

MS-based immunopeptidomics analysis is a powerful technology that allows for the direct detection of specific peptides associated with HLA class I molecules. However, the ability to detect individual peptides is influenced by their biophysical properties, it is restricted by the proteolytical activity present in the cells and HLA alleles expressed in the cell lines used for these studies. Thus, the method will not likely discover all previously identified HLA-bound peptides in a tissue or cell sample. Nevertheless, the repertoire of HLA class I-bound peptides detected will confirm the value of the fusion protein designs tested in delivering peptide epitopes from CLT Antigens.

To accomplish MS-based studies of fusion protein designs, cultured human cells are transduced with plasmid DNAs encoding the CLT Antigen fusion protein cassettes under control of suitable polII promoter and 5′ and 3′ UTRs. After expansion, the cultured cells encoding the CLT Antigen fusion protein cassettes are lysed and the HLA class I-peptide complexes are affinity purified by anti-HLA Class I antibody capture. The isolated HLA molecules and bound peptides are then separated from each other and the eluted peptides are analyzed by nUPLC-MS/MS. The MS/MS spectra acquired from these HLA Class I pull downs are then interrogated by using the PEAKS™ software (v8.5 and vX, Bioinformatics Solutions Inc). For MS/MS interpretation the software evaluates side-by-side all theoretical spectra of polypeptides contained in the human proteome with the polypeptide of the relevant fusion protein. For these studies it is essential that the repertoire of analyzed sequences contains sequences of the relevant CLT Antigen fusion protein construct AND the human proteome since the great majority of Class I HLA-bound peptides found in cells are derived from constitutively expressed proteins.

The results of these studies identify individual CLT Antigens fusion protein-derived peptides processed and presented by the HLA Class I repertoire of the transduced cells. Analyses of these data are used to demonstrate that the design has resulted in effective presentation of peptides from the individual CLT Antigens. Moreover, the results of these studies show that epitopes derived from linker regions of the tested protein fusions are not efficiently presented in CLT Antigen fusion protein cDNA transduced cells.

Taken together, these data can provide strong support for the translation, processing, and presentation of CLT Antigen epitopes from the CLT Antigen Fusion Protein constructs. Thus, enabling the use of these CLT Antigen fusion proteins in vaccines (or other therapeutic modalities) designed to elicit T cells that recognize the CLT Antigen peptides/HLA Class I complexes on tumors found in patients.

### Example 10 Killing of Fusion Protein Expressing Cell Lines

The immunogenicity of antigens derived from cells expressing CLT Antigen Fusion Protein open reading frames can be demonstrated using transfected cell lines combined with the CLT-peptide-reactive T cells described in Example 5. Killing of CLT Antigen Fusion Protein-transfected cell lines by CLT Antigen-specific CD8 T cells is used to demonstrate the existence of therapeutically relevant T-cell responses to the concatenated combination of CLT Antigens in cancer patients.

CaSki cells which have been transfected with constructs encoding the CLT Antigen Fusion Proteins 1, 2, 3 or 4 (SEQ ID NO. 76-79) described in Examples 8 and 9 are used as targets for killing assays as described in Example 5. CD8 T cell lines isolated from healthy donors and melanoma patients using HLA-pentamers derived from CLT Antigens 1, 2, 3, 4, 5, 6, 7 and 8 are tested individually for killing ability of these CLT Antigen Fusion Protein transfected target cells. The negative control cells are untransfected CaSki or CaSki cells transfected with an irrelevant construct.

### Example 11 Mouse Immunogenicity Studies

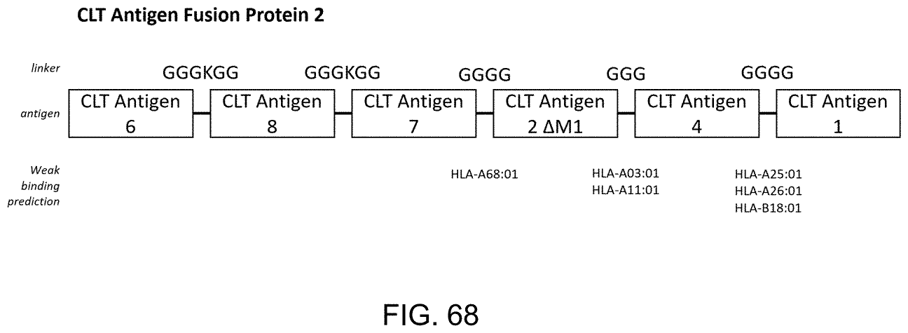

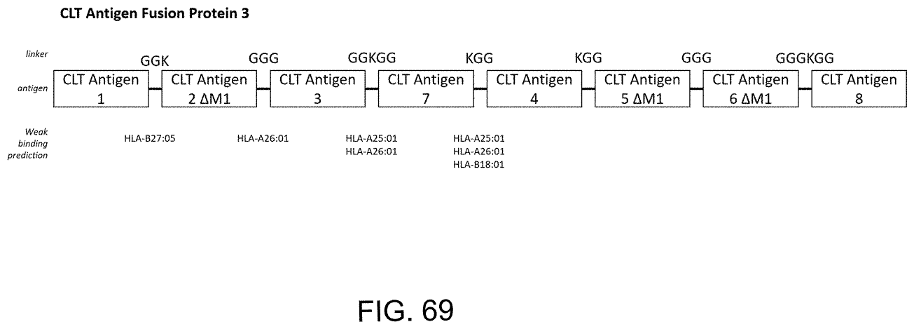

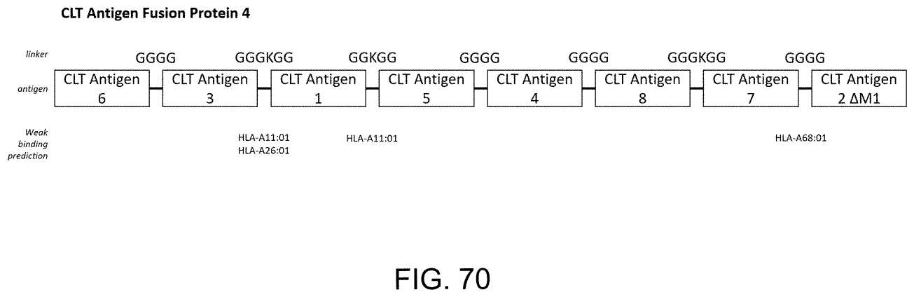

To demonstrate the immunogenicity of individual component CLT Antigens within the fusion protein constructs described in Example 8 mice can be administered a priming immunization with ChAdOx1 vectors (a replication-deficient chimpanzee adenovirus vector) encoding a priming CLT antigen fusion protein sequences (CLT Antigen Fusion Protein 1 or CLT Antigen Fusion Protein 3; FIGS. 67 and 69; SEQ ID NO. 76 and SEQ ID NO. 78), and given a booster immunization with MVA vectors (replication-deficient modified vaccinia Ankara vectors) encoding a boosting CLT Antigen fusion protein sequence (CLT Antigen Fusion Protein 2 or CLT Antigen Fusion Protein 4, respectively; FIGS. 68 and 70, SEQ ID NO. 77 and SEQ ID NO. 79).

For these studies, outbred mice (laboratory mice derived from a population with diverse Major Histocompatibility class I and II molecules) will be used, to more closely mimic the outbred-nature of humans. In addition, as referenced above, the experiment will use priming (CLT Antigen Fusion Protein 1, CLT Antigen Fusion Protein 3; FIGS. 67 and 69; SEQ ID NO. 76 and SEQ ID NO. 78) and boosting vectors (CLT Antigen Fusion Protein 2, CLT Antigen Fusion Protein 4; FIGS. 68 and 70, SEQ ID NO. 77 and SEQ ID NO. 79) to mimic the use of fusion protein antigens in a prime/boost clinical application.

Despite the utility of the design for use in man (see Example 8), many aspects of this design, including the elimination of linker-derived sequences that match the human proteome, and the elimination/reduction of linker-derived sequences predicted to bind to human HLA Class I molecules, cannot be tested in a murine immunization model.

Nevertheless, by focusing murine studies on immune responses to the CLT Antigens themselves, these studies do provide useful information on the fusion protein design and processing, since the antigen processing machinery in murine and human cells is similar (Kumanovics A, Takada T, Lindahl K F. Genomic organization of the mammalian MHC. Annu Rev Immunol 2003; 21: 629—57. DOI: 10.1146/annurev.immunol.21.090501.080116. Madi A, Poran A, Shifrut E et al. T cell receptor repertoires of mice and humans are clustered in similarity networks around conserved public CDR3 sequences. eLife 2017; 6:e22057. DOI: 10.7554/eLife.22057).

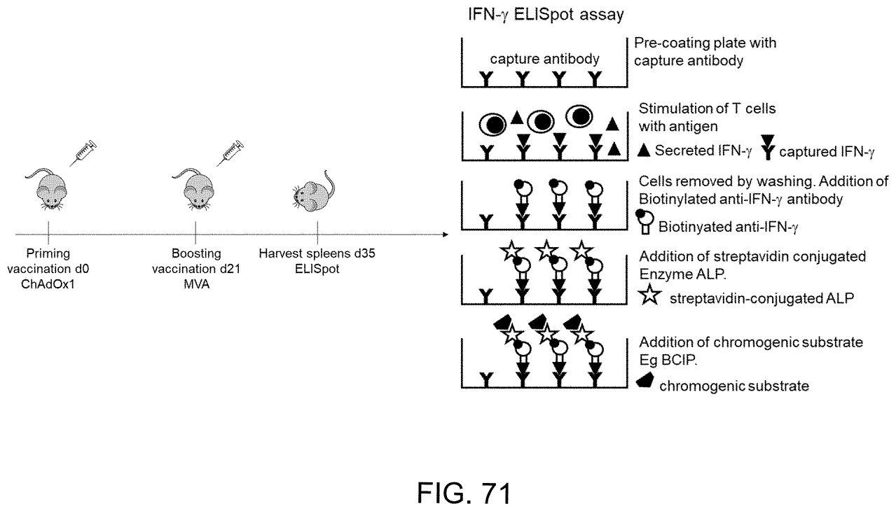

To assess cancer-relevant immunogenicity in vaccinated animals, immune cells harvested from vaccinated mice are tested for the presence of CLT antigen-specific T cells by using IFNγ ELISPOT assays (Mennuni et al., Int. J. Cancer, 2005). Briefly, mice vaccinated as described above are humanely euthanized, and spleen cells prepared from these animals are loaded into wells of a multi-well dish derivatized with monoclonal antibodies to murine IFNγ, in the presence (or absence) of overlapping peptides corresponding to sequences of one or more of the authentic CLT antigens (see schematic in FIG. 71).

Following a suitable incubation time (e.g., 16-24 hours), the immobilized IFNγ secreted by the peptide-activated T cells is stained with a second anti-IFNγ monoclonal antibody (selected to specifically detect IFNγ in the presence of the immobilizing monoclonal antibody), permitting enumeration of the cells/spots which indicate the presence of vaccine-elicited murine T cells that recognize the CLT Antigen peptides loaded into the wells. To calculate specific immune responses to the stimulating CLT Antigen peptide pools, the spot numbers are normalized to the numbers of IFNγ-stained spots found in wells of splenocytes from the same animal, that have been incubated in the absence of peptides.

Data from these studies demonstrate the immunogenicity of CTL Antigens present in the fusion protein constructs used to vaccinate the mice, confirming the utility of these fusion proteins in priming and boosting immune responses to their component CLT Antigens.

SEQUENCE LISTING
(Polypeptide sequence of CLT Antigen 1)
SEQ ID NO. 1
MWNFFRRELTSNGFPENFSLDVPANTYNALKSRL
CDPNADHTSCPSPCSLHAAGALPGTGRQRWRVEL
AHLADRKLSLRDVSRLRQGGERRSGIAVKVVRGG
AGFAARLQGSVTLVQQGWFFPRLGGCQAWWRMGA
VVWCGELLTCTS
(Polypeptide sequence of CLT Antigen 2)
SEQ ID NO. 2
MTGVLIRRGDLVTDMVACRIKTFRGHTEKAAICKT
RKESSAETSPADSLILDFQPLQLMSSFSTLASLDK
(Polypeptide sequence of CLT Antigen 3)
SEQ ID NO. 3
MNTPNIVSLRAHQPEVGIIPSVLLMRPLRIKGVFH
HIHSPLHGENQGFTLCLQGAPPSSSV
(Polypeptide sequence of CLT Antigen 4)
SEQ ID NO. 4
MAKTKGSLSVFRELHPAAAFDRAVHFLFLELWLPEP
MLSSSPPSSTAPLLGSEPLRHWEASLSR
(Polypeptide sequence of CLT Antigen 5)
SEQ ID NO. 5
MLPRTPRPDLILLQLLPAGLRQLLQTSGPDNEQPIE
QDLICNVC
(Polypeptide sequence of CLT Antigen 6)
SEQ ID NO. 6
MWNSLEARSPKSRCCSTGPWEAVQENLLWASLLAPG
GATIFPDPWLLKASPSSLPHLHRTSPCACLCPNFPF
YKDTVLLHQGPR
(Polypeptide sequence of CLT Antigen 7)
SEQ ID NO. 7
MAMGQRTPSLAELRKSSATFLTCNLGAQAEKRSRAPG
KLTYVSTIVLDAPVTKLEQGLVMKRYKIVTQGFDYT
SVES
(Polypeptide sequence of CLT Antigen 8)
SEQ ID NO. 8
MCALQGRGASPAGAGLFHWTMSPFLLGSLYGHIHNEAV
(peptide sequence derived from CLT
Antigen 1)
SEQ ID NO. 9
VQQGWFFPR
(peptide sequence derived from CLT Antigen 1)
SEQ ID NO. 10
WRGGAGFAAR
(peptide sequence derived from CLT Antigen 1)
SEQ ID NO. 11
HLADRKLSL
(peptide sequence derived from CLT Antigen 1)
SEQ ID NO. 12
ARLQGSVTL
(peptide sequence derived from CLT Antigen 1)
SEQ ID NO. 13
VPANTYNALK
(peptide sequence derived from CLT Antigen 1)
SEQ ID NO. 14
RLGGCQAWWR
(peptide sequence derived from CLT Antigen 1)
SEQ ID NO. 15
ANTYNALKSR
(peptide sequence derived from CLT Antigen 1)
SEQ ID NO. 16
RLQGSVTLV
(peptide sequence derived from CLT Antigen 1)
SEQ ID NO. 17
VPANTYNAL
(peptide sequence derived from CLT Antigen 2)
SEQ ID NO. 18
ADSLILDF
(peptide sequence derived from CLT Antigen 2)
SEQ ID NO. 19
SSFSTLASLDK
(peptide sequence derived from CLT Antigen 2)
SEQ ID NO. 20
LVTDMVACRI
(peptide sequence derived from CLT Antigen 2)
SEQ ID NO. 21
LILDFQPLQL
(peptide sequence derived from CLT Antigen 2)
SEQ ID NO. 22
MSSFSTLASL
(peptide sequence derived from CLT Antigen 2)
SEQ ID NO. 23
LMSSFSTLASL
(peptide sequence derived from CLT Antigen 2)
SEQ ID NO. 24
LMSSFSTLA
(peptide sequence derived from CLT Antigen 2)
SEQ ID NO. 25
QLMSSFSTLA
(peptide sequence derived from CLT Antigen 2)
SEQ ID NO. 26
MVACRIKTFR
(peptide sequence derived from CLT Antigen 2)
SEQ ID NO. 27
VTDMVACRIK
(peptide sequence derived from CLT Antigen 2)
SEQ ID NO. 28
SPADSLIL
(peptide sequence derived from CLT Antigen 2)
SEQ ID NO. 29
SLILDFQPL
(peptide sequence derived from CLT Antigen 2)
SEQ ID NO. 30
QLMSSFSTL
(peptide sequence derived from CLT Antigen 3)
SEQ ID NO. 31
NTPNIVSLR
(peptide sequence derived from CLT Antigen 3)
SEQ ID NO. 32
RPLRIKGVF
(peptide sequence derived from CLT Antigen 3)
SEQ ID NO. 33
NTPNIVSLRA
(peptide sequence derived from CLT Antigen 3)
SEQ ID NO. 34
VLLMRPLRIK
(peptide sequence derived from CLT Antigen 3)
SEQ ID NO. 35
MRPLRIKGVF
(peptide sequence derived from CLT Antigen 4)
SEQ ID NO. 36
KTKGSLSVFR
(peptide sequence derived from CLT Antigen 4)
SEQ ID NO. 37
AAFDRAVHF
(peptide sequence derived from CLT Antigen 4)
SEQ ID NO. 38
AFDRAVHF
(peptide sequence derived from CLT Antigen 4)
SEQ ID NO. 39
KTKGSLSVF
(peptide sequence derived from CLT Antigen 4)
SEQ ID NO. 40
FLFLELWL
(peptide sequence derived from CLT Antigen 4)
SEQ ID NO. 41
SVFRELHPA
(peptide sequence derived from CLT Antigen 4)
SEQ ID NO. 42
SPPSSTAPL
(peptide sequence derived from CLT Antigen 4)
SEQ ID NO. 43
FLELWLPEPML
(peptide sequence derived from CLT Antigen 4)
SEQ ID NO. 44
APLLGSEPL
(peptide sequence derived from CLT Antigen 5)
SEQ ID NO. 45
LPRTPRPDLIL
(peptide sequence derived from CLT Antigen 5)
SEQ ID NO. 46
TPRPDLILL
(peptide sequence derived from CLT Antigen 5)
SEQ ID NO. 47
RPDLILLQL
(peptide sequence derived from CLT Antigen 6)
SEQ ID NO. 48
ATIFPDPWLLK
(peptide sequence derived from CLT Antigen 6)
SEQ ID NO. 49
FPFYKDTVL
(peptide sequence derived from CLT Antigen 6)
SEQ ID NO. 50
FPFYKDTVLL
(peptide sequence derived from CLT Antigen 6)
SEQ ID NO. 51
TIFPDPWLLK
(peptide sequence derived from CLT Antigen 7)
SEQ ID NO. 52
IVLDAPVTK
(peptide sequence derived from CLT Antigen 8)
SEQ ID NO. 53
GHIHNEAV
(peptide sequence derived from CLT Antigen 8)
SEQ ID NO. 54
SLYGHIHNEAV
(peptide sequence derived from CLT Antigen 8)
SEQ ID NO. 55
SLYGHIHNEA
(cDNA sequence of CLT encoding CLT Antigen 1)
SEQ ID NO. 56
CGGGGCCAGTCTTTCCCGTGCTATTCTCGTGATAG
TGAATAAGTCTCACAAGATCTGATGGGTTTATCAG
GGGTTTTCATTTTGCTTCTTCCTCATTTTCTTTTG
CTGCTGTAATGTAAGAAACGCCTTTTGCCTCCTGC
CATAATTCTGAGGCCTCACAGCCATGTGGAACTTC
TTCAGGAGAGAATTAACATCCAATGGATTCCCAGA
AAACTTTTCCCTCGATGTACCAGCAAACACCTACA
ATGCCCTGAAAAGCCGCCTCTGCGACCCCAATGCA
GATCACACGTCCTGTCCCAGCCCCTGCAGCCTCCA
CGCGGCGGGTGCACTGCCAGGCACGGGAAGGCAGC
GCTGGCGAGTAGAACTGGCCCATCTCGCAGATAGG
AAGCTGAGCCTCAGGGACGTTTCACGCCTTCGlCA
AGGTGGTGAGAGGAGGAGCGGGATTGCCGTGAAGG
TGGTGAGAGGAGGAGCGGGGTTTGOTGCCCGACTT
CAGGGATCTGTCACCCTCGTCCAGCAGGGTTGGTT
CTTCCCGAGGCTGGGAGGATGCCAAGCCTGGTGGA
GGATGGGGGCGGTGGTGTGGTGTGGGGAGCTTCTG
ACTTGCACATCCTGAGGGAACCTTCTGCAGCTGAT
GTGTGAACTGGACCCCAGGCCGTGCCTCCGAGGAA
TCCCCAAGGCTATGGCCCCTCAGGTCCTGCTGGGG
TGTTGGCCCCCACCTCTGCCTCAGAATGCAGGGGT
TCTGCAGGGAAGCCGCAGACCAGCCTGCTGCCTTG
GGCCCTAGGGACACTGCAGCCCCAGAAAGTACTGT
GGGGGACAAAAGAGTTGTTTCTCGGGGGAGAAAAC
ACCTGTGAGGAAATGCAGGTGCCACAGAGGGAAAT
CCTCCTGGGGAGGAGGGTACCTGTTCCATCCTCGG
CCGACACGGGACTGCCTGGTGCCTGGTACCCACAG
CCGCTACCTGCCGCACGCATCTCTCCATGGTTTGC
TAATTACTTCCATTAGTTTTAAACAAACTTGACAA
GAGACAGAAGGGTCCAGAGAGAAATTAAATCTAAC
TGTTTAAACATGT
(cDNA sequence of CLT encoding
CLT Antigen 2)
SEQ ID NO. 57
CACCTCCATCACTGCGAATTATAATTCGACATGAG
ATTTGGGAGATGACACAAAACCAAACCATATCAGT
CTTTAAAGAGTTAAGTTAAAATAAGCTCTTTAAAG
TGGGCCCTAATCCAGTATGACTGGTGTTCTTATAA
GAAGAGGAGATTTGGTCACAGACATGGTTGCATGC
AGAATAAAGACTTTTCGAGGACACACTGAGAAGGC
AGCCATCTGCAAAACAAGGAAAGAGTCCTCAGCAG
AAACCAGTCCTGCAGACTCCTTGATCTTGGACTTC
CAGCCACTGCAATTGATGTCAAGCTTCAGCACCCT
TGCATCTCTGGATAAATGAAATGTCACCCCAGCTG
CCGTCCTTGTTCAGATCTGTGCAATAAAGAGCAAA
GCATAAAACCAAGTCAAGGCTTTGAGGGAGTGACC
TACAAAATGCATAATGTGAAACAATGCAAAAGCGA
AAGGTGCAAAATCCCCATCAAAGAGCTGGAGGCTG
ACAGATGCGCCAGTGATAATTCCCATCTTCCAACA
CAGGAGCACAGCTTCCATTTTCCATAACAGAACAA
CAGCCAGAGCAGCTGGAAGGCAGGGCCGCATCCCA
GACTTCCACCAACAATGGGATGAGACTTGACATCT
GGAATCACAACCACAACAGACCTGAGAGACCCACC
AGCTTGATGACAAGCTTCTTCTTTCAAAGAAAGGT
ATCAGTCTGGGGGACCTAGTGCTGCAAACCATGAC
AAATTAAGTGTGGCATCCCTCACTTGCATAATGGA
ACTCAGTGATATTTTTTAATTAACAAGAGTTATTT
TTATGTAAGCTTCTCTCATTCCTCCACTGTGCGTG
CTCGGGGGCTGGTGGTGAGGAAAAAGAAAACAGCT
GTGCGGGAAGCATCAAGAAAAGGCAAGTCATGAAG
TCTTAGAGATCAGTGACATGTAAGAAAAAGAGTGA
GGAGAAAAATATTCCTACTAAAGTTTTCCATTTGT
TTACCTTCCTTGTCACATAGACTTCCAAGAGTTAG
AAGTCTAGGATTTGATCTCCAAATCTTCCTGGCAG
ATTACTCATCTTCATTTCATTCATATAGTCCAGGG
GTTTGTACAAAGGAAGATGCCAGTTCTTCCCCAAT
CATAACTAAGATATCAAGAGATATTCTTTTGAAAT
GTAACAAAGGAGATCTGAAGTTCATCTGAAAAAAA
TAAATGGTTTTAGGCGGTCATGACCATGGGATGCT
GGACCAGGATGGTAAGCTTCAGGAAACAGAATCTG
GAGAATGCCCAGCTGCTCCCACAGGAAGCATCAGG
GAAGAAGAAGAAGAGGTGTGAAGTCTGCCTTCTGC
TCTGCTGGGATCCCTTTCACATCTCCTTTGCCTCC
AGGCAGTTTTGGTTCCTGGCCATTTCCAGGTGTGA
CTCACTCAGGATGGTAAGCATCTTCTCTCCTACCC
AGAGTAGAGGATGAAGACCTCATCTCAGAGGTTGA
AGGGAGCTCCAGAGAGAGGTCTCAAACTTCCAGCA
TTAACTGCTAAAGAAGCTTCATGAGCTGCTGGAGA
ACCTGGGAAATGACCAATTATAGGGACAGAGCTCA
AATACTCTGGGACACTCTAGTAGCTGAGAAAGTTC
CAACTCCAGGGTGATAGAGGACTGCCTGGCAAACC
ATCATCAAAGCAGAAGACCTGATACTAACATCACA
GGCTATGGTTTATTACTGAAGATCAGTGCTTACAC
CCTGCCAGAGGTTCAGAAGCAAACTTATCATTGTT
CTCCCTGGAGATGTTGGCCCACATTCTGAAAAGTG
TGGTCAGTAGTAGCAACAGAAAGCAATTGTGCTTG
CCAAGCACAATGTCACTGTCCCCAGCCCTTCCCCC
AACACAACCCAGTAGGTGCTTCCTGGCTGCAAACT
TGGGAAAGTCACTTGACCTGTCTGAGGTTCCACTT
CCTAATCTGGCCTGGCGAAGATAAGAAAAACAGTT
TATTTAAAGTGTCTAGCAAAGTGCTTGGACCAAAA
TAGGACCTCTGAAATGGTTATGGTAGTGCTGTTAA
GGTGATGTTTTAAGTGCTGATGAGCACAAAGATGG
GTAAGATATTCCTTCTGTTAAAATCTACAGTCTAA
TGAGAGAGAACAAGATGAATGCACAATAACTGTCA
TTCAGAACAGGATTATGAGAAGGTGTGAATTTCTG
TGAAAAATCAGAACAGGGAGTAATATGATCCCAGG
TGATTGGCAGGGGGTGGGGGTCTGGATTCAACTGG
AGAGGGAGCTGGCAGGGAAGGCTTCCTGGAGGATG
AGAGTTCAACAAGGGGCAGGTGTAGGATGTGGGTG
GCCAAGTGACTGGGCAGAAGGAGCTGCAGAAGTAA
GACCCCAAATCAGGAAGACAAGGGCCTGCTGAGAA
ACACGAGCTACAAAGTGCAAGTGCAGGAAGAGTTG
GGATGAGATTAGAAGGGGGTCTGGGGCCAGACTGT
GGAAGGCCCAAATGCCGGGCTAAGGAGTTTGTACT
TAATTCAGTGGTCAACGGGGAGTCATTGGAGGCTG
TTGAGCAGGAGAGTTGCTTTCTTTACAGCTGTGCC
AGACTAAATTAAACCTAAACAGTACTTTATAGCTG
GAAAGGGAAGGCCCAGGAATAGCTCTTGACTCAGA
AACAGGCATTGGGGAAGGTAATGAGAAACAGCCGT
GACTGATCAAAGCAGAGAGGTTAATTAAATTTGTA
ATTATTGTGAAAGGCCATTAAAAACCCTAGTTCAC
TAGAGATAACTGCTCTAGTGGGGCTTCAAAGACAA
ACGCTTCTTTTAACCTTGAATAGGGGGATGTTTGC
TTCTCTGTGGAGGAGATATGATTAAGATACTTAAT
AAATGGTAGATAAACA
(cDNA sequence of CLT encoding CLT
Antigens 3 and 4)
SEQ ID NO. 58
AAACACACTAAGGGCTTTGTTATGGACTGAAATGT
GTCCTCTCCCCAGAGTCACAGGATTATGAAGCCAT
AATCCTCAATGTGACTATATTTGGAGCAGGGGCCT
TTACAGACATAATTAAATTAAATGAGGTCATAAGA
GTGGGGCCCTAGTCTGATAGGACTGGTGTCCTTAC
AAGAAGAGGGAGAGTCCTCAGAGAGTCCTCTCTCT
CGGCATGGACACAAAAGAAAAGCCATGTGAGGACA
CAGAGAGAAGGTGGCTGTCTACGAGCTAGGAAGAA
AGGCCTCACCGGAAACCAACCCTCACAGCAGCTCC
ATCTTGGACTTCCAGCCTCCGGAACTGTGAGAAAA
TAAATGTTTGCAATTCAGGTCGGTTGTATTTTGTG
AAGGCCATCCTAGCAAATGAATACTCCTAACATTG
TCTCTTTAAGAGCTCACCAGCCTGAGGTAGGAATC
ATTCCATCTGTGTTACTAATGAGACCGCTGAGGAT
CAAAGGGGTTTTCCACCACATCCACTCACCTCTAC
ATGGCGAAAACCAAGGGTTCACTCTCTGTCTTCAG
GGAGCTCCACCCAGCAGCAGCGTTTGACAGAGCTG
TTCACTTCCTCTTCCTGGAGCTGTGGCTTCCAGAG
CCCATGCTCAGCAGTTCCCCTCCTTCTTCGACTGC
TCCTCTCTTAGGCTCAGAGCCACTCAGACATTGGG
AAGCAAGTTTGTCAAGATGACAGAGAACCGAGGTA
ATGGATTCGAGTGATGAAACAGGAAGTTCATTCAT
GAGTTTTTGGCCACACCTCCAAAGTGACGACTTAG
CCAGAAATGGGATAACTGGGTTTCCCTACTTCTCT
TTTATCATCCTCAATGAGAGTGACCAAATATTAGA
GCTAGATGGAACCTTAGTGAAAATCTGGCTACTCG
TCCCGTCCCACCAGCCTGCCACCCATTTCAAGTTT
GAAGAGACAAAGACACATGGACCTTATGTAATTAC
TGGGGATTACCCCAGGAGTCTGTGGCAAAAGTCAG
CTTCTTCCCTCCCTGCTTCCCCGCCCTGTCTCTGG
TACTTTCTACCAACACTGGGCTGTTTCTGTGATCA
CACTTAAGCGTACCTAACCTGCGAATGCTGTATAG
AAGGTGCTAATGAACATGATTTAGCTTTAACACTC
AGTTTTCTAAAGGGACACGTGGGGGCAGCAAATGT
TTAGGCAAAAACAATTCCAGTTCTAGCCTCTACTG
TCTACATATGTGTATACATTTGGGAAACGTTTGGG
AAAGGGATATTTGAGAGCTTCTTTTTCTTTTTTGT
GGTTTAGTTATTTGATGATATTGAGATTGTTTCTG
AGCCATGTGCTTCAACATCGGATTGGGGATTTCAG
AAAAAGTTTTAGTCACTGTGATTCCATTTAGCTTC
CAAATGTGTCTCTGCTAAGAGACTTAAAAGCACTC
ATAAATAGCACGTGTGTCTTCTTTGCAGTGTTTGC
TAATTTTGAGTCACATCTTTTTAGAAAATCATGAG
ATTTGGTGTCACAGAGACTGGAATAAATATAGTCA
AACTTATTGGTGAAGATTTCCTTTAGCTGTTTTCA
TAATCCATTTCCATTGTTATGATTATTGATGAATA
AAACATTTTCTTTAGGTAGATACTTCTTTTTTCCC
CCCACCTTGATTTAATGTTTCCACTCTTATTGTCA
AGTTTCTTATTACTCCCTAATAACTCTCAATAAAA
TAATGATTCCTGGGAGATTATTCCTGCTTTCCTAC
TATCACCTGTTGATTTGAAAAGACAGAACAATACC
GTAGAAGCTTCACTAATACATTGAAAGATAAAATG
ATAATACTAAATACTAAAATATGAAAAGTGATACT
AAAAGTGGAGTCCTGGCACTAGTATTTTTTTTTTT
GAGTCTTTAAATTTTATTTATTTATTTTTGAATTT
TTTAAAATTATATGTTATGTTCTGGGATACATGTG
CAGAACGTGCAGGTTTGTTACATAGGTATACAGGT
CTGGCACTAGTATTTTGTTGCCACAAAATATCAAG
CATGTATCCAAACTGCTCAAGACACATTAAAGACA
CAGGTAATCTGTAGGCATATTCAGGCTTGTAGTTT
GCATTTTTTGGTTTTCTTGTGGCTTTCAGTGCAAG
TTGAGGTAATTCATGGGAAACAGTCACCAAAGAAG
TGCCAGTATTAGAAATCCAAGAGCCATTTCTCTAG
CTTCTTCCAGAATCAAGACTTTAGAGGTAATTTCT
ATCAACACTGGACATTTCCTGTCTGCAATTAACAA
TGAACACATAGCATTATGTTTAATTGCAACCTGTT
TAAAGCAGATTGGATGCTAAGGTTTAAGAACACTC
TTCAGTCAAAAAGGTCTTTTAATCAGGTTTTTAAT
CTTGAGCACAATCTAGGACACAGCATCATAGACTA
ACTCATTCGAGAATAGGTGTTGTCATCTAATCCTA
ACCACCCCCACCACCAACAAGCTGAATAGCTCTGG
GCTCAGTATATACATTTGTACTGGGCTCAGTACAC
ACACCTAAGCTGGGTTCAGTATATGCCACTTTATA
GTGAGAGGCATTTTGTAATGAGAGCTCTGGGTTCA
CTATATACATTTGTACTGGGCTCA
(cDNA sequence of CLT encoding CLT Antigen 5)
SEQ ID NO. 59
CTTTCATCTTTAATTTGACCAAAATGGAAACCAGG
ATCATGAGAATTCCTCGGGGCTGGTGTTGAAAGGA
ATTTCCCCTGCTCTTGCCAGAGTCTCGAGGGGTGG
CCTCCTTCCACGGGTGAGTAACCACAAGTCCATGT
GACCGAACAAACAGCATATTCTTTTGTTCAAAAGA
GAAAAACAACATTGAAGGAAATCAGCTGAAGAAAA
TTGAGATGAAAGCCAGTCCACGCCTCAGCATCCTG
AGGAAATGTTCTTCCTTGATGCTCTGAGCTCTCTA
AGAAGTTACCACAAAACCAAACCCATCAGAAGTTT
GCAGGACGTCCTTGTTTAGAGCTGGGAAATAAACC
ACGAAACAGCGCAAAGGAGAGTCCAGGCCTGCCAA
TGCTTCCGCGAACTCCTCGCCCCGACCTCATCCTT
CTCCAGCTCCTACCTGCAGGCCTCAGACAACTTTT
GCAGACCTCTGGTCCGGACAATGAACAACCCATAG
AACAAGATCTGATTTGTAATGTTTGCTGATCTCCA
AAGTGTAAATACTCCCACCATGACCAATTGGAAGC
CACTGACAAGTCCTCACTAAATGCAGAATTGAGAA
GAAACACGAATAGCACATCATTGTATGGTATTTCC
ACTCTACCAATGGATGTGAATAACCTCAAGAACAC
ATAATGTTGAGATGTGGAAAAATCATCAGGAAGCC
TCTCCCCTTCACCCTGCAGTGTCTCTGCAATGCTG
GTAGAGTGGGCTGGCACAGCCCCCGCCTCTGGCCT
GGTCCGGGTCCAACCTGCCTGCCTCCTGGGGCAGC
AGCTCCCTGAACGAAGAGCCGCGGAGACCGAAGAA
CTCAGTGAATCAGCAGTTCTCCCAGATGAAACGCT
GGCCTGAAAGCAGCCTCAAGAGCTTTGGCCCGTCA
CCTTGCCTTGCCTTCTCTTCCTTCCTCGTCCTCTG
TTTGTTCATCTTTCCTGAAAAAATTAAGTCAGCTG
TTCCCCTTAACCAATTTCCCTGGCATTCTGAAGGG
TAGGCCACATGGCCCACCTGCCAGCTACTCCCACC
TGCCAAGCCTTCCTGACTATATTTACCCTGGTACT
CCCATGTCCCGGGGCTGCTTCAGCAGAGCCAAGGA
CACACCCAGGTGTTTGTTTTCTAGGTCAGATTTCC
TCAGCCATGGGTGTATCTGTGCTTGTCCCTCAAAA
TCCTCATAGCTCCTTTCCCCACCCCAACTTCCAGG
CCAGACGGGGTTCAGGGGTGTCTCAGCTAAGGTTC
CCCTGAAGCAGACACAAATTAGTACAAAAGGGACT
TATTAGGAGGTGATCCTTAAAAATTTGGGCAGGAG
AATGGAAAGGGTGGACACAGACAAAAAGGATGCCA
AGTGAGAATGTGATGATTACTGCTGCAGTTTTCTA
CAGCTGCCTGTCAAACTATCCCAACACAGTGGCAT
TGACCAGCAGCCATTTTCGCTATGCCCACAGCTTC
TGTGGGTCAGGCGTTTGGACAGGGCAATGGGAACA
GCTTGTTTCTGTTCCATAGTGTGTGAGGGAGGCCT
CAACTGGAAAACTCAAAGACCGGGGTGGCTTGATG
ACTGGAGGCTGGAACCATCCGGAAGCTTCTTGAAG
CTGGGTTGACTTACATGTGGTCTTGCATGTGAGTT
GGGCTTTTCACATCATGGCTGTTGGGTTCCAAGAG
GCAGTGTCTGGGGAACAAGAGTTCCAAGACACCAA
GGCAGAAGCTGCTAGACCTTTTTTGACCTCGCTTC
GGATGTCAGCAATGAAGTTCTGTGGATTCAACTGC
ATTCTATTGGTTACCAGCAGTCGCTTGAATCTGAT
TAGCTCCAAAACTGAGTGAGCCTCTGGCAAGAGTG
CATTGCAGAAGGGCACATCCAATGAGAGGCGCTGC
TGTAGTAACAGGGACCTCTCCAATGGCCTGTTTGA
GTGCTCTCAGAATATGGCACCTGGGTTCCTCCAGA
GCAAGTGATCTGAGAGAGAGCAAGGAGAAGGCCAC
TCTGCCTTTCATGACTCATCTCAGAAGTCACACAC
TATCACAGGTTCTGTTCTGTTCATTGGAAGTGAGT
CATGAAGTCCGGCCCACACTCAAGAGAAAGGAAAT
TATGCTCCTTCTTTTGAAATGAGTGTAAGAAAGTA
AAACTTTGTCAAATGTGTATGTGTGTGTGTGTGTG
TG
(cDNA sequence of CLT encoding CLT Antigen 6)
SEQ ID NO. 60
GAGGCCTCACAGCCATGTGGAACTCTCTGGAGGCC
AGAAGTCCAAAATCAAGATGTTGCAGCACTGGTCC
CTGGGAGGCTGTGCAGGAGAATCTGCTCTGGGCGT
CTCTCCTGGCTCCTGGTGGTGCCACAATCTTCCCT
GATCCTTGGCTTTTGAAGGCATCCCCCAGCTCTCT
GCCTCATCTTCACAGGACTTCTCCCTGTGCCTGTC
TGTGCCCAAATTTCCCCTTCTATAAGGACACAGTC
CTACTGCATCAGGGCCCACGCTAATCACTTCTTCT
TCACTAATTACATCGGCAAACCCCCTATTTCCAAG
TTAAGTCATGTTCTGAGGTTAGGATTTCAATACCT
GAATTTTAGGGGACATAGTTCAGCCCAAAGGACCT
GGGGGGACAGAAACACCCAGGAAAGTCCACAGGTC
AGCGCCTGGACCCGCCTGGCCGTGGGACATTGCTC
GGCCGAGTTCATCCTCATAAACGCTGACGTGTGCT
AGAAGATACAATATTAGTTTCATAAACAACAGTTT
CTTTTTTTGCAAACCATACCCACAGGTCCTATTGC
CACTTTTCAGTGCTGCAGCAGCTAGTCAGATGACA
ACTGGGTGAGAGCTTGGTCTCGAGTGTTTCCTGTG
AGGCCAGAATATTGCTCTGATTCCTCAGGGGTGAC
TCAGCCCTCCGGTGGGAAATATGTGACCTCATTTG
CATTTCCTAATTGAATTGCTATCAGCTTTCCTGCA
AGACTGCACCAAAAAACAGTTCTTCCAGATTCCAG
CACTGCCTGCATTGCCCACAGAAAGGCTGCTTTTA
AATAGTTGCTAAGAGAGGGCGACAAAAGAAGGAGA
AAAGTTGCCAAATTTTTATGGTTCATCTAACTGTA
AAGCTGTCAGTTTTACCCAGCTCCCCAGAGTCGCT
AAAATAATGGACAGAGGAGGCGGCACTTGAAGAAA
CCTCGACATAAGAGAAATGAAATTTACAGAACCAG
GAAATTAGGGGTCCCCATGCACCTGCCCTGGCAGA
ATCTGCTTCCAGGCTCCTCTCAATAAACACTGCCC
TGGGACTCCCAGCGCTCTGTAAACTGATGCTTTGA
AGGAGGCCATTTGTTTCTCCAGAACCTTCTGCCAG
CAGAACTCTTGCTTACCACGTGCCCCCATCAGAAC
TCCCACCTTCTCTAAACACAGTTAGGTAGCCCTCT
GCATGCATTTGTTTAAAAAAATTAAATTGTGAGTA
ATAGACTATAAGTACAACAGCGTACAGAACACCAG
CTAAAAAAATGATGGCAGAATGAGCACCTGTCTTG
CCACAAGACTCCCTCCCACCCTCTGGTGTCCCCCA
GCATGGCTTCCTGCTGCCCCCTGAAAGGAACCCTC
AACCGACTGTAACTCTGCACCTCAGTGCCACCCGA
ATTTGGACTTCATGGGAGTGCGGTCACTCGGCTGA
CACGTGTTCCTGCTGCCAGGCACCTGCCTGCTCTC
CTTCTTTGGTCTGTTTGTGAGATTCGCTGGCTTTG
TTGCTTCTAGCTGCAGTTTGTTCACTTTTACTGCC
GTATAATATTCCACTTTAGGAGCCCACTGCAATTT
ATTGACTCATTGCAATGGTGAGGGCATGTGTGTTG
TTTCCAGCTTTTGGTGATTATGCGTAAGATTTCTC
TTAACGTCCCGTACAGAGTTCTTGGTATTACCTGC
CTATACTGCTATGGGCATACACCAAAAACGAGGCA
TATGTATATTTGACTTTAATAGTAATTCTGAATTG
CTGTCTAAAGTGTTTGTACCATTGTACACTCCCAT
CAAGAGTATACGAGTGCTGCAAATGTCCTTTCCCA
GACTCTGCCTTATTGTCTCATTTTTAAGATATATG
TTTTGATGATCAGAAGTTCTTTTTTTTTTTTTTTT
TTTTTGACAGGCTTTTGCTCTGTTGCCCAGGCTGG
AGTGCAGTGGCATGATCTCAGCTCACTGCAACCTC
TACCTCTCGGGCTCAAGTGATTCTCCTGTTTGAGA
CTCCCTAGTAGCTGGGATTACAGGTGTGCACCACC
ATGCCTAGCTAATTTTTGTATTTTTAGTGGAGACA
GGGTTTCACCATGTTAGCCAGGCTGGTCTCGAACT
CCTGACCTTGGGTGATCTGCCCACCTTGGCCTCCC
AAAGTGCTGGGATTACAGGCGTGAGCCACCGCACC
TGGCAGAAGTTCTTATTTTTAATAAAGTCCAGTGA
TCATTCTTTCCCGTGTTTCACGCTTCACGTGCCAT
GTGCGAGGAAGCTTTCCACACCCGGGGGTCAGTTC
ACTCCCAGCTGCATTGCTTTGCCTTTCCTGTTTAG
GTTTTCAGTCCACCTCAAATGGATTTTGGTTGTGG
TGTGAGGTAGGGGTCATTTTACTTTTTTCTTAAAG
ATGCCCCATTGGCCCAGCATCATTTATTGAAAAGA
CAGTCTTTCCTCTGTTGTTTCTGTAGAAATGGGCA
AACTTTCCCCCAAATGGCCAGATGGTAAATATTTT
AGGCATCTCAGGTTAAGGGGCAAAATTGAGGACAT
CGTATAGGTACTTACATAACCCTTTAAAATGGAAC
CATTTAAAAATGTAAAAACCAGTCTTAGCTTGCTG
GCCATAAAAGGCCTGTGGGCTACAGCATGCTGGCA
CCTGCTCTACAGCACTGCTCTTATAATAAAAAATC
AAGCGTCTGCACCCTGGGCATTTGACAAGGCTCAT
CAGATTGTAAAGATGAACTGGGTGGATTTTGTAGT
ATGAGAGTTATACGTCCATGAAGCTGATTTTTAAA
CACATTACATGTCTTTTTCTGGGCTCATTATTTTT
GTTCCAAGTGTCTATCTAATCATCTTTGCTTCAAA
ATGACACCGTTTTAATGGCTGTAGTTCAGTGGTTC
TCAAAAGGGGGCAGTTTTGCCCCCCAGGGAACATT
TGGCAGTGGCTGGAAAGAGCTTTGGTTGCATATGG
AGGTTATGGGGCTTTACCTGGCGTTTAGTGGGTAG
AGGCCAGGAGTCTGGCTTACTATCCTGTAGCACAC
AGGACAGCCCCAACAACTTGTCTGGAAGGATGTGT
CCTCCCATCTTTTTCTTGTTCTAGGCTACCTTCGC
CCTTTCCATTTCCATTAAAAAAT
(cDNA sequence of CLT encoding CLT Antigen 7)
SEQ ID NO. 61
GTGTGCCCTTTGCAGCAAAGAGCGTGGTTCCTCCT
CTTCCTGGGTGTAAACTGCGATTCAAAAAGCCAGG
TGGGAAGCCCTGTAGTAGGGACTCTGGCTTTGTCC
CTGTTTCCCCCTTTTCTTCCTCTTCACCCACTAAA
ACCCTGTTTTACTCACGGTTCAAATTGTTTGGCAG
CCTGAATTTTCATGGCCATGGGACAAAGAACCCCG
TCTTTAGCTGAATTAAGGAAAAGTTCTGCAACATT
TTTGACGTGCAACTTGGGGGCTCAAGCGGAGAAGC
GAAGCAGAGCCCCTGGAAAACTCACATATGTGTCA
ACAATAGTCCTTGATGCCCCTGTAACAAAACTTGA
GCAAGGCCTGGTGATGAAGAGATACAAGATTGTCA
CCCAAGGATTTGATTATACCTCTGTTGAAAGTTAA
TGCACACTCAACCGAATATGGCAATTGGTACTCTC
AATTCTATATTCTCAACAATTACAGTGAAGATACA
AGTAGAATACAATCACTGCCACATTTTTTGAAATT
CTGTGTAAACTGTAATATTCTGTCAACATTAATGA
CATTTGAAGTGTCCTGTCAAAACAATTGCAGCTAC
TTTATGTATAAAACATATTAAATAGGCTCATCCAA
CTTGCATTCTTTATTAAGCTTATTTTCATATTTTT
TCCTATGGATGAACTTAAAAATAATTTTGTTTCTT
AATTAAGATTCTATGCATGAAGATGCTGAATAATT
TAAGACAATTGTTCATTCAAATAGTTGCTAATTAC
ACCCTCCTGGCATAGTTATTGTATTATTACTACAT
TTAGGAATAATATGCTGTACTACTTGGACTTGAAA
ATGTTTCTGACATTTTAATGAACACACTACTTAGT
TATATTTTACAAGGGTTTTCAGTGAACCACAGAGG
ATTAAAAAATGTCATTCAAGGGTTGTAGATAATTA
AACTGACTGAATATAAGAAGCCTCATATGGAAGTG
AAAATTATGTATGAATTTTGTTGAGCTGGAAATGT
GTTTTACTAAATGACTTCAGATTTGTTACTTTTAA
ATACAAGATGAAGGGAATCAAGGGGATACTTTCTT
CTCACCTCAATTTGTTCCATTTGCATAAGCTATTT
TATCTTTCAATATCCCTTTTGATATTTCATTTTGG
CCCTTGAATACACTAAAGATTATTTAAAATAAATA
AATGTTGACTTTGAAATACTGCTATATATATTCAT
TGCAATACATCAGGTGGGAAATTGGTGCAATTCAG
TCACACACACAACCTCATAGATCAGATTTCCAGTT
AAGTTTTAATAGAAAGAGATTTTAGATAGACATGA
CTTTCAATAAAATGGAAGAATTTTTATTAGTTTCA
AAATAATGATTTTACTTGCTTATCAACACAGTTGA
CCTTTCCCCACAGTACTATGATCTTCTTAGTACCT
CTGCTATAATTATATTAATGTCTTCCTATATCTAG
AAATTTGATTATTATTCTATTTTTATGGATCATTA
AATTTTTCTATTCAAGAATGATCCCTATAGTCAAC
TTCCTGAGGGATTTCTGCTTGCTATTTTCTGTTTC
AATTGTGTGCATTTCATATTTACTATTAAAGATTT
TAAATAATGACACATTGTTTAAGCGGTTGTAAGTC
TGATTGTTATAAAGCTCTCTGGAGAATTCTGTAAG
CTTTCACATCTGATGGAGAACTTCAATGAGAACAT
CTTTAATGTGGAATTCTTGGACAAGAAACTACAGA
TTATCCATGGTTCAGAAGATATGATGAATTAGTAA
CTATTTTTCCGTACATAAAAAGTCAATCTTCCTAA
CCAGTTTGTTGTTTTAGCTAAATGAATCGGTCAAA
TATTTTAGTTATATTAGCAATATTTTGTCTCTAAA
ATATCTTGACATAAAAC
(cDNA sequence of CLT encoding CLT Antigen 8)
SEQ ID NO: 62
GGGTCCCTGGCTCGGCTCTACCCCCATGGATCTAG
GTGAGGACAGGCGCTCCTGCTTTCCCGCCCAAATG
TTGTATTTTCCAAGCCTACCCTTGCCGGCCACGCC
CCCAACCTGGGCCTATAAAAACCGCCCCCCGCAGG
CCCTAGCGGGCAGACACACTGAAGTCGCTAGACAT
TTGAGGAACACATCCGGGGAAGAAGACACAGGTGG
CTGGTCATGGAGAGCCCGCTGGGGGAAGAGCACAC
AGACAGGCACCGGCAGGCCATTGACCAGCGGGACA
AGGTGGAGTTTGGCTGGGGCAGTGGGAGGAGAGCT
GGGGCCGCCGAGCTGCCCTACTCCAGGGGAAAACC
ACCTCCCTTCTCGCTCCCCCATCACCGGAGAGCTA
CTTCCACTCAATACAACTTTGCACTCATTCTCCAA
GCCACGTGTGACCAATTTTTCCGGTACACCAAGGC
GAGAAATCCGGGGATACAGAAAGCCCTCTGTCCTT
GCGATAAGGTAGAGGGTCCAATTGAGCTAACACAA
GCTGCCTATAGACGGCAAACTAAGAGAGCACCCAG
TAACACACGCCTGCTGGGGCTTCAGGAGCACACAC
GTCCACTGGGGCTTCGGGAGCTGTAAACAGTCAGC
CCTAGACACTGTCGTGGGATCGGAGCCCCACAGCC
TGCCTGTCTGTATGCTCCTCTAGAGGTCTGAGCAG
CGGGGCGCTGAAGAAACGAGCCACACTCCCATCAC
ACGCCCTGAGAGGAGGACAAGGGAACCCGTACCGT
TTCACTTGTGATAGAGATAAAGTTATTATTGTTGT
AATTTTAACTTATAGAACTATAATATAATGGTACA
ATGATATATATTGACAATATTCCTCTTTCACCCAC
ATTTTGTATCTGTGTTCAAGATTAAATGAAAAAGG
ATATTCTCAAAAAGCTAGCAAAACCAAAAGCAAGA
CGTTATGCCAAATGTAAACATATTTCTGTTTAGCA
AATAGCAGGAGTGTATAAAACATTTCTCTTTTCAC
ATAACAGAATGTTCTATGCTTACTGTATTAGTTAA
CAAATTCAAGTCTGTTTATTTTGTTTGAAATTCCA
CTTCATCCATAAATTACAGCATTACACAATAACAC
CAGAAGGACAATATCACCATTGTTTGCTTTTACAG
TCATCTCAGCCCACAAAATGCTTCCCATTGTCAGC
TTGCTATCATCAAGCTATTGTTGTTTTCCTCTTTC
TGGGGTCTTGTATTAATATCATTCAAATTATTTAG
ACACTCAACAGTGTTTTTGCTATCAGTGCAAACCT
CTAAAGAGCTGGAGCCCCAGCGTGATGACCAAATA
ACCCTTGACTATTTATCTTGCCGTAAGTCATTATT
TCCTGAGGCCCTGGAGAAGCTGTGTTGCCATGTGT
GCACTGCAGGGACGGGGAGCTTCTCCTGCCGGAGC
TGGTTTGTTCCACTGGACAATGAGCCCTTTTCTGC
TTGGCTCTCTGTATGGGCACATACACAATGAGGCG
GTTTAGGGAAGAGGGGTGATGTGGGTCATTGATAA
CAACAATCCCCGAACTTCTTAAAGAATTGTTGAGC
CCCCTAAAAATATGTTGTCTTTATGAATTATCCTT
AACCCTTTTTAATTGCATAAAAACTTCAGGCACTT
GAAAAAAATTAAAAAACGAAAAGTAAGTCTGTCTC
TAGTATCCCTCTTCTCCTTTGTAGAATTACATCCT
TTATTCACTCTGCCACTATTTATTATGTGCCTCCT
GTGTGTAAGACATACTGTTAGTCATTGGAAATACA
GAAATGAATGGGACAGACACACTTCTTGCCCTCAT
GGAACTTAGGGCCTAATGGGAGATACAATGTTAAG
GTTGTTTCTAGTGTTTTTTTTTTTTTTTAAGAATA
TGTTCTACATGTATACATGTAATATGTATTCTCTC
CCATTTTTAAAAAACATAAATGGTGGTTTACTATG
TGTACGCTTTTTGGTTTTGCATTATTCTTTTAACA
GTATGTAGAGGATGCATTTTCTACTGTATAGTGTT
CTGTCCCCCTTAAATAGCACTCTGATTTTATTTTG
GGGGGGAATCACGACTTTCTAATTTTGCATACTCC
TTGTGGGATTGTAAATCAGGTCCCCTGTCCTCAAG
TAGCCAATGGTTAGGTATGCAACCCAGCTTATCTC
TCTTTAGACTCAATCTCAAGCAGATCCAGAGTTAC
TCAGGATCAGAACAATATTTGAAAGGCATTACCAG
AATCCAGACAAGATGATGGAGCAATACCTGATGCC
CAGTGGTCTAGGGTAGCCGATTCCTGTTCTGCTCT
CCAGGCTCCTGTCCATTCTGTGGATCAACTCATAT
TGCTCCAATTCATTTTGTTTTGCTTGCATAAGCCA
GAATTAACTTCTGTTGCTTGTAAACAAAGAGAGTT
AACCAAAGACATACAGTATATCAGAGTATAGAGAC
CTACTTTACTCTCGGTCATTGCCTGCATGAGTCTT
CTTTATATGGATGTATCACAGTTTATTTAACCACT
CCCTTGTAAGTGGACATTTAAGTCTAGTGTTCTGT
AAATAAAAGGTCAGAATATACGTCTGTATACAGTA
TATATTCTTGCATATCCACCTTTGGACAGATGTGT
GAATGTGTTTTGTAGAAATACATTTGTAGAAATGC
AACTGCTGGGTCAAAGAATTAGTAGATTTTTAATA
ACATCAAACAGCGTTGAAGGCCCCCATATAAGAAT
AACAACTACTGACTGAAGACATAACTAATTAAAAA
AATTAATTACAGCTTATTTGTAATAACTTCTTATT
GTCACTGAGTGAAAAGGTAATTCTCGTTGAATTTA
CGAAAAGTGACTAATAGGAAATTTAGAAAACTCAG
AGAATATATAAAAACACAAAGAAAAGCCAGCCACC
AAGTCGCTTATAATTCTCTCACCAACAGTGGCAGA
ATTACATTTAGTCATCATCATTATTCTTACATCCA
GTTTTATAGTTATTTTTGAAGAGGATTATTATCAA
CTATCAACTCTGTCATAGCTGGAAGTAGAGGCCAC
TAAAACAGATTTCTTAAACTCCAAGTACTGTATAT
GGATTTTCTAC
(cDNA sequence encoding CLT Antigen 1)
SEQ ID NO. 63
ATGTGGAACTTCTTCAGGAGAGAATTAACATCCAA
TGGATTCCCAGAAAACTTTTCCCTCGATGTACCAG
CAAACACCTACAATGCCCTGAAAAGCCGCCTCTGC
GACCCCAATGCAGATCACACGTCCTGTCCCAGCCC
CTGCAGCCTCCACGCGGCGGGTGCACTGCCAGGCA
CGGGAAGGCAGCGCTGGCGAGTAGAACTGGCCCAT
CTCGCAGATAGGAAGCTGAGCCTCAGGGACGTTTC
ACGCCTTCGTCAAGGTGGTGAGAGGAGGAGCGGGA
TTGCCGTGAAGGTGGTGAGAGGAGGAGCGGGGTTT
GCTGCCCGACTTCAGGGATCTGTCACCCTCGTCCA
GCAGGGTTGGTTCTTCCCGAGGCTGGGAGGATGCC
AAGCCTGGTGGAGGATGGGGGCGGTGGTGTGGTGT
GGGGAGCTTCTGACTTGCACATCC
(cDNA sequence encoding CLT Antigen 2)
SEQ ID NO. 64
ATGACTGGTGTTCTTATAAGAAGAGGAGATTTGGT
CACAGACATGGTTGCATGCAGAATAAAGACTTTTC
GAGGACACACTGAGAAGGCAGCCATCTGCAAAACA
AGGAAAGAGTCCTCAGCAGAAACCAGTCCTGCAGA
CTCCTTGATCTTGGACTTCCAGCCACTGCAATTGA
TGTCAAGCTTCAGCACCCTTGCATCTCTGGATAAA
(cDNA sequence encoding CLT Antigen 3)
SEQ ID NO. 65
ATGAATACTCCTAACATTGTCTCTTTAAGAGCTCA
CCAGCCTGAGGTAGGAATCATTCCATCTGTGTTAC
TAATGAGACCGCTGAGGATCAAAGGGGTTTTCCAC
CACATCCACTCACCTCTACATGGCGAAAACCAAGG
GTTCACTCTCTGTCTTCAGGGAGCTCCACCCAGCA
GCAGCGTT
(cDNA sequence encoding CLT Antigen 4)
SEQ ID NO. 66
ATGGCGAAAACCAAGGGTTCACTCTCTGTCTTCAG
GGAGCTCCACCCAGCAGCAGCGTTTGACAGAGCTG
TTCACTTCCTCTTCCTGGAGCTGTGGCTTCCAGAG
CCCATGCTCAGCAGTTCCCCTCCTTCTTCGACTGC
TCCTCTCTTAGGCTCAGAGCCACTCAGACATTGGG
AAGCAAGTTTGTCAAGA
(cDNA sequence encoding CLT Antigen 5)
SEQ ID NO. 67
ATGCTTCCGCGAACTCCTCGCCCCGACCTCATCCT
TCTCCAGCTCCTACCTGCAGGCCTCAGACAACTTT
TGCAGACCTCTGGTCCGGACAATGAACAACCCATA
GAACAAGATCTGATTTGTAATGTTTGCTGA
(cDNA sequence encoding CLT Antigen 6)
SEQ ID NO. 68
ATGTGGAACTCTCTGGAGGCCAGAAGTCCAAAATC
AAGATGTTGCAGCACTGGTCCCTGGGAGGCTGTGC
AGGAGAATCTGCTCTGGGCGTCTCTCCTGGCTCCT
GGTGGTGCCACAATCTTCCCTGATCCTTGGCTTTT
GAAGGCATCCCCCAGCTCTCTGCCTCATCTTCACA
GGACTTCTCCCTGTGCCTGTCTGTGCCCAAATTTC
CCCTTCTATAAGGACACAGTCCTACTGCATCAGGG
CCCACGCTAA
(cDNA sequence encoding CLT Antigen 7)
SEQ ID NO. 69
ATGGCCATGGGACAAAGAACCCCGTCTTTAGCTGA
ATTAAGGAAAAGTTCTGCAACATTTTTGACGTGCA
ACTTGGGGGCTCAAGCGGAGAAGCGAAGCAGAGCC
CCTGGAAAACTCACATATGTGTCAACAATAGTCCT
TGATGCCCCTGTAACAAAACTTGAGCAAGGCCTGG
TGATGAAGAGATACAAGATTGTCACCCAAGGATTT
GATTATACCTCTGTTGAAAGTTAA
(cDNA sequence encoding CLT Antigen 8)
SEQ ID NO. 70
ATGTGTGCACTGCAGGGACGGGGAGCTTCTCCTGC
CGGAGCTGGTTTGTTCCACTGGACAATGAGCCCTT
TTCTGCTTGGCTCTCTGTATGGGCACATACACAAT
GAGGCGGTTTAG
(linker sequence used in CLT Antigen
Fusion Proteins 1, 2, 3 and 4)
SEQ ID NO. 71
GGG
(linker sequence used in CLT Antigen
Fusion Proteins 1, 2 and 4)
SEQ ID NO. 72
GGGG
(linker sequence used in CLT Antigen
Fusion Proteins 1 and 3)
SEQ ID NO. 73
KGG
(linker sequence used in CLT Antigen
Fusion Proteins 1, 3 and 4)
SEQ ID NO. 74
GGKGG
(linker sequence used in CLT Antigen
Fusion Proteins 1, 2, 3 and 4)
SEQ ID NO. 75
GGGKGG
(polypeptide sequence of CLT Antigen
Fusion Protein 1)
SEQ ID NO. 76
MWNFFRRELTSNGFPENFSLDVPANTYNALKSRLC
DPNADHTSCPSPCSLHAAGALPGTGRQRWRVELAH
LADRKLSLRDVSRLRQGGERRSGIAVKVVRGGAGF
AARLQGSVTLVQQGWFFPRLGGCQAWWRMGAWWCG
ELLTCTSGGGTGVLIRRGDLVTDMVACRIKTFRGH
TEKAAICKTRKESSAETSPADSLILDFQPLQLMSS
FSTLASLDKGGKGGMWNSLEARSPKSRCCSTGPWE
AVQENLLWASLLAPGGATIFPDPWLLKASPSSLPH
LHRTSPCACLCPNFPFYKDTVLLHQGPRGGGKGGM
AMGQRTPSLAELRKSSATFLTCNLGAQAEKRSRAP
GKLTYVSTIVLDAPVTKLEQGLVMKRYKIVTQGFD
YTSVESKGGMAKTKGSLSVFRELHPAAAFDRAVHF
LFLELWLPEPMLSSSPPSSTAPLLGSEPLRHWEAS
LSRGGGGMCALQGRGASPAGAGLFHWTMSPFLLGS
LYGHIHNEAV
(polypeptide sequence of CLT Antigen
Fusion Protein 2)
SEQ ID NO. 77
MWNSLEARSPKSRCCSTGPWEAVQENLLWASLLAP
GGATIFPDPWLLKASPSSLPHLHRTSPCACLCPNF
PFYKDTVLLHQGPRGGGKGGMCALQGRGASPAGAG
LFHWTMSPFLLGSLYGHIHNEAVGGGKGGMAMGQR
TPSLAELRKSSATFLTCNLGAQAEKRSRAPGKLTY
VSTIVLDAPVTKLEQGLVMKRYKIVTQGFDYTSVE
SGGGGTGVLIRRGDLVTDMVACRIKTFRGHTEKAA
ICKTRKESSAETSPADSLILDFQPLQLMSSFSTLA
SLDKGGGMAKTKGSLSVFRELHPAAAFDRAVHFLF
LELWLPEPMLSSSPPSSTAPLLGSEPLRHWEASLS
RGGGGMWNFFRRELTSNGFPENFSLDVPANTYNAL
KSRLCDPNADHTSCPSPCSLHAAGALPGTGRQRWR
VELAHLADRKLSLRDVSRLRQGGERRSGIAVKWRG
GAGFAARLQGSVTLVQQGWFFPRLGGCQAWWRMGA
WWCGELLTCTS
(polypeptide sequence of CLT Antigen
Fusion Protein 3)
SEQ ID NO. 78
MWNFFRRELTSNGFPENFSLDVPANTYNALKSRLC
DPNADHTSCPSPCSLHAAGALPGTGRQRWRVELAH
LADRKLSLRDVSRLRQGGERRSGIAVKWRGGAGFA
ARLQGSVTLVQQGWFFPRLGGCQAWWRMGAWWCGE
LLTCTSGGKTGVLIRRGDLVTDMVACRIKTFRGHT
EKAAICKTRKESSAETSPADSLILDFQPLQLMSSF
STLASLDKGGGMNTPNIVSLRAHQPEVGIIPSVLL
MRPLRIKGVFHHIHSPLHGENQGFTLCLQGAPPSS
SVGGKGGMAMGQRTPSLAELRKSSATFLTCNLGAQ
AEKRSRAPGKLTYVSTIVLDAPVTKLEQGLVMKRY
KIVTQGFDYTSVESKGGMAKTKGSLSVFRELHPAA
AFDRAVHFLFLELWLPEPMLSSSPPSSTAPLLGSE
PLRHWEASLSRKGGLPRTPRPDLILLQLLPAGLRQ
LLQTSGPDNEQPIEQDLICNVCGGGWNSLEARSPK
SRCCSTGPWEAVQENLLWASLLAPGGATIFPDPWL
LKASPSSLPHLHRTSPCACLCPNFPFYKDTVLLHQ
GPRGGGKGGMCALQGRGASPAGAGLFHWTMSPFLL
GSLYGHIHNEAV
(polypeptide sequence of CLT Antigen
Fusion Protein 4)
SEQ ID NO. 79
MWNSLEARSPKSRCCSTGPWEAVQENLLWASLLAP
GGATIFPDPWLLKASPSSLPHLHRTSPCACLCPNF
PFYKDTVLLHQGPRGGGGMNTPNIVSLRAHQPEVG
IIPSVLLMRPLRIKGVFHHIHSPLHGENQGFTLCL
QGAPPSSSVGGGKGGMWNFFRRELTSNGFPENFSL
DVPANTYNALKSRLCDPNADHTSCPSPCSLHAAGA
LPGTGRQRWRVELAHLADRKLSLRDVSRLRQGGER
RSGIAVKWRGGAGFAARLQGSVTLVQQGWFFPRLG
GCQAVWVRMGAVVWCGELLTCTSGGGGMLPRTPRP
DLILLQLLPAGLRQLLQTSGPDNEQPIEQDLICNV
CGGGGMAKTKGSLSVFRELHPAAAFDRAVHFLFLE
LWLPEPMLSSSPPSSTAPLLGSEPLRHWEASLSRG
GGGMCALQGRGASPAGAGLFHWTMSPFLLGSLYGH
IHNEAVGGGKGGMAMGQRTPSLAELRKSSATFLTC
NLGAQAEKRSRAPGKLTYVSTIVLDAPVTKLEQGL
VMKRYKIVTQGFDYTSVESGGGGTGVLIRRGDLVT
DMVACRIKTFRGHTEKAAICKTRKESSAETSPADS
LILDFQPLQLMSSFSTLASLDK
(codon-optimised cDNA sequence encoding
CLT Antigen Fusion Protein 1)
SEQ ID NO. 80
ATGTGGAATTTCTTCCGGCGCGAGCTGACCAGCAA
CGGCTTCCCTGAGAACTTCAGCCTGGACGTGCCCG
CCAACACCTACAATGCCCTGAAGTCCAGACTGTGC
GACCCCAACGCCGATCACACCAGCTGTCCATCTCC
ATGTTCTCTGCATGCCGCTGGCGCTCTGCCTGGAA
CAGGCAGACAAAGATGGCGCGTGGAACTGGCCCAT
CTGGCCGATAGAAAGCTGAGCCTGAGGGATGTGTC
CCGGCTGAGACAAGGCGGCGAGAGAAGATCTGGAA
TCGCCGTGAAGGTCGTCAGAGGCGGAGCTGGATTT
GCCGCTAGACTGCAGGGAAGCGTGACCCTGGTTCA
GCAAGGCTGGTTCTTCCCTAGACTCGGCGGTTGTC
AAGCCTGGTGGCGAATGGGAGCTGTTGTTTGGTGC
GGCGAGCTGCTGACCTGTACATCTGGCGGAGGAAC
AGGCGTGCTGATCAGAAGAGGCGACCTGGTCACAG
ACATGGTGGCCTGCAGAATCAAGACCTTCAGAGGC
CACACAGAGAAGGCCGCCATCTGCAAGACCCGGAA
AGAGTCTAGCGCCGAGACAAGCCCTGCCGACTCTC
TGATCCTGGACTTCCAGCCTCTGCAGCTGATGAGC
AGCTTTAGCACACTGGCTAGCCTGGACAAAGGCGG
AAAAGGCGGCATGTGGAACAGCCTGGAAGCCAGAT
CTCCCAAGAGCCGGTGTTGTAGCACAGGCCCTTGG
GAAGCTGTGCAAGAGAATCTGCTGTGGGCCTCTCT
GCTTGCTCCTGGCGGAGCCACCATCTTTCCAGATC
CTTGGCTGCTGAAGGCTAGCCCCAGCTCTCTGCCT
CATCTGCACAGAACAAGCCCCTGCGCCTGCCTGTG
TCCTAACTTCCCATTCTACAAGGACACCGTGCTGC
TGCATCAGGGCCCTAGAGGTGGTGGAAAAGGTGGA
ATGGCCATGGGCCAGAGAACACCTTCTCTGGCTGA
GCTGAGAAAGAGCAGCGCCACCTTCCTGACCTGCA
ATCTGGGAGCCCAGGCCGAGAAGAGATCTAGAGCC
CCTGGCAAGCTGACCTACGTGTCCACCATTGTGCT
GGACGCCCCTGTGACCAAGCTCGAACAGGGACTCG
TGATGAAGCGGTACAAGATCGTGACCCAGGGCTTC
GACTACACCAGCGTGGAATCTAAAGGCGGAATGGC
TAAGACCAAGGGCAGCCTGAGCGTGTTCAGAGAAC
TGCATCCTGCCGCCGCTTTCGACAGAGCCGTGCAC
TTCCTGTTTCTGGAACTGTGGCTGCCCGAGCCTAT
GCTGTCTAGCAGCCCTCCTAGCTCTACAGCCCCTC
TGCTGGGATCTGAGCCTCTGAGACACTGGGAAGCC
AGCCTGTCTAGAGGCGGTGGCGGAATGTGTGCTCT
GCAAGGCAGAGGCGCTTCTCCTGCTGGTGCCGGAC
TGTTCCACTGGACAATGAGCCCATTTCTGCTGGGG
AGCCTGTACGGCCACATCCACAATGAGGCCGTCTG
A
(codon-optimised cDNA sequence encoding
CLT Antigen Fusion Protein 2)
SEQ ID NO. 81
ATGTGGAACTCCCTAGAAGCGAGATCCCCGAAATC
TAGATGTTGTTCTACTGGACCGTGGGAAGCCGTAC
AAGAAAATCTACTATGGGCCTCTCTACTAGCACCA
GGTGGTGCAACAATTTTTCCAGATCCGTGGCTATT
GAAGGCCTCGCCATCTTCTTTACCGCATCTACACA
GAACATCCCCGTGTGCTTGTCTATGTCCGAACTTT
CCGTTCTACAAGGACACCGTACTACTACATCAAGG
ACCAAGAGGTGGTGGAAAAGGTGGAATGTGTGCAC
TACAAGGAAGAGGTGCTAGTCCAGCTGGTGCTGGA
TTATTCCATTGGACAATGTCCCCGTTCCTACTAGG
ATCTCTATACGGACACATCCACAACGAAGCTGTCG
GAGGTGGAAAAGGTGGAATGGCTATGGGACAAAGA
ACACCATCTCTAGCGGAGCTAAGAAAGTCCTCTGC
GACATTCCTAACCTGTAACTTGGGAGCGCAAGCGG
AGAAAAGATCTAGAGCACCTGGAAAGCTAACCTAT
GTCTCCACCATAGTACTAGATGCGCCGGTCACAAA
GCTAGAACAGGGACTAGTAATGAAGAGATACAAGA
TCGTCACCCAGGGATTCGACTACACCTCTGTAGAA
TCTGGTGGTGGTGGAACCGGTGTCCTAATTAGAAG
AGGAGATCTAGTCACCGATATGGTCGCGTGTAGAA
TCAAGACATTCAGAGGACATACAGAGAAGGCGGCG
ATCTGCAAGACAAGAAAAGAATCTTCTGCGGAAAC
CTCTCCGGCGGACTCTCTAATCTTAGATTTTCAGC
CGCTACAGCTAATGTCCTCCTTCTCTACATTGGCC
TCGCTAGACAAAGGTGGTGGAATGGCGAAAACGAA
GGGATCCTTGTCCGTATTCAGAGAACTACATCCAG
CTGCGGCTTTCGATAGAGCGGTACATTTCCTATTC
CTAGAGCTATGGCTACCGGAACCGATGCTATCTTC
TAGTCCACCATCTTCTACAGCGCCGCTATTAGGAT
CTGAACCGCTAAGACATTGGGAAGCGAGTCTATCT
AGAGGTGGTGGTGGAATGTGGAACTTCTTCAGAAG
AGAGCTAACCTCCAACGGATTCCCCGAGAACTTCT
CTTTAGATGTACCGGCGAACACCTACAACGCGCTA
AAGTCTAGATTGTGTGATCCGAACGCGGACCATAC
TTCTTGTCCATCTCCGTGTTCTTTACATGCTGCTG
GTGCTTTACCTGGAACGGGTAGACAAAGATGGAGA
GTAGAACTAGCGCACCTAGCGGACAGAAAGCTATC
CCTAAGAGATGTCTCCAGACTAAGACAAGGTGGAG
AGAGAAGATCTGGAATCGCGGTCAAAGTAGTCAGA
GGTGGTGCAGGATTTGCTGCGAGATTACAGGGATC
TGTAACCTTGGTACAGCAAGGATGGTTCTTCCCAA
GACTTGGAGGATGTCAAGCTTGGTGGAGAATGGGA
GCTGTAGTTTGGTGCGGAGAACTATTGACATGTAC
ATCTTGA
(codon-optimised cDNA sequence encoding
CLT Antigen Fusion Protein 3)
SEQ ID NO. 82
ATGTGGAATTTCTTCCGGCGCGAGCTGACCAGCAA
CGGCTTCCCTGAGAACTTCAGCCTGGACGTGCCCG
CCAACACCTACAATGCCCTGAAGTCCAGACTGTGC
GACCCCAACGCCGATCACACCAGCTGTCCATCTCC
ATGTTCTCTGCATGCCGCTGGCGCTCTGCCTGGAA
CAGGCAGACAAAGATGGCGCGTGGAACTGGCCCAT
CTGGCCGATAGAAAGCTGAGCCTGAGGGATGTGTC
CCGGCTGAGACAAGGCGGCGAGAGAAGATCTGGAA
TCGCCGTGAAGGTCGTCAGAGGCGGAGCTGGATTT
GCCGCTAGACTGCAGGGAAGCGTGACCCTGGTTCA
GCAAGGCTGGTTCTTCCCTAGACTCGGCGGTTGTC
AAGCCTGGTGGCGAATGGGAGCTGTTGTTTGGTGC
GGCGAGCTGCTGACATGTACAAGCGGCGGAAAAAC
CGGCGTGCTGATCAGAAGAGGCGACCTGGTCACAG
ACATGGTGGCCTGCAGAATCAAGACCTTCAGAGGC
CACACAGAGAAGGCCGCCATCTGCAAGACCCGGAA
AGAGTCTAGCGCCGAGACAAGCCCTGCCGACTCTC
TGATCCTGGACTTCCAGCCTCTGCAGCTGATGAGC
AGCTTTAGCACACTGGCTAGCCTGGACAAAGGCGG
CGGAATGAACACCCCTAACATCGTGTCCCTGAGAG
CCCACCAGCCTGAAGTGGGAATCATCCCTAGCGTG
CTGCTGATGCGGCCCCTGAGAATCAAAGGCGTGTT
CCACCACATTCACAGCCCTCTGCACGGCGAGAATC
AGGGCTTCACCTTGTGTCTGCAAGGCGCCCCTCCT
AGCAGTTCTGTTGGTGGCAAAGGCGGAATGGCCAT
GGGCCAGAGAACACCATCTCTGGCCGAGCTGAGAA
AGAGCAGCGCCACCTTCCTGACCTGTAACCTGGGA
GCCCAGGCCGAGAAGAGATCTAGAGCCCCTGGCAA
GCTGACCTACGTGTCCACCATTGTGCTGGACGCCC
CTGTGACCAAGCTCGAACAGGGACTCGTGATGAAG
CGGTACAAGATCGTGACCCAGGGCTTCGACTACAC
CAGCGTGGAATCTAAAGGTGGCATGGCCAAGACCA
AGGGCAGCCTGTCCGTGTTCAGAGAGCTGCATCCT
GCCGCCGCTTTCGATAGAGCCGTGCACTTCCTGTT
TCTGGAACTGTGGCTGCCCGAGCCTATGCTGTCTA
GCAGCCCTCCATCTAGCACAGCCCCACTGCTGGGA
TCTGAGCCTCTGAGACACTGGGAAGCCAGCCTGAG
CAGAAAAGGCGGCCTGCCTAGAACACCCAGACCTG
ACCTGATTCTGCTGCAGCTGCTGCCTGCTGGACTG
AGACAACTGCTGCAGACAAGCGGCCCTGACAACGA
GCAGCCTATCGAGCAGGACCTGATCTGCAATGTGT
GCGGAGGCGGCTGGAACAGCCTGGAAGCCAGATCT
CCTAAGAGCCGGTGCTGTAGCACAGGCCCTTGGGA
AGCTGTGCAAGAGAATCTGCTGTGGGCCTCTCTGC
TTGCTCCTGGCGGAGCCACCATCTTTCCAGATCCT
TGGCTGCTGAAGGCTAGCCCCTCTAGCCTGCCTCA
TCTGCACAGAACAAGCCCCTGCGCCTGCCTGTGTC
CTAACTTCCCATTCTACAAGGACACAGTGCTGCTG
CATCAGGGCCCTCGAGGTGGCGGAAAAGGTGGAAT
GTGTGCCCTTCAAGGCAGAGGCGCTTCTCCTGCTG
GTGCCGGACTGTTCCACTGGACAATGAGCCCATTT
CTGCTGGGGAGCCTGTACGGCCACATCCACAATGA
AGCCGTCTGA
(codon-optimised cDNA sequence encoding
CLT Antigen Fusion Protein 4)
SEQ ID NO. 83
ATGTGGAACTCCCTAGAAGCGAGATCCCCGAAATC
TAGATGTTGTTCTACTGGACCGTGGGAAGCCGTAC
AAGAAAATCTACTATGGGCCTCTCTACTAGCACCA
GGTGGTGCAACAATTTTTCCAGATCCGTGGCTATT
GAAGGCCTCGCCATCTTCTTTACCGCATCTACACA
GAACATCCCCGTGTGCTTGTCTATGTCCGAACTTT
CCGTTCTACAAGGACACCGTACTACTACATCAAGG
ACCAAGAGGTGGTGGTGGAATGAATACTCCGAACA
TCGTATCTCTAAGAGCGCATCAACCGGAAGTTGGA
ATTATCCCGTCCGTCCTACTAATGAGACCGCTAAG
AATCAAGGGAGTGTTCCACCATATCCACTCTCCAC
TACACGGAGAAAACCAGGGATTCACCCTATGTTTA
CAAGGTGCTCCACCGTCCTCTAGTGTTGGAGGTGG
AAAAGGTGGAATGTGGAATTTCTTCAGAAGAGAGC
TAACCTCCAACGGATTCCCCGAGAACTTCTCTTTA
GATGTACCGGCGAACACCTACAACGCGCTAAAGTC
TAGATTGTGTGATCCGAACGCGGACCATACTTCTT
GTCCATCTCCATGTTCTCTACATGCTGCTGGTGCT
TTACCTGGAACTGGTAGACAAAGATGGAGAGTAGA
ACTAGCGCACCTAGCGGACAGAAAGCTATCCCTAA
GAGATGTCTCCAGACTAAGACAAGGTGGAGAGAGA
AGATCTGGAATCGCGGTCAAAGTAGTTAGAGGTGG
TGCAGGATTTGCGGCGAGATTACAAGGATCTGTAA
CCCTAGTACAGCAAGGATGGTTCTTCCCAAGACTT
GGAGGATGTCAAGCTTGGTGGAGAATGGGAGCTGT
AGTTTGGTGCGGAGAACTACTAACATGTACCTCTG
GTGGTGGTGGAATGCTACCAAGAACACCAAGACCG
GACCTAATCCTACTACAACTACTACCAGCGGGATT
GAGACAGCTACTACAAACATCTGGACCGGATAACG
AACAGCCGATCGAACAGGATCTAATCTGTAACGTA
TGCGGAGGTGGTGGAATGGCGAAAACAAAGGGATC
CTTGTCCGTGTTCAGAGAACTACATCCAGCTGCGG
CTTTTGATAGAGCGGTACACTTCCTATTCCTAGAG
CTATGGCTACCGGAACCGATGTTATCTTCTTCCCC
ACCATCTTCTACAGCGCCGTTATTAGGATCTGAAC
CGTTGAGACATTGGGAAGCGAGTCTATCTAGAGGT
GGTGGTGGAATGTGTGCGTTACAAGGAAGAGGTGC
TAGTCCAGCTGGTGCCGGATTATTTCATTGGACAA
TGTCCCCGTTCCTACTAGGATCTCTATACGGACAC
ATCCACAACGAAGCTGTCGGTGGTGGAAAAGGTGG
AATGGCTATGGGACAAAGAACACCATCTCTAGCGG
AGCTAAGAAAGTCCTCTGCGACATTCCTAACCTGT
AACTTGGGAGCGCAAGCGGAGAAAAGATCTAGAGC
ACCTGGAAAGCTAACCTATGTCTCCACCATAGTAC
TAGATGCGCCGGTCACAAAGCTAGAACAGGGACTA
GTAATGAAGAGATACAAGATCGTCACGCAGGGATT
CGACTACACCTCTGTAGAATCCGGTGGTGGTGGTA
CAGGTGTCCTAATTAGAAGAGGAGATCTAGTCACC
GATATGGTCGCGTGTAGAATCAAGACATTCAGAGG
ACATACAGAGAAGGCGGCGATCTGCAAGACAAGAA
AAGAATCTTCTGCGGAAACCTCTCCGGCGGACTCT
CTAATCTTAGATTTTCAGCCGCTACAGCTAATGTC
CTCCTTCTCTACTTTGGCCTCCTTGGATAAGTGA
(linker sequence used in CLT Antigen
Fusion Protein 3)
SEQ ID NO: 84
GGK
(TCR VB CDR3 AA sequence)
SEQ ID NO: 85
CASSLTGGYTGELFF
(TCR VB CDR3 AA sequence)
SEQ ID NO: 86
CASNKLGYQPQHF
(TCR VB CDR3 AA sequence)
SEQ ID NO: 87
CASSLLENQPQHF

1. A fusion protein comprising:
(a) an antigenic polypeptide comprising the amino acid sequence of SEQ ID NO: 1 or a variant thereof, or an immunogenic fragment of SEQ ID NO: 1 or a variant thereof;
(b) an antigenic polypeptide comprising the amino acid sequence of SEQ ID NO: 2 or a variant thereof or an immunogenic fragment of SEQ ID NO: 2 or a variant thereof;
(c) an antigenic polypeptide comprising the amino acid sequence of SEQ ID NO: 6 or a variant thereof, or an immunogenic fragment of SEQ ID NO: 6 or a variant thereof;
(d) an antigenic polypeptide comprising the amino acid sequence of SEQ ID NO: 7 or a variant thereof, or an immunogenic fragment of SEQ ID NO: 7 or a variant thereof;
(e) an antigenic polypeptide comprising the amino acid sequence of SEQ ID NO: 4 or a variant thereof, or an immunogenic fragment of SEQ ID NO: 4 or a variant thereof; and
(f) an antigenic polypeptide comprising the amino acid sequence of SEQ ID NO: 8 or a variant thereof, or an immunogenic fragment of SEQ ID NO: 8 or a variant thereof.

2. The fusion protein according to claim 1, wherein the fusion protein further comprises:
(g) an antigenic polypeptide comprising the amino acid sequence of SEQ ID NO: 3 or a variant thereof, or an immunogenic fragment of SEQ ID NO: 3 or a variant thereof; and/or
(h) an antigenic polypeptide comprising the amino acid sequence of SEQ ID NO: 5 or a variant thereof, or an immunogenic fragment of SEQ ID NO: 5 or a variant thereof.

3. (canceled)

4. The fusion protein according to claim 1, wherein the fusion protein comprises from N-terminus to C-terminus:
(a) an antigenic polypeptide comprising the amino acid sequence of SEQ ID NO: 1 or a variant thereof, or an immunogenic fragment of SEQ ID NO: 1 or a variant thereof;
(b) an antigenic polypeptide comprising the amino acid sequence of SEQ ID NO: 2 or a variant thereof or an immunogenic fragment of SEQ ID NO: 2 or a variant thereof;
(c) an antigenic polypeptide comprising the amino acid sequence of SEQ ID NO: 6 or a variant thereof, or an immunogenic fragment of SEQ ID NO: 6 or a variant thereof;
(d) an antigenic polypeptide comprising the amino acid sequence of SEQ ID NO: 7 or a variant thereof, or an immunogenic fragment of SEQ ID NO: 7 or a variant thereof;
(e) an antigenic polypeptide comprising the amino acid sequence of SEQ ID NO: 4 or a variant thereof, or an immunogenic fragment of SEQ ID NO: 4 or a variant thereof; and
(f) an antigenic polypeptide comprising the amino acid sequence of SEQ ID NO: 8 or a variant thereof, or an immunogenic fragment of SEQ ID NO: 8 or a variant thereof.

5. The fusion protein according to claim 1, wherein the fusion protein comprises from N-terminus to C-terminus:
(a) an antigenic polypeptide comprising the amino acid sequence of SEQ ID NO: 6 or a variant thereof, or an immunogenic fragment of SEQ ID NO: 6 or a variant thereof;
(b) an antigenic polypeptide comprising the amino acid sequence of SEQ ID NO: 8 or a variant thereof, or an immunogenic fragment of SEQ ID NO: 8 or a variant thereof;
(c) an antigenic polypeptide comprising the amino acid sequence of SEQ ID NO: 7 or a variant thereof, or an immunogenic fragment of SEQ ID NO: 7 or a variant thereof;
(d) an antigenic polypeptide comprising the amino acid sequence of SEQ ID NO: 2 or a variant thereof or an immunogenic fragment of SEQ ID NO: 2 or a variant thereof;
(e) an antigenic polypeptide comprising the amino acid sequence of SEQ ID NO: 4 or a variant thereof, or an immunogenic fragment of SEQ ID NO: 4 or a variant thereof; and
(f) an antigenic polypeptide comprising the amino acid sequence of SEQ ID NO: 1 or a variant thereof, or an immunogenic fragment of SEQ ID NO: 1 or a variant thereof.

6. (canceled)

7. The fusion protein according to claim 2, wherein the fusion protein comprises from N-terminus to C-terminus:
(a) an antigenic polypeptide comprising the amino acid sequence of SEQ ID NO: 1 or a variant thereof, or an immunogenic fragment of SEQ ID NO: 1 or a variant thereof;
(b) an antigenic polypeptide comprising the amino acid sequence of SEQ ID NO: 2 or a variant thereof or an immunogenic fragment of SEQ ID NO: 2 or a variant thereof;
(c) an antigenic polypeptide comprising the amino acid sequence of SEQ ID NO: 3 or a variant thereof, or an immunogenic fragment of SEQ ID NO: 3 or a variant thereof;
(d) an antigenic polypeptide comprising the amino acid sequence of SEQ ID NO: 7 or a variant thereof, or an immunogenic fragment of SEQ ID NO: 7 or a variant thereof;
(e) an antigenic polypeptide comprising the amino acid sequence of SEQ ID NO: 4 or a variant thereof, or an immunogenic fragment of SEQ ID NO: 4 or a variant thereof;
(f) an antigenic polypeptide comprising the amino acid sequence of SEQ ID NO: 5 or a variant thereof, or an immunogenic fragment of SEQ ID NO: 5 or a variant thereof;
(g) an antigenic polypeptide comprising the amino acid sequence of SEQ ID NO: 6 or a variant thereof, or an immunogenic fragment of SEQ ID NO: 6 or a variant thereof; and
(h) an antigenic polypeptide comprising the amino acid sequence of SEQ ID NO: 8 or a variant thereof, or an immunogenic fragment of SEQ ID NO: 8 or a variant thereof.

8. The fusion protein according to claim 2, wherein the fusion protein comprises from N-terminus to C-terminus:
(a) an antigenic polypeptide comprising the amino acid sequence of SEQ ID NO: 6 or a variant thereof, or an immunogenic fragment of SEQ ID NO: 6 or a variant thereof;
(b) an antigenic polypeptide comprising the amino acid sequence of SEQ ID NO: 3 or a variant thereof, or an immunogenic fragment of SEQ ID NO: 3 or a variant thereof;
(c) an antigenic polypeptide comprising the amino acid sequence of SEQ ID NO: 1 or a variant thereof, or an immunogenic fragment of SEQ ID NO: 1 or a variant thereof;
(d) an antigenic polypeptide comprising the amino acid sequence of SEQ ID NO: 5 or a variant thereof, or an immunogenic fragment of SEQ ID NO: 5 or a variant thereof;
(e) an antigenic polypeptide comprising the amino acid sequence of SEQ ID NO: 4 or a variant thereof, or an immunogenic fragment of SEQ ID NO: 4 or a variant thereof;
(f) an antigenic polypeptide comprising the amino acid sequence of SEQ ID NO: 8 or a variant thereof, or an immunogenic fragment of SEQ ID NO: 8 or a variant thereof;
(g) an antigenic polypeptide comprising the amino acid sequence of SEQ ID NO: 7 or a variant thereof, or an immunogenic fragment of SEQ ID NO: 7 or a variant thereof; and
(h) an antigenic polypeptide comprising the amino acid sequence of SEQ ID NO: 2 or a variant thereof or an immunogenic fragment of SEQ ID NO: 2 or a variant thereof.

9. The fusion protein according to claim 1, wherein the antigenic polypeptides are joined together by one or more peptide linkers, optionally wherein the one or more peptide linkers comprise an amino acid sequence selected from the group consisting of SEQ ID NO: 71, SEQ ID NO: 72, SEQ ID NO: 73, SEQ ID NO: 74, SEQ ID NO: 75 and SEQ ID NO: 84.

10. The fusion protein according to claim 9, wherein the fusion protein comprises a peptide linker positioned immediately C-terminal to each antigenic polypeptide.

11. (canceled)

12. The fusion protein according to claim 1, wherein the fusion protein comprises or consists of an amino acid sequence selected from the group consisting of SEQ ID NO. 76, SEQ ID NO: 77, SEQ ID NO: 78 and SEQ ID NO: 79.

13. The fusion protein according to claim 1, wherein the fusion protein is fused to a second or further polypeptide selected from (i) other polypeptides which are melanoma associated antigens; (ii) polypeptide sequences which are capable of enhancing an immune response (i.e. immunostimulant sequences); and (iii) polypeptide sequences, e.g. comprising universal CD4 helper epitopes, which are capable of providing strong CD4+ help to increase CD8+ T cell responses to antigen epitopes.

14. An isolated nucleic acid encoding the fusion protein according to claim 1, optionally wherein the nucleic acid is an artificial nucleic acid sequence.

15. The nucleic acid according to claim 14, wherein the nucleic acid is DNA or RNA, optionally wherein the nucleic acid is codon optimized for expression in a human host cell.

16-18. (canceled)

19. A vector comprising the nucleic acid according to claim 14, optionally wherein the vector is a viral vector, optionally wherein the viral vector is an adenovirus, an adeno-associated virus (AAV), alphavirus, herpes virus, arena virus, measles virus, pox virus, paramyxovirus, lentivirus, rhabdovirus vector.

20-24. (canceled)

25. An immunogenic pharmaceutical composition comprising the fusion protein according to claim 1 and a pharmaceutically acceptable carrier.

26. A vaccine composition comprising the fusion protein according to claim 1 and a pharmaceutically acceptable carrier, optionally wherein vaccine further comprises one or more immunostimulants.

27. (canceled)

28. The vaccine composition according to claim 26, wherein the immunostimulant are selected from aluminium slats, saponins, immunostimulatory oligonucleotides, oil-in-water emulsions, aminoalkyl glucosaminide 4-phosphates, lipopolysaccharides and derivatives thereof and other TLR4 ligands, TLR7 ligands, TLR9 ligands, IL-12 and interferons.

29. (canceled)

30. (canceled)

31. A method of raising an immune response in a human, the method comprising administering to said human the fusion protein according to claim 1, optionally wherein the human has a cancer that expresses an amino acid sequence selected from the group consisting of SEQ ID NOs: 1-8.

32-34. (canceled)

35. A method of treating or preventing cancer in a human patient, wherein the cells of the cancer express an amino acid sequence selected from the group consisting of SEQ ID NOs: 1-8, which method comprises administering to said human a fusion protein according to claim 1.

36. (canceled)

37. The method according to claim 35, wherein the cancer is melanoma, optionally wherein the melanoma is cutaneous melanoma or uveal melanoma.

38. (canceled)

39. A method of treating or preventing cancer in a human patient, wherein the cells of the cancer express an amino acid sequence selected from the group consisting of SEQ ID NOs: 1-8, which method comprises administering to said human a nucleic acid according to claim 14.
